(19) United States
(12) Patent Application Publication
(10) Pub. No.: US 2017/0305857 A1
(43) Pub. Date: Oct. 26, 2017

(54) N-ACYLPIPERIDINE ETHER TROPOMYOSIN-RELATED KINASE INHIBITORS

(71) Applicant: Pfizer Limited

(72) Inventors: SARAH ELIZABETH SKERRATT, GREAT ABINGTON, CAMBRIDGE (UK); SHARANJEET KAUR BAGAL, GREAT ABINGTON, CAMBRIDGE (UK); NIGEL ALAN SWAIN, GREAT ABINGTON, CAMBRIDGE (UK); KIYOYUKI OMOTO, GREAT ABINGTON, CAMBRIDGE (UK); MARK DAVID ANDREWS, SANDWICH, KENT (UK)

(73) Assignee: Pfizer Limited, Sandwich, Kent (UK)

(21) Appl. No.: 15/101,476

(22) PCT Filed: Dec. 8, 2014
(86) PCT No.: PCT/IB2014/066707

§ 371 Date: Jun. 3, 2016

(51) Int. Cl. C07D 211/46; C07D 417/14; C07D 417/12; C07D 409/14; C07D 401/12; C07D 407/14; C07D 401/14; C07D 491/08; C07D 409/12; C07D 211/00; C07D 401/00

(52) U.S. Cl. CPC C07D 211/46; C07D 491/08; C07D 417/14; C07D 417/12; C07D 409/14; C07D 409/12; C07D 401/12; C07D 407/14; C07D 401/14; C07D 401/00; C07D 211/00

(57) ABSTRACT

The present invention relates to compounds of Formula (I) described herein and their pharmaceutically acceptable salts, and their use in medicine, in particular as Trk antagonists.

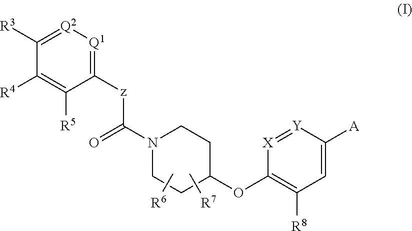

The invention described herein relates to certain piperidine compounds and the pharmaceutically acceptable salts of such compounds. The invention also relates to the processes for the preparation of the compounds, compositions containing the compounds, and the uses of such compounds and salts in treating diseases or conditions associated with tropomyosin-related kinase (Trk), activity. More specifically the invention relates to the compounds and their salts useful as inhibitors of Trk.

### BACKGROUND

Tropomyosin-related kinases (Trks) are a family of receptor tyrosine kinases activated by neurotrophins. Trks play important roles in pain sensation as well as tumour cell growth and survival signaling. Thus, inhibitors of Trk receptor kinases might provide targeted treatments for conditions such as pain and cancer. Recent developments in this field have been reviewed by Wang et al in Expert Opin. Ther. Patents (2009) 19(3): 305-319 and an extract is reproduced below.

“1.1 Trk Receptors

As one of the largest family of proteins encoded by the human genome, protein kinases are the central regulators of signal transduction as well as control of various complex cell processes. Receptor tyrosine kinases (RTKs) are a subfamily of protein kinases (up to 100 members) bound to the cell membrane that specifically act on the tyrosine residues of proteins. One small group within this subfamily is the Trk kinases, with three highly homologous isoforms: TrkA, TrkB, and TrkC. All three isoforms are activated by high affinity growth factors named neurotrophins (NT): i) nerve growth factor (NGF), which activates TrkA; ii) brain-derived neurotrophic factor (BDNF) and NT-4/5, which activate TrkB; and iii) NT-3, which activates TrkC. The binding of neurotrophins to the extracellular domain of Trks causes the Trk kinase to autophosphorylate at several intracellular tyrosine sites and triggers downstream signal transduction pathways. Trks and neurotrophins are well known for their effects on neuronal growth and survival.

1.2 Trks and Cancer

Originally isolated from neuronal tissues, Trks were thought to mainly affect the maintenance and survival of neuronal cells. However, in the past 20 years, increasing evidence has suggested that Trks play key roles in malignant transformation, chemotaxis, metastasis, and survival signaling in human tumors. The association between Trks and cancer focused on prostate cancer in earlier years and the topic has been reviewed. For example, it was reported that malignant prostate epithelial cells secrete a series of neurotrophins and at least one Trks. In pancreatic cancer, it was proposed that paracrine and/or autocrine neurotrophin-Trk interactions may influence the invasive behavior of the cancer. TrkB was also reported to be overexpressed in metastatic human pancreatic cancer cells. Recently, there have been a number of new findings in other cancer settings. For example, a translocation leads to expression of a fusion protein derived from the N-terminus of the ETV6 transcription factor and the C-terminal kinase domain of TrkC. The resulting ETV6-TrkC fusions are oncogenic in vitro and appear causative in secretory breast carcinoma and some acute myelogenous leukemias (AML). Constitutively active TrkA fusions occurred in a subset of papillary thyroid cancers and colon carcinomas. In neuroblastoma, TrkB expression was reported to be a strong predictor of aggressive tumor growth and poor prognosis, and TrkB overexpression was also associated with increased resistance to chemotherapy in neuroblastoma tumor cells in vitro. One report showed that a novel splice variant of TrkA called TrkAIII signaled in the absence of neurotrophins through the inositol phosphate-AKT pathway in a subset of neuroblastoma. Also, mutational analysis of the tyrosine kinome revealed that Trk mutations occurred in colorectal and lung cancers. In summary, Trks have been linked to a variety of human cancers, and discovering a Trk inhibitor and testing it clinically might provide further insight to the biological and medical hypothesis of treating cancer with targeted therapies.

1.3 Trks and Pain

Besides the newly developed association with cancer, Trks are also being recognized as an important mediator of pain sensation. Congenital insensitivity to pain with anhidrosis (CIPA) is a disorder of the peripheral nerves (and normally innervated sweat glands) that prevents the patient from either being able to adequately perceive painful stimuli or to sweat. TrkA defects have been shown to cause CIPA in various ethnic groups.

Currently, non-steroidal anti-inflammatory drugs (NSAIDs) and opiates have low efficacy and/or side effects (e.g., gastrointestinal/renal and psychotropic side effects, respectively) against neuropathic pain and therefore development of novel pain treatments is highly desired. It has been recognized that NGF levels are elevated in response to chronic pain, injury and inflammation and the administration of exogenous NGF increases pain hypersensitivity. In addition, inhibition of NGF function with either anti-NGF antibodies or non-selective small molecule Trk inhibitors has been shown to have effects on pain in animal models. It appears that a selective Trk inhibitor (inhibiting at least NGF's target, the TrkA receptor) might provide clinical benefit for the treatment of pain. Excellent earlier reviews have covered targeting NGF/BDNF for the treatment of pain so this review will only focus on small molecule Trk kinase inhibitors claimed against cancer and pain. However, it is notable that the NGF antibody tanezumab was very recently reported to show good efficacy in a Phase II trial against osteoarthritic knee pain.”

Further trk-mediated conditions which have been investigated and show promise include atopic dermatitis, psoriasis, eczema and prurigo nodularis, acute and chronic itch, pruritis, atopic dermatitis, inflammation, cancer, restenosis, atherosclerosis, psoriasis, thrombosis, pruritis, lower urinary tract disorder, inflammatory lung diseases such as asthma, allergic rhinitis, lung cancer, psoriatic arthritis, rheumatoid arthritis, inflammatory bowel diseases such as ulcerative colitis, Crohn's disease, fibrosis, neurodegenerative disease, diseases disorders and conditions related to dysmyelination or demyelination, certain infectious diseases such as Trypanosoma cruzi infection (Chagas disease), cancer related pain, chronic pain, neuroblastoma, ovarian cancer, colorectal cancer, melanoma, head and neck cancer, gastric carcimoma, lung carcinoma, breast cancer, glioblastoma, medulloblastoma, secratory breast cancer, salivary gland cancer, papillary thyroid carcinoma, adult myeloid leukaemia, tumour growth and metastasis, interstitial cystitis (C. Potenzieri and B. J. Undem, Clinical & Experimental Allergy, 2012 (42) 8-19; Yamaguchi J, Aihara M, Kobayashi Y, Kambara T, Ikezawa Z, J Dermatol Sci. 2009; 53:48-54; Dou Y C, Hagstromer L, Emtestam L, Johansson O., Arch Dermatol Res. 2006; 298:31-37; Johansson O, Liang Y, Emtestam L., Arch Dermatol Res. 2002; 293:614-619; Grewe M, Vogelsang K, Ruzicka T, Stege H, Krutmann J., J Invest Dermatol. 2000; 114:1108-1112; Urashima R, Mihara M., Virchows Arch. 1998; 432:363-370; Kinkelin I, Motzing S, Koltenzenburg M, Brocker E B., Cell Tissue Res. 2000; 302:31-37; Tong Liu & Ru-Rong Ji, Pflugers Arch—Eur J Physiol, DOI 10.1007/s00424-013-1284-2, published online 1 May 2013); International Patent Application publication numbers WO2012/158413, WO2013/088256, WO2013/088257 and WO2013/161919, (Brodeur, G. M., Nat. Rev. Cancer 2003, 3, 203-216), (Davidson. B., et al., Clin. Cancer Res. 2003, 9, 2248-2259), (Bardelli, A., Science 2003, 300, 949), (Truzzi, F., et al., Dermato-Endocrinology 2008, 3 (I), pp. 32-36), Yilmaz, T., et al., Cancer Biology and Therapy 2010, 10 (6), pp. 644-653), (Du, J. et al., World Journal of Gastroenterology 2003, 9 (7), pp. 1431-1434), (Ricci A., et al., American Journal of Respiratory Cell and Molecular Biology 25 (4), pp. 439-446), (Jin, W., et al., Carcinogenesis 2010, 31 (11), pp. 1939-1947), (Wadhwa, S., et al., Journal of Biosciences 2003, 28 (2), pp. 181-188), (Gruber-Olipitz, M., et al., Journal of Proteome Research 2008, 7 (5), pp. 1932-1944), (Euthus, D. M. et al., Cancer Cell 2002, 2 (5), pp. 347-348), (Li, Y.-G., et al., Chinese Journal of Cancer Prevention and Treatment 2009, 16 (6), pp. 428-430), (Greco, A., et al., Molecular and Cellular Endocrinology 2010, 321 (I), pp. 44-49), (Eguchi, M., et al., Blood 1999, 93 (4), pp. 1355-1363), (Nakagawara, A. (2001) Cancer Letters 169:107-114; Meyer, J. et al. (2007) Leukemia, 1-10; Pierottia, M. A. and Greco A., (2006) Cancer Letters 232:90-98; Eric Adriaenssens, E., et al. Cancer Res (2008) 68:(2) 346-351), (Freund-Michel, V; Frossard, N., Pharmacology ck Therapeutics (2008) 117(1), 52-76), (Hu Vivian Y; et. al. The Journal of Urology (2005), 173(3), 1016-21), (Di Mola, F. F, et. al. Gut (2000) 46(5), 670-678) (Dou, Y.-C., et. al. Archives of Dermatological Research (2006) 298(1), 31-37), (Raychaudhuri, S. P., et al., J. Investigative Dermatology (2004) 122(3), 812-819) and (de Melo-Jorge, M. et al., Cell Host ck Microbe (2007) 1(4), 251-261).

Thus Trk inhibitors have a wide variety of potential medical uses. There is a need to provide new Trk inhibitors that are good drug candidates. In particular, compounds should preferably bind potently to the Trk receptors in a selective manner compared to other receptors, whilst showing little affinity for other receptors, including other kinase and/or GPC receptors, and show functional activity as Trk receptor antagonists. They should be non-toxic and demonstrate few side-effects. Furthermore, the ideal drug candidate will exist in a physical form that is stable, non-hygroscopic and easily formulated. They should preferably be e.g. well absorbed from the gastrointestinal tract, and/or be injectable directly into the bloodstream, muscle, or subcutaneously, and/or be metabolically stable and possess favourable pharmacokinetic properties.

International Patent Application publication number WO2009/012283 refers to various fluorophenyl compounds as Trk inhibitors; International Patent Application publication numbers WO2009/152087, WO2008/080015 and WO2008/08001 and WO2009/152083 refer to various fused pyrroles as kinase modulators; International Patent Application publication numbers WO2009/143024 and WO2009/143018 refer to various pyrrolo[2,3-d]pyrimidines substituted as Trk inhibitors; International Patent Application publication numbers WO2004/056830 and WO2005/116035 describe various 4-amino-pyrrolo[2,3-d]pyrimidines as Trk inhibitors. International Patent Application publication number WO2011/133637 describes various pyrrolo[2,3-d]pyrimidines and pyrrolo[2,3-b]pyridines as inhibitors of various kinases. International Patent Application publication number WO2005/099709 describes bicyclic heterocycles as serine protease inhibitors. International Patent Application publication number WO2007/047207 describes bicyclic heterocycles as FLAP modulators.

International Patent Application publication number WO2012/137089, and International Patent Applications PCT/IB2013/058890, PCT/IB2013/058895 and PCT/IB2013/058887 describe various heterocyclic compounds as Trk inhibitors.

Among the aims of this invention are to provide orally-active, efficacious, compounds and salts which can be used as active drug substances, particularly Trk antagonists, i.e. that block the intracellular kinase activity of the Trk, e.g. TrkA (NGF) receptor. Other desirable features include good HLM/hepatocyte stability, oral bioavailability, metabolic stability, absorption, selectivity over other types of kinase, dofetilide selectivity. Preferable compounds and salts will show a lack of CYP inhibition/induction, and be CNS-sparing.

### SUMMARY

The present invention provides compounds of Formula I:

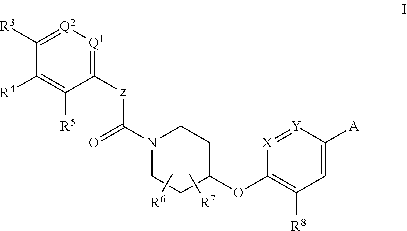

Wherein

Q¹ is N or CR¹,

Q² is N or CR²,

R¹, R², R⁴ and R⁵ are each independently H, F, CN, OH, NH₂, C₁₋₃ alkyl optionally substituted by one or more F, or C₁₋₃ alkoxy optionally substituted by one or more F, R³ is H, F, Cl, CN, C₁₋₄ alkyl optionally substituted by one or more F, C₁₋₄ alkoxy optionally substituted by one or more F , or C₃₋₇ cycloalkyloxy optionally substituted by one or more F, or C₁₋₄ alkylthio optionally substituted by one or more F,

With the proviso that at least 2 of R¹, R², R³, R⁴ and R⁵ are H,

R⁶ and R⁷ can be attached at any point on the piperidine ring and are independently H, F, CN, OH, NH₂, C₁₋₃ alkyl optionally substituted by one or more F, or C₁₋₃ alkoxy optionally substituted by one or more F,

or R⁶ and R⁷ can be taken together, with the atoms to which they are attached, to form a 3- to 7-membered cycloalkane ring or a 3- to 7-membered saturated heterocyclic ring (containing 1 ring hetero atom selected from O, S and N),

R⁸ is CONR¹⁰¹R¹⁰²,

X is CR¹⁰¹ or N,

Y is CR¹⁰² or N,

Z is CH₂, CH(CH₃), NH or O,

A is a phenyl or a 5- or 6-membered saturated or unsaturated heterocyclic ring containing 1, 2 or 3 hetero-atoms selected from S, N and O,

each of which is optionally fused to a further 5- or 6-membered saturated or unsaturated heterocyclic ring containing 1, 2 or 3 hetero-atoms selected from S, N and O,

and which phenyl or heterocyclic ring or fused ring system is optionally substituted by 1, 2 or 3 substituents independently selected from ═O, CN and C₀₋₆ alkyl optionally substituted by 1 or more F or by 1 or 2 substituents independently selected from OH, CO₂R⁹, NH₂, SO₂CH₃, C₁₋₄ alkoxy, CON(R¹⁰³)(R¹⁰⁴) and a group selected from

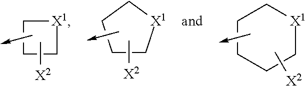

Where X¹ is selected from NR¹⁰¹, O and SO₂,

X² is H, OH or F,

R⁹ is H or C₁₋₆ alkyl,

R¹⁰¹ and R¹⁰² are each independently selected from H and C₁₋₃ alkyl,

R¹⁰³ and R¹⁰⁴ are each independently selected from H, (C₁₋₆ alkyl optionally substituted by OH, C₁₋₆ alkoxy or by one or more F), and (C₃₋₇ cycloalkyl optionally substituted by OH, C₁₋₆ alkoxy or by one or more F),

and pharmaceutically acceptable salts thereof.

The invention also comprises pharmaceutical compositions comprising a therapeutically effective amount of a compound of formula I as defined herein, or a pharmaceutically acceptable salt thereof, and a pharmaceutically acceptable carrier.

The invention is also directed to a method of treating a disease or condition indicated for treatment with a Trk antagonist, in a subject, by administering to a subject in need thereof a therapeutically effective amount of one or more of the compounds herein, or a pharmaceutically acceptable salt thereof.

Other aspects of the invention will be apparent from the remaining description and claims.

Preferably, the compounds of the present invention are potent antagonists at Trk receptors, and have a suitable PK profile to enable once daily dosing.

The compounds of the present invention are potentially useful in the treatment of a range of disorders where a Trk antagonist is indicated, particularly pain indications. Depending on the disease and condition of the patient, the term “treatment” as used herein may include one or more of curative, palliative and prophylactic treatment.

According to the invention a compound of the present invention may be useful to treat any physiological pain such as inflammatory pain, nociceptive pain, neuropathic pain, acute pain, chronic pain, musculo-skeletal pain, on-going pain, central pain, heart and vascular pain, head pain, orofacial pain. Other pain conditions which may be treated include intense acute pain and chronic pain conditions which may involve the same pain pathways driven by pathophysiological processes and as such cease to provide a protective mechanism and instead contribute to debilitating symptoms associated with a wide range of disease states.

Pain is a feature of many trauma and disease states. When a substantial injury, via disease or trauma, to body tissue occurs the characteristics of nociceptor activation are altered, this leads to hypersensitivity at the site of damage and in nearby normal tissue. In acute pain the sensitivity returns to normal once the injury has healed. However, in many chronic pain states, the hypersensitivity far outlasts the healing process and is normally due to nervous system injury due to maladaptation of the afferent fibres (Woolf & Salter 2000 Science 288: 1765-1768). Clinical pain is present when discomfort and abnormal sensitivity feature among the patient's symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Pain can be divided into a number of different areas because of differing pathophysiology, these include nociceptive, inflammatory, neuropathic pain among others. It should be noted that some types of pain have multiple aetiologies and thus can be classified in more than one area, e.g. Back pain, Cancer pain have both nociceptive and neuropathic components.

Disorders for which a trk inhibitor may be indicated include pain. Pain may be either acute or chronic and additionally may be of central and/or peripheral origin. Pain may be of a neuropathic and/or nociceptive and/or inflammatory nature, such as pain affecting either the somatic or visceral systems, as well as dysfunctional pain affecting multiple systems.

Physiological pain is an important protective mechanism designed to warn of danger from potentially injurious stimuli from the external environment. The system operates through a specific set of primary sensory neurones and is activated by noxious stimuli via peripheral transducing mechanisms (see Meyer et al., 2006, Wall and Melzack's Textbook of Pain (5^{th} Ed), Chapter1). These sensory fibres are known as nociceptors, and are characteristically small diameter axons with slow conduction velocities, of which there are two main types, A-delta fibres (myelinated) and C fibres (non-myelinated). Nociceptors encode the intensity, duration and quality of noxious stimulus and by virtue of their topographically organised projection to the spinal cord, the location of the stimulus. The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually, although not always, associated with a specific cause such as a defined injury, is often sharp and severe and can result from numerous origins such as surgery, dental work, a strain or a sprain. Acute pain does not generally result in any persistent psychological response. When a substantial injury occurs to body tissue, via disease or trauma, the characteristics of nociceptor activation may be altered such that there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury or alteration which can be associated with maladaptation and aberrant activity (Woolf & Salter, 2000, Science, 288, 1765-1768). As such, chronic pain is long-term pain, typically persisting for more than three months and leading to significant psychological and emotional problems. Common examples of chronic pain are neuropathic pain (e.g. painful diabetic neuropathy or postherpetic neuralgia), carpal tunnel syndrome, back pain, headache, cancer pain, arthritic pain and chronic post-surgical pain, but may include any chronic painful condition affecting any system, such as those described by the International Association for the Study of Pain (Classification of Chronic Pain, a publication freely available for download at http://www.iasp-pain.org).

The clinical manifestation of pain is present when discomfort and abnormal sensitivity feature among the patient's symptoms. Patients tend to be quite heterogeneous and may present with various pain symptoms. Such symptoms can include: 1) spontaneous pain which may be dull, burning, or stabbing; 2) exaggerated pain responses to noxious stimuli (hyperalgesia); and 3) pain produced by normally innocuous stimuli (allodynia) (Meyer et al., 2006, Wall and Melzack's Textbook of Pain (5^{th} Ed), Chapter1). Although patients suffering from various forms of acute and chronic pain may have similar symptoms, the underlying mechanisms may be different and may, therefore, require different treatment strategies. Apart from acute or chronic, pain can also be broadly categorized into: nociceptive pain, affecting either the somatic or visceral systems, which can be inflammatory in nature (associated with tissue damage and the infiltration of immune cells); or neuropathic pain.

Nociceptive pain can be defined as the process by which intense thermal, mechanical, or chemical stimuli are detected by a subpopulation of peripheral nerve fibers, called nociceptors, and can be induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 2006, Wall and Melzack's Textbook of Pain (5^{th} Ed), Chapter1). Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from strains/sprains, burns, myocardial infarction and acute pancreatitis, post-operative pain (pain following any type of surgical procedure), posttraumatic pain, pain associated with gout, cancer pain and back pain. Cancer pain may be chronic pain such as tumour related pain (e.g. bone pain, headache, facial pain or visceral pain) or pain associated with cancer therapy (e.g. in response to chemotherapy, immunotherapy, hormonal therapy or radiotherapy). Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Nociceptive pain can also be related to inflammatory states. The inflammatory process is a complex series of biochemical and cellular events, activated in response to tissue injury or the presence of foreign substances, which results in swelling and pain (McMahon et al., 2006, Wall and Melzack's Textbook of Pain (5^{th} Ed), Chapter3). A common inflammatory condition associated with pain is arthritis. It has been estimated that almost 27 million Americans have symptomatic osteoarthritis (OA) or degenerative joint disease (Lawrence et al., 2008, Arthritis Rheum, 58, 15-35); most patients with osteoarthritis seek medical attention because of the associated pain. Arthritis has a significant impact on psychosocial and physical function and is known to be the leading cause of disability in later life. Rheumatoid arthritis is an immune-mediated, chronic, inflammatory polyarthritis disease, mainly affecting peripheral synovial joints. It is one of the commonest chronic inflammatory conditions in developed countries and is a major cause of pain.

In regard to nociceptive pain of visceral origin, visceral pain results from the activation of nociceptors of the thoracic, pelvic, or abdominal organs (Bielefeldt and Gebhart, 2006, Wall and Melzack's Textbook of Pain (5^{th} Ed), Chapter48). This includes the reproductive organs, spleen, liver, gastrointestinal and urinary tracts, airway structures, cardiovascular system and other organs contained within the abdominal cavity. As such visceral pain refers to pain associated with conditions of such organs, such as painful bladder syndrome, interstitial cystitis, prostatitis, ulcerative colitis, Crohn's disease, renal colic, irritable bowl syndrome, endometriosis and dysmenorrheal (Classification of Chronic Pain, available at http://www.iasp-pain.org). Currently the potential for a neuropathic contribution (either through central changes or nerve injury/damage) to visceral pain states is poorly understood but may play a role in certain conditions (Aziz et al., 2009, Dig Dis 27, Suppl 1, 31-41)

Neuropathic pain is currently defined as pain arising as a direct consequence of a lesion or disease affecting the somatosensory system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Dworkin, 2009, Am J Med, 122, S1-S2; Geber et al., 2009, Am J Med, 122, S3-S12; Haanpaa et al., 2009, Am J Med, 122, S13-S21). The symptoms of neuropathic pain are difficult to treat, as they are often heterogeneous even between patients with the same disease (Dworkin, 2009, Am J Med, 122, S1-S2; Geber et al., 2009, Am J Med, 122, S3-S12; Haanpaa et al., 2009, Am J Med, 122, S13-S21). They include spontaneous pain, which can be continuous, and paroxysmal or abnormal evoked pain, such as hyperalgesia (increased sensitivity to a noxious stimulus) and allodynia (sensitivity to a normally innocuous stimulus).

It should be noted that some types of pain have multiple aetiologies and thus can be classified in more than one area, e.g. back pain, cancer pain and even migraine headaches may include both nociceptive and neuropathic components.

Similarly other types of chronic pain, perhaps less well understood, are not easily defined by the simplistic definitions of nociceptive or neuropathic. Such conditions include in particular fibromyalgia and chronic regional pain syndrome, which are often described as dysfunctional pain states e.g. fibromyalgia or complex regional pain syndrome (Woolf, 2010, J Clin Invest, 120, 3742-3744), but which are included in classifications of chronic pain states (Classification of Chronic Pain, available at http://www.iasp-pain.org).

### DETAILED DESCRIPTION

Embodiment 1 of the invention is a compound of Formula I:

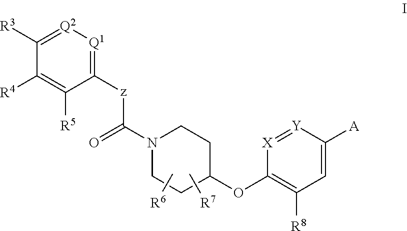

Wherein

Q¹ is N or CR¹,

Q² is N or CR²,

R¹, R², R⁴ and R⁵ are each independently H, F, CN, OH, NH₂, C₁₋₃ alkyl optionally substituted by one or more F, or C₁₋₃ alkoxy optionally substituted by one or more F, R³ is H, F, Cl, CN, C₁₋₄ alkyl optionally substituted by one or more F, C₁₋₄ alkoxy optionally substituted by one or more F, or C₃₋₇ cycloalkyloxy optionally substituted by one or more F, or C₁₋₄ alkylthio optionally substituted by one or more F,

With the proviso that at least 2 of R¹, R², R³, R⁴ and R⁵ are H,

R⁶ and R⁷ can be attached at any point on the piperidine ring and are independently H, F, CN, OH, NH₂, C₁₋₃ alkyl optionally substituted by one or more F, or C₁₋₃ alkoxy optionally substituted by one or more F,

or R⁶ and R⁷ can be taken together, with the atoms to which they are attached, to form a 3- to 7-membered cycloalkane ring or a 3- to 7-membered saturated heterocyclic ring (containing 1 ring hetero atom selected from O, S and N),

R⁸ is CONR¹⁰¹R¹⁰²,

X is CR¹⁰¹ or N,

Y is CR¹⁰² or N,

Z is CH₂, CH(CH₃), NH or O,

A is a phenyl or a 5- or 6-membered saturated or unsaturated heterocyclic ring containing 1, 2 or 3 hetero-atoms selected from S, N and O,

each of which is optionally fused to a further 5- or 6-membered saturated or unsaturated heterocyclic ring containing 1, 2 or 3 hetero-atoms selected from S, N and O,

and which phenyl or heterocyclic ring or fused ring system is optionally substituted by 1, 2 or 3 substituents independently selected from ═O, CN and C₀₋₆ alkyl optionally substituted by 1 or more F or by 1 or 2 substituents independently selected from OH, CO₂R⁹, NH₂, SO₂CH₃, C₁₋₄ alkoxy, CON(R¹⁰³)(R¹⁰⁴) and a group selected from

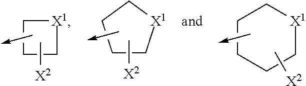

where X¹ is selected from NR¹⁰¹, O and SO₂,

X² is H, OH or F,

R⁹ is H or C₁₋₆ alkyl,

R¹⁰¹ and R¹⁰² are each independently selected from H and C₁₋₃ alkyl,

R¹⁰³ and R¹⁰⁴ are each independently selected from H, (C₁₋₆ alkyl optionally substituted by OH, C₁₋₆ alkoxy or by one or more F), and (C₃₋₇ cycloalkyl optionally substituted by OH, C₁₋₆ alkoxy or by one or more F),

Or a pharmaceutically acceptable salt thereof.

Embodiment 1A: A compound or salt according to embodiment 1 wherein R⁶ and R⁷ can be taken together, with the atoms to which they are attached, to form a 5- to 7-membered cycloalkane ring.

Embodiment 2: A compound or salt according to embodiment 1 or 1A wherein X is CH or N.

Embodiment 3: A compound or salt according to embodiment 1, 1A or 2 wherein Y is CH, N or C—CH₃.

Embodiment 4: A compound or salt according to embodiment 1, 1A, 2 or 3 wherein Z is CH₂, CH(CH₃) or NH.

Embodiment 5: A compound or salt according to embodiment 1, 1A, 2, 3 or 4 wherein R⁸ is CONH₂.

Embodiment 6: A compound or salt according to embodiment 1, 1A, 2, 3, 4 or 5 wherein R⁶ is H, F or CH₃.

Embodiment 7: A compound or salt according to embodiment 1, 1A, 2, 3, 4, 5 or 6 wherein

Q¹ is CH or N.

Embodiment 8: A compound or salt according to embodiment 1, 1A, 2, 3, 4, 5, 6 or 7 wherein Q² is CH or N.

Embodiment 9: A compound or salt according to embodiment 1, 1A, 2, 3, 4, 5, 6, 7 or 8 wherein R⁷ is F, H or CH₃.

Embodiment 10: A compound or salt according to embodiment 1, 1A, 2, 3, 4, 5, 6, 7, 8 or 9 wherein R³ is OCF₃, CF₃, C(CH₃)₃, SCF₃, CH(CH₃)₂ or cycloprolyoxy

Embodiment 11: A compound or salt according to embodiment 1, 1A, 2, 3, 4, 5, 6, 7, 8, 9 or 10 wherein A is an imidazolyl, pyrrolidinyl, thiazolyl, pyridyl, phenyl, or pyrazolyl group optionally substituted by 1 or 2 substituents independently selected from CO₂R⁹ and C₀₋₆ alkyl optionally substituted by 1 or 2 substituents independently selected from OH, NH₂, SO₂CH₃, C₁₋₄ alkoxy, CON(R¹⁰³)(R¹⁰⁴) and a group selected from

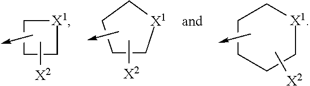

Embodiment 12: a compound or alt according to embodiment 11 where A is an imidazolyl, pyrrolidinyl, thiazolyl, pyridyl, phenyl, or pyrazolyl group optionally substituted by CH₃, CH₂SO₂CH₃ or by

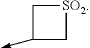

Embodiment 13: A compound according to Embodiment 1 which has the formula IA

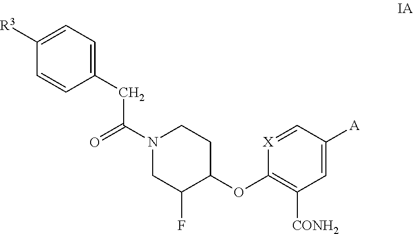

wherein

R³ is OCF₃ or cyclopropyloxy,

And X is CH or N,

Or a pharmaceutically acceptable salt thereof.

Embodiment 14 : A compound or salt according to Embodiment 12 wherein

A is a C-linked imidazolyl or pyrazolyl group optionally substituted by CH₃, CH₂SO₂CH₃ or by

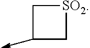

Embodiment 15: A compound selected from any of the Examples below, or a pharmaceutically acceptable salt thereof.

Embodiment 16: A compound according to Embodiment 1, selected from

5-[1-(1,1-dioxidothietan-3-yl)-1H-pyrazol-4-yl]-2-{[(3S,4R)-3-fluoro-1-{[4-(trifluoromethoxy)phenyl]acetyl}piperidin-4-yl]oxy}benzamide;

2-{[(3S,4S)-3-fluoro-1-{[4-(trifluoromethoxy)phenyl]acetyl}piperidin-4-yl]oxy}-5-(1-methyl-1H-imidazol-4-yl)pyridine-3-carboxamide;

2-{[(3R,4S)-3-fluoro-1-{[4-(trifluoromethoxy)phenyl]acetyl}piperidin-4-yl]oxy}-5-(1-methyl-1H-imidazol-4-yl)pyridine-3-carboxamide;

2-{[(3R,4S)-3-fluoro-1-{[4-(trifluoromethoxy)phenyl]acetyl}piperidin-4-yl]oxy}-5-(1-methyl-1H-pyrazol-4-yl)pyridine-3-carboxamide;

2-{[(3R,4S)-3-fluoro-1-{[4-(trifluoromethoxy)phenyl]acetyl}piperidin-4-yl]oxy}-5-(1H-pyrazol-4-yl)benzamide;

2-{[(3S,4R)-3-fluoro-1-(2-(4-(trifluoromethoxy)phenyl)acetyl)piperidin-4-yl]oxy}-5-(1-methyl-1H-imidazol-4-yl)benzamide;

2-{[(3S,4R)-1-{[4-(cyclopropyloxy)phenyl]acetyl}-3-fluoropiperidin-4-yl]oxy}-5-(1-methyl-1H-imidazol-4-yl)pyridine-3-carboxamide;

2-{[(3S,4S)-3-fluoro-1-{[4-(trifluoromethoxy)phenyl]acetyl}piperidin-4-yl]oxy}-5-(1-methyl-1H-imidazol-4-yl)benzamide;

2-{[(3S,4R)-3-fluoro-1-{[4-(trifluoromethoxy)phenyl]acetyl}piperidin-4-yl]oxy}-5-(1-methyl-1H-imidazol-4-yl)pyridine-3-carboxamide;

2-(pyrrolidin-1-yl)-5-((1-(2-(4-(trifluoromethoxy)phenyl)acetyl)piperidin-4-yl)oxy)isonicotinamide;

2-{[(3R,4S)-3-fluoro-1-{[4-(trifluoromethoxy)phenyl]acetyl}piperidin-4-yl]oxy}-5-(2-methyl-1H-imidazol-4-yl)pyridine-3-carboxamide

Or a pharmaceutically acceptable salt thereof.

Embodiment 17: A pharmaceutical composition comprising a compound of the formula (I) or a pharmaceutically acceptable salt thereof, as defined in any one of the preceding embodiments 1, 1A to 16, and a pharmaceutically acceptable carrier.

Embodiment 18: A compound of the formula (I) or a pharmaceutically acceptable salt thereof, as defined in any one of embodiments 1, 1A to 16, for use as a medicament.

Embodiment 19: A compound of formula (I) or a pharmaceutically acceptable salt thereof, as defined in any one of embodiments 1, 1A to 16 for use in the treatment of a disease for which an Trk receptor antagonist is indicated.

Embodiment 20: A compound of formula (I) or a pharmaceutically acceptable salt thereof, as defined in any one of embodiments 1, 1A to 16 for use in the treatment of pain or cancer.

Embodiment 21: The use of a compound of the formula (I) or a pharmaceutically acceptable salt or composition thereof, as defined in any one of embodiments 1, 1A to 16, for the manufacture of a medicament to treat a disease for which an Trk receptor antagonist is indicated

Embodiment 22: The use of a compound of the formula (I) or a pharmaceutically acceptable salt or composition thereof, as defined in any one of embodiments 1, 1A to 16, for the manufacture of a medicament to treat pain or cancer.

Embodiment 23: A method of treatment of a mammal, to treat a disease for which an Trk receptor antagonist is indicated, comprising treating said mammal with an effective amount of a compound of the formula (I) or a pharmaceutically acceptable salt thereof, as defined in any one of embodiments 1, 1A to 16.

Embodiment 24: A method of treatment of pain or cancer in a mammal, comprising treating said mammal with an effective amount of a compound of the formula (I) or a pharmaceutically acceptable salt thereof, as defined in any one of embodiments 1, 1A to 16.

Embodiment 25: A compound or salt according to any one of embodiments 1, 1A to 16 for use in a medical treatment in combination with a further drug substance.

Further embodiments include:

A compound or salt according to any one of embodiments wherein Q¹ has the value of Q¹ in any of the Examples;

A compound or salt according to any one of embodiments wherein Q² has the value of Q² in any of the Examples;

A compound or salt according to any one of embodiments wherein R³ has the value of R³ in any of the Examples;

A compound or salt according to any one of embodiments wherein R⁴ has the value of R⁴ in any of the Examples;

A compound or salt according to any one of embodiments wherein R⁵ has the value of R⁵ in any of the Examples;

A compound or salt according to any one of embodiments wherein R⁶ has the value of R⁶ in any of the Examples;

A compound or salt according to any one of embodiments wherein R⁷ has the value of R⁷ in any of the Examples;

A compound or salt according to any one of embodiments wherein R⁸ has the value of R⁸ in any of the Examples;

A compound or salt according to any one of embodiments wherein has the value of R³ in any of the Examples;

A compound or salt according to any one of embodiments wherein Z has the value of Z in any of the Examples;

A compound or salt according to any one of embodiments wherein X has the value of X in any of the Examples;

A compound or salt according to any one of embodiments wherein Y has the value of Y in any of the Examples;

A compound or salt according to any one of embodiments wherein A has the value of A in any of the Examples;

Any novel genus of intermediates described in the Schemes below;

Any novel specific intermediate described in the Preparations below;

Any novel process described herein.

“Halogen” means a fluoro, chloro, bromo or iodo group.

“Alkyl” groups, containing the requisite number of carbon atoms, can be unbranched or branched. Examples of alkyl include methyl, ethyl, n-propyl, i-propyl, n-butyl, i-butyl, sec-butyl and t-butyl.

“Pharmaceutically acceptable salts” of the compounds of formula I include the acid addition and base addition salts (including disalts, hemisalts, etc.) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base addition salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

The compounds of the invention include compounds of formula I and salts thereof as hereinbefore defined, polymorphs, and isomers thereof (including optical, geometric and tautomeric isomers) as hereinafter defined and isotopically-labelled compounds of formula I.

The compounds of the invention may be administered as prodrugs. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in a compound of formula (I) with certain moieties known to those skilled in the art as ‘pro-moieties’ as described, for example, in “Design of Prodrugs” by H Bundgaard (Elsevier, 1985).

Examples of prodrugs include phosphate prodrugs, such as dihydrogen or dialkyl (e.g. di-tert-butyl) phosphate prodrugs. Further examples of replacement groups in accordance with the foregoing examples and examples of other prodrug types may be found in the aforementioned references.

Also included within the scope of the invention are metabolites of compounds of formula (I), that is, compounds formed in vivo upon administration of the drug. Some examples of metabolites in accordance with the invention include, where the compound of formula (I) contains a phenyl (Ph) moiety, a phenol derivative thereof.

Unless otherwise specified, compounds of formula (I) containing one or more asymmetric carbon atoms can exist as two or more stereoisomers. Where a compound of formula (I) contains for example, a keto or guanidine group or an aromatic moiety, tautomeric isomerism (‘tautomerism’) can occur. It follows that a single compound may exhibit more than one type of isomerism.

Included within the scope of the claimed compounds of the present invention are all stereoisomers, geometric isomers and tautomeric forms of the compounds of formula (I), including compounds exhibiting more than one type of isomerism, and mixtures of one or more thereof. Also included are acid addition or base addition salts wherein the counterion is optically active, for example, D-lactate or L-lysine, or racemic, for example, DL-tartrate or DL-arginine.

Examples of types of potential tautomerisms shown by the compounds of the invention include hydroxypyridine [symbol] pyridone; amide [symbol] hydroxyl-imine and keto [symbol] enol tautomersims:

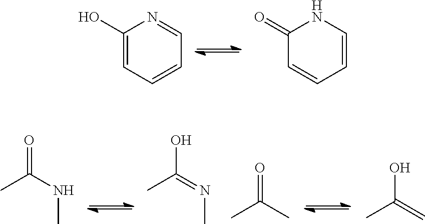

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or other derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagents in place of the non-labelled reagent previously employed.

The routes below, including those mentioned in the Examples and Preparations, illustrate methods of synthesising compounds of formula (I). The skilled person will appreciate that the compounds of the invention, and intermediates thereto, could be made by methods other than those specifically described herein, for example by adaptation of the methods described herein, for example by methods known in the art. Suitable guides to synthesis, functional group interconversions, use of protecting groups, etc., are for example: “Comprehensive Organic Transformations” by R C Larock, VCH Publishers Inc. (1989); Advanced Organic Chemistry” by J. March, Wiley Interscience (1985); “Designing Organic Synthesis” by S Warren, Wiley Interscience (1978); “Organic Synthesis—The Disconnection Approach” by S Warren, Wiley Interscience (1982); “Guidebook to Organic Synthesis” by R K Mackie and D M Smith, Longman (1982); “Protective Groups in Organic Synthesis” by T W Greene and P G M Wuts, John Wiley and Sons, Inc. (1999); and “Protecting Groups” by P J, Kocienski, Georg Thieme Verlag (1994); and any updated versions of said standard works.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

In the general synthetic methods below, unless otherwise specified, the substituents are as defined above with reference to the compounds of formula (I) above.

Where ratios of solvents are given, the ratios are by volume.

General Schemes

The compounds of the invention may be prepared by any method known in the art for the preparation of compounds of analogous structure. In particular, the compounds of the invention can be prepared by the procedures described by reference to the Schemes that follow, or by the specific methods described in the Examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of formula (I). It will be further appreciated that it may be necessary or desirable to carry out the transformations in a different order from that described in the schemes, or to modify one or more of the transformations, to provide the desired compound of the invention.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

All of the derivatives of the formula (I) can be prepared by the procedures described in the general methods presented below or by routine modifications thereof. The present invention also encompasses any one or more of these processes for preparing the derivatives of formula (I), in addition to any novel intermediates used therein.

According to a first process, compounds of formula (I) may be prepared from compounds of formula (VII) as illustrated by Scheme 1,

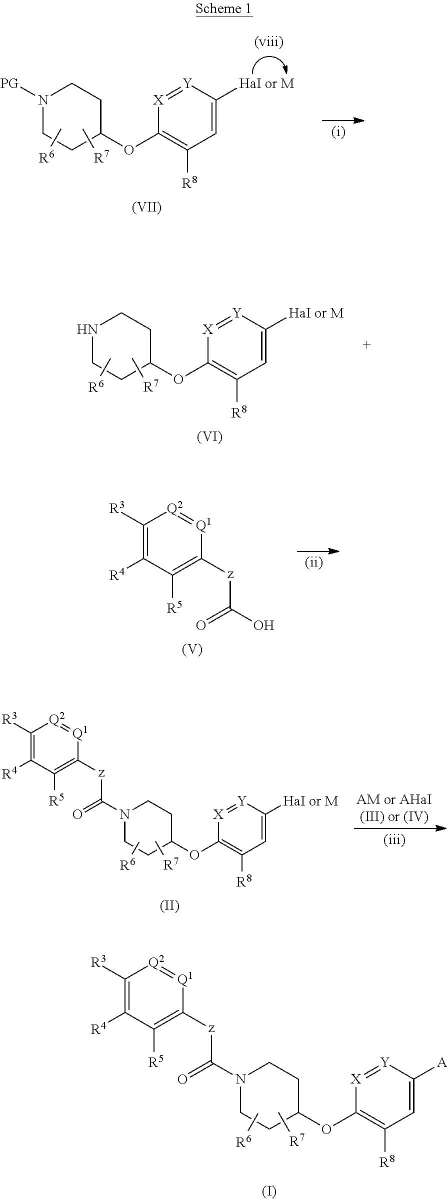

wherein Hal is chloro, bromo or iodo; M is a boronic ester or boronic acid; PG is a protecting group such as tert-butoxy carbonyl, benzyl or benzyloxycarbonyl.

Compounds of formulae (III), (IV) and (V) are commercially available or may be synthesized by those skilled in the art according to the literature or preparations described herein.

Compounds of formula (I) may be prepared from compounds of formula (II) according to process step (iii), a palladium catalysed Suzuki reaction with compounds of formula (III) or (IV). Suzuki cross-coupling is conveniently effected in the presence of a suitable catalyst eg: palladium in the presence of a phosphine ligand and an inorganic base such as sodium, potassium or cesium carbonate. Typical conditions comprise a boronic acid or ester, an aromatic halogen and a palladium catalyst with phosphine ligands in an organic solvent at elevated temperatures. Preferred Suzuki conditions comprise tris(dibenzylideneacetone)dipalladium (0) with tri-tertbutylphosphine tetrafluoroborate salt with potassium or sodium carbonate in dioxane/water, or 1,1-bis(diphenylphosphino)ferrocene palladium (II) dichloride with potassium carbonate in DMF/water or tetrakis(triphenylphosphine)palladium (0) with cesium carbonate in DMF/water all at elevated temperatures of between 100-110° C., either thermally or under microwave irradiation.

Compounds of formula (II) may be prepared from compounds of formula (V) and (VI) according to process step (ii) an amide bond formation reaction with activation of the carboxylic acid via an acid chloride or using a suitable base such as DIPEA and suitable coupling agents such as EDCl/HOBt, COMU and HATU. Preferred conditions when employing an acid chloride comprise DIPEA in DCM at room temperature, or using aqueous NaHCO₃ in THF at 0° C. Preferred conditions when employing a carboxylic acid include EDCl/HOBt or COMU or HATU, all with DIPEA in DCM or DMF at from 0° C. to room temperature.

Compounds of formula (VI) may be prepared from compounds of formula (VII) according to reaction step (i) a deprotection step mediated either by acid or palladium catalysis. Wherein PG is tert-butoxycarbonyl, preferred conditions comprise 4M HCl in dioxane or neat TFA; wherein PG is benzyl or benzyloxycarbonyl, preferred conditions comprise 10% palladium on carbon in acetic acid under hydrogenation.

Wherein compounds of formulae (VII), (VI) and (II) contain Hal, Hal may be converted to M when desired, according to process step (viii), a cross coupling reaction in the presence of a palladium catalyst with bispinacolatodiboron. Preferred conditions comprise potassium acetate with 1,1-bis(diphenylphosphino)ferrocene palladium (II) dichloride with bispinacolatodiboron in dioxane at 100° C.

According to a second process, certain compounds of formula (I) may be prepared from compounds of formula (II) as illustrated by Scheme 2,

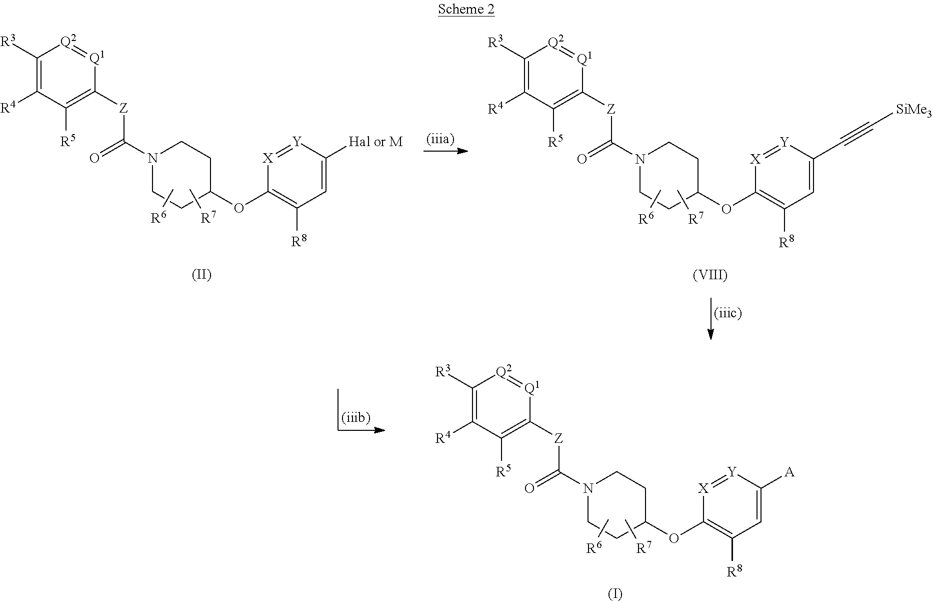

wherein Hal is chloro, bromo or iodo; M is a boronic ester or boronic acid.

Compounds of formula (I) may be prepared from compounds of formula (II) according to process step (iiia) a Stille cross coupling reaction followed by process step (iiic) a cyclisation reaction. Preferred conditions comprise trimethyl-tributyl-stannylethylnylsilane in the presence of bis-triphenylphosphinepalladium (II) dichloride in toluene at 130° C. followed by cyclisation with azidomethylpivalate with sodium ascorbate and copper sulfate hydrate in tert-butanol and water at room temperature.

Wherein A is N-linked, compounds of formula (I) may be prepared from compounds of formula (II) according to process step (iiib), a Buchwald reaction or an aromatic nucleophilic substitution reaction. Typical conditions comprise tris(dibenzylideneacetone)dipalladium (0) with xantphos and cesium carbonate in dioxane at elevated temperatures or heating with an amine in a sealed tube at 100° C. either with or without copper oxide either with or without a suitable organic solvent such as NMP.

According to a third process, compounds of formula (I) may be prepared from compounds of formula (IX) as illustrated by Scheme 3,

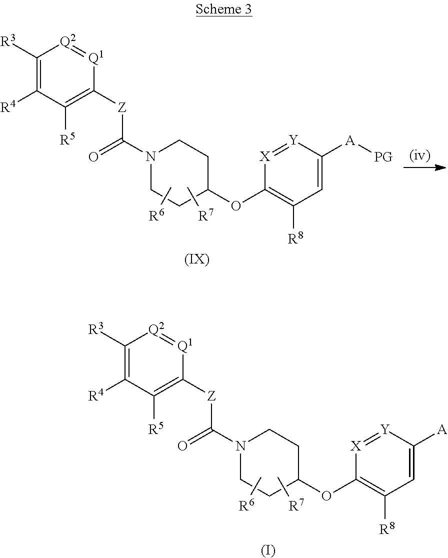

wherein PG is a suitable protecting group such as silylethoxymethyl, dimethyldioxolane, triphenylmethyl or benzyl.

Wherein A is protected with a protecting group such as silylethoxymethyl, dimethyldioxolane, triphenylmethyl or benzyl, a suitable deprotection step may be employed to obtain compounds of formula (I). Typical conditions are either acid, base or hydrogenation mediated depending on the nature of the protecting group present. Where an acid-labile protecting group is employed, preferred conditions comprise either between 2-6N aqueous HCl in a water miscible organic solvent such as THF or dioxane, or TFA in an organic solvent such as DCM, or para-toluenesulfonic acid in MeOH, all at room temperature. Where a hydrogenation-labile protecting group is employed, preferred conditions comprise 10% Pd/C in EtOAc at room temperature. Where a base-labile protecting group is used, preferred conditions comprise 1N NaOH in MeOH at room temperature. Alternatively the protecting group may fall off simultaneously in final process step (iii).

Compounds of formula (IX) may be prepared as described for compounds of formula (I) in Scheme 1.

Certain compounds of formula (I) may be interconverted to other compounds of formula (I) as illustrated below. Other functional group interconversions etc are also possible.

Wherein compounds of formula (I) are racemates, the racemic mixture may be separated into enantiomers through chiral separation chromatography as described in the Examples herein.

Wherein a free NH is present in A, compounds of formula (I) may be converted by an alkylation reaction to an N-alkyl compound, under basic reaction conditions. Preferred conditions comprise an alkylating agent with e.g. either cesium or potassium carbonate in DMF or acetonitrile at elevated temperatures of 110° C. for 16-18 hours.

Wherein A contains a methyl ester as a substituent, compounds of formula (I) where A contains a CON(R¹⁰³)(R¹⁰⁴) may be prepared by hydrolysis of the methyl ester under basic reaction conditions such as LiOH in THF, followed by an amide bond formation reaction with compounds of formula HN(R¹⁰³)(R¹⁰⁴) using a coupling agent such as propylphosphonic anhydride or according to reaction step (ii) as described in Scheme 1.

Wherein a free OH is present in A, compounds of formula (I) may be interconverted to a fluoro according to an electrophilic fluorination reaction. Preferred conditions comprise triethylamine trihydrofluoride in DCM with morpholinodifluorosulfinium tetrafluoroborate at from −78° C. to room temperature.

Wherein compounds of formula (I) contain a ketone, the ketone may be reduced to an alcohol under reducing reaction conditions such as sodium borohydride in ethanol.

According to a fifth process, compounds of formula (I) may be prepared from compounds of formula (V) and (X) as illustrated by Scheme 5.

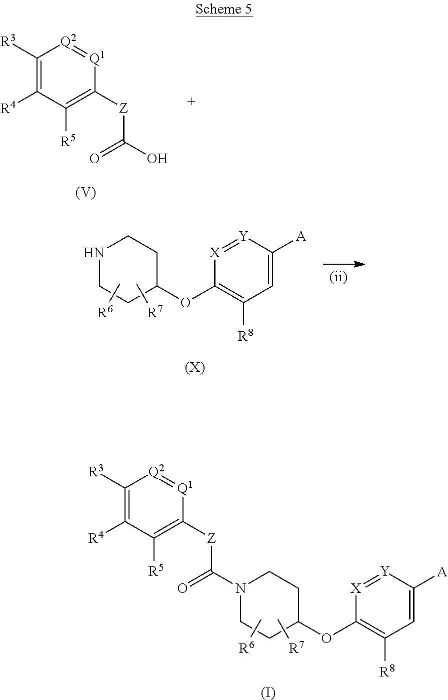

Compounds of formula (I) may be prepared from compounds of formula (V) and (X) according to process step (ii), an amide bond formation step as described in Scheme 1.

Compounds of formula (V) are commercially available or may be synthesized by those skilled in the art according to the literature or preparations described herein.

Compounds of formula (X) may be prepared as described in Scheme 9.

According to a sixth process, compounds of formula (I) may be prepared from compounds of formula (XII) and (XIA)/(XIB) as illustrated by Scheme 6.

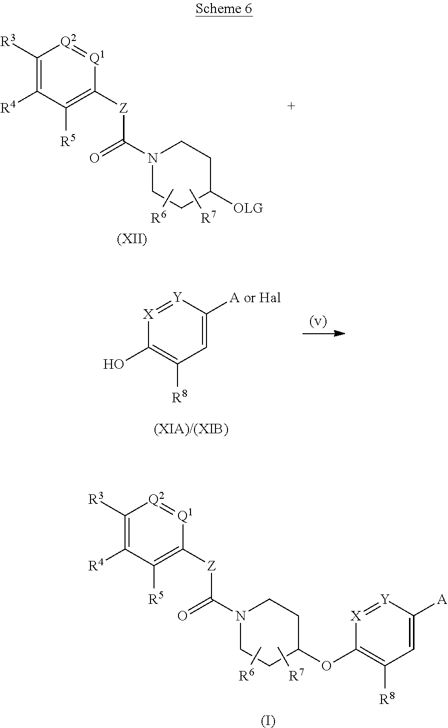

Wherein LG is a leaving group such as mesylate, tosylate, triflate, Hal is chloro, bromo or iodo;

Compounds of formula (XIA) and (XIB) are commercially available or may be synthesized by those skilled in the art according to the literature or preparations described herein or as described in Scheme 10.

Compounds of formula (XII) may be prepared as described in Scheme 12.

Compounds of formula (I) may be prepared from compounds of formulae (XII) and (XIA) or (XIB) according to process step (v), a nucleophilic substitution reaction in the presence of an inorganic base. Preferred conditions comprise cesium carbonate in an organic solvent such as DMF at elevated temperatures of 60° C. for 18 hours.

According to a seventh process, compounds of formula (I) wherein z is NH, may be prepared from compounds of formula (X) and (XIII)—as illustrated by Scheme 7.

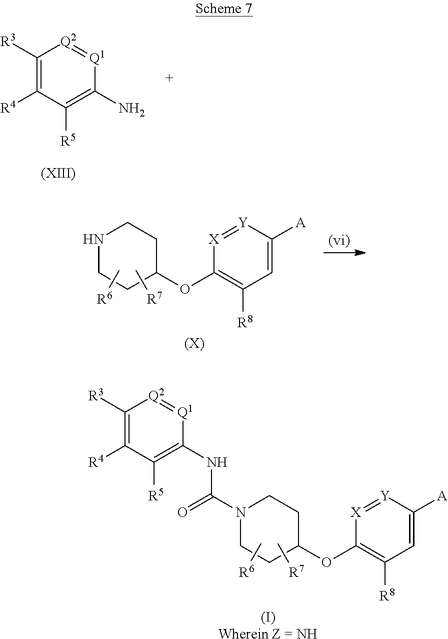

Compounds of formula (XIII) are commercially available or may be synthesized by those skilled in the art according to the literature or preparations described herein.

Compounds of formula (X) may be prepared as described in Scheme 9.

Compounds of formula (I) wherein Z═NH may be prepared from compounds of formula (X) according to process step (vi) a urea formation reaction with compounds of formula (XIII). Preferred conditions comprise DIPEA with triphosgene at 0° C., triethylamine with phosgene in THF, or triehtylamine with phenylchloroformate in THF.

According to an eighth process, compounds of formula (VII) may be prepared from compounds of formula (XIV) as illustrated by Scheme 8.

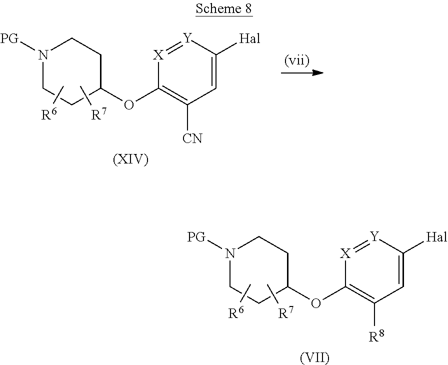

Wherein Hal is chloro, bromo, iodo;

Compounds of formula (VII) may be prepared from compounds of formula (XIV) according to process step (vii), a functional group interconversion of a nitrile to a primary carboxamide through a hydrolysis reaction under basic reaction conditions. Preferred conditions comprise potassium hydroxide in tert-butanol at elevated temperatures of 80° C. This functional group interconversion may be performed at any stage in Scheme 1. Alternatively the nitrile may be hydrolysed to a carboxylic acid using lithium hydroxide in methanol and THF followed by formation of a primary carboxamide via a mixed anhydride with isobutylchloroformate and ammonium hydroxide.

Compounds of formula (XIV) may be prepared in a similar manner to compounds of formula (VII) as described in Schemes 10 and 11 below.

According to a ninth process, compounds of formula (X) may be prepared from compounds of formula (VII) as illustrated by Scheme 9,

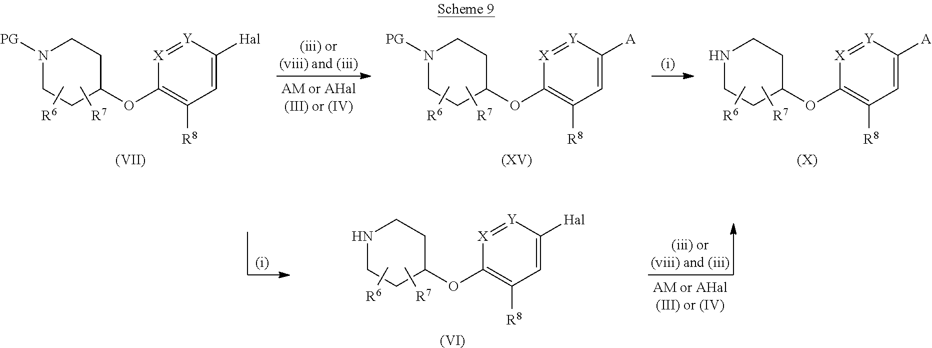

wherein Hal is chloro, bromo or iodo; M is a boronic acid or ester; PG is a protecting group such as tert-butoxy carbonyl, benzyl.

Compounds of formulae (III) and (IV) are commercially available or may be synthesized by those skilled in the art according to the literature or preparations described herein.

Compounds of formula (X) may be prepared from compound of formula (XV) according to process step (i), a deprotection reaction as described in Scheme 1.

Compounds of formula (XV) may be prepared from compounds of formula (VII) according to process step (iii), a Suzuki reaction or process steps (viii) and (iii), a conversion of Hal into M using bispinacolatodiboron followed by a Suzuki reaction with compounds of formula (III) or (IV) as described in Scheme 1.

Compounds of formula (X) may also be prepared from compounds of formula (VI) according to process step (iii), a Suzuki reaction or process steps (viii) and (iii), a conversion of Hal into M using bispinacolatodiboron followed by a Suzuki reaction with compounds of formula (III) or (IV) as described in Scheme 1.

Compounds of formula (VI) may be prepared from compound of formula (VII) according to process step (i), a deprotection reaction as described in Scheme 1.

Compounds of formula (VII) may be prepared according to the processes described in Schemes 8, 10 and 11.

According to an tenth process, compounds of formula (XVI) may be prepared from compounds of formula (XIA) as illustrated by Scheme 10.

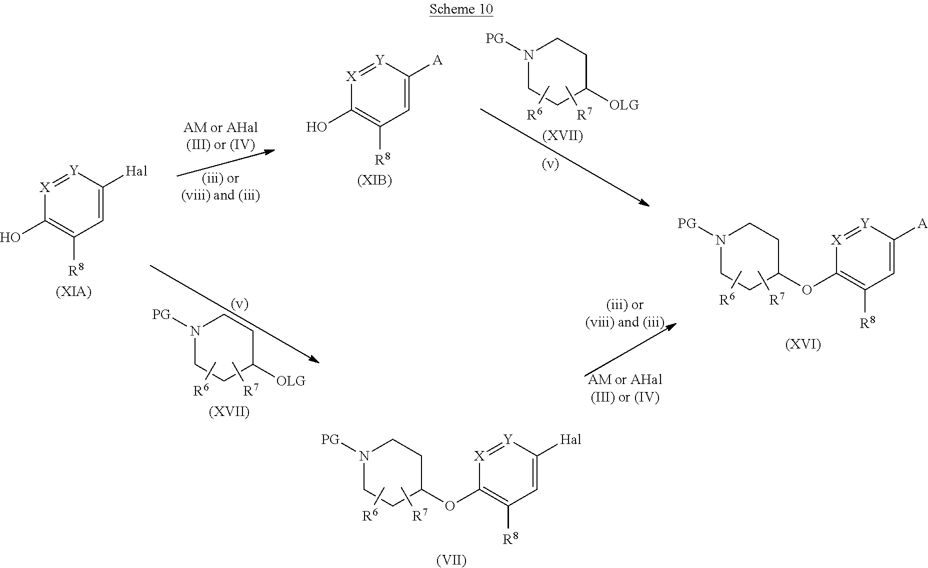

Wherein Hal is chloro, bromo, iodo; M is boronic acid or ester; PG is a protecting group such as tert-butoxy carbonyl, benzyl; LG is a leaving group such as mesylate, tosylate, triflate;

Compounds of formulae (XIA), (XVII), (III) and (IV) are commercially available or may be synthesized by those skilled in the art according to the literature or preparations described herein.

Compounds of formula (XVI) may be prepared from compounds of formula (XIB) and (XVII) according to process step (v), a nucleophilic substitution reaction as described in Scheme 6.

Compounds of formula (XIB) may also be prepared from compounds of formula (XIA) according to process step (iii), a Suzuki reaction or process steps (viii) and (iii), a conversion of Hal into M using bispinacolatodiboron followed by a Suzuki reaction with compounds of formula (III) or (IV) as described in Scheme 1.

Compounds of formula (XVI) may also be prepared from compounds of formula (VII) according to process step (iii), a Suzuki reaction or process steps (viii) and (iii), a conversion of Hal into M using bispinacolatodiboron followed by a Suzuki reaction with compounds of formula (III) or (IV) as described in Scheme 1.

Compounds of formula (VII) may be prepared from compounds of formula (XIA) and (XVII) according to process step (v), a nucleophilic substitution reaction as described in Scheme 6.

According to a eleventh process, compounds of formula (VII) may be prepared from compounds of formula (XIX) and (XVIII) as illustrated by Scheme 11,

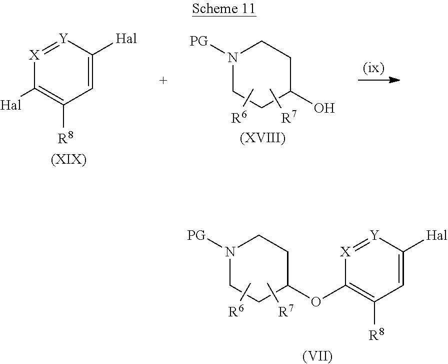

wherein Hal is fluoro, chloro, bromo, iodo; and PG is a protecting group such as tert-butoxy carbonyl, benzyl;

Compounds of formulae (XVIII) and (XIX) are commercially available or may be synthesized by those skilled in the art according to the literature or preparations described herein.

Compounds of formula (VII) may be prepared from compounds of formula (XVIII) and (XIX) according to process step (ix), an aromatic substitution reaction in the presence of an inorganic base. Preferred conditions comprise potassium tert-butoxide or cesium carbonate in DMF at from room temperature to 110° C.

According to a twelfth process, compounds of formula (XII) may be prepared from compounds of formulae (V) and (XXII) as illustrated by Scheme 12,

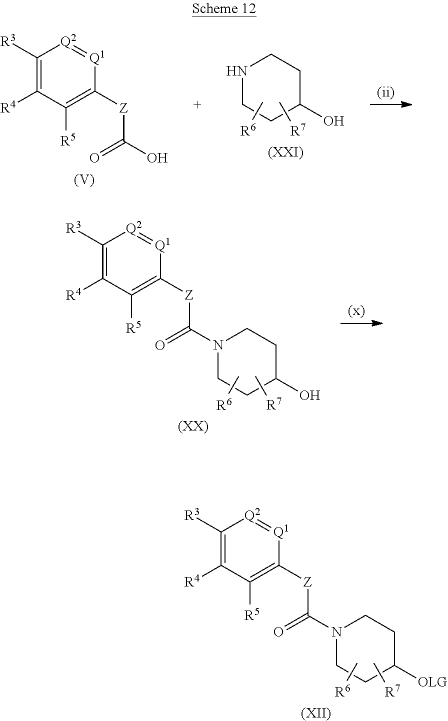

wherein LG is a leaving group such as mesylate, tosylate, triflate;

Compounds of formulae (V) and (XXI) are commercially available or may be synthesized by those skilled in the art according to the literature or preparations described herein.

Compounds of formula (XII) may be prepared from compounds of formula (XX) according to process step (x), a reaction transforming an alcohol into a suitable leaving group. Preferred conditions comprise mesyl chloride with triethylamine in DCM.

Compounds of formula (XX) may be prepared from compounds of formulae (XXI) and (V) according to process step (ii) as described in Scheme 1. Alternatively the acid chloride of compound of formula (V) maybe used with triethylamine in DCM.

Where A contains an ethyl ester group (—CO₂Et), this may be reduced to a primary alcohol group (—CH₂OH) using a stepwise approach, viz. saponification (preferably with LiOH in THF/water) followed by mixed anhydride formation (preferably using isobutylchloroformate and triethylamine in THF) followed by hydride reduction (preferably using NaBH₄ in water).

According to a further embodiment the present invention provides novel intermediate compounds described herein.

Pharmaceutically acceptable salts of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

A trk antagonist may be usefully combined with another pharmacologically active compound, or with two or more other pharmacologically active compounds, particularly in the treatment of pain. The skilled person will appreciate that such combinations offer the possibility of significant advantages, including patient compliance, ease of dosing and synergistic activity.

In the combinations that follow the compound of the invention may be administered simultaneously, sequentially or separately in combination with the other therapeutic agent or agents.

A trk antagonist compound of formula (I), or a pharmaceutically acceptable salt thereof, as defined above, may be administered in combination with one or more agents selected from:
a selective Nav1.3 channel modulator, such as a compound disclosed in WO2008/118758;
a selective Nav1.7 channel modulator, such as a compound disclosed in WO2010/079443, e.g. 4-[2-(5-amino-1H-pyrazol-4-yl)-4-chlorophenoxy]-5-chloro-2-fluoro-N-1,3-thiazol-4-ylbenzenesulfonamide or 4-[2-(3-amino-1H-pyrazol-4-yl)-4-(trifluoromethyl)phenoxy]-5-chloro-2-fluoro-N-1,3-thiazol-4-ylbenzenesulfonamide, or a pharmaceutically acceptable salt of either;
a selective Nav1.8 channel modulator;
a selective Nav1.9 channel modulator;
a compound which modulates activity at more than one Nav channel, including a non-selective modulator such as bupivacaine, carbamazepine, lamotrigine, lidocaine, mexiletine or phenytoin;
any inhibitor of nerve growth factor (NGF) signaling, such as: an agent that binds to NGF and inhibits NGF biological activity and/or downstream pathway(s) mediated by NGF signaling (e.g. tanezumab), a TrkA antagonist or a p75 antagoinsist, or an agent that inhibits downstream signaling in regard to NGF stimulated TrkA or P75 signalling;
a compound which increases the levels of endocannabinoid, such as a compound with fatty acid amid hydrolase inhibitory (FAAH) or monoacylglycerol lipase (MAGL) activity;
an analgesic, in particular paracetamol;
an opioid analgesic, such as: buprenorphine, butorphanol, cocaine, codeine, dihydrocodeine, fentanyl, heroin, hydrocodone, hydromorphone, levallorphan levorphanol, meperidine, methadone, morphine, nalmefene, nalorphine, naloxone, naltrexone, nalbuphine, oxycodone, oxymorphone, propoxyphene or pentazocine;
an opioid analgesic which preferentially stimulates a specific intracellular pathway, for example G-protein as opposed to beta arrestin recruitment, such as TRV130; an opioid analgesic with additional pharmacology, such as: noradrenaline (norepinephrine) reuptake inhibitory (NRI) activity, e.g. tapentadol; serotonin and norepinephrine reuptake inhibitory (SNRI) activity, e.g. tramadol; or nociceptin receptor (NOP) agonist activity, such as GRT6005;
a nonsteroidal antiinflammatory drug (NSAID), such as a non-selective cyclooxygenase (COX) inhibitor, e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac; or a COX-2 selective inhibitor, e.g. celecoxib, deracoxib, etoricoxib, mavacoxib or parecoxib;
a prostaglandin E₂ subtype 4 (EP4) antagonist;
a microsomal prostaglandin E synthase type 1 (mPGES-1) inhibitor;
a sedative, such as glutethimide, meprobamate, methaqualone or dichloralphenazone;
a GABA_{A} modulator with broad subtype modulatory effects mediated via the benzodiazepine binding site, such as chlordiazepoxide, alprazolam, diazepam, lorazepam, oxazepam, temazepam, triazolam, clonazepam or clobazam;
a GABA_{A} modulator with subtype-selective modulatory effects mediated via the benzodiazepine binding site with reduced adverse effects, for example sedation, such as TPA023, TPA023B, L-838,417, CTP354 or NSD72;
a GABA_{A} modulator acting via alternative binding sites on the receptor, such as barbiturates, e.g. amobarbital, aprobarbital, butabital, mephobarbital, methohexital, pentobarbital, phenobartital, secobarbital, or thiopental; neurosteroids such as alphaxalone, alphadolone or ganaxolone; β-subunit ligands, such as etifoxine; or δ-preferring ligands, such as gaboxadol;
a GlyR3 agonist or positive allosteric modulator;
a skeletal muscle relaxant, e.g. baclofen, carisoprodol, chlorzoxazone, cyclobenzaprine, metaxolone, methocarbamol or orphrenadine;
a glutamate receptor antagonist or negative allosteric modulator, such as an NMDA receptor antagonist, e.g. dextromethorphan, dextrorphan, ketamine or, memantine; or an mGluR antagonist or modulator;
an alpha-adrenergic, such as clonidine, guanfacine or dexmetatomidine;
a beta-adrenergic such as propranolol;
a tricyclic antidepressant, e.g. desipramine, imipramine, amitriptyline or nortriptyline;
a tachykinin (NK) antagonist, such as aprepitant or maropitant; a muscarinic antagonist, e.g oxybutynin, tolterodine, propiverine, tropsium chloride, darifenacin, solifenacin, temiverine and ipratropium;
a Transient Receptor Potential V1 (TRPV1) receptor agonist (e.g. resinferatoxin or capsaicin) or antagonist (e.g. capsazepine or mavatrap);
a Transient Receptor Potential A1 (TRPA1) receptor agonist (e.g. cinnamaldehyde or mustard oil) or antagonist (e.g. GRC17536 or CB-625);
a Transient Receptor Potential M8 (TRPM8) receptor agonist (e.g. menthol or icilin) or antagonist;
a Transient Receptor Potential V3 (TRPV3) receptor agonist or antagonist (e.g. GRC-15300);
a corticosteroid such as dexamethasone;
a 5-HT receptor agonist or antagonist, particularly a 5-HT_{1B/1D} agonist, such as eletriptan, sumatriptan, naratriptan, zolmitriptan or rizatriptan;
a 5-HT_{2A} receptor antagonist;
a cholinergic (nicotinic) analgesic, such as ispronicline (TC-1734), vareniclineor nicotine;
a PDEV inhibitor, such sildenafil, tadalafilor vardenafil;
an alpha-2-delta ligand such as gabapentin, gabapentin enacarbil or pregabalin;
a serotonin reuptake inhibitor (SRI) such as sertraline, demethylsertraline, fluoxetine, norfluoxetine, fluvoxamine, paroxetine, citalopram, desmethylcitalopram, escitalopram, d,l-fenfluramine, femoxetine, ifoxetine, cyanodothiepin, litoxetine, dapoxetine, nefazodone, cericlamine and trazodone;
anNRI, such as maprotiline, lofepramine, mirtazepine, oxaprotiline, fezolamine, tomoxetine, mianserin, buproprion, buproprion metabolite hydroxybuproprion, nomifensine and viloxazine, especially a selective noradrenaline reuptake inhibitor such as reboxetine;
an SNRI, such as venlafaxine, O-desmethylvenlafaxine, clomipramine, desmethylclom ipram ine, duloxetine, m ilnacipran and imipramine;
an inducible nitric oxide synthase (iNOS) inhibitor;
a leukotriene B4 antagonist;
a 5-lipoxygenase inhibitor, such as zileuton;
a potassium channel opener or positive modulator, such as an opener or positive modulator of KCNQ/Kv7 (e.g. retigabine or flupirtine), a G protein-coupled inwardly-rectifying potassium channel (GIRK), a calcium-activated potassium channel (Kca) or a potassium voltage-gated channel such as a member of subfamily A (e.g. Kv1.1), subfamily B (e.g. Kv2.2) or subfamily K (e.g. TASK, TREK or TRESK);
a P2X₃ receptor antagonist (e.g. AF219) or an antagonist of a receptor which contains as one of its subunits the P2X₃ subunit, such as a P2X_{2/3} heteromeric receptor;
a Caᵥ2.2 calcium channel blocker (N-type), such as ziconotide; and
a Caᵥ3.2 calcium channel blocker (T-type), such as ethosuximide.

Pharmaceutical compositions suitable for the delivery of compounds and salts of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

Compounds and salts of the invention intended for pharmaceutical use may be prepared and administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavoring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration discussed above may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

Parenteral Administration

The compounds and salts of the invention may be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) and salts used in the preparation of parenteral solutions may be increased by the use of appropriate formulation techniques, such as the incorporation of solubility-enhancing agents.

Formulations for parenteral administration may be formulated to be immediate and/or modified release. Thus, compounds and salts of the invention may be formulated as a solid, semi-solid, or thixotropic liquid for administration as an implanted depot providing modified release of the active compound. An example of such formulations include drug-coated stents.

Topical Administration

The compounds and salts of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated [see, for example, Finnin and Morgan, J Pharm Sci, 88 (10), 955-958 (October 1999).] Other means of topical administration include delivery by electroporation, iontophoresis, phonophoresis, sonophoresis and microneedle or needle-free (e.g. Powderject™, Bioject™, etc.) injection.

Inhaled/Intranasal Administration

The compounds and salts of the invention may also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

A pressurised container, pump, spray, atomizer, or nebuliser may contain a solution or suspension of the compound(s) or salt(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound or salt of the invention, a suitable powder base such as lactose or starch and a performance modifier such as /-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound or salt of the invention per actuation and the actuation volume may vary from 1 μl to 100 μl. A typical formulation may comprise a compound of formula (I) or salt thereof, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by a prefilled capsule, blister or pocket or by a system that utilises a gravimetrically fed dosing chamber. Units in accordance with the invention are typically arranged to administer a metered dose or “puff” containing from 1 to 5000 μg of the compound or salt. The overall daily dose will typically be in the range 1 μg to 20 mg which may be administered in a single dose or, more usually, as divided doses throughout the day.

Rectal/Intravaginal Administration

The compounds and salts of the invention may be administered rectally or vaginally, for example, in the form of a suppository, pessary, or enema. Cocoa butter is a traditional suppository base, but various well known alternatives may be used as appropriate.

Ocular and Aural Administration

The compounds and salts of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid; a cellulosic polymer, for example, hydroxypropylmethylcellulose, hydroxyethylcellulose, or methyl cellulose; or a heteropolysaccharide polymer, for example, gelan gum, may be incorporated together with a preservative, such as benzalkonium chloride. Such formulations may also be delivered by iontophoresis.

Other Technologies

The compounds and salts of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds and salts of the invention is typically in the range 0.1 mg to 200 mg depending, of course, on the mode of administration, preferred in the range 1 mg to 100 mg and more preferred in the range 1 mg to 50 mg. The total daily dose may be administered in single or divided doses.

These dosages are based on an average human subject having a weight of about 65 kg to 70 kg. The physician will readily be able to determine doses for subjects whose weight falls outside this range, such as infants and the elderly.

For the above-mentioned therapeutic uses, the dosage administered will, of course, vary with the compound or salt employed, the mode of administration, the treatment desired and the disorder indicated. The total daily dosage of the compound of formula (I)/salt/solvate (active ingredient) will, generally, be in the range from 1 mg to 1 gram, preferably 1 mg to 250 mg, more preferably 10 mg to 100 mg. The total daily dose may be administered in single or divided doses. The present invention also encompasses sustained release compositions.

The pharmaceutical composition may, for example, be in a form suitable for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

For parenteral dosages, this may conveniently be prepared as a solution or as a dry powder requiring dissolution by a pharmacist, medical practitioner or the patient. It may be provided in a bottle or sterile syringe. For example it may be provided as a powder in a multicompartment syringe which allows the dry powder and solvent to be mixed just prior to administration (to aid long-term stability and storage). Syringes could be used which allow multiple doses to be administered from a single device.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations as discussed below. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems.

Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, (1978). Pharmaceutical compositions are preferably manufactured under GMP conditions.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

The precise dosage administered of each active ingredient will vary depending upon any number of factors, including but not limited to, the type of animal and type of disease state being treated, the age of the animal, and the route(s) of administration.

The following non-limiting Preparations and Examples illustrate the preparation of compounds and salts of the present invention.

In the non-limiting Examples and Preparations that are set out later in the description, and in the aforementioned Schemes, the following the abbreviations, definitions and analytical procedures may be referred to:

t-Bu₃PHBF₄ is tri-tert-butylphosphinetetrafluoroborate salt

t-BuOH is tert-butanol;

° C. is degrees centigrade;

COMU® is (1-cyano-2-ethoxy-2-oxoethylideneaminooxy)dimethylamino-morpholino-carbenium hexafluorophosphate;

Cs₂CO₃ is cesium carbonate;

CuSO₄.5H₂O is copper sulphate pentahydrate;

DCM is dichloromethane; methylene chloride;

DEA is diethylamine

DIPEA is N-ethyldiisopropylamine, N,N-diisopropylethylamine;

DMF is N,N-dimethylformamide;

DMSO is dimethyl sulfoxide;

EDCI is 1-(3-dimethylaminopropyl)-3-ethylcarbodiimide hydrochloride;

EtOAc is ethyl acetate;

EtOH is ethanol;

H₂SO₄ is sulphuric acid;

HATU is 1-[Bis(dimethylamino)methylene]-1H-1,2,3-triazolo[4,5-b]pyridinium 3-oxid hexafluorophosphate;

HCl is hydrochloric acid;

HOBt is hydroxybenzotriazole;

HPLC is high-performance liquid chromatography

IPA is isopropanol;

KOH is potassium hydroxide;

KOAc is potassium acetate;

LCMS is liquid chromatography mass spectrometry (Rₜ=retention time)

Me is methyl

MeCN is acetonitrile;

MeOH is methanol;

MgSO₄ is magnesium sulphate;

MS is mass spectrometry;

NaHCO₃ is sodium hydrogen carbonate;

NaOH is sodium hydroxide;

Na₂SO₄ is sodium sulphate;

NH₃ is ammonia;

Pd/C is palladium on carbon;

Pd(PPh₃)₄ is palladium tetrakis;

PdCl₂(PPh₃)₂ is bis(triphenylphosphine)palladium (II) dichloride;

Pd₂(dba)₃ is tris(dibenzylideneacetone)dipalladium (0);

Pd(dppf)₂Cl₂ is [1,1′-bis(diphenylphosphino)ferrocene]dichloropalladium(II), complex with dichloromethane;

SEM is 2-[(trimethylsilyl)ethoxy]methyl;

TFA is trifluoroacetate;

THF is tetrahydrofuran;

HBr is hydrobromic acid;

CH2I2 is diiodomethane;

NaBH4 is sodium borohydride;

TBAF is tertbutylammonium fluoride;

CuI is copper iodide;

TMS is trimethylsilane;

LDA is lithium diisopropylamide;

AIBN is azobisisobutyronitrile;

KF is potassium fluoride;

THP is tetrahydropyran and

TLC is thin layer chromatography;

¹H and ¹⁹F Nuclear magnetic resonance (NMR) spectra were in all cases consistent with the proposed structures. Characteristic chemical shifts (δ) are given in parts-per-million downfield from tetramethylsilane (for ¹H-NMR) and upfield from trichloro-fluoro-methane (for ¹⁹F NMR) using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d₆-DMSO, deuterodimethylsulphoxide; and CD₃OD, deuteromethanol.

Mass spectra, MS (m/z), were recorded using either electrospray ionisation (ESI) or atmospheric pressure chemical ionisation (APCI).

Where relevant and unless otherwise stated the m/z data provided are for isotopes ¹⁹F, ³⁵Cl, ⁷⁹Br and ¹²⁷I.

Preparative HPLC:

Where singleton compounds are purified by preparative HPLC, there are two methods used, shown below:

| Method 1 | acidic conditions |
|---|---|
| Column | Gemini NX C18, 5 um 21.2 × 100 mm |
| Temperature | Ambient |
| Detection | ELSD-MS |
| Mobile Phase A | 0.1% formic acid in water |
| Mobile Phase B | 0.1% formic acid in acetonitrile |
| Gradient initial | 0% B, 1 mins-5% B; 7 mins-98% B; 9 mins-98% B; |
| | 9.1 mins-5% B; 10 mins-5% B |
| Flow rate | 18 mL/min |
| Injection volume | 1000 uL |

| Method 2 | basic conditions |
|---|---|
| Column | Gemini NX C18, 5 um 21.2 × 100 mm |
| Temperature | Ambient |
| Detection | ELSD-MS |
| Mobile Phase A | 0.1% diethylamine in water |
| Mobile Phase B | 0.1% diethylamine in acetonitrile |
| Gradient initial | 0% B, 1 mins-5% B; 7 mins-98% B; 9 mins-98% B; |
| | 9.1 mins-5% B; 10 mins-5% B |
| Flow rate | 18 mL/min |
| Injection volume | 1000 uL |

### EXAMPLE 1

### 5-[1-(1,1-dioxidothietan-3-yl)-1H-pyrazol-4-yl]-2-{[(3S,4R)-3-fluoro-1-{[4-(trifluoromethoxy)phenyl]acetyl}piperidin-4-yl]oxy}benzamide

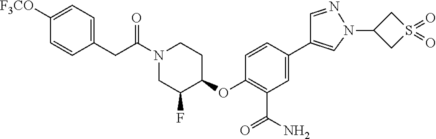 [SMILES: NC(=O)c1cc(-c2cnn(C3CS(=O)(=O)C3)c2)ccc1O[C@@H]1CCN(C(=O)Cc2ccc(OC(F)(F)F)cc2)C[C@@H]1F]

Method 1

To a solution of 5-bromo-2-{[(3S,4R)-3-fluoro-1-{[4-(trifluoromethoxy)phenyl]acetyl}piperidin-4-yl]oxy}benzamide (Preparation 28, 270 mg, 0.52 mmol) and 1-(1,1-dioxidothietan-3-yl)-4-(4,4,5,5-tetramethyl-1,3,2-dioxaborolan-2-yl)-1H-pyrazole (Preparation 169, 155 mg, 0.52 mmol) in dioxane (5 mL) was added a solution of potassium carbonate (179 mg, 1.30 mmol) in water (1 mL). The mixture was degassed with argon for 15 minutes before the addition of tris(dibenzylideneacetone)dipalladium (0) (23 mg, 0.03 mmol) and tri-tertbutylphosphine tetrafluoroborate salt (30 mg, 0.10 mmol) and heating the reaction to 100° C. for 16 hours. The reaction was cooled, diluted with EtOAc, washed with water, brine, dried over sodium sulphate and concentrated in vacuo. The residue was purified using silica gel column chromatography eluting with 6% MeOH in DCM to afford the title compound as a white solid (60 mg, 19%). NMR exhibits rotameric behaviour: ¹H NMR (400 MHz, DMSO-d₆): δ ppm 1.75 (m, 1H), 1.96 (m, 1H), 2.88-3.20 (m, 1H), 3.45-3.68 (m, 1H), 3.69-4.00 (m, 3H), 4.32 (m, 1H), 4.56-5.12 (m, 6H), 5.35 (m, 1H), 7.33 (m, 5H), 7.56 (br s, 1H), 7.68 (m, 2H), 8.08 (m, 2H), 8.41 (s, 1H).

MS m/z 611 [M+H]⁺

### EXAMPLE 2

### 2-{[(3S,4S)-3-fluoro-1-{[4-(trifluoromethoxy)phenyl]acetyl}piperidin-4-yl]oxy}-5-(1-methyl-1H-imidazol-4-yl)pyridine-3-carboxamide

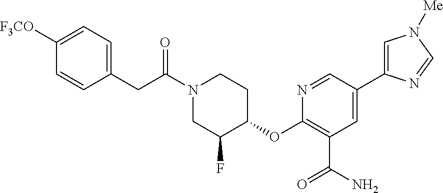 [SMILES: Cn1cnc(-c2cnc(O[C@H]3CCN(C(=O)Cc4ccc(OC(F)(F)F)cc4)C[C@@H]3F)c(C(N)=O)c2)c1]

Method 2

To a solution of 2-{[(3S,4S)-3-fluoro-1-{[4-(trifluoromethoxy)phenyl]acetyl}piperidin-4-yl]oxy}-5-(4,4,5,5-tetramethyl-1,3,2-dioxaborolan-2-yl)pyridine-3-carboxamide (Preparation 12, 650 mg, 1.15 mmol) and 4-iodo-1-methyl-1H-imidazole (239 mg, 1.15 mmol) in DMF (10 mL) was added a solution of potassium carbonate (318 mg, 2.31 mmol) in water (1 mL). The mixture was degassed with argon for 15 minutes followed by the addition of 1,1-bis(diphenylphosphino)ferrocene palladium (II) dichloride (47 mg, 0.06 mmol) and heating at 100° C. for 16 hours. The reaction was cooled, diluted with EtOAc, washed with water, brine, dried over sodium sulphate and concentrated in vacuo. The residue was purified using silica gel column chromatography eluting with 3-5% MeOH in DCM followed by preparative TLC eluting with 5% MeOH in DCM to afford the title compound as a white solid (32 mg, 5%).

NMR exhibits rotameric behaviour: ¹H NMR (400 MHz, DMSO-d₆): δ ppm 1.77 (m, 1H), 2.09 (m, 1H), 3.43-3.62 (m, 2H), 3.68 (s, 3H), 3.83 (s, 2H), 3.87-4.04 (m, 2H), 4.83-4.99 (m, 1H), 5.51 (m, 1H), 7.29-7.37 (m, 4H), 7.59 (br s, 1H), 7.67-7.74 (m, 3H), 8.46 (s, 1H), 8.62 (s, 1H).

MS m/z 522 [M+H]⁺

### EXAMPLE 3

### 2-{[(3R,4S)-3-fluoro-1-{[4-(trifluoromethoxy)phenyl]acetyl}piperidin-4-yl]oxy}-5-(1-methyl-1H-imidazol-4-yl)pyridine-3-carboxamide

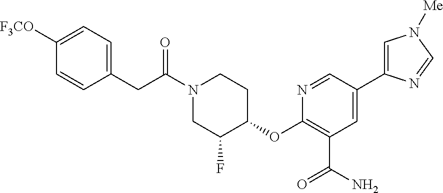 [SMILES: Cn1cnc(-c2cnc(O[C@H]3CCN(C(=O)Cc4ccc(OC(F)(F)F)cc4)C[C@H]3F)c(C(N)=O)c2)c1]

Method 3

To a solution of 2-{[(3R,4S)-3-fluoropiperidin-4-yl]oxy}-5-(1-methyl-1H-imidazol-4-yl)pyridine-3-carboxamide hydrochloride (Preparation 42, 4.22 g, 10.78 mmol) and DIPEA (5.56 g, 43.1 mmol) in DCM (150 mL) was added a solution of 2-(4-(trifluoromethoxy)phenyl)acetyl chloride (Preparation 170, 0.25M in DCM, 10.78 mmol) dropwise. The reaction was stirred at room temperature for 10 minutes before quenching with water (200 mL). The organic layer was collected, the aqueous washed with DCM twice (2×500 mL) and the organic layers were combined and concentrated in vacuo. The residue was purified using reverse phase column chromatography eluting with 0-80% ammonium hydroxide in MeCN/water to afford the title compound as a white powder (2.19 g, 39%).

NMR exhibits rotameric behaviour: ¹H NMR (400 MHz, DMSO-d₆): δ ppm 1.81 (m, 1H), 2.00 (m, 1H), 2.93 (t, 0.5H), 3.17 (dd,0.5H), 3.58 (dd, 0.5H), 3.63-3.74 (m, 3.5H), 3.81-3.88 (m, 1.5H), 4.01 (m,0.5H), 4.32 (m, 1H), 4.59 (m, 0.5H), 5.09 (dd, 1H), 5.51 (m, 1H), 7.26-7.31 (m, 2H), 7.34 (d, 2H), 7.52 (s, 1H), 7.67 (d, 2H), 7.82 (s, 1H), 8.52 (t, 1H), 8.62 (t, 1H).

MS m/z 522 [M+H]⁺

### EXAMPLE 4

### 2-{[(3R,4S)-3-fluoro-1-{[4-(trifluoromethoxy)phenyl]acetyl}piperidin-4-yl]oxy}-5-(1-methyl-1H-pyrazol-4-yl)pyridine-3-carboxamide

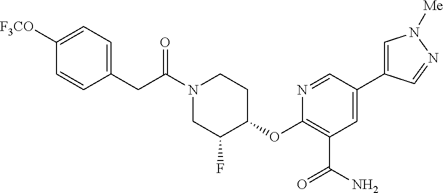 [SMILES: Cn1cc(-c2cnc(O[C@H]3CCN(C(=O)Cc4ccc(OC(F)(F)F)cc4)C[C@H]3F)c(C(N)=O)c2)cn1]

Method 4

To a solution of 5-bromo-2-{[(3R,4S)-3-fluoro-1-{[4-(trifluoromethoxy)phenyl]acetyl}piperidin-4-yl]oxy}pyridine-3-carboxamide (Preparation 26, 250 mg, 0.48 mmol) and 1-methyl-4-(4,4,5,5-tetramethyl-1,3,2-dioxaborolan-2-yl)-1H-pyrazole (200 mg, 0.96 mmol) in dioxane (4 mL) was added a solution of cesium carbonate (391 mg, 1.20 mmol) in water (1 mL). The mixture was degassed with argon for 15 minutes followed by the addition of tris(dibenzylideneacetone)dipalladium (0) (21 mg, 0.024 mmol) and tri-tertbutylphosphine tetrafluoroborate salt (27 mg, 0.096 mmol). The reaction was heated to 110° C. for 16 hours before cooling, diluting with EtOAc and washing with water. The organic extract was collected, dried over sodium sulphate and concentrated in vacuo. The residue was purified using silica gel column chromatography eluting with 8% MeOH in DCM followed by preparative TLC eluting with 5% MeOH in DCM to afford the title compound (55 mg, 22%).

NMR exhibits rotameric behaviour: ¹H NMR (400 MHz, DMSO-d₆): δ ppm 1.83 (m, 1H), 2.01 (m, 1H), 2.95 (m, 1H), 3.13-3.32 (m, 2H), 3.54-4.03 (m, 5.2H), 4.32-4.59 (m, 1.8H), 5.03-5.15 (m, 1H), 5.53 (m, 1H), 7.34 (m, 4H), 7.53 (br s, 1H), 7.84-7.91 (m, 2H), 8.23 (br s, 1H), 8.34 (br s, 1H), 8.53 (br s, 1H).

MS m/z 520 [M−H]⁻

### EXAMPLE 5

### 2-{[(3R,4S)-3-fluoro-1-{[4-(trifluoromethoxy)phenyl]acetyl}piperidin-4-yl]oxy}-5-(1H-pyrazol-4-yl)benzamide

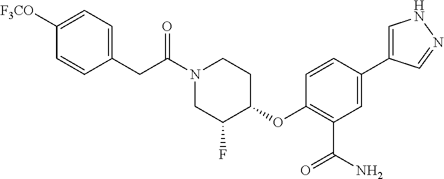 [SMILES: NC(=O)c1cc(-c2cn[nH]c2)ccc1O[C@H]1CCN(C(=O)Cc2ccc(OC(F)(F)F)cc2)C[C@H]1F]

Method 5

To a solution of 5-bromo-2-{[(3R,4S)-3-fluoro-1-{[4-(trifluoromethoxy)phenyl]acetyl}piperidin-4-yl]oxy}benzamide (Preparation 36, 400 mg, 0.77 mmol) and 4-(4,4,5,5-tetramethyl-1,3,2-dioxaborolan-2-yl)-1H-pyrazole (299 mg, 1.54 mmol) in dioxane (8 mL) was added a solution of sodium carbonate (245 mg, 1.06 mmol) in water (2 mL) and the mixture was degassed with argon for 15 minutes. Tris(dibenzylideneacetone)dipalladium (0) (35 mg, 0.039 mmol) and tri-tertbutylphosphine tetrafluoroborate salt (44 mg, 0.154 mmol) were added and the reaction heated to 100° C. for 16 hours. The reaction was cooled, diluted with EtOAc, washed with water, brine, dried over sodium sulphate and concentrated in vacuo. The residue was purified using silica gel column chromatography eluting with 3% MeOH in DCM to afford the title compound as a white solid (100 mg, 26%).

NMR exhibits rotameric behaviour: ¹H NMR (400 MHz, DMSO-d₆): δ ppm 1.75 (m, 1H), 1.95 (m, 1H), 2.88-3.27 (m, 1H), 3.45-3.57 (m, 0.6H), 3.72-4.00 (m, 2.4H), 4.33 (m, 1H), 4.58 (m, 0.6H), 4.88-5.12 (m, 2.4H), 7.26-7.35 (m, 5H), 7.56 (br s, 1H), 7.64-7.70 (m, 2H), 7.87 (br s, 1H), 8.01 (m, 1H), 8.17 (br s, 1H).

MS m/z 507 [M+H]⁺

### EXAMPLE 6

### 2-{[(3S,4R)-3-fluoro-1-(2-(4-(trifluoromethoxy)phenyl)acetyl)piperidin-4-yl]oxy}-5-(1-methyl-1H-imidazol-4-yl)benzamide

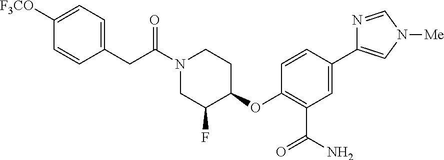 [SMILES: Cn1cnc(-c2ccc(O[C@@H]3CCN(C(=O)Cc4ccc(OC(F)(F)F)cc4)C[C@@H]3F)c(C(N)=O)c2)c1]

Method 6

To a solution of 2-{[(3S,4R)-3-fluoropiperidin-4-yl]oxy}-5-(1-methyl-1H-imidazol-4-yl)benzamide hydrochloride (Preparation 43, 7.30 g, 20.57 mmol) in saturated aqueous NaHCO₃ (100 mL) and THF (100 mL) at 0° C. was added a solution of [4-(trifluoromethoxy)phenyl]acetyl chloride in THF (Preparation 170, 100 mL, 22.00 mmol) dropwise over 30 minutes. The reaction was stirred at this temperature for 1 hour. The reaction was extracted into MEK (3×500 mL) and the combined extracts concentrated in vacuo. The residue was purified using silica gel column chromatography eluting with 5% MeOH in DCM to afford the title compound (6.48 g, 61%).

NMR exhibits rotameric behaviour: ¹H NMR (400 MHz, DMSO-d₆): δ ppm 1.66-1.76 (m, 1H), 1.93-1.99 (m, 1H), 2.86-2.92 (m, 0.5H), 3.04-3.35 (m, 1.5H), 3.44-3.57 (m, 0.5H), 3.60-3.73 (m, 3H), 3.80-3.87 (m, 1.5H), 3.94-4.03 (m, 0.5H), 4.26-4.36 (m, 1H), 4.56-4.62 (m, 0.5H), 4.84-5.15 (m, 2H).

MS m/z 521 [M+H]⁺

¹⁹F NMR (376 MHz, DMSO-d₆): δ ppm −57.26.

### EXAMPLE 7

### 2-{[(3S,4R)-1-{[4-(cyclopropyloxy)phenyl]acetyl}-3-fluoropiperidin-4-yl]oxy}-5-(1-methyl-1H-imidazol-4-yl)pyridine-3-carboxamide

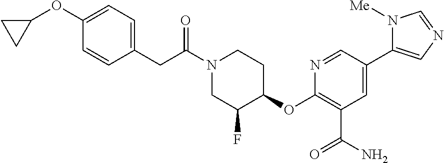 [SMILES: Cn1cncc1-c1cnc(O[C@@H]2CCN(C(=O)Cc3ccc(OC4CC4)cc3)C[C@@H]2F)c(C(N)=O)c1]

Method 7

To a solution of 2-{[(3S,4R)-3-fluoropiperidin-4-yl]oxy}-5-(1-methyl-1H-imidazol-4-yl)pyridine-3-carboxamide hydrochloride (Preparation 48, 200 mg, 0.51 mmol) in DCM (5 mL) was added DIPEA (0.451 mL, 2.55 mmol) at 0° C. (4-cyclopropoxyphenyl)acetic acid (Preparation 173, 97 mg, 0.51 mmol), EDCI (146 mg, 0.76 mmol) and HOBt (103 mg, 0.76 mmol) were added and the reaction stirred at room temperature for 16 hours. The reaction was diluted with EtOAc, washed with water, brine, dried over sodium sulphate and concentrated in vacuo. The residue was purified using silica gel column chromatography eluting with 5% MeOH in DCM followed by preparative TLC eluting with 5% MeOH in DCM to afford the title compound as a white solid (73 mg, 29%).

NMR exhibits rotameric behaviour: ¹H NMR (400 MHz, DMSO-d₆): δ ppm 0.60 (m, 2H), 0.80 (m, 2H), 1.70-1.82 (m, 1H), 1.95-2.03 (m, 1H), 2.92 (m, 0.5H), 3.09-3.39 (m, 1.5H), 3.49-3.80 (m, 6H), 4.00 (m, 0.5H), 4.35 (m, 1H), 4.61 (m, 0.5H), 5.01-5.14 (m, 1H), 5.46-5.54 (m, 1H), 6.96 (m, 2H), 7.14 (m, 2H), 7.52 (m, 1H), 7.67 (m, 2H), 7.82 (br s, 1H), 8.52 (m, 1H), 8.63 (m, 1H).

MS m/z 494 [M+H]⁺

### EXAMPLE 8

### 2-{[(3S,4S)-3-fluoro-1-{[4-(trifluoromethoxy)phenyl]acetyl}piperidin-4-yl]oxy}-5-(1-methyl-1H-imidazol-4-yl)benzamide

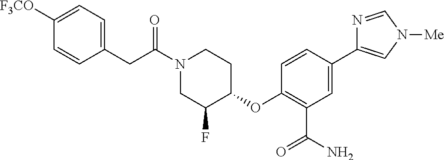 [SMILES: Cn1cnc(-c2ccc(O[C@H]3CCN(C(=O)Cc4ccc(OC(F)(F)F)cc4)C[C@@H]3F)c(C(N)=O)c2)c1]

Method 8

To a suspension of 2-{[(3S,4S)-3-fluoropiperidin-4-yl]oxy}-5-(1-methyl-1H-imidazol-4-yl)benzamide hydrochloride (Preparation 47, 320 mg, 0.905 mmol) in DCM (15 mL) was added triethylamine (0.634 mL, 4.526 mmol) at 0° C. (4-trifluoromethoxy)phenylacetic acid (199 mg, 0.905 mmol) was added followed by EDCI (268 mg, 1.358 mmol) and HOBt (183 mg, 1.358 mmol). The reaction was stirred at room temperature for 16 hours before diluting with EtOAc (30 mL). The solution was washed with water (10 mL), brine (10 mL), dried over sodium sulphate and concentrated in vacuo. The residue was purified using silica gel column chromatography eluting with 2-3% MeOH in DCM followed by preparative HPLC to afford the title compound (120 mg, 25%).

NMR exhibits rotameric behaviour: ¹H NMR (400 MHz, DMSO-d₆): δ ppm 1.70 (m, 1H), 2.02 (m, 1H), 3.31-3.87 (m, 8H), 4.03 (m, 1H), 4.73-4.95 (m, 2H), 7.23-7.36 (m, 5H), 7.53-7.60 (m, 4H), 7.75 (m, 1H), 8.05 (br s, 1H).

MS m/z 521 [M+H]⁺

### EXAMPLE 9

### 2-{[(3S,4R)-3-fluoro-1-{[4-(trifluoromethoxy)phenyl]acetyl}piperidin-4-yl]oxy}-5-(1-methyl-1H-imidazol-4-yl)pyridine-3-carboxamide

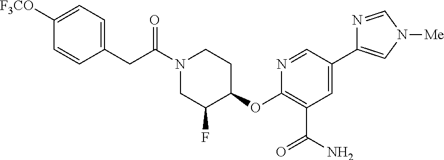 [SMILES: Cn1cnc(-c2cnc(O[C@@H]3CCN(C(=O)Cc4ccc(OC(F)(F)F)cc4)C[C@@H]3F)c(C(N)=O)c2)c1]

To a solution of 2-{[(3S,4R)-3-fluoro-1-{[4-(trifluoromethoxy)phenyl]acetyl}piperidin-4-yl]oxy}-5-(4,4,5,5-tetramethyl-1,3,2-dioxaborolan-2-yl)pyridine-3-carboxamide (Preparation 15, 320 mg, 0.56 mmol) and 4-iodo-1-methyl-1H-imidazole (175 mg, 0.85 mmol) in dioxane (8 mL) was added a solution of sodium carbonate (149 mg, 1.41 mmol) in water (2 mL) before degassing with argon for 15 minutes. Tris(dibenzylideneacetone)dipalladium (0) (25 mg, 0.03 mmol) and tri-tertbutylphosphine tetrafluoroborate salt (32 mg, 0.12 mmol) were added and the reaction heated to 100° C. for 16 hours. The reaction was cooled, diluted with EtOAc (100 mL), washed with water (2×30 mL), brine (20 mL), dried over sodium sulphate and concentrated in vacuo. The residue was purified using preparative HPLC to afford the title compound (30 mg, 10%). NMR exhibits rotameric behaviour: ¹H NMR (400 MHz, DMSO-d₆): δ ppm 1.76-1.90 (m, 1H), 2.02 (m, 1H), 2.94 (m, 1H), 3.12-3.36 (m, 1H), 3.49-3.73 (3H), 3.87 (m, 1.2H), 4.03 (m, 0.8H), 4.36 (m, 1.2H), 4.63 (m, 0.8H), 5.03-5.17 (m, 1H), 5.49-5.55 (m, 1H), 7.30 (m, 4H), 7.53 (br s, 1H), 7.70 (m, 2H), 7.82 (br s, 1H), 8.54 (s, 1H), 8.63 (s, 1H).

MS m/z 522 [M+H]⁺

### EXAMPLE 10

### 5-(1-methyl-1H-pyrazol-4-yl)-2-[(1-{[5-(trifluoromethoxy)pyridin-2-yl]acetyl}piperidin-4-yl)oxy]benzamide

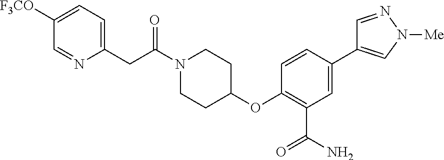 [SMILES: Cn1cc(-c2ccc(OC3CCN(C(=O)Cc4ccc(OC(F)(F)F)cn4)CC3)c(C(N)=O)c2)cn1]

Method 9

To a solution of [5-(trifluoromethoxy)pyridin-2-yl]acetic acid (Preparation 200, 5 mg, 0.019 mmol) and 5-(1-methyl-1H-pyrazol-4-yl)-2-(piperidin-4-yloxy)benzamide hydrochloride (Preparation 71, 10 mg, 0.027 mmol) in DMF (1 mL) was added DIPEA (26 uL, 0.152 mmol) and COMU® (11 mg, 0.025 mmol) and the reaction was stirred at room temperature for 2 hours. The reaction was concentrated in vacuo and purified using preparative HPLC to afford the title compound.

Rt=2.21 minutes MS m/z 504 [M+H]⁺

### EXAMPLE 11

### 5-(1H-pyrazol-4-yl)-2-[(1-{[4-(trifluoromethoxy)phenyl]acetyl}piperidin-4-yl)oxy]benzamide

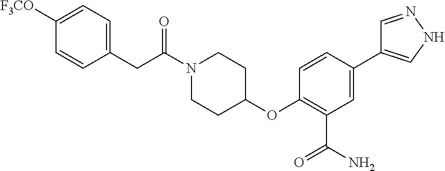 [SMILES: NC(=O)c1cc(-c2cn[nH]c2)ccc1OC1CCN(C(=O)Cc2ccc(OC(F)(F)F)cc2)CC1]

Method 10

A solution of 5-bromo-2-[(1-{[4-(trifluoromethoxy)phenyl]acetyl}piperidin-4-yl)oxy]benzamide (Preparation 39, 500 mg, 0.99 mmol), pyrazole-4-boronic acid pinacol ester (193 mg, 0.99 mmol) and cesium carbonate (647.5 mg, 1.99 mmol) in DMF:water (5:1, 10 mL) was degassed with argon for 15 minutes. Tetrakis(triphenylphosphine)palladium(0) (57.5 mg, 0.05 mmol) was added and the reaction heated to 110° C. in a sealed tube for 16 hours. The reaction was cooled, diluted with ethyl acetate (30 mL), washed with water (2×15 mL), brine (10 mL), dried over sodium sulphate and concentrated in vacuo. The residue was then purified using silica gel column chromatography eluting with 5-6% MeOH in DCM to afford the title compound as brown solid (120 mg, 25%).

¹H NMR (400 MHz, DMSO-d₆): δ ppm 1.64 (m, 2H), 1.90 (m, 2H), 3.43 (m, 2H), 3.71-3.79 (m, 4H), 4.76 (m, 1H), 7.19 (d, 1H), 7.29 (d, 2H), 7.35 (d, 2H), 7.55 (br s, 2H), 7.64 (dd, 1H), 7.85 (s, 1H), 7.88 (d, 1H), 8.14 (s, 1H), 12.9 (s, 1H).

MS m/z 489 [M+H]⁺

### EXAMPLE 12

### Methyl (4-{3-carbamoyl-4-[(1-{[4-(trifluoromethoxy)phenyl]acetyl}piperidin-4-yl)oxy]phenyl}-1H-imidazol-1-yl)acetate

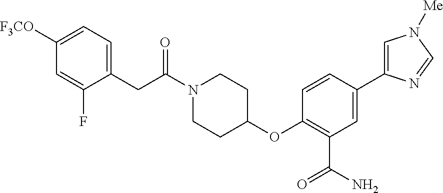 [SMILES: Cn1cnc(-c2ccc(OC3CCN(C(=O)Cc4ccc(OC(F)(F)F)cc4F)CC3)c(C(N)=O)c2)c1]

Method 11

To a stirred suspension of 5-(1-methyl-1H-imidazol-4-yl)-2-(piperidin-4-yloxy)benzamide hydrochloride (Preparation 45, 136 mg, 0.404 mmol) in THF (3.5 mL) was added DIPEA (0.36 mL, 2.02 mmol) at 0° C. phenyl acetic acid (90 mg, 0.404 mmol) and HATU (230.5 mg, 0.606 mmol) were added and the reaction stirred at room temperature for 40 hours. The reaction was diluted with EtOAc (20 mL), washed with water (2×10 mL), brine (10 mL), dried over sodium sulphate and concentrated in vacuo. The residue was purified by silica gel column chromatography eluting with 3% MeOH in DCM to afford the title compound as light brown solid (50 mg, 24%).

¹H NMR (400 MHz, DMSO-d₆): δ ppm 1.64-1.67 (m, 1H), 1.76-1.79 (m, 1H), 1.94-2.02 (m, 2H), 3.33-3.39 (m, 1H), 3.46-3.51 (m, 1H), 3.67 (s, 3H), 3.79-3.83 (m, 2H), 3.90 (s, 2H), 4.79-4.81 (m, 1H), 7.23 (d, 1H), 7.50-7.63 (m, 6H), 7.66 (s, 1H), 7.78 (dd, 1H), 8.11 (d, 1H).

The following Examples 13-55 were prepared according to Methods 7, 8, 9 or 11 (Examples 7,8,10 or 12) using compounds of formulae (V) and (X), and Purification Method (PM) below if different from the method described:

Purification Method A: Silica gel column chromatography eluting with 2-5% MeOH in EtOAc;

Purification Method B: Preparative TLC eluting with 3-5% MeOH in EtOAc;

Purification Method C: Preparative TLC eluting with between 2-6% MeOH in DCM;

Purification Method D: Silica gel column chromatography eluting with 4% MeOH in DCM followed by preparative HPLC;

Purification Method E: Silica gel column chromatography eluting with 2-8% MeOH in DCM.

| | | | Starting materials |
|---|---|---|---|
| Example | Structure and Name | Data | & PM |
| 13 | 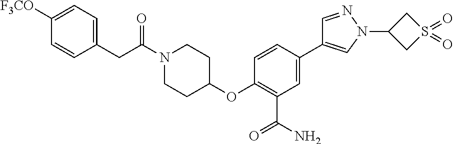 [SMILES: NC(=O)c1cc(-c2cnn(C3CS(=O)(=O)C3)c2)ccc1OC1CCN(C(=O)Cc2ccc(OC(F)(F)F)cc2)CC1] | MS m/z 593 [M + H]⁺ ¹H NMR (400 MHz, CD₃OD): δ ppm 1.70-1.88 (m, 2H), 1.94-2.10 (m, 2H), 3.48-3.60 (m, 2H), 3.80-3.86 (m, 1H), 3.86 (s, 2H), 3.90-3.98 (m, 1H), 4.72 (d, 4H), 4.80-4.84 (m, 1H), 5.36 (m, 1H), 7.22 (d, 1H), 7.26 (d, 2H), 7.38 (d, | 5-[1-(1,1- dioxidothietan-3-yl)- 1H-pyrazol-4-yl]-2- (piperidin-4- yloxy)benzamide trifluoroacetate (Preparation 76), 4-trifluoromethoxy- phenylacetic acid and PM A. |
| | 5-[1-(1,1-dioxidothietan-3-yl)-1H-pyrazol-4-yl]-2- | 2H), 7.68 (d, 1H), 7.94 (s, | |
| | [(1-{[4-(trifluoromethoxy)phenyl] | 1H), 8.06 (s, 1H), 8.16 (s, | |
| | acetyl}piperidin-4-yl)oxy]benzamide | 1H). | |
| 14 | 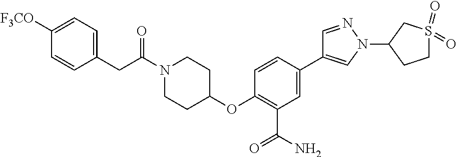 [SMILES: NC(=O)c1cc(-c2cnn(C3CCS(=O)(=O)C3)c2)ccc1OC1CCN(C(=O)Cc2ccc(OC(F)(F)F)cc2)CC1] | MS m/z 607 [M + H]⁺ ¹H NMR (400 MHz, DMSO-d₆): δ ppm 1.65 (m, 2H), 1.91 (m, 2H), 2.67 (m, 2H), 3.43 (m, 3H), 3.55 (m, 1H), 3.72-3.79 (m, 5H), 4.77 (m, 1H), 5.25 (m, 1H), 7.21 (m, 1H), 7.28-7.36 (m, 4H), 7.53-7.64 (m, 3H), 7.90 (s, 1H), 7.94 (s, 1H), 8.32 (s, | 5-[1-(1,1- dioxidotetrahydro- thiophen-3-yl)-1H- pyrazol-4-yl]-2- (piperidin-4- yloxy)benzamide hydrochloride (Preparation 44), 4- trifluoromethoxy- phenylacetic acid and PM B. |
| | Racemic 5-[1-(1,1-dioxidotetrahydrothiophen- | 1H). | |
| | 3-yl)-1H-pyrazol-4-yl]-2-[(1-{[4-(trifluoromethoxy)phenyl] | | |
| | acetyl}piperidin-4-yl)oxy]benzamide | | |
| 15 | 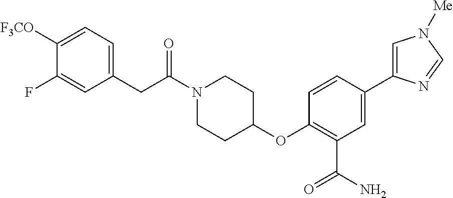 [SMILES: Cn1cnc(-c2ccc(OC3CCN(C(=O)Cc4ccc(OC(F)(F)F)c(F)c4)CC3)c(C(N)=O)c2)c1] | MS m/z 521 [M + H]⁺ ¹H NMR (400 MHz, DMSO-d₆): δ ppm 1.65- 1.74 (m, 2H), 1.95-1.99 (m, 2H), 3.36-3.37 (m, 1H), 3.42-3.46 (m, 1H), 3.68 (s, 3H), 3.74-3.82 (m, 4H), 4.77-4.79 (m, 1H), 7.17 (d, 1H), 7.22 (d, 1H), 7.37 (dd, 1H), 7.477.53 (m, 3H), 7.59 (s, 1H), 7.72 (br s, 1H), 7.77 (dd, 1H), 8.10 (d, 1H). | 5-(1-methyl-1H- imidazol-4-yl)-2- (piperidin-4- yloxy)benzamide hydrochloride (Preparation 45), 3- fluoro-4- (trifluoromethoxy) phenylacetic acid. |
| | 2-[(1-{[3-fluoro-4-(trifluoromethoxy)phenyl] | | |
| | acetyt}piperidin-4-yl)oxy]- | | |
| | 5-(1-methyl-1H-imidazol-4-yl)benzamide | | |
| 16 | 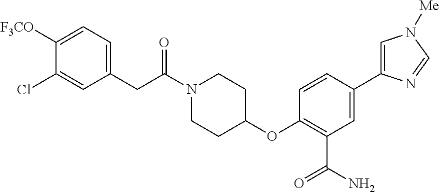 [SMILES: Cn1cnc(-c2ccc(OC3CCN(C(=O)Cc4ccc(OC(F)(F)F)c(Cl)c4)CC3)c(C(N)=O)c2)c1] | MS m/z 537 [M³⁵Cl + H]⁺ ¹H NMR (400 MHz, DMSO-d₆): δ ppm 1.63- 1.73 (m, 2H), 1.90-1.97 (m, 2H), 3.33-3.34 (m, 2H), 3.66 (s, 3H), 3.75- 3.83 (m, 4H), 4.77-4.79 (m, 1H), 7.20 (d, 1H), 7.32 (dd, 1H), 7.49-7.55 (m, 5H), 7.60 (s, 1H), 7.77 (dd, 1H), 8.10 (d, 1H). | 5-(1-methyl-1H- imidazol-4-yl)-2- (piperidin-4- yloxy)benzamide hydrochloride (Preparation 45), 3- chloro-4- (trifluoromethoxy) phenylacetic acid. |
| | 2-[(1-{[3-chloro-4-(trifluoromethoxy)phenyl] | | |
| | acetyl}piperidin-4-yl)oxy]-5-(1-methyl-1H-imidazol- | | |
| | 4-yl)benzamide | | |
| 17 | 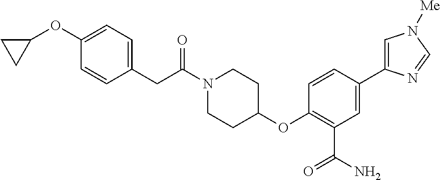 [SMILES: Cn1cnc(-c2ccc(OC3CCN(C(=O)Cc4ccc(OC5CC5)cc4)CC3)c(C(N)=O)c2)c1] | MS m/z 475 [M + H]⁺ ¹H NMR (400 MHz, DMSO-d₆): δ ppm 0.60- 0.64 (m, 2H), 0.73-0.78 (m, 2H), 1.60 (m, 2H), 1.88 (m, 2H), 3.39-3.42 (m, 2H), 3.74-3.81 (m, 8H), 4.75 (m, 1H), 6.96- 6.99 (m, 2H), 7.14-7.19 (m, 3H), 7.51-7.53 (m, 3H), 7.59 (s, 1H), 7.76- 7.77 (m, 1H), 8.09 (s, 1H). | 5-(1-methyl-1H- imidazol-4-yl)-2- (piperidin-4- yloxy)benzamide hydrochloride (Preparation 45), 2- (4- cyclopropoxyphenyl) acetic acid (WO2012137089). |
| | 2-[(1-{[4-(cyclopropyloxy)phenyl] | | |
| | acetyl}piperidin-4-yl)oxy]- | | |
| | 5-(1-methyl-1H-imidazol-4-yl)benzamide | | |
| 18 | 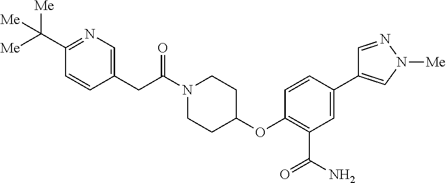 [SMILES: Cn1cc(-c2ccc(OC3CCN(C(=O)Cc4ccc(C(C)(C)C)nc4)CC3)c(C(N)=O)c2)cn1] | MS m/z 476 [M + H]⁺ ¹H NMR (400 MHz, DMSO-d₆): δ ppm 1.29 (s, 9H), 1.64 (m, 2H), 1.94 (m, 2H), 3.43 (m, 2H), 3.73-3.84 (m, 7H), 4.78 (m, 1H), 7.20 (m, 1H), 7.36 (m, 1H), 7.53-7.61 (m, 4H), 7.79 (br s, 1H), 7.86 (m, 1H), 8.10 (br s, 1H), 8.34 (m, 1H). | 5-(1-methyl-1H- pyrazol-4-yl)-2- (piperidin-4- yloxy)benzamide hydrochloride (Preparation 71), 2- (6-tertbutylpyridin- 3-yl)acetic acid and PM C. |
| | 2-({1-[(6-tert-butylpyridin-3-yl)acetyl]piperidin-4- | | |
| | yl}oxy)-5-(1-methyl-1H-pyrazol-4-yl)benzamide | | |
| 19 | 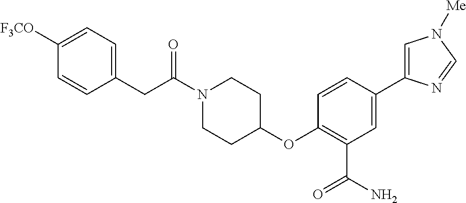 [SMILES: Cn1cnc(-c2ccc(OC3CCN(C(=O)Cc4ccc(OC(F)(F)F)cc4)CC3)c(C(N)=O)c2)c1] | MS m/z 503 [M + H]⁺ ¹H NMR (400 MHz, DMSO-d₆): δ ppm 1.62- 1.67 (m, 2H), 1.90-1.94 (m, 2H), 3.30-3.50 (m, 2H), 3.66 (s, 3H), 3.75- 3.83 (m, 4H), 4.77 (m, 1H), 7.18-7.20 (d, 1H), 7.28-7.36 (m, 4H), 7.52- 7.54 (m, 3H), 7.60 (s, 1H), 7.75-7.77 (dd, 1H), 8.10 (d, 1H). | 5-(1-methyl-1H- imidazol-4-yl)-2- (piperidin-4- yloxy)benzamide hydrochloride (Preparation 45), 4- trifluoromethoxy- phenylacetic acid and PM B. |
| | 5-(1-methyl-1H-imidazol-4-yl)-2-[(1-{[4- | | |
| | (trifluoromethoxy)phenyl] | | |
| | acetyl}piperidin-4-yl)oxy]benzamide | | |
| 20 | 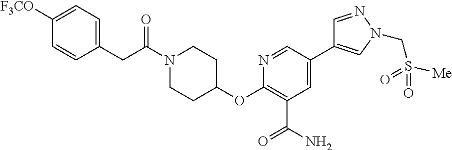 [SMILES: CS(=O)(=O)Cn1cc(-c2cnc(OC3CCN(C(=O)Cc4ccc(OC(F)(F)F)cc4)CC3)c(C(N)=O)c2)cn1] | MS m/z 582 [M + H]⁺ ¹H NMR (400 MHz, DMSO-d₆): δ ppm 1.72 (m, 2H), 1.97 (m, 2H), 3.05 (s, 3H), 3.33 m, 1H), 3.46 (m, 1H), 3.76-3.80 (m, 4H), 5.40 (m, 1H), 5.75 (s, 2H), 7.29-7.37 (m, 4H), 7.59 (br s, 1H), 7.77 (br s, 1H), 8.17 (s, 1H), 8.32 (m, 1H), | 5-{1-[(methylsulfonyl) methyl]-1H-pyrazol-4- yl}-2-(piperidin-4- yloxy)pyridine-3- carboxamide hydrochloride (Preparation 49) and 4-trifluoro- methoxyphenylacetic acid and PM B. |
| | 5-{1-[(methylsulfonyl)methyl]-1H-pyrazol-4-yl}- | 8.37 (s, 1H), 8.58 (d, 1H). | |
| | 2-[(1-{[4-(trifluoromethoxy)phenyl] | | |
| | acetyl}pjperidin-4-yl)oxy]pyridine-3-carboxamide | | |
| 21 | 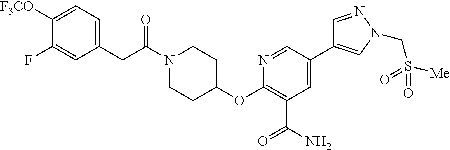 [SMILES: CS(=O)(=O)Cn1cc(-c2cnc(OC3CCN(C(=O)Cc4ccc(OC(F)(F)F)c(F)c4)CC3)c(C(N)=O)c2)cn1] | MS m/z 600 [M + H]⁺ ¹H NMR (400 MHz, DMSO-d₆): δ ppm 1.71 (m, 2H), 1.98 (m, 2H), 3.05 (s, 3H), 3.32 (m, 1H), 3.48 (m, 1H), 3.83 (m, 4H), 5.41 (m, 1H), 5.75 (s, 2H), 7.18 (d, 1H), 7.37 (d, 1H), 7.49 (t, 1H), 7.59 (br s, 1H), 7.76 (br s, 1H), 8.16 | 5-{1-[(methylsulfonyl) methyl]-1H-pyrazol-4- yl}-2-(piperidin-4- yloxy)pyridine-3- carboxamide hydrochloride (Preparation 49) and 3-fluoro-4- (trifluoromethoxy) phenylacetic acid and |
| | 2-[(1-{[3-fluoro-4-(trifluoromethoxy)phenyl] | (s, 1H), 8.32 (d, 1H), 8.37 | purification method |
| | acetyl}piperidin-4-yl)oxy]-5-{1-[(methylsulfonyl)methyl]- | (s, 1H), 8.58 (d, 1H). | C. |
| | 1H-pyrazol-4-yl}pyridine-3-carboxamide | | |
| 22 | 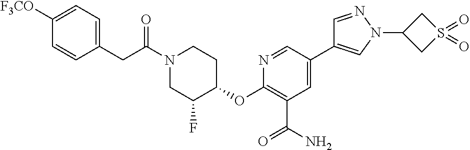 [SMILES: NC(=O)c1cc(-c2cnn(C3CS(=O)(=O)C3)c2)cnc1O[C@H]1CCN(C(=O)Cc2ccc(OC(F)(F)F)cc2)C[C@H]1F] | MS m/z 612 [M + H]⁺ ¹H NMR (400 MHz, DMSO-d₆): δ ppm 1.81- 1.87 (m, 1H), 2.02 *m, 1H), 2.95 (m, 1H), 3.26 (m, 1H), 3.55-3.87 (m, 3H), 4.36 (m, 1H), 4.55- 4.85 (m, 5H), 5.03-5.20 (m, 1H), 5.34-5.60 (m, 2H), 7.30-7.36 (m, 4H), 7.53 (br s, 1H), 7.86 (br s, | 5-[1-(1,1- dioxidothietan-3-yl)- 1H-pyrazol-4-yl]-2- {[(3R,4S)-3- fluoropiperidin-4- yl]oxy}pyridine-3- carboxamide hydrochloride (Preparation 50) and 4- trifluoromethoxy- |
| | 5-[1-(1,1-dioxidothietan-3-yl)-1H-pyrazol-4-yl]-2- | 1H), 8.13 (s, 1H), 8.40 (s, | phenylacetic acid. |
| | {[(3R,4S)-3-fluoro-1-{[4-(trifluoromethoxy)phenyl] | 1H), 8.52 (s, 1H), 8.57 (s, | |
| | acetyl}piperidin-4-yl]oxy}pyridine-3-carboxamide | 1H). | |
| 23 | 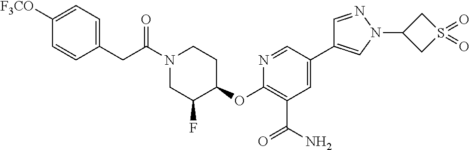 [SMILES: NC(=O)c1cc(-c2cnn(C3CS(=O)(=O)C3)c2)cnc1O[C@@H]1CCN(C(=O)Cc2ccc(OC(F)(F)F)cc2)C[C@@H]1F] | MS m/z 612 [M + H]⁺ ¹H NMR (400 MHz, DMSO-d₆): δ ppm 1.81- 1.87 (m, 1H), 2.02 *m, 1H), 2.95 (m, 1H), 3.26 (m, 1H), 3.55-3.87 (m, 3H), 4.36 (m, 1H), 4.55- 4.85 (m, 5H), 5.03-5.20 (m, 1H), 5.34-5.60 (m, 2H), 7.30-7.36 (m, 4H), 7.53 (br s, 1H), 7.86 (br s, | 5-[1-(1,1- dioxidothietan-3-yl)- 1H-pyrazol-4-yl]-2- {[(3S,4R)-3- fluoropiperidin-4- yl]oxy}pyridine-3- carboxamide hydrochloride (Preparation 51) and 4- trifluoromethoxy- |
| | 5-[1-(1,1-dioxidothietan-3-yl)-1H-pyrazol-4-yl]-2- | 1H), 8.13 (s, 1H), 8.40 (s, | phenylacetic acid. |
| | {[(3S,4R)-3-fluoro-1-{[4-(trifluoromethoxy)phenyl] | 1H), 8.52 (s, 1H), 8.57 (s, | |
| | acetyl}piperidin-4-yl]oxy}pyridine-3-carboxamide | 1H). | |
| 24 | 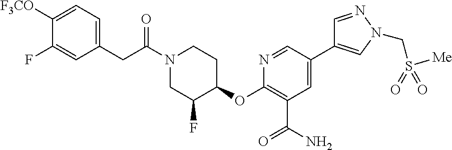 [SMILES: CS(=O)(=O)Cn1cc(-c2cnc(O[C@@H]3CCN(C(=O)Cc4ccc(OC(F)(F)F)c(F)c4)C[C@@H]3F)c(C(N)=O)c2)cn1] | MS m/z 618 [M + H]⁺ ¹H NMR (400 MHz, DMSO-d₆): δ ppm 1.75- 1.95 (m, 1H), 2.03 (m, 1H), 2.96 (m, 0.5H), 3.05 (s, 3H), 3.19 (m, 0.5H), 3.56-4.03 (m, 3H), 4.32 (m, 1H), 4.58 (m, 1H), 5.03-5.19 (m, 1H), 5.48- 5.56 (m, 1H), 5.75 (s, 2H), 7.19 (m, 1H), 7.34 (m, | 2-{[(3S,4R)-3- fluoropiperidin-4- yl]oxy}-5-{1- [(methylsulfonyl) methyl]-1H-pyrazol- 4-yl}pyridine-3- carboxamide hydrochloride (Preparation 46) and 3-fluoro-4- (trifluoromethoxy) |
| | 2-{[(3S,4R)-3-fluoro-1-{[3-fluoro-4-(trifluoromethoxy)phenyl] | 1H), 7.48-7.54 (m, 2H), | phenylacetic acid. |
| | acetyl}pipertdin-4-yl]oxy}-5-{1-[(methylsulfonyl)methyl]- | 7.84 (br s, 1H), 8.18 (s, | |
| | 1H-pyrazol-4-yl}pyridine-3-carboxamide | 1H), 8.39 (s, 1H), 8.59 (s, | |
| | | 1H). | |
| 25 | 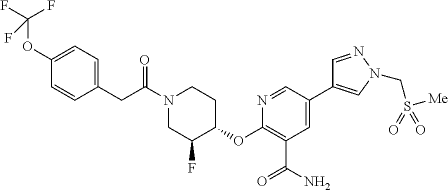 [SMILES: CS(=O)(=O)Cn1cc(-c2cnc(O[C@H]3CCN(C(=O)Cc4ccc(OC(F)(F)F)cc4)C[C@@H]3F)c(C(N)=O)c2)cn1] | MS m/z 600 [M + H]⁺ ¹H NMR (400 MHz, DMSO-d₆): δ ppm 1.77 (m, 1H), 2.08 (m, 1H), 3.05 (s, 3H), 3.40-4.10 (m, 6H), 4.80-5.05 (m, 1H), 5.51 (m, 1H), 5.75 (s, 2H), 7.29-7.37 (m, 4H), 7.63 (br s, 1H), 7.76 (br d, 1H), 8.18 (br s, 1H), 8.33 (m, 1H), 8.38 (br s, 1H), 8.58 (d, 1H). | tert-butyl (3S,4S)-4- [(3-carbamoyl-5-{1- [(methylsulfonyl) methyl]-1H-pyrazol- 4-yl}pyridin-2-yl)oxy]- 3-fluoropiperidine-1- carboxylate hydrochloride (Preparation 54) and 4- trifluoromethoxy- phenylacetic acid. |
| | 2-{[(3S,4S)-3-fluoro-1-{[4-(trifluoromethoxy)phenyl] | | |
| | acetyl}piperidin-4-yl]oxy}-5-{1-[(methylsulfonyl)methyl]- | | |
| | 1H-pyrazol-4-yl}pyridine-3-carboxamide | | |
| 26 | 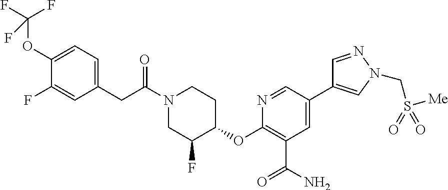 [SMILES: CS(=O)(=O)Cn1cc(-c2cnc(O[C@H]3CCN(C(=O)Cc4ccc(OC(F)(F)F)c(F)c4)C[C@@H]3F)c(C(N)=O)c2)cn1] | MS m/z 618 [M + H]⁺ ¹H NMR (400 MHz, DMSO-d₆): δ ppm 1.77 (m, 1H), 2.11 (m, 1H), 3.05 (s, 3H), 3.47-4.07 (m, 6H), 4.83-5.01 (m, 1H), 5.52 (m, 1H), 5.75 (s, 2H), 7.19 (m, 1H), 7.35 (m, 1H), 7.50 (m, 1H), 7.63 (br s, 1H), 7.78 (m, 1H), 8.18 (br s, 1H), 8.33 (m, 1H), 8.39 (br s, 1H), 8.59 (d, 1H). | tert-butyl (3S,4S)-4- [(3-carbamoyl-5-{1- [(methylsulfonyl) methyl]-1H-pyrazol- 4-yl}pyridin-2-yl)oxy]- 3-fluoropiperidine-1- carboxylate hydrochloride (Preparation 54) and 3-fluoro-4- (trifluoromethoxy) phenylacetic acid. |
| | 2-{[(3S,4S)-3-fluoro-1-{[3-fluoro-4-(trifluoromethoxy)phenyl] | | |
| | acetyl}piperidin-4-yl]oxy}-5-{1-[(methylsulfonyl)methyl]-1H- | | |
| | pyrazol-4-yl}pyridine-3-carboxamide | | |
| 27 | 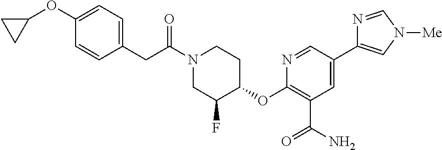 [SMILES: Cn1cnc(-c2cnc(O[C@H]3CCN(C(=O)Cc4ccc(OC5CC5)cc4)C[C@@H]3F)c(C(N)=O)c2)c1] | MS m/z 494 [M + H]⁺ ¹H NMR (400 MHz, DMSO-d₆): δ ppm 0.63 (m, 2H), 0.76 (m, 2H), 1.73 (m, 1H), 2.08 (m, 1H), 3.50-4.05 (m, 9H), 4.80- 4.93 (m, 1H), 5.49 (m, 1H), 6.99 (m, 2H), 7.17 (m, 1H), 7.59 (br s, 1H), 7.67-7.73 (m, 4H), 8.46 (s, 1H), 8.62 (s, 1H). | 2-{[(3S,4S)-3- fluoropiperidin-4- yl]oxy}-5-(1-methyl- 1H-imidazol-4- yl)pyridine-3- carboxamide hydrochloride (Preparation 55) and 4- cyclopropoxyphenyl acetic acid. |
| | 2-{[(3S,4S)-1-{[4-(cyclopropyloxy)phenyl] | | |
| | acetyl}-3-fluoropiperidin-4-yl]oxy}-5-(1- | | |
| | methyl-1H-imidazol-4-yl)pyridine-3-carboxamide | | |
| 28 | 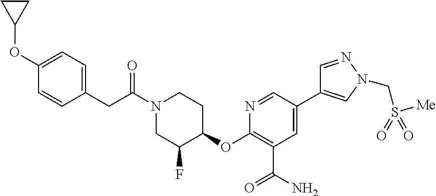 [SMILES: CS(=O)(=O)Cn1cc(-c2cnc(O[C@@H]3CCN(C(=O)Cc4ccc(OC5CC5)cc4)C[C@@H]3F)c(C(N)=O)c2)cn1] | MS m/z 572 [M + H]⁺ ¹H NMR (400 MHz, DMSO-d₆): δ ppm 0.63 (m, 2H), 0.78 (m, 2H), 1.77 (m, 1H), 1.99 (m, 1H), 2.90-3.30 (5H), 3.50-4.01 (m, 4H), 4.01-4.59 (m, 1H), 5.02-5.14 (m, 1H), 5.53 (m, 1H), 5.75 (s, 2H), 6.98 (m, 2H), 7.14 (m, 1H), 7.55 (m, 1H), 7.86 (br s, 1H), 8.18 (s, 1H), 8.40 (m, 2H), 8.60 (m, 1H). | 2-{[(3S,4R)-3- fluoropiperidin-4- yl]oxy}-5-{1- [(methylsulfonyl) methyl]-1H-pyrazol- 4-yl}pyridine-3- carboxamide hydrochloride (Preparation 46) and 4- cyclopropoxyphenyl acetic acid. |
| | 2-{[(3S,4R)-1-{[4-(cyclopropyloxy)phenyl]acetyl}-3- | | |
| | fluoropiperidin-4-yl]oxy}-5-{1-[(methylsulfonyl)methyl]- | | |
| | 1H-pyrazol-4-yl}pyridine-3-carboxamide | | |
| 29 | 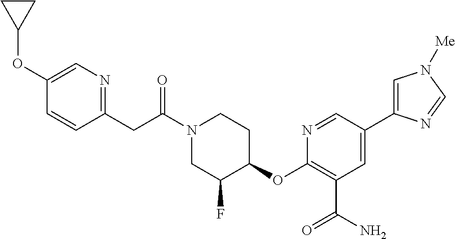 [SMILES: Cn1cnc(-c2cnc(O[C@@H]3CCN(C(=O)Cc4ccc(OC5CC5)cn4)C[C@@H]3F)c(C(N)=O)c2)c1] | MS m/z 595 [M + H]⁺ ¹H NMR (400 MHz, DMSO-d₆): δ ppm 0.68 (m, 2H), 0.80 (m, 2H), 1.83 (m, 1H), 2.00 (m, 1H), 2.95-3.38 (m, 2H), 3.52- 3.91 (m, 7H), 4.30-4.70 (m, 1H), 5.01-5.16 (m, 1H), 5.55 (m, 1H), 7.25 (m, 1H), 7.45 (m, 1H), 7.53 (br s, 1H), 7.69 (m, 2H), 7.82 (br s, 1H), 8.25 (s, 1H), 8.54 (s, 1H), 8.63 (s, 1H). | 2-{[(3S,4R)-3- fluoropiperidin-4- yl]oxy}-5-(1-methyl- 1H-imidazol-4- yl)pyridine-3- carboxamide hydrochloride (Preparation 48) and [5- (cyclopropyloxy) pyridin-2-yl]acetic acid (Preparation 211). |
| | 2-{[(3S,4R)-1-{[5-(cyclopropyloxy)pyridin-2-yl]acetyl}-3- | | |
| | fluoropiperidin-4-yl]oxy}-5-(1-methyl-1H-imidazol- | | |
| | 4-yl)pyridine-3-carboxamide | | |
| 30 | 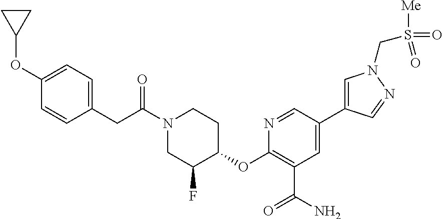 [SMILES: CS(=O)(=O)Cn1cc(-c2cnc(O[C@H]3CCN(C(=O)Cc4ccc(OC5CC5)cc4)C[C@@H]3F)c(C(N)=O)c2)cn1] | MS m/z 572 [M + H]⁺ ¹H NMR (400 MHz, DMSO-d₆): δ ppm 0.63 (m, 2H), 0.769 (m, 2H), 1.74 (m, 1H), 2.08 (m, 1H), 3.05 (s, 3H), 3.29-3.80 (m, 6H), 4.00 (m, 1H), 4.80- 4.92 (m, 1H), 5.48 (m, 1H), 5.75 (s, 2H), 6.97 (m, 2H), 7.15 (m, 2H), 7.62 (br s, 1H), 7.75 (m, 1H), 8.17 (s, 1H), 8.33 (br s, 1H), 8.38 (s, 1H), 8.58 (s, 1H). | 2-{[(3S,4S)-3- fluoropiperidin-4- yl]oxy}-5-{1- [(methylsulfonyl) methyl]-1H-pyrazol- 4-yl}pyridine-3- carboxamide hydrochloride (Preparation 54) and 4- cyclopropoxyphenyl acetic acid. |
| | 2-{[(3S,4S)-1-{[4-(cyclopropyloxy)phenyl]acetyl}-3- | | |
| | fluoropiperidin-4-yl]oxy}-5-{1-[(methylsulfonyl)methyl]- | | |
| | 1H-pyrazol-4-yl}pyridine-3-carboxamide | | |
| 31 | 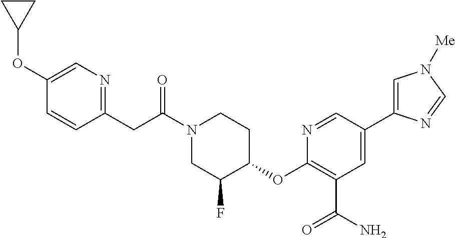 [SMILES: Cn1cnc(-c2cnc(O[C@H]3CCN(C(=O)Cc4ccc(OC5CC5)cn4)C[C@@H]3F)c(C(N)=O)c2)c1] | MS m/z 495 [M + H]⁺ ¹H NMR (400 MHz, DMSO-d₆): δ ppm 0.67 (m, 2H), 0.80 (m, 2H), 1.76 (m, 1H), 2.10 (m, 1H), 3.29-3.39 (m, 1H), 3.55- 4.20 (m, 9H), 4.80-5.00 (m, 1H), 5.50 (m, 1H), 7.23 (m, 1H), 7.47 (m, 1H), 7.59 (br s, 1H), 7.68 (m, 1H), 7.74 (m, 1H), 8.25 *m, 1H), 8.46 (m, 1H), 8.62 (m, 1H). | 2-{[(3S,4S)-3- fluoropiperidin-4- yl]oxy}-5-(1-methyl- 1H-imidazol-4- yl)pyridine-3- carboxamide hydrochloride (Preparation 55) and [5- (cyclopropyloxy) pyridin-2-yl]acetic acid (Preparation 211). |
| | 2-{[(3S,4S)-1-{[5-(cyclopropyloxy)pyridin- | | |
| | 2-yl]acetyl}-3-fluoropiperidin-4-yl]oxy}- | | |
| | 5-(1-methyl-1H-imidazol-4-yl)pyridine-3-carboxamide | | |
| 32 | 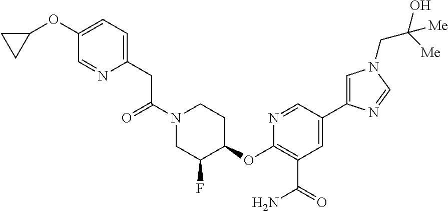 [SMILES: CC(C)(O)Cn1cnc(-c2cnc(O[C@@H]3CCN(C(=O)Cc4ccc(OC5CC5)cn4)C[C@@H]3F)c(C(N)=O)c2)c1] | MS m/z 553 [M + H]⁺ ¹H NMR (400 MHz, DMSO-d₆): δ ppm 0.67 (m, 2H), 0.79 (m, 2H), 1.08 (s, 6H), 1.80 (m, 1H), 1.99 (m, 1H), 2.90-3.20 (m, 1H), 3.50-4.70 (m, 7H), 5.00-5.20 (m, 1H), 5.50 (m, 1H), 7.23 (d, 1H), 7.47 (m, 1H), 7.53 (br s, 1H), 7.83 (br s, 1H), 7.93 (s, 1H), 8.17 (s, 1H), 8.25 (m, 1H), 8.35 (m, 1H), 8.54 (m, 1H). | 2-{[(3S,4R)-3- fluoropiperidin-4- yl]oxy}-5-[1-(2- hydroxy-2- methylpropyl)-1H- pyrazol-4-yl]pyridine- 3-carboxamide hydrochloride (Preparation 56) and [5- (cyclopropyloxy) pyridin-2-yl]acetic acid (Preparation 211). |
| | 2-{[(3S,4R)-1-{[5-(cyclopropyloxy)pyridin-2-yl]acetyl}-3- | | |
| | fluoropiperidin-4-yl]oxy}-5-[1-(2-hydroxy-2- | | |
| | methylpropyl)-1H-pyrazol-4-yl]pyridine-3-carboxamide | | |
| 33 | 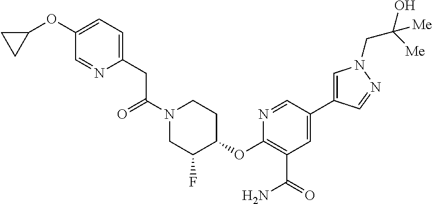 [SMILES: CC(C)(O)Cn1cc(-c2cnc(O[C@H]3CCN(C(=O)Cc4ccc(OC5CC5)cn4)C[C@H]3F)c(C(N)=O)c2)cn1] | MS m/z 553 [M + H]⁺ ¹H NMR (400 MHz, DMSO-d₆): δ ppm 0.67 (m, 2H), 0.79 (m, 2H), 1.08 (s, 6H), 1.80 (m, 1H), 1.99 (m, 1H), 2.90-3.20 (m, 1H), 3.50-4.70 (m, 7H), 5.00-5.20 (m, 1H), 5.50 (m, 1H), 7.23 (d, 1H), 7.47 (m, 1H), 7.53 (br s, 1H), 7.83 (br s, 1H), 7.93 (s, 1H), 8.17 (s, 1H), 8.25 (m, 1H), 8.35 (m, 1H), 8.54 (m, 1H). | 2-{[(3R,4S)-3- fluoropiperidin-4- yl]oxy}-5-[1-(2- hydroxy-2- methylpropyl)-1H- pyrazol-4-yl]pyridine- 3-carboxamide hydrochloride (Preparation 57) and [5-(cyclopropyl oxy)pyridin-2- yl]acetic acid (Preparation 211). |
| | 2-{[(3R,4S)-1-{[5-(cyclopropyloxy)pyridin-2-yl]acetyl}-3- | | |
| | fluoropiperidin-4-yl]oxy}-5-[1-(2-hydroxy-2-methylpropyl)- | | |
| | 1H-pyrazol-4-yl]pyridine-3-carboxamide | | |
| 34 | 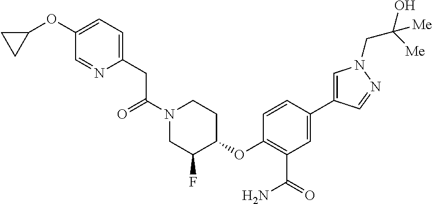 [SMILES: CC(C)(O)Cn1cc(-c2ccc(O[C@H]3CCN(C(=O)Cc4ccc(OC5CC5)cn4)C[C@@H]3F)c(C(N)=O)c2)cn1] | MS m/z 552 [M + H]⁺ ¹H NMR (400 MHz, DMSO-d₆): δ ppm 0.67 (m, 2H), 0.79 (m, 2H), 1.68 (m, 1H), 2.01 (m, 1H), 3.40-4.15 (m, 9H), 4.72- 4.08 (m, 3H), 7.26 (m, 2H), 7.47 (m, 1H), 7.54 (m, 2H), 7.63 (m, 1H), 7.82 (m, 2H), 8.05 (s, 1H), 8.26 (s, 1H). | 2-{[(3S,4S)-3-fluoro piperidin-4-yl]oxy}-5- [1-(2-hydroxy-2- methyl propyl)-1H- pyrazol-4- yl]benzamide hydrochloride (Preparation 58) and [5-(cyclopropyl oxy)pyridin-2- yl]acetic acid (Preparation 211) and PM D. |
| | 2-{[(3S,4S)-1-{[5-(cyclopropyloxy)pyridin-2-yl]acetyl}-3- | | |
| | fluoropiperidin-4-yl]oxy}-5-[1-(2-hydroxy-2- | | |
| | methylpropyl)-1H-pyrazol-4-yl]benzamide | | |
| 35 | 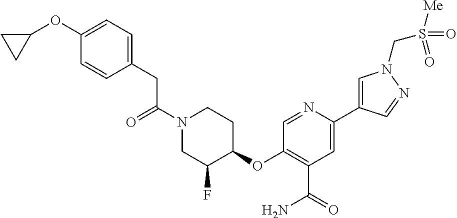 [SMILES: CS(=O)(=O)Cn1cc(-c2cc(C(N)=O)c(O[C@@H]3CCN(C(=O)Cc4ccc(OC5CC5)cc4)C[C@@H]3F)cn2)cn1] | MS m/z 572 [M + H]⁺ ¹H NMR (400 MHz, DMSO-d₆): δ ppm 0.62 (m, 2H), 0.76 (m, 2H), 1.70 (m, 1H), 1.98 (m, 1H), 2.90-3.45 (m, 5H), 3.46- 3.95 (m, 3H), 4.20-4.50 (m, 1H), 4.90-5.10 (m, 2H), 5.78 (s, 2H), 6.96 (m, 2H), 7.13 (m, 2H), 7.67 (br s, 1H), 7.88 (br s, 2H), 8.20 (s, 1H), 8.40 (s, 1H), 8.60 (d, 1H). | 5-{[(3S,4R)-3- fluoropiperidin-4- yl]oxy}-2-{1- [(methylsulfonyl) methyl]-1H-pyrazol-4- yl}pyridine-4- carboxamide hydrochloride (Preparation 59) and 4-cyclopropoxy phenylacetic acid. |
| | 5-{[(3S,4R)-1-{[4-(cyclopropyloxy)phenyl]acetyl}-3- | | |
| | fluoropiperidin-4-yl]oxy}-2-{1-[(methylsulfonyl)methyl]- | | |
| | 1H-pyrazol-4-yl}pyridine-4-carboxamide | | |
| 36 | 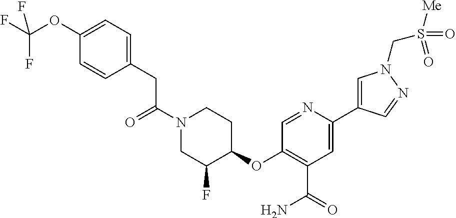 [SMILES: CS(=O)(=O)Cn1cc(-c2cc(C(N)=O)c(O[C@@H]3CCN(C(=O)Cc4ccc(OC(F)(F)F)cc4)C[C@@H]3F)cn2)cn1] | MS m/z 600 [M + H]⁺ ¹H NMR (400 MHz, DMSO-d₆): δ ppm 1.77 (m, 1H), 1.97 (m, 1H), 2.90- 3.35 (m, 4H), 3.45-4.00 (m, 3H), 4.94-5.12 (m, 2H), 5.77 (s, 2H), 7.29- 7.36 (m, 4H), 7.68 (br s, 1H), 7.88 (s, 2H), 8.20 (s, 1H), 8.41 (s, 1H), 8.60 (m, 1H). | 5-{[(3S,4R)-3- fluoropiperidin-4- yl]oxy}-2-{1- [(methylsulfonyl) methyl]-1H-pyrazol-4- yl}pyridine-4- carboxamide hydrochloride (Preparation 59) and 4- trifluoromethoxy- phenylacetic acid. |
| | 5-{[(3S,4R)-3-fluoro-1-{[4-(trifluoromethoxy)phenyl] | | |
| | acetyl}piperidin-4-yl]oxy}-2-{1-[(methylsulfonyl)methyl]- | | |
| | 1H-pyrazol-4-yl}pyridine-4-carboxamide | | |
| 37 | 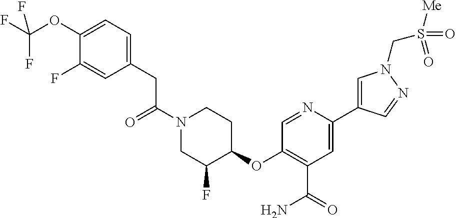 [SMILES: CS(=O)(=O)Cn1cc(-c2cc(C(N)=O)c(O[C@@H]3CCN(C(=O)Cc4ccc(OC(F)(F)F)c(F)c4)C[C@@H]3F)cn2)cn1] | MS m/z 618 [M + H]⁺ ¹H NMR (400 MHz, DMSO-d₆): δ ppm 1.78 (m, 2H), 1.98 (m, 2H), 2.95- 3.40 (m, 4H), 3.50-4.00 (m, 3H), 4.23-4.56 (m, 2H), 4.95-5.15 (m, 2H), 5.78 (s, 2H), 7.16 (m, 1H), 7.35 (m, 1H), 7.50 (m, 1H), 7.68 (br s, 1H), 7.88 (br s, 2H), 8.20 (s, 1H), 8.41 (s, 1H), 8.61 (s, 1H). | 5-{[(3S,4R)-3- fluoropiperidin-4- yl]oxy}-2-{1- [(methylsulfonyl) methyl]-1H-pyrazol- 4-yl}pyridine-4- carboxamide hydrochloride (Preparation 59) and 4- trifluoromethoxy-3- fluorophenylacetic acid. |
| | 5-{[(3S,4R)-3-fluoro-1-{[3-fluoro-4-(trifluoromethoxy)phenyl] | | |
| | acetyl}piperidin-4-yl]oxy}-2-{1-[(methylsulfonyl)methyl]- | | |
| | 1H-pyrazol-4-yl}pyridine-4-carboxamide | | |
| 38 | 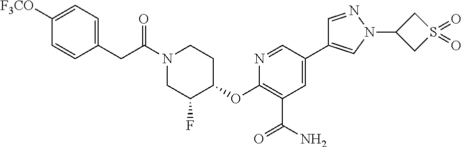 [SMILES: NC(=O)c1cc(-c2cnn(C3CS(=O)(=O)C3)c2)cnc1O[C@H]1CCN(C(=O)Cc2ccc(OC(F)(F)F)cc2)C[C@H]1F] 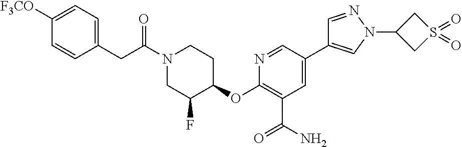 [SMILES: NC(=O)c1cc(-c2cnn(C3CS(=O)(=O)C3)c2)cnc1O[C@@H]1CCN(C(=O)Cc2ccc(OC(F)(F)F)cc2)C[C@@H]1F] | MS m/z 612 [M + H]⁺ ¹H NMR (400 MHz, DMSO-d₆): δ ppm 1.77- 2.02 (m, 1H), 2.32 (m, 1H), 2.95 (m, 1H), 3.40 (m, 1H), 3.87-4.00 (m, 2H), 4.35 (m, 1H), 4.59 (m, 1H), 4.68 (m, 2H), 4.78 (m, 2H), 5.02 (m, 0.5H), 5.33 (m, 0.5H), 5.33-5.38 (m, 1H), 5.45- 5.60 (m, 1H), 7.28-7.36 (m, 4H), 7.54 (br s, 1H), 7.86 (br s, 1H), 8.13 (s, 1H), 8.40 (d, 1H), 8.52 (s, 1H), 8.57 (d, 1H). | 1:1 Cis-racemic 5-[1- (1,1-dioxidothietan- 3-yl)-1H-pyrazol-4- yl]-2-{[3- fluoropiperidin-4- yl]oxy}pyridine-3- carboxamide hydrochloride (Preparation 60) and 4- trifluoromethoxy- phenylacetic acid and PM C. |
| | 1:1 Cis-racemic 5-[1-(1,1-dioxidothietan-3-yl)-1H- | | |
| | pyrazol-4-yl]-2-{[3-fluoro-1-{[4-(trifluoromethoxy)phenyl] | | |
| | acetyl}piperidin-4-yl]oxy}pyridine-3-carboxamide | | |
| 39 | 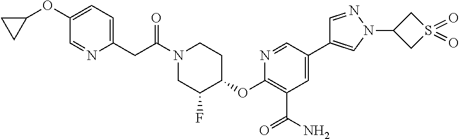 [SMILES: NC(=O)c1cc(-c2cnn(C3CS(=O)(=O)C3)c2)cnc1O[C@H]1CCN(C(=O)Cc2ccc(OC3CC3)cn2)C[C@H]1F] 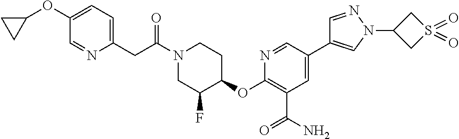 [SMILES: NC(=O)c1cc(-c2cnn(C3CS(=O)(=O)C3)c2)cnc1O[C@@H]1CCN(C(=O)Cc2ccc(OC3CC3)cn2)C[C@@H]1F] | MS m/z 585 [M + H]⁺ ¹H NMR (400 MHz, DMSO-d₆): δ ppm 1.80- 1.90 (m, 1H), 2.32 (m, 1H), 3.17 (m, 1H), 3.32 (m, 1H), 3.75-4.08 (m, 4H), 4.28 (m, 1H), 4.56 (m, 1H), 4.72 (m, 2H), 4.81 (m, 2H), 5.00 (m, 0.5H), 5.14 (m, 0.5H), 5.33-5.38 (m, 1H), 5.48- 5.54 (m, 1H), 7.22 (m, 1H), 7.47 (m, 1H), 7.54 (br s, 1H), 7.86 (br s, 1H), 8.13 (s, 1H), 8.25 (d, 1H), 8.40 (d, 1H), 8.51 (s, 1H), 8.57 (s, 1H). | 1:1 Cis-racemic 5-[1- (1,1-dioxidothietan- 3-yl)-1H-pyrazol-4- yl]-2-{[3- fluoropiperidin-4- yl]oxy}pyridine-3- carboxamide hydrochloride (Preparation 60) and [5- (cyclopropyloxy) pyridin-2-yl]acetic acid (Preparation 211) and PM C. |
| | 1:1 Cis-racemic 2-{[1-{[5-(cyclopropyloxy)pyridin-2-yl]acetyl}-3- | | |
| | fluoropiperidin-4-yl]oxy}-5-[1-(1,1-dioxidothietan- | | |
| | 3-yl)-1H-pyrazol-4-yl]pyridine-3-carboxamide | | |
| 40 | 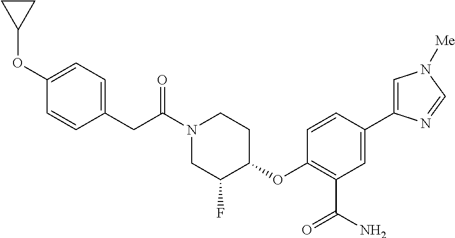 [SMILES: Cn1cnc(-c2ccc(O[C@H]3CCN(C(=O)Cc4ccc(OC5CC5)cc4)C[C@H]3F)c(C(N)=O)c2)c1] 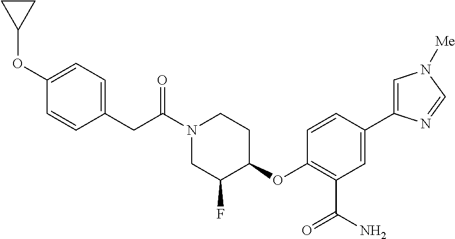 [SMILES: Cn1cnc(-c2ccc(O[C@@H]3CCN(C(=O)Cc4ccc(OC5CC5)cc4)C[C@@H]3F)c(C(N)=O)c2)c1] | MS m/z 493 [M + H]⁺ ¹H NMR (400 MHz, DMSO-d₆): δ ppm 0.60 (m, 2H), 0.78 (m, 2H), 1.68 (m, 1H), 2.00 (m, 1H), 3.31-3.79 (m, 9H), 4.01 (m, 1H), 4.70-4.80 (m, 2H), 6.98 (m, 2H), 7.14 (m, 2H), 7.24 (m, 1H), 7.51 (br s, 2H), 7.51 (s, 1H), 7.60 (s, 1H), 7.77 (m, 1H), 8.04 (s, 1H). | 1:1 Cis-racemic 2- {[3-fluoropiperidin-4- yl]oxy}-5-(1-methyl- 1H-imidazol-4- yl)benzamide hydrochloride (Preparation 61) and 4- cyclopropoxyphenyl acetic acid. |
| | 1:1 Cis-racemic 2-{[1-{[4-(cyclopropyloxy)phenyl] | | |
| | acetyl}-3-fluoropiperidin-4-yl]oxy}-5-(1-methyl-1H- | | |
| | imidazol-4-yl)benzamide | | |
| 41 | 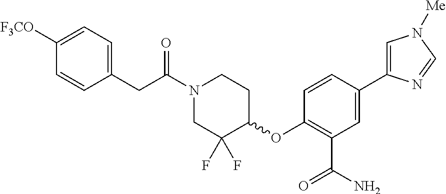 [SMILES: Cn1cnc(-c2ccc(OC3CCN(C(=O)Cc4ccc(OC(F)(F)F)cc4)CC3(F)F)c(C(N)=O)c2)c1] | MS m/z 539 [M + H]⁺ ¹H NMR (400 MHz, DMSO-d₆): δ ppm 1.70- 1.85 (m, 1H), 2.00-2.10 (m, 1H), 3.39 (m, 0.5H), 3.52 (m, 0.5H), 3.65 (s, 3H), 3.70-3.95 (m, 4H), 4.15-4.30 (m, 1H), 5.10 (m, 1H), 7.25-7.35 (m, 5H), 7.45 (br s, 1H), 7.60 (m, 2H), 7.61 (s, 1H), 7.80 (m, 1H), 8.05 (s, 1H). | Racemic 2-((3,3- difluoropiperidin-4- yl)oxy)-5-(1-methyl- 1H-imidazol-4- yl)benzamide hydrochloride (Preparation 62) and 4- trifluoromethoxy- phenylacetic acid and PM E. |
| | Racemic 2-[(3,3-difluoro-1-{[4-(trifluoromethoxy)phenyl] | | |
| | acetyl}piperidin-4-yl)oxy]-5-(1-methyl-1H-imidazol- | | |
| | 4-yl)benzamide | | |
| 42 | 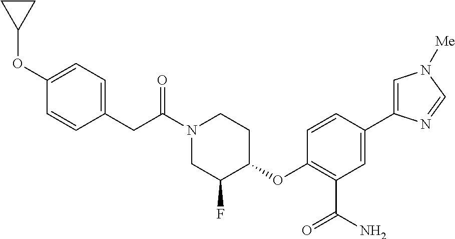 [SMILES: Cn1cnc(-c2ccc(O[C@H]3CCN(C(=O)Cc4ccc(OC5CC5)cc4)C[C@@H]3F)c(C(N)=O)c2)c1] | MS m/z 493 [M + H]⁺ ¹H NMR (400 MHz, DMSO-d₆): δ ppm 0.63 (m, 2H), 0.75 (m, 2H), 1.66 (m, 1H), 2.00 (m, 1H), 3.43 (m, 1H), 3.55-3.79 (m, 8H), 4.01 (m, 1H), 4.70-4.80 (m, 2H), 6.99 (m, 2H), 7.16 (m, 2H), 7.22 (m, 1H), 7.52-7.60 (m, 3H), 7.77 (m, 1H), 8.04 (s, 1H). | 2-{[(3S,4S)-3- fluoropiperidin-4- yl]oxy}-5-(1-methyl- 1H-imidazol-4- yl)benzamide hydrochloride (Preparation 47) and 4- cyclopropoxyphenyl acetic acid and PM E. |
| | 2-{[(3S,4S)-1-{[4-(cyclopropyloxy)phenyl]acetyl}- | | |
| | 3-fluoropiperidin-4-yl]oxy}-5-(1-methyl-1H- | | |
| | imidazol-4-yl)benzamide | | |
| 43 | 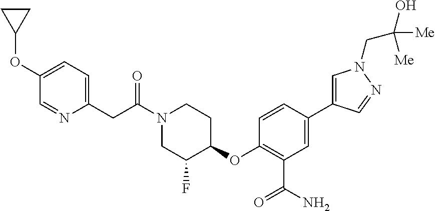 [SMILES: CC(C)(O)Cn1cc(-c2ccc(O[C@@H]3CCN(C(=O)Cc4ccc(OC5CC5)cn4)C[C@H]3F)c(C(N)=O)c2)cn1] 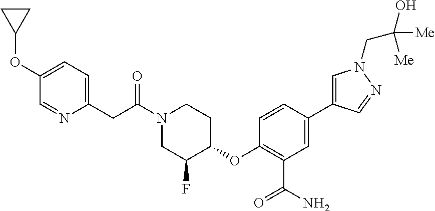 [SMILES: CC(C)(O)Cn1cc(-c2ccc(O[C@H]3CCN(C(=O)Cc4ccc(OC5CC5)cn4)C[C@@H]3F)c(C(N)=O)c2)cn1] | MS m/z 552 [M + H]⁺ ¹H NMR (400 MHz, MeOH-d₄): δ ppm 0.72 (m, 2H), 0.85 (m, 2H), 1.15 (s, 6H), 1.83 (m, 1H), 2.14 (m, 1H), 3.30 (m, 1H), 3.60-4.15 (m, 10H), 7.23- 7.30 (m, 2H), 7.52 (m, 1H), 7.66 (m, 1H), 7.80 (s, 1H), 7.96 (m, 2H), 8.23 (s, 1H). | 1:1 Trans-racemic-2- ((3-fluoropiperidin-4- yl)oxy)-5-(1-(2- hydroxy-2- methylpropyl)-1H- pyrazol-4- yl)benzamide hydrochloride (Preparation 63) and [5- (cyclopropyloxy) pyridin-2-yl]acetic acid (Preparation 211). |
| | 1:1 Trans-racemic-2-{[5-(cyclopropyloxy)pyridin-2-yl]acetyl}-3- | | |
| | fluoropiperidin-4-yl]oxy}-5-[1-(2-hydroxy-2- | | |
| | methylpropyl)-1H-pyrazol-4-yl]benzamide | | |
| 44 | 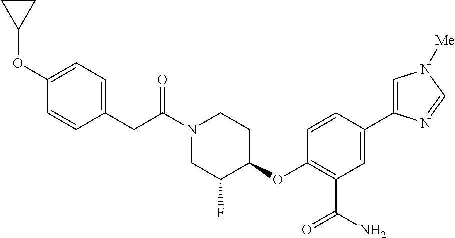 [SMILES: Cn1cnc(-c2ccc(O[C@@H]3CCN(C(=O)Cc4ccc(OC5CC5)cc4)C[C@H]3F)c(C(N)=O)c2)c1] 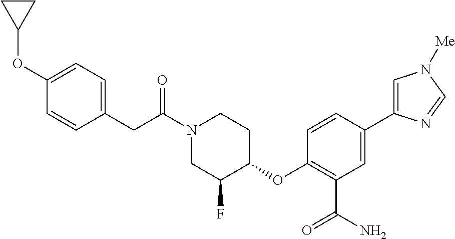 [SMILES: Cn1cnc(-c2ccc(O[C@H]3CCN(C(=O)Cc4ccc(OC5CC5)cc4)C[C@@H]3F)c(C(N)=O)c2)c1] | MS m/z 493 [M + H]⁺ ¹H NMR (400 MHz, DMSO-d₆): δ ppm 0.62 (m, 2H), 0.78 (m, 2H), 1.68 (m, 1H), 2.00 (m, 1H), 3.31-4.05 (m, 10H), 4.70- 4.82 (m, 2H), 6.98 (m, 2H). 7.14 (m, 2H), 7.24 (m, 1H), 7.51-7.60 (m, 4H), 7.77 (m, 1H), 8.04 (s, 1H). | 1:1 Trans-racemic 2- {[3-fluoropiperidin-4- yl]oxy}-5-(1-methyl- 1H-imidazol-4- yl)benzamide hydrochloride (Preparation 67) and 4- cyclopropoxyphenyl acetic acid and PM E. |
| | 1:1 Trans-racemic-2-{[4-(cyclopropyloxy)phenyl] | | |
| | acetyl}-3-fluoropiperidin-4-yl]oxy}-5-(1-methyl- | | |
| | 1H-imidazol-4-yl)benzamide | | |
| 45 | 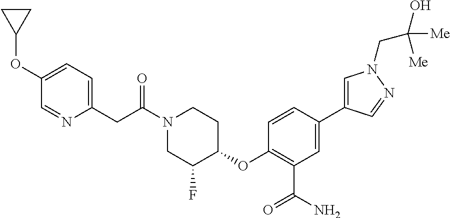 [SMILES: CC(C)(O)Cn1cc(-c2ccc(O[C@H]3CCN(C(=O)Cc4ccc(OC5CC5)cn4)C[C@H]3F)c(C(N)=O)c2)cn1] 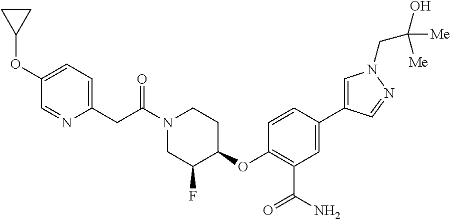 [SMILES: CC(C)(O)Cn1cc(-c2ccc(O[C@@H]3CCN(C(=O)Cc4ccc(OC5CC5)cn4)C[C@@H]3F)c(C(N)=O)c2)cn1] | MS m/z 552 [M + H]⁺ 1H NMR (400 MHz, DMSO-d₆): δ ppm 0.67 (m, 2H), 0.80 (m, 2H), 1.07 (s, 6H), 1.69 (m, 1H), 2.03 (m, 1H), 3.49-4.10 (m, 9H), 4.71-4.83 (m, 2H), 7.26 (m, 2H), 7.47 (m, 1H), 7.54 (m, 2H), 7.63 (m, 1H), 7.83 (m, 2H), 8.05 (s, 1H), 8.26 (m, 1H). | 1:1 Cis-racemic-2- ((3-fluoropiperidin-4- yl)oxy)-5-(1-(2- hydroxy-2- methylpropyl)-1H- pyrazol-4- yl)benzamide hydrochloride (Preparation 65) and [5- (cyclopropyloxy) pyridin-2-yl]acetic acid (Preparation 211). |
| | 1:1 Cis-racemic-2-{[5-(cyclopropyloxy)pyridin- | | |
| | 2-yl]acetyl}-3-fluoropiperidin-4-yl]oxy}- | | |
| | 5-[1-(2-hydroxy-2-methylpropyl)-1H-pyrazol-4-yl]benzamide | | |
| 46 | 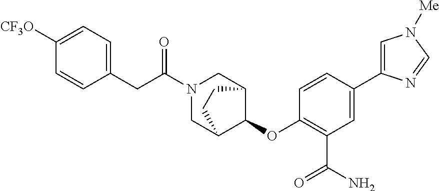 | MS m/z 529 [M + H]⁺ 1H NMR (400 MHz, DMSO-d₆): δ ppm 1.23- 1.38 (m, 4H), 1.72 (m, 2H), 2.32-2.40 (m, 2H), 3.06 (m, 1H), 3.48 (m, 1H), 3.59 (m, 1H), 3.66 (s, 3H), 3.99 (m, 1H), 4.66 (m, 1H), 7.22 (m, 1H), 7.28-7.36 (m, 4H), 7.51- 7.60 (m, 4H), 7.75 (m, 1H), 7.98 (m, 1H). | 2-((1R,5S,8r)-3- azabicyclo[3.2.1]oct an-8-yloxy)-5-(1- methyl-1H-imidazol- 4-yl)benzamide (Preparation 73) and 4- trifluoromethoxy- phenylacetic acid and PM C. |
| | 5-(1-methyl-1H-imidazol-4-yl)-2-(((1R,5S,8r)-3-(2- | | |
| | (4-(trifluoromethoxy)phenyl)acetyl)-3- | | |
| | azabicyclo[3.2.1]octan-8-yl)oxy)benzamide | | |
| 47 | 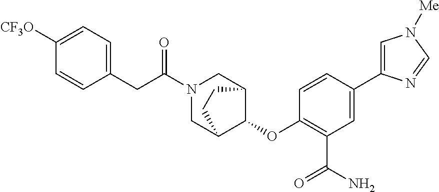 | MS m/z 529 [M + H]⁺ ¹H NMR (400 MHz, DMSO-d₆): δ ppm 1.32 (m, 2H), 1.71 (m, 2H), 2.41 (m, 2H), 2.86 (m, 1H), 3.28 (m, 1H), 3.66 (s, 3H), 3.79-3.84 (m, 3H), 4.25 (m, 1H), 4.82 (s, 1H), 7.24-7.49 (m, 6H), 7.53- 7.59 (m, 3H), 7.78 (m, 1H), 8.13 (m, 1H). | 2-((1R,5S,8s)-3- azabicyclo[3.2.1] octan-8-yloxy)-5-(1- methyl-1H-imidazol- 4-yl)benzamide (Preparation 74) and 4- trifluoromethoxy- phenylacetic acid and PM C. |
| | 5-(1-methyl-1H-imidazol-4-yl)-2-(((1R,5S,8s)-3-(2- | | |
| | (4-(trifluoromethoxy)phenyl)acetyl)-3- | | |
| | azabicyclo[3.2.1]octan-8-yl)oxy)benzamide | | |
| 48 | 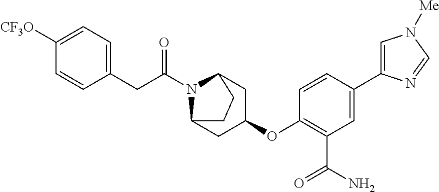 | MS m/z 529 [M + H]⁺ ¹H NMR (400 MHz, DMSO-d₆): δ ppm 1.56- 1.65 (m, 2H), 1.82 (m, 2H), 1.92 (m, 1H), 2.23 (m, 2H), 3.68-3.84 (m, 5H), 4.54 (m, 2H), 4.97 (m, 1H), 7.31 (m, 3H), 7.39 (m, 2H), 7.51 (m, 2H), 7.59 (br s, 1H), 7.75- 7.78 (m, 2H), 8.14 (s, 1H). | 2-((1R,3s,5S)-8- azabicyclo[3.2.1] octan-3-yloxy)-5-(1- methyl-1H-imidazol- 4-yl)benzamide hydrochloride (Preparation 72) and 4- trifluoromethoxy- phenylacetic acid. |
| | 5-(1-methyl-1H-imidazol-4-yl)-2-(((1R,3s,5S)-8-(2- | | |
| | (4-(trifluoromethoxy)phenyl)acetyl)-8- | | |
| | azabicyclo[3.2.1]octan-3-yl)oxy)benzamide | | |
| 49 | 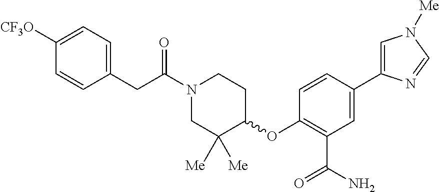 | MS m/z 531 [M + H]⁺ ¹H NMR (400 MHz, DMSO-d₆): δ ppm 0.98 (s, 3H), 1.01 (s, 3H), 1.64- 1.67 (m, 1H), 1.86-1.89 (m, 1H), 3.18 (m, 1H), 3.36 (m, 1H), 3.63-3.66 (m, 1H), 3.74 (s, 3H), 3.79 (s, 2H), 3.85 (m, 1H), 4.41-4.44 (m, 1H), 7.22- 7.27 (m, 5H), 7.39 (d, 2H), 7.58 (s, 1H), 7.76 (dd, 1H), 7.93 (br s, 1H), 8.06 (d, | Racemic 2-((3,3- dimethylpiperidin-4- yl)oxy)-5-(1-methyl- 1H-imidazol-4- yl)benzamide (Preparation 75) and 4- trifluoromethoxy- phenylacetic acid and PM E. |
| | Racemic 2-((3,3-dimethyl-1-(2-(4-(trifluoromethoxy) | 1H). | |
| | phenyl)acetyl)piperidin-4-yl)oxy)-5-(1-methyl- | | |
| | 1H-imidazol-4-yl)benzamide | | |
| 50 | 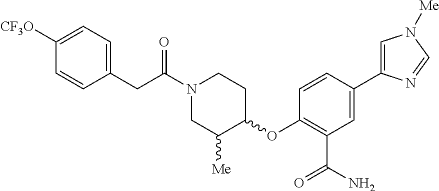 | MS m/z 517 [M + H]⁺ ¹H NMR (400 MHz, DMSO-d₆): δ ppm 0.97 (m, 3H), 1.46 (m, 1H), 1.63 (m, 1H), 1.83 (m, 1H), 2.04 (m, 1H), 3.20 (m, 1H), 3.66 (s, 3H), 3.74- 3.95 (m, 3H), 4.36 (m, 1H), 4.73 (m, 1H), 7.15 (m, 1H), 7.28-7.40 (m, 4H), 7.47-7.59 (m, 4H), 7.75 (m, 1H), 8.02-8.12 (m, 1H). | Racemic 5-(1- methyl-1H-imidazol- 4-yl)-2-((3- methylpiperidin-4- yl)oxy)benzamide hydrochloride (Preparation 66) and 4- trifluoromethoxy- phenylacetic acid and PM |
| | Racemic 2-((3-methyl-1-(2-(4-(trifluoromethoxy) | | |
| | phenyl)acetyl)piperidin-4-yl)oxy)-5- | | |
| | (1-methyl-1H-imidazol-4-yl)benzamide | | |
| 51 | 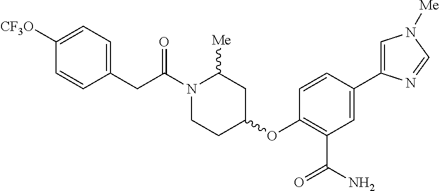 | MS m/z 517 [M + H]⁺ ¹H NMR (400 MHz, DMSO-d₆): δ ppm 1.10 (m, 3H), 1.40 (m, 1H), 1.67 (m, 1H), 2.00-2.20 (m, 2H), 3.28 (m, 1H), 3.66 (s, 3H), 3.74-3.92 (m, 3H), 4.46 (m, 1H), 4.88 (m, 1H), 7.23-7.34 (m, 5H), 7.49 (br s, 1H), 7.53 (s, 1H), 7.59 (s, 1H), 7.76 (dd, 1H), 8.15 (d, 1H). | Racemic 5-(1- methyl-1H-imidazol- 4-yl)-2-((2- methylpiperidin-4- yl)oxy)benzamide hydrochloride (Preparation 67) and 4- trifluoromethoxy- phenylacetic acid and PM |
| | Racemic 2-((2-methyl-1-(2-(4-(trifluoromethoxy) | | |
| | phenyl)acetyl)piperidin-4-yl)oxy)- | | |
| | 5-(1-methyl-1H-imidazol-4-yl)benzamide | | |
| 52 | 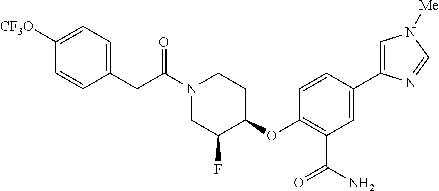 | MS m/z 521 [M + H]⁺ ¹H NMR (400 MHz, DMSO-d₆): δ ppm 1.72 (m, 1H), 2.01 (m, 1H), 3.37- 3.48 (m, 1H), 3.58-3.76 (m, 4H), 3.81-3.83 (m, 2H), 4.03 (m, 1H), 4.83 (m, 2H), 7.24 (d, 1H), 7.29 (d, 2H), 7.35 (d, 2H), 7.52 (br s, 2H), 7.55 (s, 1H), 7.60 (s, 1H), 7.77 (d, 1H), 8.05 (s, 1H). | 1:1 racemic mixture of 2-{[(3S,4R)-3- fluoropiperidin-4- yl]oxy}-5-(1-methyl- 1H-imidazol-4- yl)benzamide hydrochloride and (3R,4S) enantiomer) (analogous to Preparation 43) and 4-trifluoromethoxy- phenylacetic acid and PM C. |
| | 1:1 racemic mixture of 2-(((3S,4R)-3-fluoro-1-(2-(4- | | |
| | (trifluoromethoxy)phenyl)acetyl)piperidin-4-yl)oxy)- | | |
| | 5-(1-methyl-1H-imidazol-4-yl)benzamide + (3R,4S) enantiomer | | |
| 53 | 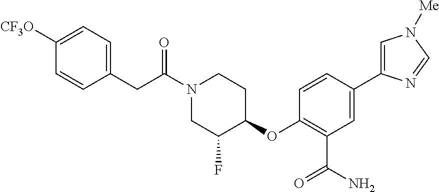 | MS m/z 521 [M + H]⁺ ¹H NMR (400 MHz, DMSO-d₆): δ ppm 1.83- 1.88 (m, 1H), 1.96-1.99 (m, 1H), 3.17 (br s, 1H), 3.42 (br s, 1H), 3.69 (s, 3H), 3.81 (d, 2H), 4.13 (br s, 1H), 4.39 (br s, 1H), 4.83-4.91 (m, 1H), 4.90 (m, 0.5H), 5.08 (m, 0.5H), 7.23-7.27 (m, 3H), 7.37 (d, 2H), 7.46 (s, 1H), 7.57 (s, 1H), 7.80 (d, 1H), 8.21 (s, | 2-(((3R,4R)-3- fluoropiperidin-4- yl)oxy)-5-(1-methyl- 1H-imidazol-4- yl)benzamide hydrochloride (Preparation 68) and 4-trifluoro- methoxy- phenylacetic acid and PM C. |
| | 2-(((3R,4R)-3-fluoro-1-(2-(4-(trifluoromethoxy)phenyl) | 1H). | |
| | acetyl)piperidin-4-yl)oxy)-5-(1-methyl-1H-imidazol- | | |
| | 4-yl)benzamide | | |
| 54 | 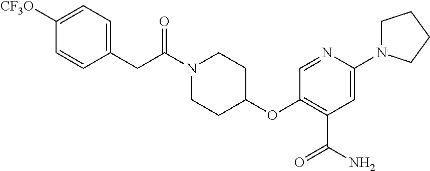 | MS m/z 493 [M + H]⁺ ¹H NMR (400 MHz, DMSO-d₆): δ ppm 1.56 (m, 2H), 1.83 (m, 2H), 1.92 (m, 4H), 3.77 (m, 5H), 4.40 (m, 1H), 6.57 (s, 1H), 7.28-7.35 (m, 4H), 7.67 (m, 2H), 8.01 (s, 1H). | 5-(piperidin-4-yloxy)- 2-(pyrrolidin-1- yl)isonicotinamide hydrochloride (Preparation 70) and 4- trifluoromethoxy- phenylacetic acid and PM C. |
| | 2-(pyrrolidin-1-yl)-5-((1-(2-(4- | | |
| | (trifluoromethoxy)phenyl) | | |
| | acetyl)piperidin-4-yl)oxy)isonicotinamide | | |
| 55 | 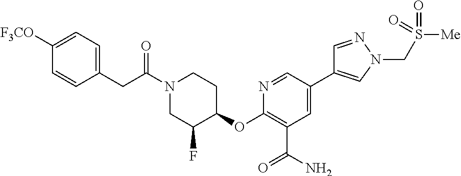 [SMILES: CS(=O)(=O)Cn1cc(-c2cnc(O[C@@H]3CCN(C(=O)Cc4ccc(OC(F)(F)F)cc4)C[C@@H]3F)c(C(N)=O)c2)cn1] | NMR exhibits rotameric behaviour: ¹H NMR (400 MHz, DMSO-d₆): δ ppm 1.90-1.98 (m, 1H), 2.00- 2.05 (m, 1H), 2.95-3.23 (m, 4H), 3.50-4.00 (m, 3H), 4.32 (m, 1H), 4.60 (m, 1H), 5.03-5.18 (m, 1H), 5.48-5.55 (m, 1H), 5.76 (d, 2H), 7.31-7.36 (m, 4H), 7.55 (br s, 1H), 7.87 (br s, 1H), 8.19 (s, 1H), | 2-{[(3S,4R)-3- fluoropiperidin-4- yl]oxy}-5-{1- [(methylsulfonyl) methyl]-1H-pyrazol-4- yl}pyridine-3- carboxamide hydrochloride (Preparation 46) and (4-trifluoromethoxy) phenylacetic acid. |
| | 2-{[(3S,4R)-3-fluoro-1-{[4-(trifluoromethoxy)phenyl] | 8.40 (m, 2H), 8.60 (br s, | |
| | acetyl}piperidin-4-yl]oxy}-5-{1-[(methylsulfonyl)methyl]- | 1H). | |
| | 1H-pyrazol-4-yl}pyridine-3-carboxamide | MS m/z 600 [M + H]⁺ | |

The following Examples 56-93 were prepared according to Methods 1, 2, 4, 5 and 10 (Examples 1,2,4,5 or 11) using compounds of formulae (II) and either (III) or (IV), and Purification Method (PM) below if different from the method described. Sodium, potassium or cesium carbonate may be used as base in these methods.

Purification Method A: Reverse phase column chromatography eluting with 0.1% formic acid in acetonitrile and 0.1% formic acid in water;

Purification Method B: Silica gel column chromatography eluting with 90:10:0.2 EtOAc:MeOH:NH₃followed by preparative HPLC;

Purification Method C: Silica gel column chromatography eluting with 5-6% MeOH in DCM or EtOAc;

Purification Method D: Preparative TLC eluting with 2-6% MeOH in DCM;

Purification Method E: Silica gel column chromatography eluting with 3% MeOH in DCM followed by preparative TLC eluting with 7% IPA in EtOAc;

Purification Method F: Silica gel column chromatography eluting with 3% MeOH in DCM;

Purification Method G: Silica gel column chromatography eluting with 5% MeOH in DCM followed by preparative TLC eluting with 10% MeOH in DCM.

| | | | Starting materials |
|---|---|---|---|
| Example | Structure and Name | Data | & PM |
| 56 | 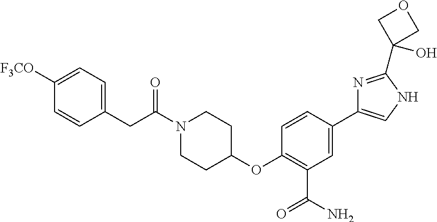 [SMILES: NC(=O)c1cc(-c2c[nH]c(C3(O)COC3)n2)ccc1OC1CCN(C(=O)Cc2ccc(OC(F)(F)F)cc2)CC1] | MS m/z 561 [M + H]⁺ ¹H NMR (400 MHz, Acetone-d₆): δ ppm 1.75- 1.85 (m, 2H), 2.05-2.33 (m, 2H), 3.38-3.55 (m, 2H), 3.85 (s, 2H), 3.89- 4.06 (m, 2H), 4.87-4.92 (m, 1H), 4.72-5.09 (m, 4H), 6.78 (br s, 1H), 7.23-7.28 (m, 3H), 7.41- 7.47 (m, 3H), 7.61 (br s, 1H), 7.93 (d, 1H), 8.47 (s, 1H). | 3-(5-bromo-1H- imidazol-2-yl)oxetan- 3-ol (Preparation 186), 5-(4,4,5,5- tetramethyl-1,3,2- dioxaborolan-2-yl)-2- [(1-{[4- (trifluoromethoxy) phenyl]acetyl} piperidin-4- yl)oxy]benzamide (Preparation 14) and PM A. |
| | 5-[2-(3-hydroxyoxetan-3-yl)-1H-imidazol- | | |
| | 5-yl]-2-[(1-{[4-(trifluoromethoxy)phenyl] | | |
| | acetyl}piperidin-4-yl)oxy]benzamide | | |
| 57 | 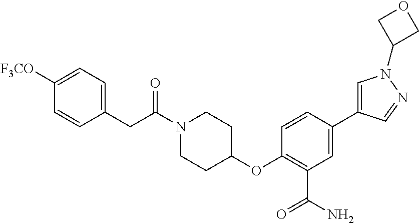 [SMILES: NC(=O)c1cc(-c2cnn(C3COC3)c2)ccc1OC1CCN(C(=O)Cc2ccc(OC(F)(F)F)cc2)CC1] | MS m/z 545 [M + H]⁺ ¹H NMR (400 MHz, CD₃OD): δ ppm 1.62- 1.82 (m, 2H), 1.90-2.09 (m, 2H), 3.48-3.58 (m, 2H), 3.78-3.98 (m, 4.59-4.62 (m, 1H), 4.05 (d, 4H), 5.56-5.61 (m, 1H), 7.12-7.22 (m, 3H), 7.58 (d, 1H), 7.63 (d, 1H), 7.90 (s, 1H), 8.03 (s, 1H) and 8.15 (s, 1H). | 4-bromo-1-(oxetan-3- yl)-1H-pyrazole (Preparation 189), 5- (4,4,5,5-tetramethyl- 1,3,2-dioxaborolan-2- yl)-2-[(1-{[4- (trifluoromethoxy) phenyl]acetyl} piperidin-4- yl)oxy]benzamide (Preparation 14) and PM B. |
| | 5-[1-(oxetan-3-yl)-1H-pyrazol-4-yl]-2-[(1-{[4- | | |
| | (trifluoromethoxy)phenyl] | | |
| | acetyl}piperidin-4-yl)oxy]benzamide | | |
| 58 | 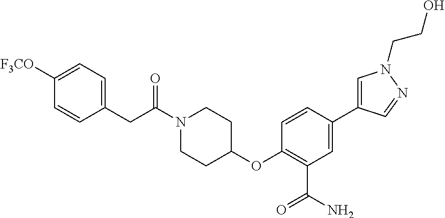 [SMILES: NC(=O)c1cc(-c2cnn(CCO)c2)ccc1OC1CCN(C(=O)Cc2ccc(OC(F)(F)F)cc2)CC1] | MS m/z 533 [M + H]⁺ ¹H NMR (400 MHz, DMSO-d₆): δ ppm 1.62 (m, 2H), 1.93 (m, 2H), 3.29-3.35 (m, 2H), 3.74- 3.79 (m, 6H), 4.14 (m, 2H), 4.77 (m, 1H), 4.92 (t, 1H), 7.19 (d, 1H), 7.28-7.36 (m, 4H), 7.53-7.62 (m, 3H), 7.81 (d, 1H), 8.10 (s, 1H). | 2-(4-bromo-1H- pyrazol-1-yl)ethanol, 5-(4,4,5,5-tetramethyl- 1,3,2-dioxaborolan-2- yl)-2-[(1-{[4- (trifluoromethoxy) phenyl]acetyl}piperidin- 4-yl)oxy]benzamide (Preparation 14) and PM C. |
| | 5-[1-(2-hydroxyethyl)-1H-pyrazol-4-yl]-2-[(1-{[4- | | |
| | (trifluoromethoxy)phenyl] | | |
| | acetyl}piperidin-4-yl)oxy]benzamide | | |
| 59 | 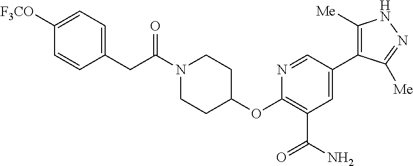 [SMILES: Cc1n[nH]c(C)c1-c1cnc(OC2CCN(C(=O)Cc3ccc(OC(F)(F)F)cc3)CC2)c(C(N)=O)c1] | MS m/z 518 [M + H]⁺ ¹H NMR (400 MHz, DMSO-d₆): δ ppm 1.70 (m, 2H), 1.90 (m, 2H), 2.20 (m, 6H), 3.30-3.40 (m, 2H), 3.80 (m, 4H), 5.40 (m, 1H), 7.29 (m, 2H), 7.35 (m, 2H), 7.57 (s, 1H), 7.73 (s, 1H), 8.00 (m, 1H), 8.20 (m, 1H). | 5-bromo-2-({1-[4- (trifluoromethoxy) benzyl]piperidin-4- yl}oxy)pyridine-3- carboxamide (Preparation 37), 3,5- dimethylpyrazole-4- boronic acid pinacol ester and PM D. |
| | 5-(3,5-dimethyl-1H-pyrazol-4-yl)-2-[(1-{[4- | | |
| | (trifluoromethoxy)phenyl]acetyl}piperidin-4- | | |
| | yl)oxy]pyridine-3-carboxamide | | |
| 60 | 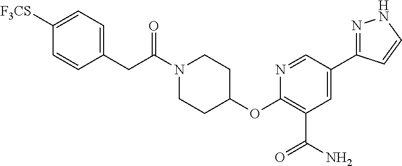 | MS m/z 506 [M + H]⁺ ¹H NMR (400 MHz, DMSO-d₆): δ ppm 1.70 (m, 2H), 1.90 (m, 2H), 3.30 (m, 1H), 3.47 (m, 1H), 3.80 (m, 4H), 5.40 (m, 1H), 6.70 (s, 1H), 7.40 (m, 2H), 7.59 (s, 1H), 7.60 (m, 2H), 7.80 (m, 2H), 12.90 (s, 1H). | 5-bromo-2-{[1-({4- [(trifluoromethyl) sulfanyl]phenyl}acetyl) piperidin-4-yl] oxy}pyridine- 3-carboxamide (Preparation 38), 1H- pyrazol-3-yl boronic acid and PM D. |
| | 5-(1H-pyrazol-3-yl)-2-{[1-({4-[(trifluoromethyl)sulfanyl] | | |
| | phenyl}acetyl)piperidin-4-yl]oxy}pyridine-3-carboxamide | | |
| 62 | 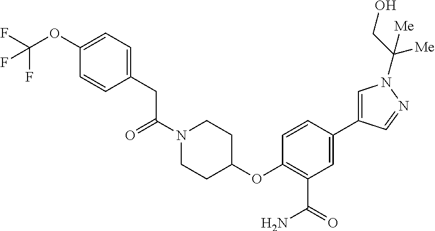 [SMILES: CC(C)(CO)n1cc(-c2ccc(OC3CCN(C(=O)Cc4ccc(OC(F)(F)F)cc4)CC3)c(C(N)=O)c2)cn1] | MS m/z 561 [M + H]⁺ ¹H NMR (400 MHz, DMSO-d₆): δ ppm 1.48 (s, 6H), 1.64 (m, 2H), 1.90 (m, 2H), 3.30-3.43 (m, 2H), 3.59 (d, 2H), 3.79 (m, 4H), 4.76 (m, 1H), 4.96 (t, 1H), 7.19 (d, 1H), 7.29 (7.35 (m, 4H), 7.53 (m, 2H), 7.63 (m, 1H), 7.80 (s, 1H), 7.89 (d, 1H), 8.16 (s, 1H). | 5-(4,4,5,5-tetramethyl- 1,3,2-dioxaborolan-2- yl)-2-[(1-{[4- (trifluoromethoxy) phenyl]acetyl} piperidin-4- yl)oxy]benzamide (Preparation 14) and 2-(4-bromo-1H- pyrazol-4-yl]-2- methylpropan-1-ol (Preparation 206) and PM E. |
| | 5-[1-(1-hydroxy-2-methylpropan-2-yl)-1H- | | |
| | pyrazol-4-yl]-2-[(1-{[4-(trifluoromethoxy)phenyl] | | |
| | acetyl}piperidin-4-yl)oxy]benzamide | | |
| 63 | 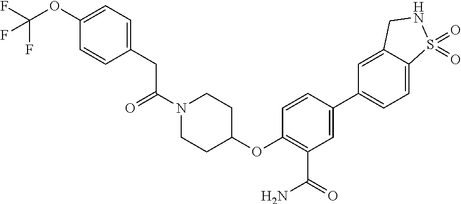 [SMILES: NC(=O)c1cc(-c2ccc3c(c2)CNS3(=O)=O)ccc1OC1CCN(C(=O)Cc2ccc(OC(F)(F)F)cc2)CC1] | MS m/z 590 [M + H]⁺ ¹H NMR (400 MHz, DMSO-d₆): δ ppm 1.69 (m, 2H), 1.92-1.98 (m, 2H), 3.32-3.50 (m, 2H), 3.76-3.79 (m, 4H), 4.45 (s, 2H), 4.87 (m, 1H), 7.29-7.37 (m, 5H), 7.58 (br s, 1H), 7.63 (br s, 1H), 7.80-7.87 (m, 5H), 8.07 (br s, 1H). | 5-(4,4,5,5-tetramethyl- 1,3,2-dioxaborolan-2- yl)-2-[(1-{[4- (trifluoromethoxy) phenyl]acetyl}piperidin- 4-yl)oxy]benzamide (Preparation 14) and 5-bromo-2,3-dihydro- 1,2-benzothiazole 1,1- dioxide and PM C. |
| | 5-(1,1-dioxido-2,3-dihydro-1,2- | | |
| | benzothiazol-5-yl)-2-[(1-{[4-(trifluoromethoxy)phenyl] | | |
| | acetyl}piperidin-4-yl)oxy]benzamide | | |
| 64 | 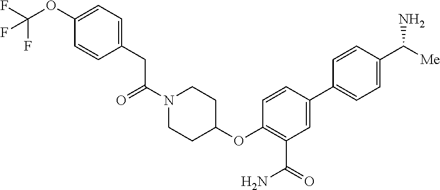 [SMILES: C[C@@H](N)c1ccc(-c2ccc(OC3CCN(C(=O)Cc4ccc(OC(F)(F)F)cc4)CC3)c(C(N)=O)c2)cc1] | MS m/z 542 [M + H]⁺ ¹H NMR (400 MHz, DMSO-d₆): δ ppm 1.27 (d, 3H), 1.67 (m, 2H), 1.92 (m, 2H), 3.39-3.47 (m, 2H), 3.75-3.79 (m, 4H), 4.02 (m, 1H), 4.82 (m, 1H), 7.28-7.37 (m, 5H), 7.45 (m, 2H), 7.55 (m, 4H), 7.69 (m, 1H), 7.97 (s, 1H). | 5-(4,4,5,5-tetramethyl- 1,3,2-dioxaborolan-2- yl)-2-[(1-{[4- (trifluoromethoxy) phenyl]acetyl} piperidin-4- yl)oxy]benzamide (Preparation 14) and (1R)-1-(4- bromophenyl) ethanamine and PM C. |
| | 4′-[(1R)-1-aminoethyl]-4-[(1-{[4- | | |
| | (trifluoromethoxy)phenyl]acetyl}piperidin-4- | | |
| | yl)oxy]biphenyl-3-carboxamide | | |
| 65 | 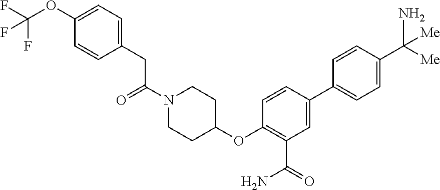 [SMILES: CC(C)(N)c1ccc(-c2ccc(OC3CCN(C(=O)Cc4ccc(OC(F)(F)F)cc4)CC3)c(C(N)=O)c2)cc1] | MS m/z 556 [M + H]⁺ ¹H NMR (400 MHz, DMSO-d₆): δ ppm 1.43 (s, 6H), 1.67 (m, 2H), 1.92 (m, 2H), 3.32-3.47 (m, 2H), 3.80 (m, 4H), 4.83 (m, 1H), 7.28-7.37 (m, 4H), 7.58 (m, 5H), 7.70 (m, 1H), 7.97 (d, 1H). | 5-(4,4,5,5-tetramethyl- 1,3,2-dioxaborolan-2- yl)-2-[(1-{[4- (trifluoromethoxy) phenyl]acetyl}piperidin- 4-yl)oxy]benzamide (Preparation 14) and 2-(4-bromophenyl) propan- 2-amine and PM C. |
| | 4'-(2-aminopropan-2-yl)-4-[(1-{[4- | | |
| | (trifluoromethoxy)phenyl]acetyl}piperidin- | | |
| | 4-yl)oxy]biphenyl-3-carboxamide | | |
| 66 | 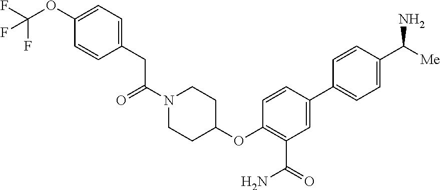 [SMILES: C[C@H](N)c1ccc(-c2ccc(OC3CCN(C(=O)Cc4ccc(OC(F)(F)F)cc4)CC3)c(C(N)=O)c2)cc1] | MS m/z 542 [M + H]⁺ ¹H NMR (400 MHz, DMSO-d₆): δ ppm 1.27 (d, 3H), 1.67 (m, 2H), 1.92 (m, 2H), 3.39-3.47 (m, 2H), 3.75-3.79 (m, 4H), 4.02 (m, 1H), 4.82 (m, 1H), 7.28-7.37 (m, 5H), 7.45 (m, 2H), 7.55 (m, 4H), 7.69 (m, 1H), 7.97 (s, 1H). | 5-(4,4,5,5-tetramethyl- 1,3,2-dioxaborolan-2- yl)-2-[(1-{[4- (trifluoromethoxy) phenyl]acetyl}piperidin- 4-yl)oxy]benzamide (Preparation 14) and (1S)-1-(4- bromophenyl) ethanamine and PM C. |
| | 4'-[(1S)-1-aminoethyl]-4-[(1-{[4- | | |
| | (trifluoromethoxy)phenyl]acetyl}piperidin-4- | | |
| | yl)oxy]biphenyl-3-carboxamide | | |
| 67 | 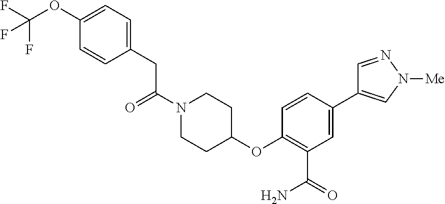 [SMILES: Cn1cc(-c2ccc(OC3CCN(C(=O)Cc4ccc(OC(F)(F)F)cc4)CC3)c(C(N)=O)c2)cn1] | MS m/z 503 [M + H]⁺ ¹H NMR (400 MHz, DMSO-d₆): δ ppm 1.64 (m, 2H), 1.90 (m, 2H), 3.29-3.50 (m, 2H), 3.79- 3.84 (7H), 4.76 (m, 1H), 7.19 (d, 1H), 7.28-7.36 (m, 4H), 7.53-7.61 (3H), 7,78 (s, 1H), 7.86 (d, 1H), 8.09 (s, 1H). | 5-bromo-2-[(1-{[4- (trifluoromethoxy) phenyl]acetyl}piperidin- 4-yl)oxy]benzamide (Preparation 39), 1- methyl-4-(4,4,5,5- tetramethyl-1,3,2- dioxaborolan-2-yl)- 1H-pyrazole and PM F. |
| | 5-(1-methyl-1H-pyrazol-4-yl)-2-[(1-{[4- | | |
| | (trifluoromethoxy)phenyl] | | |
| | acetyl}piperidin-4-yl)oxy]benzamide | | |
| 68 | 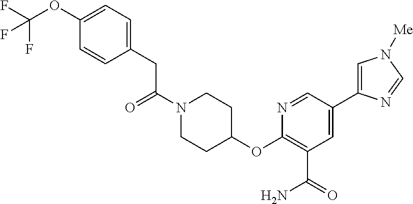 [SMILES: Cn1cnc(-c2cnc(OC3CCN(C(=O)Cc4ccc(OC(F)(F)F)cc4)CC3)c(C(N)=O)c2)c1] | MS m/z 503 [M + H]⁺ ¹H NMR (400 MHz, DMSO-d₆): δ ppm 1.71 (m, 2H), 1.96 (m, 2H), 3.30-3.50 (m, 2H), 3.68 (s, 3H), 3.80 (m, 5H), 5.40 (m, 1H), 7.28-7.37 (m, 4H), 7.56 (br s, 1H), 7.66 (m, 2H), 7.72 (br s, 1H), 8.45 (m, 1H), 8.61 (m, 1H). | 5-(4,4,5,5-tetramethyl- 1,3,2-dioxaborolan-2- yl)-2-({1-[4- (trifluoromethoxy) benzyl]piperidin-4- yl}oxy)pyridine-3- carboxamide (Preparation 13) and 4-bromo-1-methyl-1H- imidazole and PM F. |
| | 5-(1-methyl-1H-imidazol-4-yl)-2-[(1-{[4- | | |
| | (trifluoromethoxy)phenyl]acetyl}piperidin-4- | | |
| | yl)oxy]pyridine-3-carboxamide | | |
| 69 | 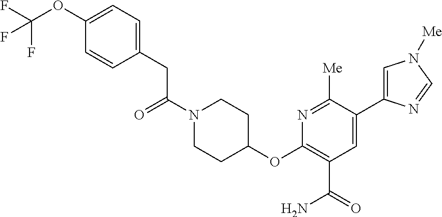 [SMILES: Cc1nc(OC2CCN(C(=O)Cc3ccc(OC(F)(F)F)cc3)CC2)c(C(N)=O)cc1-c1cn(C)cn1] | MS m/z 518 [M + H]⁺ ¹H NMR (400 MHz, DMSO-d₆): δ ppm 1.72 (m, 2H), 1.98 (m, 2H), 2.59 (s, 3H), 3.36-3.47 (m, 1H), 3.70 (s, 3H), 3.76-3.80 (m, 4H), 5.45 (m, 1H), 7.29-7.37 (m, 4H), 7.43 (s, 1H), 7.48 (m, 1H), 7.66 (s, 1H), 7.69 (s, 1H). | 6-methyl-5-(4,4,5,5- tetramethyl-1,3,2- dioxaborolan-2-yl)-2- [(1-{[4- (trifluoromethoxy) phenyl]acetyl}piperidin- 4-yl)oxy]pyridine-3- carboxamide (Preparation 16) and 4-bromo-1-methyl-1H- imidazole and PM D. |
| | 6-methyl-5-(1-methyl-1H-imidazol-4-yl)-2-[(1-{[4- | | |
| | (trifluoromethoxy)phenyl]acetyl}piperidin-4- | | |
| | yl)oxy]pyridine-3-carboxamide | | |
| 70 | 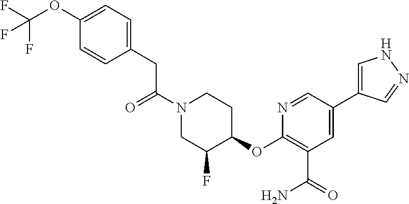 [SMILES: NC(=O)c1cc(-c2cn[nH]c2)cnc1O[C@@H]1CCN(C(=O)Cc2ccc(OC(F)(F)F)cc2)C[C@@H]1F] | MS m/z 508 [M + H]⁺ ¹H NMR (400 MHz, DMSO-d₆): δ ppm 1.77- 1.86 (m, 1H), 2.02 (m, 1H), 2.92-2.97 (m, 1H), 3.13-3.26 (m, 1H), 3.54- 4.03 (m, 3H), 4.32 (m, 1H), 4.59 (m, 1H), 5.03- 5.17 (m, 1H), 5.54 (m, 1H), 7.30-7.36 (m, 4H), 7.54 (s, 1H), 7.84 (s, 1H), 7.98 (br s, 1H), 8.29 (br s, 1H), 8.38 (s, 1H), 8.57 (s, 1H). | 5-bromo-2-{[(3S,4R)- 3-fluoro-1-{[4- (trifluoromethoxy) phenyl]acetyl}piperidin- 4-yl]oxy}pyridine-3- carboxamide (Preparation 27) and 4-(4,4,5,5-tetramethyl- 1,3,2-dioxaborolan-2- yl)-1H-pyrazole and PM C. |
| | 2-{[(3S,4R)-3-fluoro-1-{[4- | | |
| | (trifluoromethoxy)phenyl]acetyl}piperidin- | | |
| | 4-yl]oxy}-5-(1H-pyrazol-4-yl)pyridine-3-carboxamide | | |
| 71 | 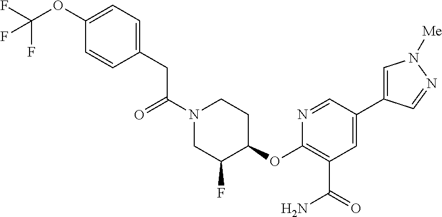 [SMILES: Cn1cc(-c2cnc(O[C@@H]3CCN(C(=O)Cc4ccc(OC(F)(F)F)cc4)C[C@@H]3F)c(C(N)=O)c2)cn1] | MS m/z 522 [M + H]⁺ ¹H NMR (400 MHz, MeOD): δ ppm 1.88-2.13 (m, 2H), 2.94-3.18 (m, 1H), 3.38-3.62 (m, 1H), 3.79-4.09 (m, 6H), 4.41- 4.60 (m, 1H), 5.00-5.16 (m, 1H), 5.56-5.63 (m, 1H), 7.22 (m, 2H), 7.35 (m, 2H), 7.83 (s, 1H), 8.01 (s, 1H), 8.49 (d, 1H). | 5-bromo-2-{[(3S,4R)- 3-fluoro-1-{[4- (trifluoromethoxy) phenyl]acetyl}piperidin- 4-yl]oxy}pyridine-3- carboxamide (Preparation 27) and 1-methyl-4-(4,4,5,5- tetramethyl-1,3,2- dioxaborolan-2-yl)-1H- pyrazole and PM C. |
| | 2-{[(3S,4R)-3-fluoro-1-{[4-(trifluoromethoxy)phenyl] | | |
| | acetyl}piperidin-4-yl]oxy}-5-(1-methyl-1H-pyrazol- | | |
| | 4-yl)pyridine-3-carboxamide | | |
| 72 | 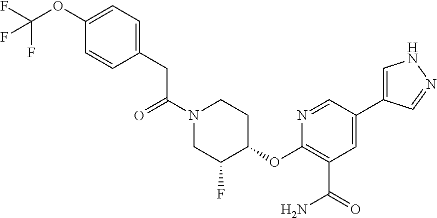 [SMILES: NC(=O)c1cc(-c2cn[nH]c2)cnc1O[C@H]1CCN(C(=O)Cc2ccc(OC(F)(F)F)cc2)C[C@H]1F] | MS m/z 508 [M + H]⁺ ¹H NMR (400 MHz, DMSO-d₆): δ ppm 1.77- 1.86 (m, 1H), 2.02 (m, 1H), 2.92-2.97 (m, 1H), 3.13-3.26 (m, 1H), 3.54- 4.03 (m, 3H), 4.32 (m, 1H), 4.59 (m, 1H), 5.03- 5.17 (m, 1H), 5.54 (m, 1H), 7.30-7.36 (m, 4H), 7.54 (s, 1H), 7.84 (s, 1H), 7.98 (br s, 1H), 8.29 (br s, 1H), 8.38 (s, 1H), 8.57 (s, 1H). | 5-bromo-2-{[(3R,4S)- 3-fluoro-1-{[4- (trifluoromethoxy) phenyl]acetyl}piperidin- 4-yl]oxy}pyridine-3- carboxamide (Preparation 26) and 4-(4,4,5,5-tetramethyl- 1,3,2-dioxaborolan-2- yl)-1H-pyrazole and PM C. |
| | 2-{[(3R,4S)-3-fluoro-1-{[4-(trifluoromethoxy)phenyl] | | |
| | acetyl}piperidin-4-yl]oxy}-5-(1H-pyrazol-4- | | |
| | yl)pyridine-3-carboxamide | | |
| 73 | 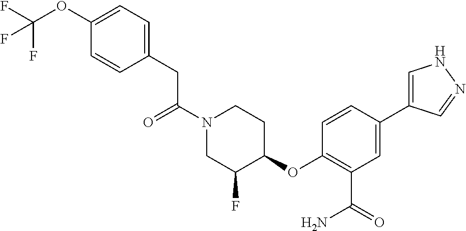 [SMILES: NC(=O)c1cc(-c2cn[nH]c2)ccc1O[C@@H]1CCN(C(=O)Cc2ccc(OC(F)(F)F)cc2)C[C@@H]1F] | MS m/z 507 [M + H]⁺ ¹H NMR (400 MHz, DMSO-d₆): δ ppm 1.69- 1.77 (m, 1H), 1.98 (m, 1H), 2.94 (m, 1H), 3.07- 3.27 (m, 1H), 3.45-3.57 (m, 1H), 3.68-4.00 (m, 2H), 4.30 (m, 1H), 4.58 (m, 0.5H), 4.88-5.12 (m, 1.5H), 7.26-7.36 (m, 5H), 7.56 (br s, 1H), 7.64-7.70 (m, 1H), 7.87 (br s, 1H), 8.00 (s, 1H), 8.17 (s, 1H). | 5-bromo-2-{[(3S,4R)- 3- fluoro-1-{[4- (trifluoromethoxy) phenyl]acetyl}piperidin- 4-yl]oxy}benzamide (Preparation 28) and 4-(4,4,5,5-tetramethyl- 1,3,2-dioxaborolan-2- yl)-1H-pyrazole and PM C. |
| | 2-{[(3S,4R)-3-fluoro-1-{[4-(trifluoromethoxy) | | |
| | phenyl]acetyl}piperidin-4-yl]oxy}- | | |
| | 5-(1H-pyrazol-4-yl)benzamide | | |
| 74 | 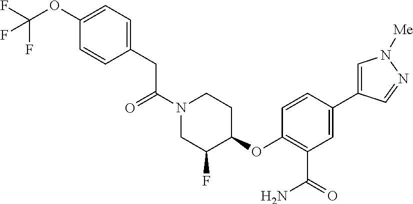 [SMILES: Cn1cc(-c2ccc(O[C@@H]3CCN(C(=O)Cc4ccc(OC(F)(F)F)cc4)C[C@@H]3F)c(C(N)=O)c2)cn1] | MS m/z 521 [M + H]⁺ ¹H NMR (400 MHz, DMSO-d₆): δ ppm 1.70- 1.77 (m, 1H), 1.92-1.98 (m, 1H), 2.94 (m, 1H), 3.07-3.27 (m, 1H), 3.45- 3.57 (m, 1H), 3.68-4.00 (m, 5H), 4.30 (m, 1H), 4.58 (m, 0.5H), 4.88-5.12 (m, 1.5H), 7.26-7.36 (m, 5H), 7.56 (br s, 1H), 7.64-7.70 (m, 1H), 7.87 (br s, 1H), 8.00 (s, 1H), 8.17 (s, 1H). | 5-bromo-2-{[(3S,4R)- 3-fluoro-1-{[4- (trifluoromethoxy) phenyl]acetyl}piperidin- 4-yl]oxy}benzamide (Preparation 28) and 1-methyl-4-{4,4,5,5- tetramethyl-1,3,2- dioxaborolan-2-yl)-1H- pyrazole and PM C. |
| | 2-{[(3S,4R)-3-fluoro-1-{[4-(trifluoromethoxy) | | |
| | phenyl]acetyl}piperidin-4-yl]oxy}- | | |
| | 5-(1-methyl-1H-pyrazol-4-yl)benzamide | | |
| 75 | 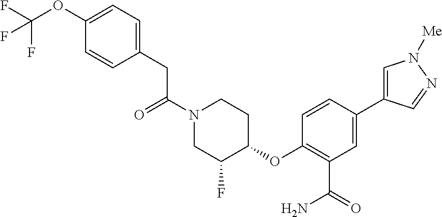 [SMILES: Cn1cc(-c2ccc(O[C@H]3CCN(C(=O)Cc4ccc(OC(F)(F)F)cc4)C[C@H]3F)c(C(N)=O)c2)cn1] | MS m/z 521 [M + H]⁺ ¹H NMR (400 MHz, DMSO-d₆): δ ppm 1.70- 1.77 (m, 1H), 1.92-1.98 (m, 1H), 2.94 (m, 1H), 3.07-3.27 (m, 1H), 3.45- 3.57 (m, 1H), 3.68-4.00 (m, 5H), 4.30 (m, 1H), 4.58 (m, 0.5H), 4.88-5.12 (m, 1.5H), 7.26-7.36 (m, 5H), 7.56 (br s, 1H), 7.64-7.70 (m, 1H), 7.87 (br s, 1H), 8.00 (s, 1H), 8.17 (s, 1H). | 5-bromo-2-{[(3R,4S)- 3-fluoro-1-{[4- (trifluoromethoxy) phenyl]acetyl}piperidin- 4-yl]oxy}benzamide (Preparation 36) and 1-methyl-4-(4,4,5,5- tetramethyl-1,3,2- dioxaborolan-2-yl)-1H- pyrazole and PM C. |
| | 2-{[(3R,4S)-3-fluoro-1-{[4-{trifluoromethoxy) | | |
| | phenyl]acetyl}piperidin-4-yl]oxy}- | | |
| | 5-(1-methyl-1H-pyrazol-4-yl)benzamide | | |
| 76 | 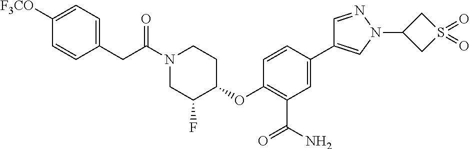 [SMILES: NC(=O)c1cc(-c2cnn(C3CS(=O)(=O)C3)c2)ccc1O[C@H]1CCN(C(=O)Cc2ccc(OC(F)(F)F)cc2)C[C@H]1F] | NMR exhibits rotameric behaviour: ¹H NMR (400 MHz, DMSO-d₆): δ ppm 170-1.75 (m, 1H), 1.96 (m, 1H), 2.91-3.24 (m, 1H), 3.45-3.68 (m, 1H), 3.69-4.00 (m, 3H), 4.32 (m, 1H), 4.58-5.12 (m, 6H), 5.35 (m, 1H), 7.33 (m, 5H), 7.56 (br s, 1H), | 5-bromo-2-{[(3R,4S)- 3-fluoro-1-{[4- (trifluoromethoxy) phenyl]acetyl}piperidin- 4-yl]oxy}benzamide (Preparation 36) and 1-(1,1-dioxidothietan- 3-yl)-4-(4,4,5,5- tetramethyl-1,3,2- dioxaborolan-2-yl)-1H- |
| | 5-[1-(1,1-dioxidothietan-3-yl)-1H-pyrazol-4-yl]-2- | 7.68 (m, 2H), 8.08 (m, | pyrazole (Preparation |
| | {[(3R,4S)-3-fluoro-1-{[4-(trifluoromethoxy)phenyl] | 2H), 8.41 (s, 1H). | 169) and PM C. |
| | acetyl}pipertdin-4-yl]oxy}benzamide | MS m/z 611 [M + H]⁺ | |
| 77 | 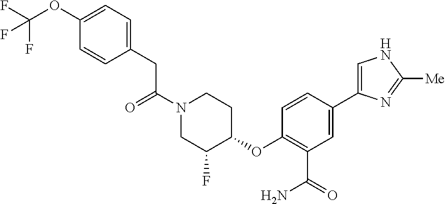 [SMILES: Cc1nc(-c2ccc(O[C@H]3CCN(C(=O)Cc4ccc(OC(F)(F)F)cc4)C[C@H]3F)c(C(N)=O)c2)c[nH]1] | NMR exhibits rotameric behaviour: ¹H NMR (400 MHz, DMSO-d₆): δ ppm 1.74 (m, 1H), 1.96 (m, 1H), 2.31 (s, 3H), 2.90- 3.60 (m, 2H), 3.68-4.05 (m, 2H), 4.34 (m, 1H), 4.59 (m, 1H), 4.90-5.12 (m, 2H), 7.26-7.39 (m, 6H), 7.55 (br s, 1H), 7.62 (br s, 1H), 7.78 (d, 1H), 8.18 (s, 1H). MS m/z 519 [M − H]⁻ | 2-{[(3R,4S)-3-fluoro-1- {[4-(trifluoromethoxy) phenyl]acetyl}piperidin- 4-yl]oxy}-5-(4,4,5,5- tetramethyl-1,3,2- dioxaborolan-2- yl)benzamide (Preparation 17) and tert-butyl 4-bromo-2- methyl-1H-imidazole- 1-carboxylate and PM C. |
| | 2-{[(3R,4S)-3-fluoro-1-{[4-(trifluoromethoxy)phenyl] | | |
| | acetyl}pipertdin-4-yl]oxy}- | | |
| | 5-(2-methyl-1H-imidazol-4-yl)benzamide | | |
| 78 | 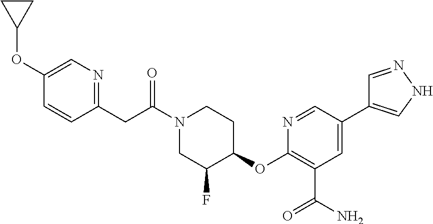 [SMILES: NC(=O)c1cc(-c2cn[nH]c2)cnc1O[C@@H]1CCN(C(=O)Cc2ccc(OC3CC3)cn2)C[C@@H]1F] | NMR exhibits rotameric behaviour: ¹H NMR (400 MHz, DMSO-d₆): δ ppm 0.67 (m, 2H), 0.80 (m, 2H), 1.80 (m, 1H), 1.99 (m, 1H), 2.80-3.35 (m, 2H), 3.53-4.10 (m, 4H), 4.09-4.56 (m, 1H), 5.01- 5.15 (m, 1H), 5.47-5.75 (m, 1H), 7.23 (m, 1H), 7.44-7.48 (m, 1H), 7.54 (br s, 1H), 7.84 (br s, 1H), 7.98 (s, 1H), 8.29 (m, 1H), 8.38 (s, 1H), 8.57 (s, 1H). | 5-bromo-2-{[(3S,4R)- 1-{[5-(cyclopropyloxy) pyridin-2-yl]acetyl}-3- fluoropiperidin-4- yl]oxy}pyridine-3- carboxamide (Preparation 34) and 4-(4,4,5,5-tetramethyl- 1,3,2-dioxaborolan-2- yl)-1H-pyrazole and PM C. |
| | 2-{[(3S,4R)-1-{[5-(cyclopropyloxy)pyridin- | MS m/z 481 [M + H]⁺ | |
| | 2-yl]acetyl}-3-fluoropiperidin-4-yl]oxy}- | | |
| | 5-(1H-pyrazol-4-yl)pyridine-3-carboxamide | | |
| 79 | 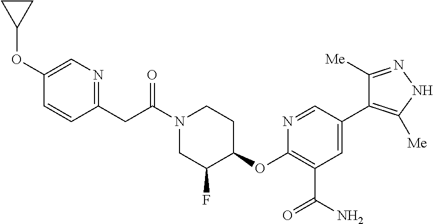 [SMILES: Cc1n[nH]c(C)c1-c1cnc(O[C@@H]2CCN(C(=O)Cc3ccc(OC4CC4)cn3)C[C@@H]2F)c(C(N)=O)c1] | MS m/z 509 [M + H]⁺ NMR exhibits rotameric behaviour: ¹H NMR (400 MHz, DMSO-d₆): δ ppm 0.70 (m, 2H), 0.80 (m, 2H), 1.85 (m, 1H), 2.00 (m, 1H), 2.15 (s, 3H), 2.32 (s, 3H), 2.92-3.32 (m, 2H), 3.52-4.11 (m, 4H), 4.33-4.58 (m, 1H), 5.03-5.15 (m, 1H), 5.49- 5.75 (m, 1H), 7.25 (m, 1H), 7.46 (m, 1H), 7.54 (br s, 1H), 7.85 (br s, 1H), 8.09 (m, 1H), 8.24 | 5-bromo-2-{[(3S,4R)- 1-{[5-(cyclopropyloxy) pyridin-2-yl]acetyl}-3- fluoropiperidin-4- yl]oxy}pyridine-3- carboxamide (Preparation 34) and 3,5-dimethylpyrazole- 4-boronic acid pinacol ester and PM C. |
| | 2-{[(3S,4R)-1-{[5-(cyclopropyloxy)pyridin- | (2H), 12.41 (br s, 1H). | |
| | 2-yl]acetyl}-3-fluoropiperidin-4-yl]oxy}- | | |
| | 5-(3,5-dimethyl-1H-pyrazol-4-yl)pyridine-3-carboxamide | | |
| 80 | 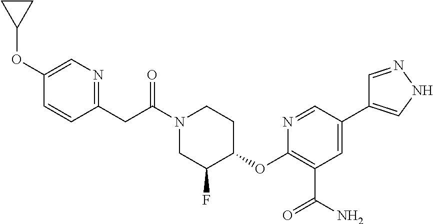 [SMILES: NC(=O)c1cc(-c2cn[nH]c2)cnc1O[C@H]1CCN(C(=O)Cc2ccc(OC3CC3)cn2)C[C@@H]1F] | MS m/z 481 [M + H]⁺ NMR exhibits rotameric behaviour: ¹H NMR (400 MHz, DMSO-d₆): δ ppm 0.68 (m, 2H), 0.79 (m, 2H), 1.73 (m, 1H), 2.10 (m, 1H), 3.30-4.08 (m, 7H), 4.80-4.92 (m, 1H), 4.80-4.92 (m, 1H), 7.26 (d, 1H), 7.47 (dd, 1H), 7.61 (br s, 1H), 7.75 (d, 1H), 7.97 (br s, 1H), 8.26-8.31 (m, 3H), 8.56 (d, 1H), 13.02 (br s, 1H). | 5-bromo-2-{[(3S,4S)- 1-{[5-(cyclopropyloxy) pyridin-2-yl]acetyl}-3- fluoropiperidin-4- yl]oxy}pyridine-3- carboxamide (Preparation 23) and 4-(4,4,5,5-tetramethyl- 1,3,2-dioxaborolan-2- yl)-1H-pyrazole and PM C. |
| | 2-{[(3S,4S)-1-{[5-(cyclopropyloxy)pyridin- | | |
| | 2-yl]acetyl}-3-fluoropiperidin-4-yl]oxy}- | | |
| | 5-(1H-pyrazol-4-yl)pyridine-3-carboxamide | | |
| 81 | 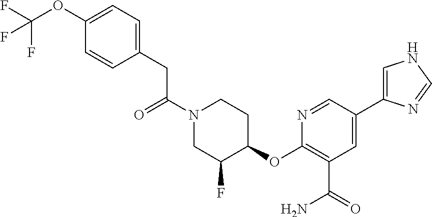 [SMILES: NC(=O)c1cc(-c2c[nH]cn2)cnc1O[C@@H]1CCN(C(=O)Cc2ccc(OC(F)(F)F)cc2)C[C@@H]1F] | MS m/z 508 [M + H]⁺ NMR exhibits rotameric behaviour: ¹H NMR (400 MHz, DMSO-d₆): δ ppm 1.80 (m, 1H), 2.03 (m, 1H), 2.92-3.25 (m, 1H), 3.58-4.03 (m, 3H), 4.34 (m, 1H), 4.60 (m, 1H), 5.03-5.18 (m, 1H), 5.49- 5.56 (m, 1H), 7.31 (m, 4H), 7.53 (br s, 1H), 7.72 (m, 2H), 7.82 (br s, 1H), 8.57 (s, 1H), 8.68 (s, 1H), 12.26 (br s, 1H). | 2-{[(3S,4R)-3-fluoro-1- {[4-(trifluoromethoxy) phenyl]acetyl}piperidin- 4-yl]oxy}-5-(4,4,5,5- tetramethyl-1,3,2- dioxaborolan-2- yl)pyridine-3- carboxamide (Preparation 15) and tert-butyl 4-bromo-1H- imidazole-1-carboxylate. Deprotection occurs in-situ. |
| | 2-{[(3S,4R)-3-fluoro-1-{[4-(trifluoromethoxy)phenyl] | | |
| | acetyl}piperidin-4-yl]oxy}-5-(1H-imidazol-4- | | |
| | yl)pyridine-3-carboxamide | | |
| 82 | 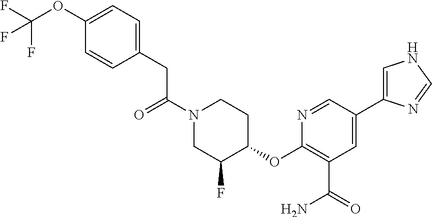 [SMILES: NC(=O)c1cc(-c2c[nH]cn2)cnc1O[C@H]1CCN(C(=O)Cc2ccc(OC(F)(F)F)cc2)C[C@@H]1F] | MS m/z 508 [M + H]⁺ NMR exhibits rotameric behaviour: ¹H NMR (400 MHz, DMSO-d₆): δ ppm 1.77 (m, 1H), 2.10 (m, 1H), 3.37-4.03 (m, 6H), 4.82-4.99 (m, 1H), 5.52 (m, 1H), 7.31 (m, 4H), 7.60 (br s, 1H), 7.74 (m, 3H), 8.50 (s, 1H), 8.67 (s, 1H), 12.25 (br s, 1H). | 2-{[(3S,4S)-3-fluoro-1- {[4-(trifluoromethoxy) phenyl]acetyl}piperidin- 4-yl]oxy}-5-(4,4,5,5- tetramethyl-1,3,2- dioxaborolan-2- yl)pyridine-3- carboxamide (Preparation 22) and tert-butyl 4-bromo-1H- imidazole-1-carboxylate. Deprotection occurs in-situ. |
| | 2-{[(3S,4S)-3-fluoro-1-{[4-(trifluoromethoxy)phenyl] | | |
| | acetyl}piperidin-4-yl]oxy}-5-(1H-imidazol-4- | | |
| | yl)pyridine-3-carboxamide | | |
| 83 | 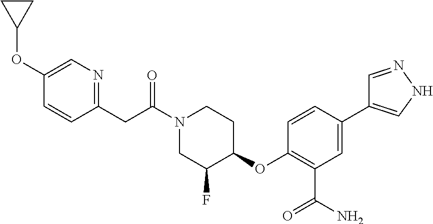 [SMILES: NC(=O)c1cc(-c2cn[nH]c2)ccc1O[C@@H]1CCN(C(=O)Cc2ccc(OC3CC3)cn2)C[C@@H]1F] | MS m/z 479 [M + H]⁺ NMR exhibits rotameric behaviour: ¹H NMR (400 MHz, DMSO-d₆): δ ppm 0.67 (m, 2H), 0.82 (m, 2H), 1.76 (m, 1H), 1.94 (m, 1H), 2.90-3.60 (m, 2H), 3.70-4.06 (m, 3H), 4.25 (m, 1H), 4.55 (m, 1H), 4.89-5.11 (m, 2H), 7.24-7.28 (m, 2H), 7.44 (m, 1H), 7.56 (br s, 1H), 7.66 (m, 2H), 7.90 (br s, 1H) 8.00 (d, 1H), 8.20 (br s, 1H), 8.25 (d, 1H), | 5-bromo-2-{[(3S,4R)- 1-{[5-(cyclopropyloxy) pyridin-2-yl]acetyl}-3- fluoropiperidin-4- yl]oxy}benzamide (Preparation 25) and 4-(4,4,5,5-tetramethyl- 1,3,2-dioxaborolan-2- yl)-1H-pyrazole and PM C. |
| | 2-{[(3S,4R)-1-{[5-(cyclopropyloxy)pyridin- | 12.92 (br s, 1H). | |
| | 2-yl]acetyl}-3-fluoropiperidin-4-yl]oxy}- | | |
| | 5-(1H-pyrazol-4-yl)benzamide | | |
| 84 | 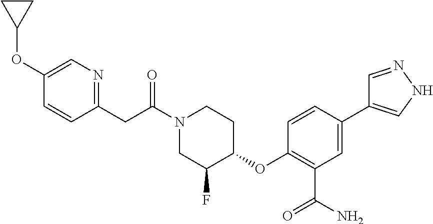 [SMILES: NC(=O)c1cc(-c2cn[nH]c2)ccc1O[C@H]1CCN(C(=O)Cc2ccc(OC3CC3)cn2)C[C@@H]1F] | MS m/z 480 [M + H]⁺ NMR exhibits rotameric behaviour: ¹H NMR (400 MHz, DMSO-d₆): δ ppm 0.67 (m, 2H), 0.81 (m, 2H), 1.70 (m, 1H), 2.01 (m, 1H), 3.45-4.15 (m, 7H), 4.70-4.90 (m, 2H), 7.23 (m, 2H), 7.45 (m, 1H), 7.54 (m, 1H), 7.64 (m, 1H), 7.86 (m, 1H), 8.16 (br s, 1H), 8.26 (m, 1H), 12.91 (br s, 1H). | 5-bromo-2-{[(3S,4S)- 1-{[5-(cyclopropyloxy) pyridin-2-yl]acetyl}-3- fluoropiperidin-4- yl]oxy}benzamide (Preparation 24) and 4-(4,4,5,5-tetramethyl- 1,3,2-dioxaborolan-2- yl)-1H-pyrazole and PM G. |
| | 2-{[(3S,4S)-1-{[5-(cyclopropyloxy)pyridin- | | |
| | 2-yl]acetyl}-3-fluoropiperidin-4-yl]oxy}- | | |
| | 5-(1H-pyrazol-4-yl)benzamide | | |
| 85 | 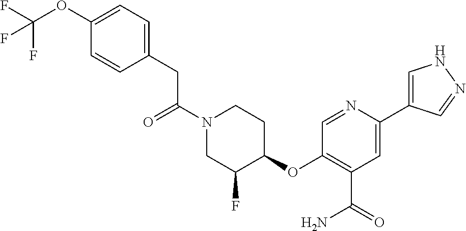 [SMILES: NC(=O)c1cc(-c2cn[nH]c2)ncc1O[C@@H]1CCN(C(=O)Cc2ccc(OC(F)(F)F)cc2)C[C@@H]1F] | MS m/z 508 [M + H]⁺ NMR exhibits rotameric behaviour: ¹H NMR (400 MHz, DMSO-d₆): δ ppm 1.75 (m, 2H), 1.96 (m, 2H), 2.96-3.32 (m, 2H), 3.50-4.00 (m, 2H), 4.22- 4.48 (m, 2H), 4.90-5.11 (m, 2H), 7.31 (m, 4H), 7.67 (br s, 1H), 7.84 (s, 1H), 7.86 (br s, 1H), 8.00 (br s, 1H), 8.30 (br s, 1H), 8.55 (d, 1H). | 2-bromo-5-{[(3S,4R)- 3-fluoro-1-{[4- (trifluoromethoxy) phenyl]acetyl}piperidin- 4-yl]oxy}pyridine-4- carboxamide (Preparation 32) and 4-(4,4,5,5-tetramethyl- 1,3,2-dioxaborolan-2- yl)-1H-pyrazole and PM C. |
| | 5-{[(3S,4R)-3-fluoro-1-{[4-(trifluoromethoxy)phenyl] | | |
| | acetyl}piperidin-4-yl]oxy}-2-(1H-pyrazol-4- | | |
| | yl)pyridine-4-carboxamide | | |
| 86 | 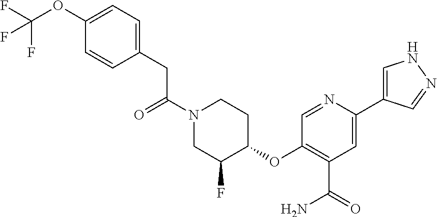 [SMILES: NC(=O)c1cc(-c2cn[nH]c2)ncc1O[C@H]1CCN(C(=O)Cc2ccc(OC(F)(F)F)cc2)C[C@@H]1F] | MS m/z 508 [M + H]⁺ NMR exhibits rotameric behaviour: ¹H NMR (400 MHz, DMSO-d₆): δ ppm 1.73 (m, 1H), 2.02 (m, 1H), 3.45-4.10 (m, 6H), 4.70-4.95 (m, 2H), 7.34 (m, 4H), 7.76 (m, 3H), 8.00 (br s, 1H), 8.29 (br s, 1H), 8.53 (s, 1H). | 2-bromo-5-{[(3S,4S)- 3-fluoro-1-{[4- (trifluoromethoxy) phenyl]acetyl}piperidin- 4-yl]oxy}pyridine-4- carboxamide (Preparation 31) and 4-(4,4,5,5-tetramethyl- 1,3,2-dioxaborolan-2- yl)-1H-pyrazole and PM C. |
| | 5-{[(3S,4S)-3-fluoro-1-{[4-(trifluoromethoxy)phenyl] | | |
| | acetyl}piperidin-4-yl]oxy}-2-(1H-pyrazol-4- | | |
| | yl)pyridine-4-carboxamide | | |
| 87 | 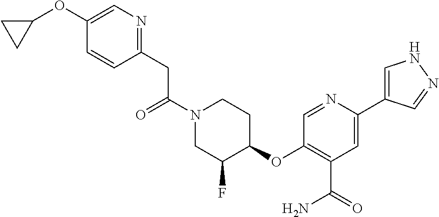 [SMILES: NC(=O)c1cc(-c2cn[nH]c2)ncc1O[C@@H]1CCN(C(=O)Cc2ccc(OC3CC3)cn2)C[C@@H]1F] | MS m/z 481 [M + H]⁺ NMR exhibits rotameric behaviour: ¹H NMR (400 MHz, DMSO-d₆): δ ppm 0.68 (m, 2H), 0.80 (m, 2H), 1.79 (m, 1H), 1.94 (m, 1H), 2.98-3.30 (m, 2H), 3.50-4.05 (m, 3H), 4.15-4.50 (m, 2H), 4.93- 5.08 (m, 2H), 7.24 (m, 1H), 7.45 (m, 1H), 7.67 (br s, 1H), 7.84 (s, 1H), 7.86 (br s, 1H), 8.00 (s, 1H), 8.25 (m, 1H), 8.30 | 2-bromo-5-{[(3S,4R)- 1-{[5-(cyclopropyloxy) pyridin-2-yl]acetyl}-3- fluoropiperidin-4- yl]oxy}pyridine-4- carboxamide (Preparation 33) and 4-(4,4,5,5-tetramethyl- 1,3,2-dioxaborolan-2- yl)-1H-pyrazole and PM G. |
| | 5-{[(3S,4R)-1-{[5-(cyclopropyloxy)pyridin- | (s, 1H), 8.55 (m, 1H). | |
| | 2-yl]acetyl}-3-fluoropiperidin-4-yl]oxy}- | | |
| | 2-(1H-pyrazol-4-yl)pyridine-4-carboxamide | | |
| 88 | 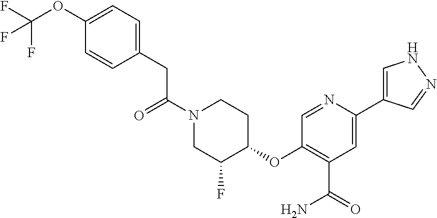 [SMILES: NC(=O)c1cc(-c2cn[nH]c2)ncc1O[C@H]1CCN(C(=O)Cc2ccc(OC(F)(F)F)cc2)C[C@H]1F] | MS m/z 508 [M + H]⁺ NMR exhibits rotameric behaviour: ¹H NMR (400 MHz, DMSO-d₆): δ ppm 1.75 (m, 2H), 1.96 (m, 2H), 2.96-3.32 (m, 2H), 3.50-4.00 (m, 2H), 4.22- 4.48 (m, 2H), 4.90-5.11 (m, 2H), 7.31 (m, 4H), 7.67 (br s, 1H), 7.84 (s, 1H), 7.86 (br s, 1H), 8.00 (br s, 1H), 8.30 (br s, 1H), 8.55 (d, 1H). | 2-bromo-5-{[(3R,4S)- 3-fluoro-1-{[4- (trifluoromethoxy) phenyl]acetyl}piperidin- 4-yl]oxy}pyridine-4- carboxamide (Preparation 41) and 4-(4,4,5,5-tetramethyl- 1,3,2-dioxaborolan-2- yl)-1H-pyrazole and PM C. |
| | 5-{[(3R,4S)-3-fluoro-1-{[4-(trifluoromethoxy)phenyl] | | |
| | acetyl}piperidin-4-yl]oxy}-2-(1H-pyrazol-4- | | |
| | yl)pyridine-4-carboxamide | | |
| 89 | 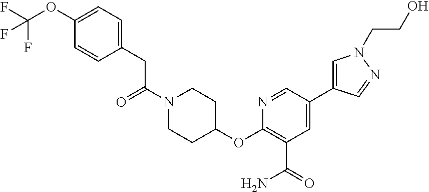 [SMILES: NC(=O)c1cc(-c2cnn(CCO)c2)cnc1OC1CCN(C(=O)Cc2ccc(OC(F)(F)F)cc2)CC1] | MS m/z 534 [M + H]⁺ ¹H NMR (400 MHz, DMSO-d₆): δ ppm 1.64- 1.75 (m, 2H), 1.90-1.99 (m, 2H), 3.35-3.48 (m, 3H), 3.74-3.83 (m, 5H), 4.15 (t, 2H), 4.92 (t, 1H), 5.37-5.39 (m, 1H), 7.30 (d, 2H), 7.36 (d, 2H), 7.57 (brs, 1H), 7.74 (br s, 1H), 7.91 (s, 1H), 8.21 (s, 1H), 8.27 (d, 1H), 8.51 (d, 1H). | 5-(4,4,5,5-tetramethyl- 1,3,2-dioxaborolan-2- yl)-2-({1-[4- (trifluoromethoxy) benzyl]piperidin-4- yl}oxy)pyridine-3- carboxamide (Preparation 13) and 2-(4-bromo-1H- pyrazol-1-yl)ethanol. |
| | 5-[1-(2-hydroxyethyl)-1H-pyrazol-4-yl]-2-[(1-{[4- | | |
| | (trifluoromethoxy)phenyl]acetyl}piperidin-4- | | |
| | yl)oxy]pyridine-3-carboxamide | | |
| 90 | 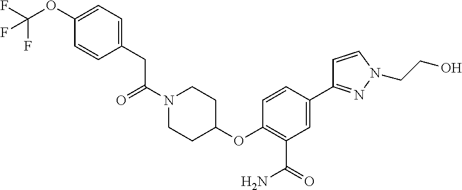 [SMILES: NC(=O)c1cc(-c2ccn(CCO)n2)ccc1OC1CCN(C(=O)Cc2ccc(OC(F)(F)F)cc2)CC1] | MS m/z 533 [M + H]⁺ ¹H NMR (400 MHz, DMSO-d₆): δ ppm 1.66 (m, 2H), 1.93 (m, 2H), 3.33-3.42 (m, 2H), 3.74- 3.79 (m, 6H), 4.16 (t, 2H), 4.80 (m, 1H), 4.89 (t, 1H), 6.61 (d, 1H), 7.23 (d, 1H), 7.29 (d, 2H), 7.35 (d, 2H), 7.54 (br, 1H), 7.56 (br, 1H), 7.71 (d, 1H), 7.80 (dd, 1H), 8.15 (d, 1H). | 5-(4,4,5,5-tetramethyl- 1,3,2-dioxaborolan-2- yl)-2-[(1-{[4- (trifluoromethoxy) phenyl]acetyl} piperidin-4- yl)oxy]benzamide (Preparation 14) and 2-(4-bromo-1H- pyrazol-1-yl)ethanol. |
| | 5-[1-(2-hydroxyethyl)-1H-pyrazol-3-yl]-2-[(1-{[4- | | |
| | {trifluoromethoxy)phenyl] | | |
| | acetyl}piperidin-4-yl)oxy]benzamide | | |
| 91 | 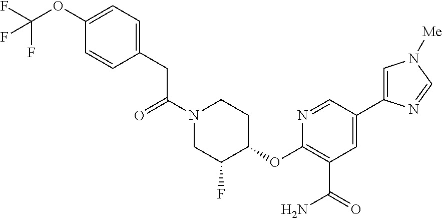 [SMILES: Cn1cnc(-c2cnc(O[C@H]3CCN(C(=O)Cc4ccc(OC(F)(F)F)cc4)C[C@H]3F)c(C(N)=O)c2)c1] 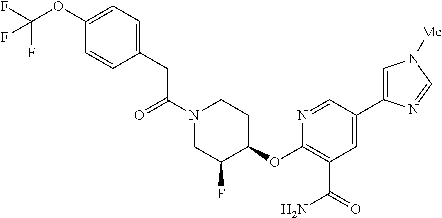 [SMILES: Cn1cnc(-c2cnc(O[C@@H]3CCN(C(=O)Cc4ccc(OC(F)(F)F)cc4)C[C@@H]3F)c(C(N)=O)c2)c1] | MS m/z 522 [M + H]⁺ ¹H NMR (400 MHz, DMSO-d₆): δ ppm 1.70- 1.90 (m, 1H), 1.98-2.10 (m, 1H), 2.94-3.25 (m, 1H), 3.54-4.03 (m, 6.5H), 4.00 (m, 1H), 4.60 (m, 0.5H), 5.03-5.17 (m, 1H), 5.49-5.55 (m, 1H), 7.29- 7.36 (m, 4H), 7.53 (br s, 1H), 7.70 (m, 2H), 7.83 (br s, 1H), 8.53 (m, 1H), 8.64 (m, 1H). | Cis-racemic 2-((3- fluoro-1-(2-(4- (trifluoromethoxy) phenyl)acetyl) piperidin-4- yl)oxy)-5-(4,4,5,5- tetramethyl-1,3,2- dioxaborolan-2- yl)nicotinamide (Preparation 18) and 4-iodo-1-methyl-1H- imidazole. |
| | 1:1 Cis-racemic-2-{[3-fluoro-1-{[4-(trifluoromethoxy) | | |
| | phenyl]acetyl}piperidin-4-yl]oxy}-5-(1-methyl-1H- | | |
| | imidazol-4-yl)pyridine-3-carboxamide | | |
| 92 | 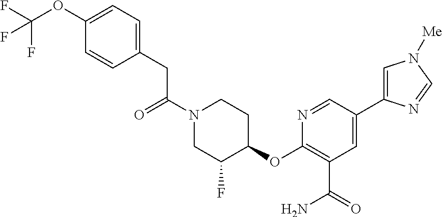 [SMILES: Cn1cnc(-c2cnc(O[C@@H]3CCN(C(=O)Cc4ccc(OC(F)(F)F)cc4)C[C@H]3F)c(C(N)=O)c2)c1] 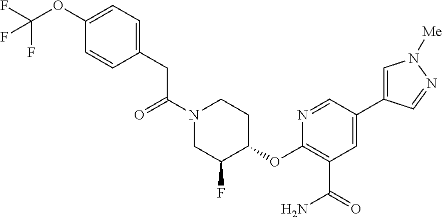 [SMILES: Cn1cc(-c2cnc(O[C@H]3CCN(C(=O)Cc4ccc(OC(F)(F)F)cc4)C[C@@H]3F)c(C(N)=O)c2)cn1] | MS m/z 522 [M + H]⁺ ¹H NMR (400 MHz, DMSO-d₆): δ ppm 1.77- 1.86 (m, 1H), 2.02 (m, 1H), 2.94 (m, 1H), 3.12- 3.21 (m, 1H), 3.69 (s, 3H), 3.84 (m, 1H), 4.00- 4.03 (m, 1H), 4.33 (m, 1H), 4.60 (m, 1H), 5.05 (m, 0.5H), 5.15 (m, 0.5H), 5.45-5.60 (m, 1H), 7.27-7.40 (m, 4H), 7.53 (br s, 1H), 7.70 (m, 2H), 7.85 (br s, 1H), 8.53 (s, 1H), 8.65 (s, 1H). | 1:1 Trans-racemic 2- ((3-fluoro-1-(2-(4- (trifluoromethoxy) phenyl)acetyl) piperidin-4- yl)oxy)-5-(4,4,5,5- tetramethyl-1,3,2- dioxaborolan-2- yl)nicotinamide (Preparation 19) and 4-iodo-1-methyl-1H- imidazole. |
| | 1:1 Trans-racemic-2-{[3-fluoro-1-{[4-{trifluoromethoxy) | | |
| | phenyl]acetyl}piperidin-4-yl]oxy}-5-(1-methyl-1H- | | |
| | imidazol-4-yl)pyridine-3-carboxamide | | |
| 93 | 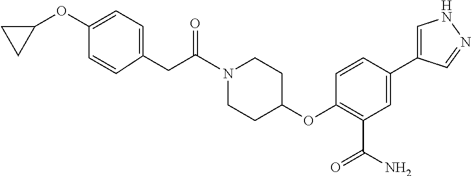 [SMILES: NC(=O)c1cc(-c2cn[nH]c2)ccc1OC1CCN(C(=O)Cc2ccc(OC3CC3)cc2)CC1] | MS m/z 461 [M + H]⁺ ¹H NMR (400 MHz, DMSO-d₆): δ ppm 0.61- 0.63 (m, 2H), 0.75-0.76 (m, 2H), 1.59-161 (m, 2H), 1.86-1.88 (m, 2H), 3.32-3.45 (m, 2H), 3.66- 3.80 (m, 5H), 4.74 (t, 1H), 6.97 (d, 2H), 7.14- 7.19 (m, 3H), 7.53 (br s, 2H), 7.63 (dd, 1H), 7.85 (s, 1H), 7.89 (d, 1H), | 4-(4,4,5,5-tetramethyl- 1,3,2-dioxaborolan-2- yl)-1H-pyrazole and 5- bromo-2-((1-(2-(4- cyclopropoxyphenyl) acetyl)piperidin-4- yl)oxy)benzamide (Preparation 40) and PM C. |
| | 2-((1-(2-(4-cyclopropoxyphenyl) | 8.14 (br s, 1H), 12.89 | |
| | acetyl)piperidin-4-yl)oxy)-5- | (br, 1H). | |
| | (1H-pyrazol-4-yl)benzamide | | |

Library Protocol 1

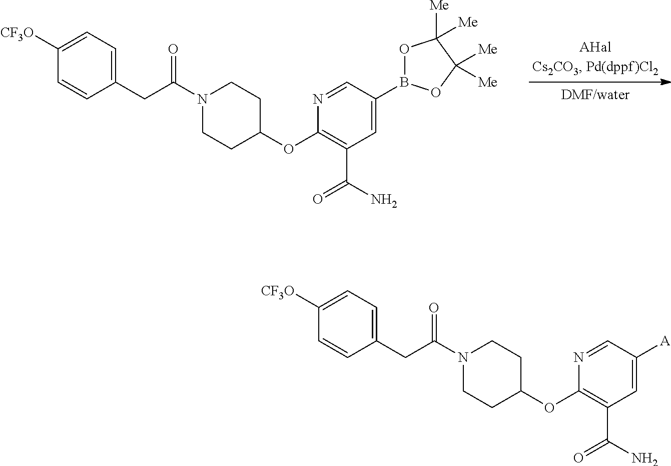

To a 0.2M solution of 5-(4,4,5,5-tetramethyl-1,3,2-dioxaborolan-2-yl)-2-[(1-{[4-(trifluoromethoxy)phenyl]acetyl}piperidin-4-yl)oxy]pyridine-3-carboxamide (Preparation 13, 500 μL, 100 μmol) in DMF was added a 0.2M solution of compounds of formula (IV) (500 μL, 100 μmol) in DMF with argon purging. A 2M solution of cesium carbonate (100 μL, 200 μmol) in degassed water was added followed by tetrakis(triphenylphosphine)palladium (0) (5.7 mg, 5 μmol) and the reaction was heated to 110° C. under microwave irradiation for 15 minutes. The reaction was cooled and concentrated in vacuo. the residue was dissolved in DMSO (1 mL) and purified using preparative HPLC using one of the Purification Methods (PM) below:

Preparative HPLC Method A: Xterra C18 250×19 mm, 10 um eluting with a gradient of between 10-65% acetonitrile in 0.1% formic acid in water. Gradient time: 18 minutes, hold time 1 minute, flow rate 16 mL/min.

Preparative HPLC Method B: Gemini C18 50×21.1 mm, 5 um eluting with a gradient of between 10-65% acetonitrile in 10 mM ammonium hydroxide. Gradient time: 18 minutes, hold time 2 minutes, flow rate 16 mL/min.

Preparative HPLC Method C: Luna Phenyl Hexyl 150×21.1 mm, 10 um eluting with a gradient of between 10-60% acetonitrile in 0.1% formic acid in water. Gradient time: 15 minutes, hold time 1 minute, flow rate 20 mL/min.

Preparative HPLC Method D: XBRIDGE 50×19 mm, 5 um eluting with a gradient of between 10-60% acetonitrile in 0.1% ammonium hydroxide in water. Gradient time: 7 minutes, hold time 1 minute, flow rate 20 mL/min.

LCMS QC:

A: 10 nM ammonium acetate in water; B: MeCN

Column: Zorbax Extend C18 50×4.6 mm×5 um

Gradient: From 95% [A] and 5% [B] to 85% [A] and 15% [B] in 1.5 min, further to 90% [B] in 3.0 min and finally back to initial condition in 5.00 min, 1.5 mL/min flow rate

Examples 94-98 were prepared according to Library Protocol 1 using 5-(4,4,5,5-tetramethyl-1,3,2-dioxaborolan-2-yl)-2-[(1-{[4-(trifluoromethoxy)phenyl]acetyl}piperidin-4-yl)oxy]pyridine-3-carboxamide (Preparation 13) and compounds of formula (IV).

| Example | Structure | Name | MS Data, PM & (IV) |
|---|---|---|---|
| 94 | 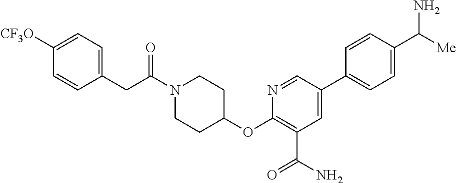 | Racemic 5-[4-(1- aminoethyl)phenyl]-2-[(1- {[4-(trifluoromethoxy) phenyl]acetyl}piperidin-4- yl)oxy]pyridine-3- carboxamide | MS m/z 543 [M + H]⁺ Rt = 1.43 minutes. PM: Method A. Using 1-(4- bromophenyl) ethanamine. |
| 95 | 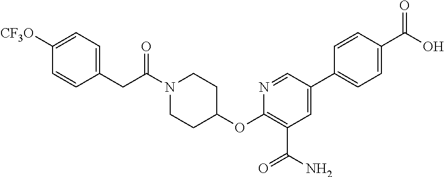 | 4-{5-carbamoyl-6-[(1-{[4- (trifluoromethoxy)phenyl] acetyl}piperidin-4- yl)oxy]pyridin-3-yl} benzoic acid | MS m/z 544 [M + H]⁺ Rt = 1.64 minutes. PM: Method B. Using 4- iodobenzoic acid. |
| 96 | 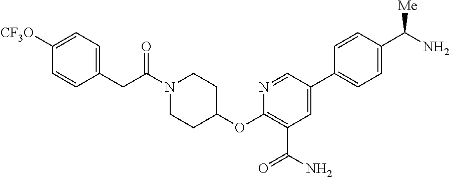 | 5-{4-[(1S)-1- aminoethyl]phenyl}-2-[(1- {[4-(trifluoromethoxy) phenyl]acetyl}piperidin-4- yl)oxy]pyridine-3- carboxamide | MS m/z 543 [M + H]⁺ Rt = 2.50 minutes. PM: Method D. Using (1S)-1-(4- bromophenyl) ethanamine. |
| 97 | 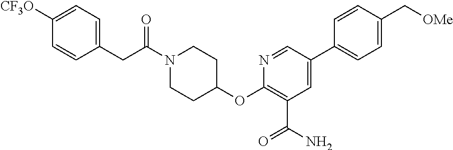 | 5-[4-(methoxymethyl) phenyl]-2-[(1-{[4- (trifluoromethoxy)phenyl] acetyl}piperidin-4- yl)oxy]pyridine-3- carboxamide | MS m/z 544 [M + H]⁺ Rt = 1.63 minutes. PM: Method C. Using 4- chlorobenzyl methyl ether. |
| 98 | 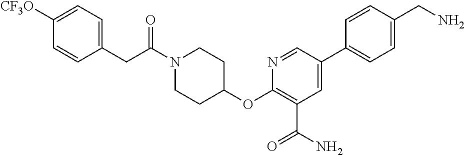 | 5-[4-(aminomethyl)phenyl]- 2-[(1-{[4-(trifluoromethoxy) phenyl]acetyl}piperidin-4- yl)oxy]pyridine-3- carboxamide | MS m/z 529 [M + H]⁺ Rt = 1.41 minutes. PM: Method A. Using 4- iodobenzylamine. |

Library Protocol 2

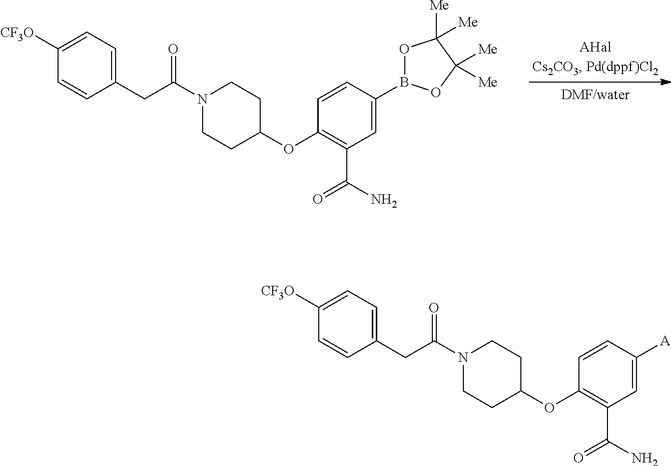

To a 0.2M solution of 5-(4,4,5,5-tetramethyl-1,3,2-dioxaborolan-2-yl)-2-[(1-{[4-(trifluoromethoxy)phenyl]acetyl}piperidin-4-yl)oxy]benzamide (Preparation 14, 500 μL, 100 μmol) in DMF was added a 0.2M solution of compounds of formula (IV) (500 μL, 100 μmol) in DMF with argon purging. A 2M solution of cesium carbonate (100 μL, 200 μmol) in degassed water was added followed by tetrakis(triphenylphosphine)palladium (0) (5.7 mg, 5 μmol) and the reaction was heated to 130° C. under microwave irradiation for 15 minutes. The reaction was cooled and concentrated in vacuo. the residue was dissolved in DMSO (1 mL) and purified using preparative HPLC:

Preparative HPLC Method: XBRIDGE (50 or 250)×19 mm, 5 um eluting with a gradient of between 5-80% acetonitrile in 0.1% ammonium hydroxide in water. Gradient time: 7 or 18 minutes, hold time 1 minute, flow rate 16 or 20 mL/min.

LCMS QC:

A: 0.05% formic acid in water; B: MeCN

Column: RESTEK C18 30×2.1 mm×3 um

Gradient: From 98% [A] and 2% [B] to 90% [A] and 10% [B] in 1.0 min, further to 98% [B] in 2.0 min and finally back to initial condition in 3.00 min, 1.5 mL/min flow rate

Examples 99-102 were prepared according to Library Protocol 2 using 5-(4,4,5,5-tetramethyl-1,3,2-dioxaborolan-2-yl)-2-[(1-{[4-(trifluoromethoxy)phenyl]acetyl}piperidin-4-yl)oxy]benzamide (Preparation 14) and compounds of formula (IV).

| Example | Structure | Name | MS Data and (IV) |
|---|---|---|---|
| 99 | 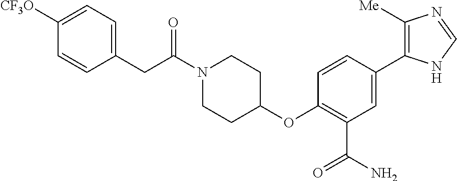 | 5-(4-methyl-1H-imidazol-5- yl)-2-[(1-{[4- (trifluoromethoxy)phenyl] acetyl}piperidin-4- yl)oxy]benzamide | MS m/z 503 [M + H]⁺ Rt = 1.41 minutes. Using 4-iodo-5- methylimidazole. |
| 100 | 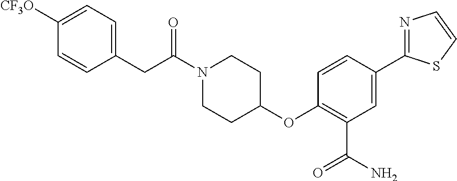 | 5-(1,3-thiazol-2-yl)-2-[(1-{[4- (trifluoromethoxy)phenyl] acetyl}piperidin-4- yl)oxy]benzamide | MS m/z 506 [M + H]⁺ Rt = 1.67 minutes. Using 2- iodothiazole. |
| 101 | 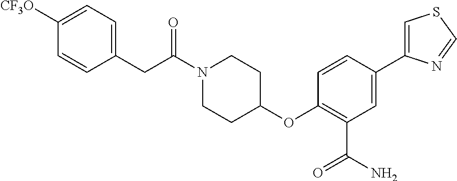 | 5-(1,3-thiazol-4-yl)-2-[(1-{[4- (trifluoromethoxy)phenyl] acetyl}piperidin-4- yl)oxy]benzamide | MS m/z 506 [M + H]⁺ Rt = 1.65 minutes. Using 4- bromothiazole. |
| 102 | 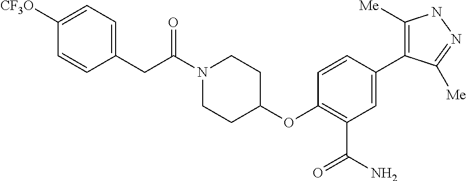 | 5-(3,5-dimethyl-1H-pyrazol- 4-yl)-2-[(1-{[4- (trifluoromethoxy)phenyl] acetyl}piperidin-4- yl)oxy]benzamide | MS m/z 517 [M + H]⁺ Rt = 1.59 minutes. Using 4-bromo-3,5- dimethylpyrazole. |

Library Protocol 3

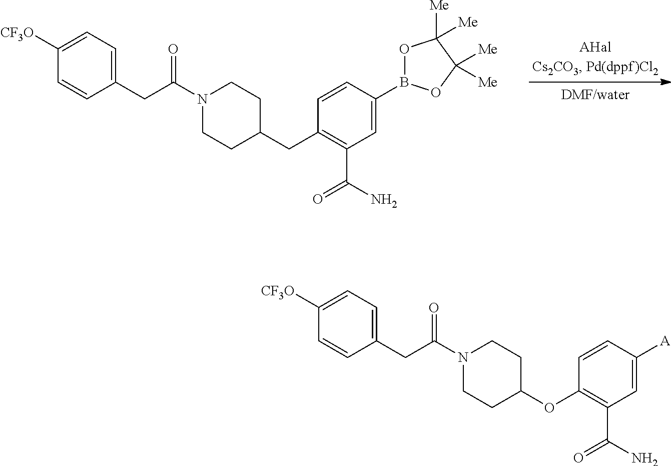

To a 0.2M solution of 5-(4,4,5,5-tetramethyl-1,3,2-dioxaborolan-2-yl)-2-[(1-{[4-(trifluoromethoxy)phenyl]acetyl}piperidin-4-yl)oxy]benzamide (Preparation 14, 500 μL, 100 μmol) in DMF was added a 0.2M solution of compounds of formula (IV) (500 μL, 100 μmol) in DMF with argon purging. A 2M solution of cesium carbonate (100 μL, 200 μmol) in degassed water was added followed by tetrakis(triphenylphosphine)palladium (0) (5.7 mg, 5 μmol) and the reaction was heated to 130° C. under microwave irradiation for 15 minutes. The reaction was cooled and concentrated in vacuo. The residue was dissolved in DMSO (1 mL) and purified using preparative HPLC using one of the Purification Methods (PM) below:

Preparative HPLC Method A: REPROSIL 250×21.1 mm, 5 um eluting with a gradient of between 10-80% acetonitrile in 0.1% formic acid in water. Gradient time: 10 minutes, hold time 1 minute, flow rate 24 mL/min.

Preparative HPLC Method B: XBRIDGE 50×19 mm, 5 um eluting with a gradient of between 10-60% acetonitrile in 0.1% ammonium hydroxide in water. Gradient time: 12 minutes, hold time 1 minute, flow rate 20 mL/min.

Preparative HPLC Method C: REPROSIL 250×21.1 mm, 5 um eluting with a gradient of between 25-65% acetonitrile in 10 mM ammonium acetate in water. Gradient time: 25 minutes, hold time 1 minute, flow rate 10 mL/min.

LCMS QC:

A: 0.05% formic acid in water; B: MeCN

Column: RESTEK C18 30×2.1 mm×3 um

Gradient: From 98% [A] and 2% [B] to 90% [A] and 10% [B] in 1.0 min, further to 98% [B] in 2.0 min and finally back to initial condition in 3.00 min, 1.5 mL/min flow rate

Examples 103-106 were prepared according to Library Protocol 3 using 5-(4,4,5,5-tetramethyl-1,3,2-dioxaborolan-2-yl)-2-[(1-{[4-(trifluoromethoxy)phenyl]acetyl}piperidin-4-yl)oxy]benzamide (Preparation 14) and compounds of formula (IV).

| Exam- | | | |
|---|---|---|---|
| ple | Structure | Name | MS Data and (IV) |
| 103 | 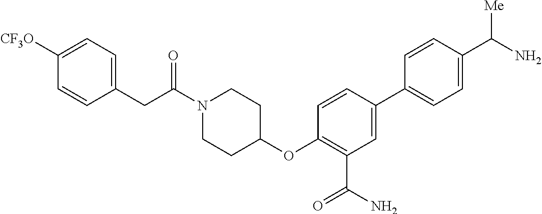 | Racemic 4′-(1- aminoethyl)-4-[(1-{[4- (trifluoromethoxy) phenyl]acetyl}piperidin- 4-yl)oxy]biphenyl-3- carboxamide | MS m/z 542 [M + H]⁺ Rt = 1.43 minutes. PM: Method B. Using 1-(4- bromophenyl) ethanamine. |
| 104 | 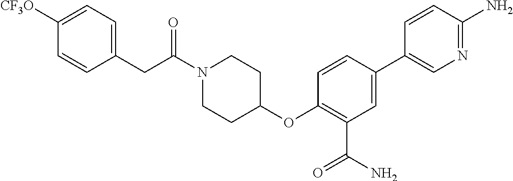 | 5-(6-aminopyridin-3-yl)- 2-[(1-{[4- (trifluoromethoxy) phenyl]acetyl}piperidin- 4-yl)oxy]benzamide | MS m/z 515 [M + H]⁺ Rt = 1.40 minutes. PM: Method B. Using 2-amino-4- iodopyridine. |
| 105 | 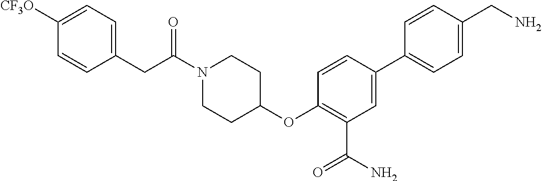 | 4′-(aminomethyl)-4-[(1- {[4-(trifluoromethoxy) phenyl]acetyl}piperidin- 4-yl)oxy]biphenyl-3- carboxamide | MS m/z 528 [M + H]⁺ Rt = 1.42 minutes. PM: Method A. Using 4- iodobenzylamine. |
| 106 | 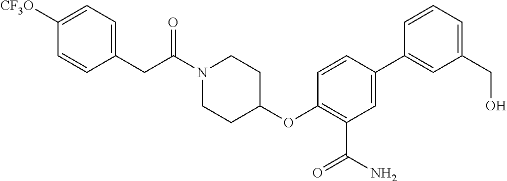 | 3′-(hydroxymethyl)-4- [(1-{[4- (trifluoromethoxy) phenyl]acetyl}piperidin- 4-yl)oxy]biphenyl-3- carboxamide | MS m/z 529 [M + H]⁺ Rt = 1.63 minutes. PM: Method C. Using 3- hydroxymethyliodo benzene. |

Library Protocol 4

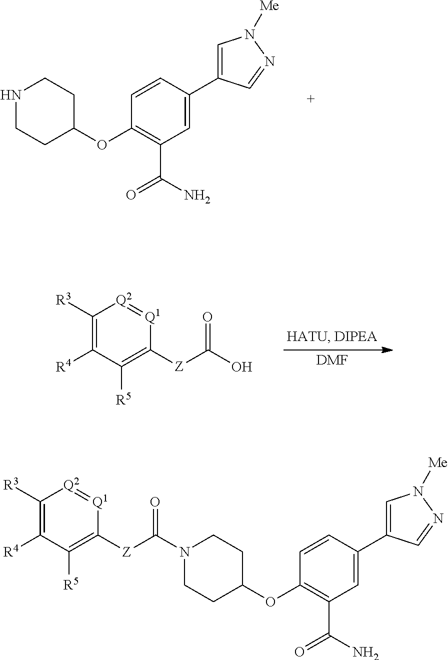

To a 0.2M solution of compounds of formula (V) (500 uL, 100 umol) in DMF was added a 0.2M solution of 5-(1-methyl-1H-pyrazol-4-yl)-2-(piperidin-4-yloxy)benzamide hydrochloride (Preparation 71, 500 uL, 100 umol) in DMF followed by HATU (130 umol) and DIPEA (250 umol). The reaction was stirred at room temperature for 16 hours. The reaction was concentrated in vacuo. The residue was dissolved in DMSO (1 mL) and purified using preparative HPLC using the Purification Method below:

Preparative HPLC Method: XBRIDGE 50×19 mm, 5 um eluting with a gradient of between 10-55% acetonitrile in 0.1% ammonium hydroxide in water. Gradient time: 7 minutes, hold time 1 minute, flow rate 20 mL/min.

LCMS QC:

A: 0.05% formic acid in water; B: MeCN

Column: RESTEK C18 30×2.1 mm×3 um

Gradient: From 98% [A] and 2% [B] to 90% [A] and 10% [B] in 1.0 min, further to 98% [B] in 2.0 min and finally back to initial condition in 3.00 min, 1.5 mL/min flow rate

Examples 107 and 108 were prepared according to Library Protocol 4 using 5-(1-methyl-1H-pyrazol-4-yl)-2-(piperidin-4-yloxy)benzamide hydrochloride (Preparation 71) and compounds of formula (V).

| Example | Structure | Name | MS Data and (V) |
|---|---|---|---|
| 107 | 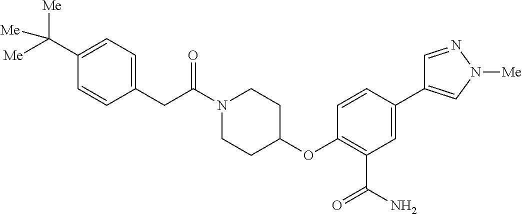 [SMILES: Cn1cc(-c2ccc(OC3CCN(C(=O)Cc4ccc(C(C)(C)C)cc4)CC3)c(C(N)=O)c2)cn1] | 2-({1-[(4-tert- butylphenyl)acetyl] piperidin-4-yl}oxy)-5- (1-methyl-1H- pyrazol-4-yl)benzamide | MS m/z 475 [M + H]⁺ Rt = 1.70 minutes. Using 4-tert- butylphenylacetic acid. |
| 108 | 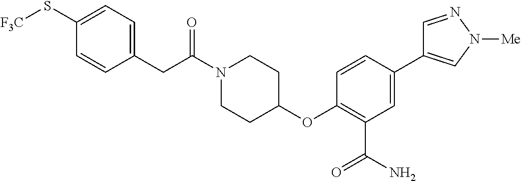 [SMILES: Cn1cc(-c2ccc(OC3CCN(C(=O)Cc4ccc(SC(F)(F)F)cc4)CC3)c(C(N)=O)c2)cn1] | 5-(1-methyl-1H- pyrazol-4- yl)-2-{[1-({4- [(trifluoromethyl) sulfanyl]phenyl}acetyl) piperidin-4- yl]oxy}benzamide | MS m/z 519 [M + H]⁺ Rt = 1.67 minutes. Using 4- [(trifluoromethyl) sulfanyl]phenylacetic acid. |

Library Protocol 5

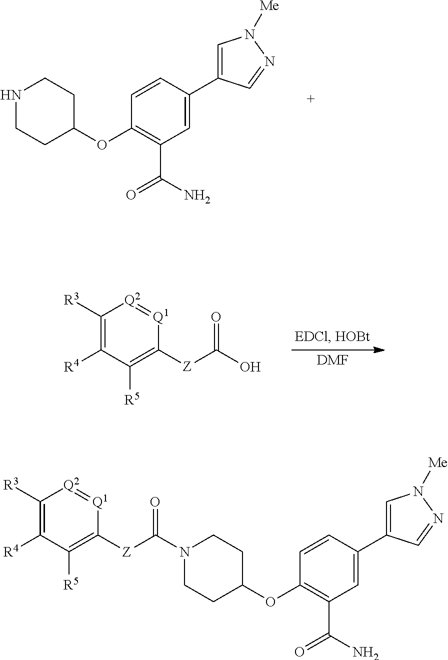

To a 0.2M solution of compounds of formula (V) (500 uL, 100 umol) in DMF was added a 0.2M solution of 5-(1-methyl-1H-pyrazol-4-yl)-2-(piperidin-4-yloxy)benzamide hydrochloride (Preparation 71, 500 uL, 100 umol) in DMF followed by EDCI (48 mg, 250 umol), HOBt (20 mg, 150 umol) and DIPEA (43 uL, 250 umol). The reaction was shaken at 60° C. for 16 hours. The reaction was concentrated in vacuo. The residue was dissolved in DMSO (1 mL) and purified using preparative HPLC using the Purification Method below:

Preparative HPLC Method: XBRIDGE 50×19 mm, 5 um eluting with a gradient of between 10-70% acetonitrile in 0.1% ammonium hydroxide in water. Gradient time: 7 or 10 minutes, hold time 1 minute, flow rate 20 mL/min.

LCMS QC:

A: 0.05% formic acid in water; B: MeCN

Column: RESTEK C18 30×2.1 mm×3 um

Gradient: From 98% [A] and 2% [B] to 90% [A] and 10% [B] in 1.0 min, further to 98% [B] in 2.0 min and finally back to initial condition in 3.00 min, 1.5 mL/min flow rate

Examples 109-111 were prepared according to Library Protocol 5 using 5-(1-methyl-1H-pyrazol-4-yl)-2-(piperidin-4-yloxy)benzamide hydrochloride (Preparation 72) and compounds of formula (V).

| Example | Structure | Name | MS Data and (V) |
|---|---|---|---|
| 109 | 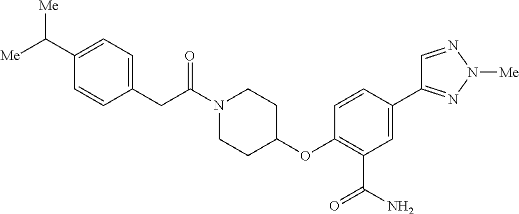 [SMILES: CC(C)c1ccc(CC(=O)N2CCC(Oc3ccc(-c4cnn(C)n4)cc3C(N)=O)CC2)cc1] | 5-(1-methyl-1H-pyrazol-4- yl)-2-[(1-{[4-(propan-2- yl)phenyl]acetyl}piperidin- 4-yl)oxy]benzamide | MS m/z 461 [M + H]⁺ Rt = 1.68 minutes. Using 4-(propan-2- yl)phenylacetic acid. |
| 110 | 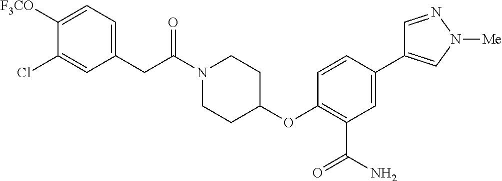 [SMILES: Cn1cc(-c2ccc(OC3CCN(C(=O)Cc4ccc(OC(F)(F)F)c(Cl)c4)CC3)c(C(N)=O)c2)cn1] | 2-[(1-{[3-chloro-4- (trifluoromethoxy)phenyl] acetyl}piperidin-4-yl)oxy]- 5-(1-methyl-1H-pyrazol- 4-yl)benzamide | MS m/z 537 [M + H]⁺ Rt = 1.69 minutes. Using 3-chloro-4- (trifluoromethoxy) phenylacetic acid. |
| 111 | 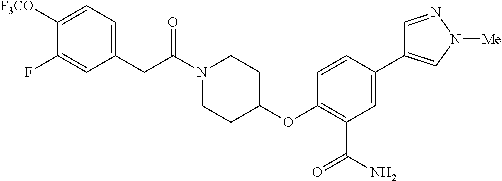 [SMILES: Cn1cc(-c2ccc(OC3CCN(C(=O)Cc4ccc(OC(F)(F)F)c(F)c4)CC3)c(C(N)=O)c2)cn1] | 2-[(1-{[3-fluoro-4- (trifluoromethoxy)phenyl] acetyl}piperidin-4-yl)oxy]- 5-(1-methyl-1H-pyrazol-4- yl)benzamide | MS m/z 521 [M + H]⁺ Rt = 1.65 minutes. Using 3-fluoro-4- (trifluoromethoxy) phenylacetic acid. |

### EXAMPLE 112

### 2-{[(3R,4R)-3-fluoro-1-{[4-(trifluoromethoxy)phenyl]acetyl}piperidin-4-yl]oxy}-5-(1-methyl-1H-imidazol-4-yl)benzamide

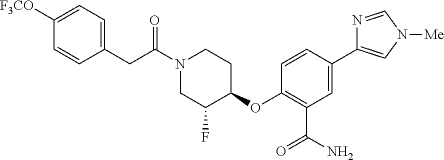 [SMILES: Cn1cnc(-c2ccc(O[C@@H]3CCN(C(=O)Cc4ccc(OC(F)(F)F)cc4)C[C@H]3F)c(C(N)=O)c2)c1]

The title compound may be prepared according to the methods described by Method 8 (Example 8) and Preparations 47, 92 and 157 using trans-1-boc-3-fluoro-4-hydroxypiperidine.

The residue was purified by silica gel column chromatography eluting with 3% MeOH in DCM to afford the racemic compound. The enantiomers were separated by chiral preparative HPLC to afford the title compound, 2-{[(3R,4R)-3-fluoro-1-{[4-(trifluoromethoxy)phenyl]acetyl}piperidin-4-yl]oxy}-5-(1-methyl-1H-imidazol-4-yl)benzamide as the first eluting compound (65 mg, 24%).

¹H NMR (400 MHz, DMSO-d₆): δ ppm 1.72-1.78 (m, 1H), 1.94-1.99 (m, 1H), 2.90-2.94 (m, 1H), 3.05-3.23 (m, 0.5H), 3.45-3.60 (m, 0.5H), 3.67 (s, 3H), 3.84 (d, 2H), 3.97-4.01 (m, 0.5H), 4.31-4.35 (m, 1H), 4.55-4.60 (m, 0.5H), 4.87-4.91 (m, 1H), 5.00-5.13 (m, 1H), 7.26-7.34 (m, 5H), 7.55-7.61 (m, 4H), 7.81 (d, 1H), 8.22 (d, 1H).

MS m/z 521 [M+H]⁺

The second eluting fraction also afforded Example 8, 2-{[(3S,4S)-3-fluoro-1-{[4-(trifluoromethoxy)phenyl]acetyl}piperidin-4-yl]oxy}-5-(1-methyl-1H-imidazol-4-yl)benzamide (70 mg, 26%).

### EXAMPLES 113 AND 114

### (S) and (R)-5-(1-methyl-1H-imidazol-4-yl)-2-((1-(2-(4-(trifluoromethoxy)phenyl)propanoyl)piperidin-4-yl)oxy)benzamide

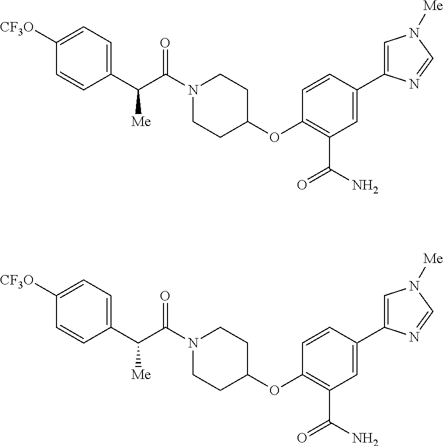

The title compounds were prepared according to Method 7 (Example 7) using 5-(1-methyl-1H-imidazol-4-yl)-2-(piperidin-4-yloxy)benzamide hydrochloride (Preparation 45) and racemic 2-(4-trifluoromethoxyphenyl)propionic acid. The residue was purified using silica gel column chromatography eluting with 2.5% MeOH in DCM. The racemate was then separated into its enantiomers using chiral separation:

(CHIRALPAK-IA (4.6×250) 5μ, Mobile Phase: MeCN/EtOH/DEA: 90/10/0.1, Flow rate: 1.0 ml/min, Solubility: EtOH).

First eluting enantiomer: Example 113: (S)-5-(1-methyl-1H-imidazol-4-yl)-2-((1-(2-(4-(trifluoromethoxy)phenyl)propanoyl)piperidin-4-yl)oxy)benzamide

Second eluting enantiomer: Example 114: (R)-5-(1-methyl-1H-imidazol-4-yl)-2-((1-(2-(4-(trifluoromethoxy)phenyl)propanoyl)piperidin-4-yl)oxy)benzamide

¹H NMR (400 MHz, DMSO-d₆): δ ppm 1.15-1.37 (m, 7H), 1.80-1.95 (m, 2H), 3.10 (m, 1H), 3.66 (s, 3H), 3.80 (m, 1H), 4.25 (m, 1H), 4.70 (m, 1H), 7.13 (m, 1H), 7.33 (m, 2H), 7.42 (m, 3H), 7.54 (m, 2H), 7.62 (br s, 1H), 7.75 (m, 1H), 8.08 (m, 1H).

MS m/z 517 [M+H]⁺

### EXAMPLE 115

### 5-[1-(2-hydroxyethyl)-1H-imidazol-4-yl]-2-[(1-{[4-(trifluoromethoxy)phenyl]acetyl}piperidin-4-yl)oxy]benzamide

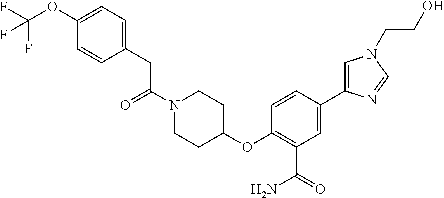 [SMILES: NC(=O)c1cc(-c2cn(CCO)cn2)ccc1OC1CCN(C(=O)Cc2ccc(OC(F)(F)F)cc2)CC1]

The title compound was prepared according to the method described for Method 10 (Example 11) using 5-(4,4,5,5-tetramethyl-1,3,2-dioxaborolan-2-yl)-2-[(1-{[4-(trifluoromethoxy)phenyl]acetyl}piperidin-4-yl)oxy]benzamide (Preparation 14) and 4-bromo-1-[2-(triphenylmethoxy)ethyl]-1H-imidazole (WO 2006028029). The residue was dissolved in THF (1.5 mL) and 3N HCl (0.6 mL) was added. The reaction was stirred at room temperature for 3 hours before concentrating in vacuo. The residue was dissolved in EtOAc (20 mL), washed with saturated aqueous NaHCO₃ solution (5 mL), brine, dried over sodium sulphate and concentrated in vacuo. The residue was purified using preparative TLC eluting with 5% MeOH in DCM to afford the title compound (15 mg, 44%).

¹H NMR (400 MHz, DMSO-d₆): δ ppm 1.65 (m, 2H), 1.92 (m, 2H), 3.37-3.45 (m, 2H), 3.69 (q, 2H), 3.70-3.79 (m, 4H), 4.00 (t, 2H), 4.76 (m, 1H), 4.97 (t, 1H), 7.19 (d, 1H), 7.29 (d, 2H), 7.35 (d, 2H), 7.52 (br s, 2H), 7.58 (s, 1H), 7.62 (s, 1H), 7.77 (dd, 1H), 8.10 (d, 1H).

MS m/z 533 [M+H]⁺

### EXAMPLE 116

### 5-[1-(2-hydroxyethyl)-1H-imidazol-4-yl]-2-[(1-{[4-(trifluoromethoxy)phenyl]acetyl}piperidin-4-yl)oxy]pyridine-3-carboxamide

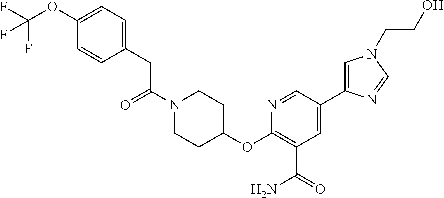 [SMILES: NC(=O)c1cc(-c2cn(CCO)cn2)cnc1OC1CCN(C(=O)Cc2ccc(OC(F)(F)F)cc2)CC1]

The title compound was prepared according to the method described for Example 115 using 5-(4,4,5,5-tetramethyl-1,3,2-dioxaborolan-2-yl)-2-({1-[4-(trifluoromethoxy)benzyl]piperidin-4-yl}oxy)pyridine-3-carboxamide (Preparation 13) and 4-bromo-1-[2-(triphenylmethoxy)ethyl]-1H-imidazole (WO 2006028029).

¹H NMR (400 MHz, DMSO-d₆): δ ppm 1.72 (m, 2H), 1.97 (m, 2H), 3.35-3.46 (m, 2H), 3.69 (q, 2H), 3.77-3.85 (m, 4H), 4.02 (t, 2H), 4.99 (t, 1H), 5.40 (m, 1H), 7.30 (d, 2H), 7.36 (d, 2H), 7.56 (br s, 1H), 7.68 (s, 1H), 7.71 (s, 1H), 7.72 (br s, 1H), 8.46 (s, 1H), 8.62 (s, 1H).

MS m/z 534 [M+H]⁺

### EXAMPLE 117

### 5-[2-(3-fluorooxetan-3-yl)-1H-imidazol-4-yl]-2-[(1-{[4-(trifluoromethoxy)phenyl]acetyl}piperidin-4-yl)oxy]benzamide

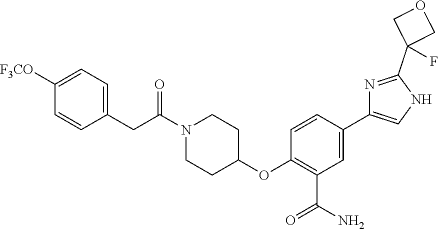 [SMILES: NC(=O)c1cc(-c2c[nH]c(C3(F)COC3)n2)ccc1OC1CCN(C(=O)Cc2ccc(OC(F)(F)F)cc2)CC1]

Morpholinodifluorosulfinium terafluoroborate (52 mg, 0.21 mmol) and 5-[2-(3-hydroxyoxetan-3-yl)-1H-imidazol-5-yl]-2-[(1-{[4-(trifluoromethoxy)phenyl]acetyl}piperidin-4-yl)oxy]benzamide (Example 56, 52 mg, 0.14 mmol) were added to a mixture of triethylamine (20 μL, 0.14 mmol) and triethylamine trihydrofluoride (47 μL, 0.29 mmol) in dichloromethane (3 mL) at −78° C. The reaction mixture was stirred for 15 minutes before warming to room temperature for 18 hours. The reaction was cooled back to −78° C. and a further solution of triethylamine (20 μL, 0.14 mmol) and triethylamine trihydrofluoride (47 μL, 0.29 mmol) in dichloromethane (1 mL) followed by morpholinodifluorosulfinium terafluoroborate (52 mg, 0.21 mmol) were added. The cooling bath was removed and the reaction was stirred for 2 hours. The reaction was quenched by the addition of saturated sodium bicarbonate (10 mL) and stirred for 15 minutes before extracting with dichloromethane (2×10 mL). The organic layers were combined, dried over MgSO₄ and concentrated in vacuo. The residue was purified by silica gel column chromatography eluting with 10% methanol in dichloromethane to afford the title compound as a white solid (25 mg, 32%).

¹H NMR (400 MHz, Acetone-d₆): δ ppm 1.75-1.84 (m, 2H), 2.04-2.10 (m, 2H), 3.38-3.56 (m, 2H), 3.85 (s, 2H), 3.90-4.06 (m, 2H), 4.91-5.22 (m, 5H), 6.73 (br s, 1H), 7.25-7.29 (m, 3H), 7.42-7.44 (m, 2H), 7.60 (br s, 1H), 7.65 (s, 1H), 7.95-7.97 (dd, 1H), 8.50 (d, 1H).

MS m/z 563 [M+H]⁺

### EXAMPLE 118

### 2-{[(3R,4S)-3-fluoro-1-{[4-(trifluoromethoxy)phenyl]acetyl}piperidin-4-yl]oxy}-5-(2-methyl-1H-imidazol-4-yl)pyridine-3-carboxamide

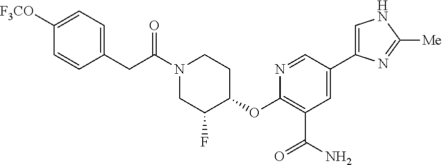 [SMILES: Cc1nc(-c2cnc(O[C@H]3CCN(C(=O)Cc4ccc(OC(F)(F)F)cc4)C[C@H]3F)c(C(N)=O)c2)c[nH]1]

To a solution of regioisomeric mixture 2-{[(3R,4S)-3-fluoro-1-{[4-(trifluoromethoxy)phenyl]acetyl}piperidin-4-yl]oxy}-5-(2-methyl-1-{[2-(trimethylsilyl)ethoxy]methyl}-1H-imidazol-5-yl)pyridine-3-carboxamide and 2-{[(3R,4S)-3-fluoro-1-{[4-(trifluoromethoxy)phenyl]acetyl}piperidin-4-yl]oxy}-5-(2-methyl-1-{[2-(trimethylsilyl)ethoxy]methyl}-1H-imidazol-4-yl)pyridine-3-carboxamide (Preparation 2, 120 mg, 0.18 mmol) in dioxane (3 mL) was added 6N HCl (aqueous, 3 mL) and the reaction was stirred at room temperature for 48 hours. The reaction was diluted with saturate aqueous sodium carbonate solution, extracted into EtOAc, washed with brine, dried over sodium sulphate and concentrated in vacuo. To a solution of the residue in MeOH was added ethylene diamine (0.019 mL) and the reaction was stirred at room temperature for 40 hours. The reaction was concentrated in vacuo, dissolved in EtOAc, washed with water, brine, dried over sodium sulphate and concentrated in vacuo. The residue was purified using silica gel column chromatography eluting with 5% MeOH in DCM to afford the title compound as a white solid (50 mg, 66%).

NMR exhibits rotameric behaviour: ¹H NMR (400 MHz, DMSO-d₆): δ ppm 1.75-1.86 (m, 1H), 1.99-1.23 (m, 1H), 2.31 (s, 3H), 2.94 (t, 0.5H), 3.12-3.25 (m, 0.5H), 3.54-3.73 (m, 1H), 3.84 (s, 2H), 4.00-4.04 (m, 0.5H), 4.33-4.37 (m, 1H), 4.57-4.61 (m, 0.5H), 5.02-5.17 (m, 1H), 5.48-5.56 (m, 1H), 7.25-7.36 (m, 4H), 7.52 (br s, 1H), 7.55 (s, 1H), 7.80 (br s, 1H), 8.53 (s, 1H), 8.63 (s, 1H), 11.89 (s, 1H).

MS m/z 522 [M+H]⁺

### EXAMPLE 119

### 5-{1-[(2S)-2,3-dihydroxypropyl]-1H-pyrazol-4-yl}-2-[(1-{[4-(trifluoromethoxy)phenyl]acetyl}piperidin-4-yl)oxy]benzamide

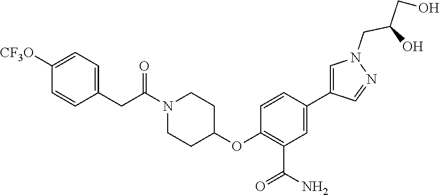

To a stirred solution of 5-(1-{[(4S)-2,2-dimethyl-1,3-dioxolan-4-yl]methyl}-1H-pyrazol-4-yl)-2-[(1-{[4-(trifluoromethoxy)phenyl]acetyl}piperidin-4-yl)oxy]benzamide (Preparation 11, 60 mg, 0.1 mmol) in THF (2 mL) was added aqueous 2N HCl (2 mL) at 0° C. and the solution was allowed to warm to room temperature and stirred for 2 hours. The reaction mixture was quenched with aqueous saturated NaHCO₃ solution (5 mL) and extracted with ethyl acetate (2×10 mL). The organic layer dried over sodium sulphate and concentrated in vacuo. The residue was purified using preparative TLC eluting with 5% MeOH in DCM to afford the title compound as white solid (17 mg, 30%).

¹H NMR (400 MHz, DMSO-d₆): δ ppm 1.64 (m, 2H), 1.90 (m, 2H), 3.31-3.43 (m, 4H), 3.75-3.82 (m, 5H), 3.96-4.01 (m, 1H), 4.19-4.23 (m, 1H), 4.71-4.76 (m, 2H), 4.98 (d, 1H), 7.20 (d, 1H), 7.29 (d, 2H), 7.35 (d, 2H), 7.53 (br s, 1H), 7.55 (br s, 1H), 7.60 (dd, 1H), 7.80 (s, 1H), 7.87 (d, 1H), 8.06 (s, 1H).

MS m/z 563 [M+H]⁺

The following Examples 120-125 were prepared according to Example 119 using the appropriately protected precursor as described below:

| Exam- | | | |
|---|---|---|---|
| ple | Structure and Name | Data | Starting materials |
| 120 | 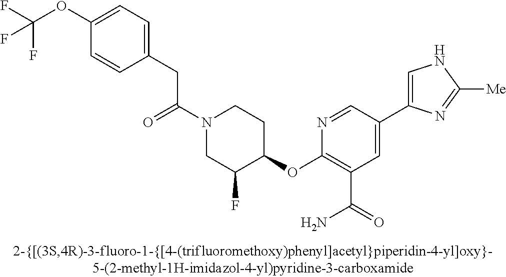 [SMILES: Cc1nc(-c2cnc(O[C@@H]3CCN(C(=O)Cc4ccc(OC(F)(F)F)cc4)C[C@@H]3F)c(C(N)=O)c2)c[nH]1] | MS m/z 522 [M + H]⁺ ¹H NMR (400 MHz, DMSO-d₆): δ ppm 2.01 (m, 1H), 2.05 (m, 1H), 2.33 (s, 3H), 3.19-3.58 (m, 2H), 3.76 (m, 2H), 4.14-4.40 (m, 2H), 5.01- 5.13 (m, 1H), 5.50-5.59 (m, 1H), 7.25 (m, 2H), 7.36 (m, 4H), 8.54 (br s, 1H), 8.60 (br s, 1H), 11.61 (br s, 1H). | 2-{[(3S,4R)-3-fluoro- 1-{[4- (trifluoromethoxy) phenyl]acetyl} piperidin-4-yl]oxy}- 5-(2-methyl-1-{[2- (trimethylsilyl) ethoxy]methyl}-1H- imidazol-4- yl)pyridine-3- carboxamide (Preparation 8). |
| 121 | 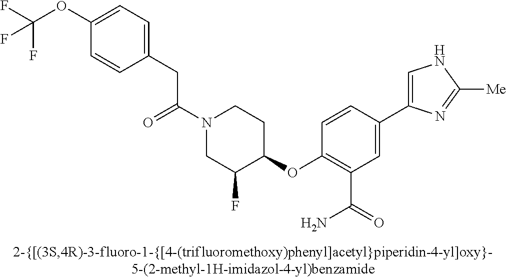 [SMILES: Cc1nc(-c2ccc(O[C@@H]3CCN(C(=O)Cc4ccc(OC(F)(F)F)cc4)C[C@@H]3F)c(C(N)=O)c2)c[nH]1] | ¹H NMR (400 MHz, DMSO-d₆): δ ppm 1.71 (m, 1H), 1.96 (m, 1H), 2.29 (s, 3H), 2.90-3.26 (m, 2H), 3.44-3.97 (m, 2H), 4.32 (m, 1H), 4.59 (m, 1H), 4.87-5.12 (m, 2H), 7.29-7.39 (m, 5H), 7.55 (m, 2H), 7.76 (br s, 1H), 8.19 (br s, 1H), 11.78 (br s, 1H). | 2-{[(3S,4R)-3-fluoro- 1-{[4- (trifluoromethoxy) phenyl]acetyl} piperidin- 4-yl]oxy}-5-(2- methyl-1-{[2- (trimethylsilyl)ethoxy] methyl}-1H- imidazol-4- yl)benzamide (Preparation 7). |
| 122 | 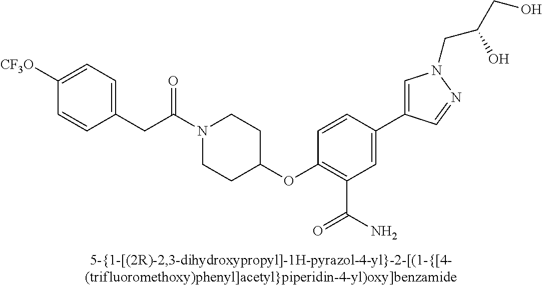 | MS m/z 563 [M + H]⁺ ¹H NMR (400 MHz, DMSO-d₆): δ ppm 1.64 (m, 2H), 1.90 (m, 2H), 3.31-3.43 (m, 4H), 3.75-3.82 (m, 5H), 3.96-4.01 (m, 1H), 4.19-4.23 (m, 1H), 4.71-4.76 (m, 2H), 4.98 (d, 1H), 7.20 (d, 1H), 7.29 (d, 2H), 7.35 (d, 2H), 7.53 (br s, 1H), 7.55 (br s, 1H), 7.60 (dd, 1H), 7.80 (s, 1H), 7.87 (d, 1H), 8.06 (s, 1H). | 5-(1-{[(4R)-2,2- dimethyl-1,3- dioxolan-4- yl]methyl}-1H- pyrazol-4-yl)-2-[(1- {[4- (trifluoromethoxy) phenyl]acetyl} piperidin- 4-yl)oxy]benzamide (Preparation 3). |
| 123 | 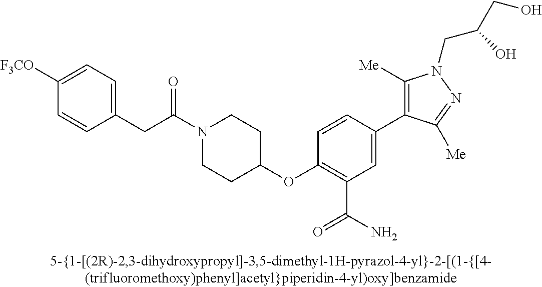 [SMILES: Cc1nn(C[C@@H](O)CO)c(C)c1-c1ccc(OC2CCN(C(=O)Cc3ccc(OC(F)(F)F)cc3)CC2)c(C(N)=O)c1] | MS m/z 591 [M + H]⁺ ¹H NMR (400 MHz, DMSO-d₆): δ ppm 1.66 (m, 2H), 1.93 (m, 2H), 2.11 (s, 3H), 2.20 (s, 3H), 3.34-3.50 (m, 3H), 3.75-3.90 (m, 7H), 4.04 (m, 1H), 4.75 (m, 2H), 4.95 (m, 1H), 7.23-7.37 (m, 6H), 7.57 (m, 3H). | 5-(1-{[(4R)-2,2- dimethyl-1,3- dioxolan-4- yl]methyl}-3,5- dimethyl-1H-pyrazol- 4-yl)-2-[(1-{[4- (trifluoromethoxy) phenyl]acetyl} piperidin- 4-yl)oxy]benzamide (Preparation 4), and eluting with 10% MeOH in DCM. |
| 124 | 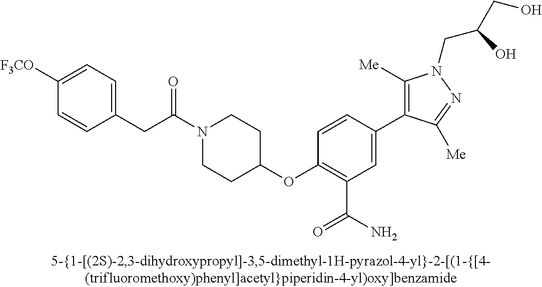 [SMILES: Cc1nn(C[C@H](O)CO)c(C)c1-c1ccc(OC2CCN(C(=O)Cc3ccc(OC(F)(F)F)cc3)CC2)c(C(N)=O)c1] | MS m/z 591 [M + H]⁺ ¹H NMR (400 MHz, DMSO-d₆): δ ppm 1.64 (m, 2H), 1.93 (m, 2H), 2.08 (s, 3H), 2.18 (s, 3H), 3.34-3.50 (m, 3H), 3.75-3.90 (m, 7H), 4.02 (m, 1H), 4.65-4.80 (m, 2H), 4.89 (m, 1H), 7.20-7.35 (m, 6H), 7.51 (m-7.55, 3H). | 5-(1-{[(4S)-2,2- dimethyl-1,3- dioxolan-4- yl]methyl}-3,5- dimethyl-1H-pyrazol- 4-yl)-2-[(1-{[4- (trifluoromethoxy) phenyl]acetyl} piperidin- 4-yl)oxy]benzamide (Preparation 5), and eluting with 10% MeOH in DCM. |
| 125 | 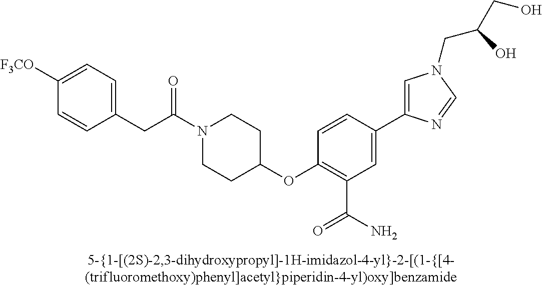 [SMILES: NC(=O)c1cc(-c2cn(C[C@H](O)CO)cn2)ccc1OC1CCN(C(=O)Cc2ccc(OC(F)(F)F)cc2)CC1] | MS m/z 563 [M + H]⁺ ¹H NMR (400 MHz, DMSO-d₆): δ ppm 1.65 (m, 2H), 1.91 (m, 2H), 3.22-3.45 (m, 4H), 3.74-3.90 (m, 6H), 4.07 (m, 1H), 4.76 (m, 2H), 5.07 (d, 1H), 7.18 (d, 1H), 7.28-7.36 (m, 4H), 7.52 (m, 2H), 7.76-7.78 (m, 1H), 8.10 (d, 1H). | 5-(1-{[(4S)-2,2- dimethyl-1,3- dioxolan-4- yl]methyl}-1H- imidazol-4-yl)-2-[(1- {[4-(trifluoromethoxy) phenyl]acetyl} piperidin- 4-yl)oxy]benzamide (Preparation 6). |

### EXAMPLE 126

### 5-{1-[(2R)-2,3-dihydroxypropyl]-1H-imidazol-4-yl}-2-[(1-{[4-(trifluoromethoxy)phenyl]acetyl}piperidin-4-yl)oxy]benzamide

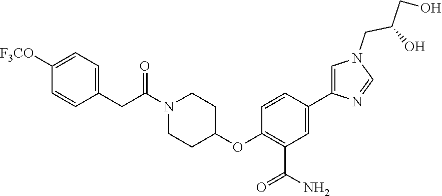 [SMILES: NC(=O)c1cc(-c2cn(C[C@@H](O)CO)cn2)ccc1OC1CCN(C(=O)Cc2ccc(OC(F)(F)F)cc2)CC1]

The title compound was prepared according to the method described for Example 119, Preparations 6 and 193 using [(4R)-2,2-dimethyl-1,3-dioxolan-4-yl]methyl methanesulfonate (Preparation 194).

¹H NMR (400 MHz, DMSO-d₆): δ ppm 1.65 (m, 2H), 1.91 (m, 2H), 3.20-3.42 (m, 4H), 3.74-3.90 (6H), 4.06 (m, 1H), 4.76-4.81 (m, 2H), 5.07 (m, 1H), 7.18 (d, 1H), 7.28-7.36 (m, 4H), 7.52-7.58 (m, 4H), 7.76 (m, 1H), 8.10 (d, 1H).

### EXAMPLE 127

### 5-(1H-imidazol-4-yl)-2-[(1-{[4-(trifluoromethoxy)phenyl]acetyl}piperidin-4-yl)oxy]benzamide

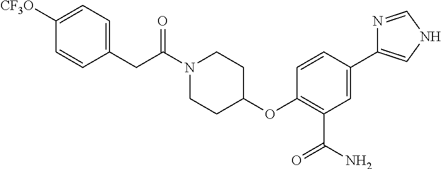

To a stirred solution of 5-(4,4,5,5-tetramethyl-1,3,2-dioxaborolan-2-yl)-2-[(1-{[4-(trifluoromethoxy)phenyl]acetyl}piperidin-4-yl)oxy]benzamide (Preparation 14, 400 mg, 0.73 mmol) and 5-bromo-1-{[2-(trimethylsilyl)ethoxy]methyl}-1H-imidazole and 4-bromo-1-{[2-(trimethylsilyl)ethoxy]methyl}-1H-imidazole (Preparation 196, 202 mg, 0.73 mmol) in DMF-water (9:1, 5 mL) in a sealed tube was added cesium carbonate (475 mg, 1.46 mmol) and the reaction was degassed with argon for 15 minutes. Tetrakis(triphenylphosphine)palladium(0) (42 mg, 0.36 mmol) was added and the reaction was heated to 110° C. for 16 hours before cooling to room temperature and diluting with ethyl acetate (20 mL). The solution was washed with water (2×20 mL), brine (10 mL), dried over sodium sulphate and concentrated in vacuo. The residue was purified using silica gel column chromatography eluting with 3% MeOH in DCM to afford a brown solid. The solid was dissolved in DCM (1 mL), TFA (1 mL) was added at 0° C. and the reaction was stirred at room temperature for 24 hours. The reaction was concentrated in vacuo and diluted with EtOAc (10 mL). The solution was washed saturated aqueous NaHCO₃ (10 mL), washed with water (2×10 mL), dried over sodium sulfate and concentrated in vacuo. The residue was purified using silica gel column chromatography eluting with 5% MeOH in DCM followed by preparative TLC eluting with 6% MeOH in DCM to afford the title compound as light brown solid (22 mg, 19%).

¹H NMR (400 MHz, DMSO-d₆): δ ppm 1.65 (m, 2H), 1.90 (m, 2H), 3.39-3.43 (m, 2H), 3.75-3.83 (m, 4H), 4.76 (m, 1H), 7.19 (d, 1H), 7.29 (d, 2H), 7.35 (d, 2H), 7.53 (m, 3H), 7.67 (s, 1H), 7.81 (d, 1H), 8.13 (s, 1H), 12.10 (s, 1H).

MS m/z 489 [M+H]⁺

### EXAMPLE 128

### 5-{1-[2-(methylamino)-2-oxoethyl]-1H-imidazol-4-yl}-2-[(1-{[4-(trifluoromethoxy)phenyl]acetyl}piperidin-4-yl)oxy]benzamide

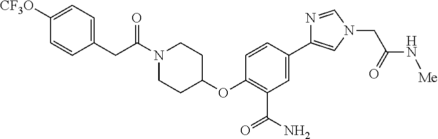

A mixture of methyl (4-{3-carbamoyl-4-[(1-{[4-(trifluoromethoxy)phenyl]acetyl}piperidin-4-yl)oxy]phenyl}-1H-imidazol-1-yl)acetate (Preparation 1, 61.6 mg, 0.110 mmol) and 1M lithium hydroxide in water (0.22 mL, 0.220 mmol) in 2-methyltetrahydrofuran (2 mL) and water (0.30 mL) was stirred under nitrogen at room temperature for one hour. The mixture was concentrated in vacuo and the crude residue was taken up in 2-methyltetrahydrofuran (5 mL). 2M methylamine in tetrahydrofuran (0.06 mL, 0.120 mmol) and pyridine (26.6 μL, 0.330 mmol) was added and the mixture was heated to 85° C. under nitrogen. Once at 85° C., propylphosphonic anhydride (55.7 μL, 0.187 mmol) was added and the reaction stirred at 85° C. for 18 hours. Additional 2M methylamine in tetrahydrofuran (0.18 mL, 0.360 mmol) was added followed by propylphosphonic anhydride (111.4 μL, 0.374 mmol)and the reaction continued for a further 24 hours. The reaction was cooled to room temperature and concentrated in vacuo. The residue was purified using Preparative HPLC to afford the title compound as a colourless residue (15.4 mg, 25%).

¹H NMR (400 MHz, CD₃OD):δ ppm 1.70-1.84 (m, 2H), 1.93-2.06 (m, 2H), 2.77 (s, 3H), 3.49-3.58 (m, 2H), 3.79-3.84 (m, 3H), 3.88-3.95 (m, 1H), 4.75 (s, 2H), 4.80-4.83 (m, 1H), 7.20 (d, 1H), 7.24 (d, 2H), 7.36 (d, 2H), 7.44 (d, 1H), 7.70 (d, 1H), 7.84 (dd, 1H), 8.20 (d, 1H).

MS m/z 560 [M+H]⁺

### EXAMPLE 129

### 5-{1-[(3-hydroxyoxetan-3-yl)methyl]-1H-pyrazol-4-yl}-2-[(1-{[4-(trifluoromethoxy)phenyl]acetyl}piperidin-4-yl)oxy]benzamide

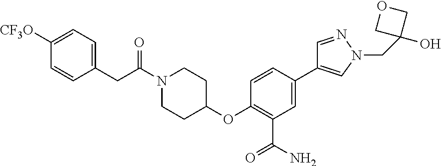

To a solution of 2-hydroxy-5-(1-((3-hydroxyoxetan-3-yl)methyl)-1H-pyrazol-4-yl)benzamide (Preparation 167, 42 mg, 0.14 mmol) in DMF (5 mL) was added 1-{[4-(trifluoromethoxy)phenyl]acetyl}piperidin-4-yl methanesulfonate (Preparation 181, 277 mg, 0.72 mmol) and cesium carbonate (95 mg, 0.29 mmol). The reaction was heated to 80° C. for 3 hours then at 60° C. for 18 hours. The reaction was cooled, poured onto water (20 mL) and extracted with EtOAc (3×20 mL). The combined organic layers were dried over MgSO₄ and concentrated in vacuo. The residue was purified using preparative HPLC to afford the title compound as a white solid (34 mg, 41%).

¹H NMR (400 MHz, DMSO-d₆): δ ppm 1.70 (m, 1H), 1.84 (m, 1H), 1.91 (m, 1H), 2.07 (m, 1H), 3.39 (m, 1H), 3.52 (m, 1H), 3.70 (m, 1H), 3.75 (s, 2H), 3.97 (m, 1H), 4.44 (d, 2H), 4.53 (s, 2H), 4.65 (d, 2H), 4.67 (m, 1H), 6.02 (br s, 1H), 6.97 (d, 1H), 7.17 (d, 2H), 7.27 (d, 2H), 7.52 (d, 1H), 7.54 (br s, 1H), 7.77 (s, 2H), 8.27 (d, 1H).

MS m/z 575 [M+H]⁺

### EXAMPLE 130

### 2-[(1-{[4-(cyclopropyloxy)phenyl]acetyl}piperidin-4-yl)oxy]-5-[1-(2-hydroxyethyl)-1H-pyrazol-4-yl]benzamide

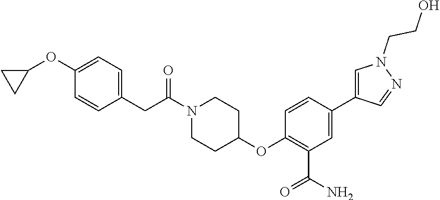 [SMILES: NC(=O)c1cc(-c2cnn(CCO)c2)ccc1OC1CCN(C(=O)Cc2ccc(OC3CC3)cc2)CC1]

To a solution of 2-((1-(2-(4-cyclopropoxyphenyl)acetyl)piperidin-4-yl)oxy)-5-(1H-pyrazol-4-yl)benzamide (Example 93, 120 mg, 0.26 mmol) in MeCN (5 mL) was added 2-(2-bromo-ethoxy)-tetrahydropyran (65 mg, 0.31 mmol) followed by cesium carbonate and the reaction was heated to 100° C. for 16 hours. The reaction was cooled and partitioned between EtOAc and water. The organic layer was collected, dried over sodium sulphate and concentrated in vacuo. The residue was purified using silica gel column chromatography eluting with 1-2% MeOH in DCM. The residue was dissolved in MeOH (2 mL) and treated with pTSA (5 mg, 0.03 mmol) and stirred at room temperature for 6 hours. The reaction was concentrated in vacuo and diluted with DCM, washed with saturated aqueous NaHCO₃ solution, brine, dried over sodium sulphate and concentrated in vacuo. The residue was purified using preparative TLC eluting with 5% MeOH in DCM to afford the title compound (25 mg, 35%).

¹H NMR (400 MHz, DMSO-d₆): δ ppm 0.60-0.64 (m, 2H), 0.75-0.78 (m, 2H), 1.55-1.64 (m, 2H), 1.82-1.92 (m, 2H), 3.36-3.42 (m, 2H), 3.70 (s, 2H), 3.73-3.80 (m, 5H), 4.14 (t, 2H), 4.70-4.76 (m, 1H), 4.91 (t, 1H), 6.97 (d, 2H), 7.15 (d, 2H), 7.18 (d, 1H), 7.52 (br, 1H), 7.54 (br, 1H), 7.60 (dd, 1H), 7.80 (s, 1H), 7.86 (d, 1H), 8.10 (s, 1H).

MS m/z 505 [M+H]⁺

### EXAMPLE 131

### 5-{1-[(2S)-1-hydroxypropan-2-yl]-1H-pyrazol-4-yl}-2-[(1-{[4-(trifluoromethoxy)phenyl]acetyl}piperidin-4-yl)oxy]benzamide

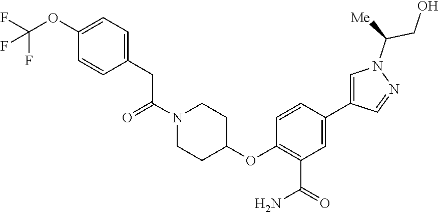 [SMILES: C[C@@H](CO)n1cc(-c2ccc(OC3CCN(C(=O)Cc4ccc(OC(F)(F)F)cc4)CC3)c(C(N)=O)c2)cn1]

To a solution of (2R)-1-{[tert-butyl(diphenyl)silyl]oxy}propan-2-yl methanesulfonate (Preparation 203, 280 mg, 0.714 mmol) in DMF (3 mL) was added cesium carbonate (348 mg, 1.070 mmol) and 5-(1H-pyrazol-4-yl)-2-[(1-{[4-(trifluoromethoxy)phenyl]acetyl}piperidin-4-yl)oxy]benzamide (Example 11, 174 mg, 0.357 mmol). The reaction was heated to 110° C. for 18 hours before cooling and diluting with EtOAc. The solution was washed with water, dried over sodium sulphate and concentrated in vacuo. The residue was purified using silica gel column chromatography eluting with 4% MeOH in DCM followed by preparative HPLC to afford the title compound (25 mg, 6%).

¹H NMR (400 MHz, DMSO-d₆): δ ppm 1.40 (d, 3H), 1.64 (m, 2H), 1.90 (m, 2H), 3.43-3.51 (m, 2H), 3.60-3.85 (m, 6H), 4.32 (m, 1H), 4.76 (m, 1H), 4.92 (m, 1H), 7.20 (d, 1H), 7.28-7.36 (m, 4H), 7.54 (m, 2H), 7.60-7.63 (m, 1H), 7.80 (br s, 1H), 7.88 (m, 1H), 8.13 (br s, 1H).

MS m/z 547 [M+H]⁺

The following Examples 132-135 were prepared according to Example 131 using 5-(1H-pyrazol-4-yl)-2-[(1-{[4-(trifluoromethoxy)phenyl]acetyl}piperidin-4-yl)oxy]benzamide (Example 11) in either acetonitrile or DMF and the appropriate pyrazole as described below:

| Example | Structure and Name | Data | Starting materials |
|---|---|---|---|
| 132 | 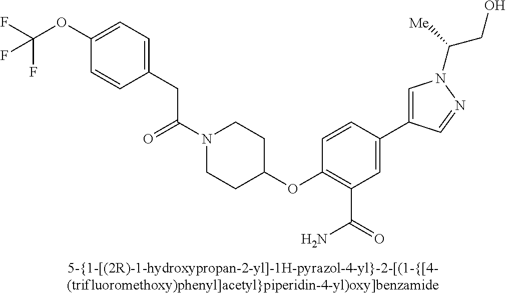 [SMILES: C[C@H](CO)n1cc(-c2ccc(OC3CCN(C(=O)Cc4ccc(OC(F)(F)F)cc4)CC3)c(C(N)=O)c2)cn1] | MS m/z 547 [M + H]⁺ ¹H NMR (400 MHz, DMSO-d₆): δ ppm 1.40 (d, 3H), 1.64 (m, 2H), 1.90 (m, 2H), 3.43-3.51 (m, 2H), 3.60-3.85 (m, 6H), 4.32 (m, 1H), 4.76 (m, 1H), 4.92 (m, 1H), 7.20 (d, 1H), 7.28-7.36 (m, 4H), 7.54 (m, 2H), 7.60- 7.63 (m, 1H), 7.80 (br s, 1H), 7.88 (m, 1H), 8.13 (br s, 1H). | (2S)-1-{[tert- butyl(diphenyl) silyl]oxy}propan- 2-yl methanesulfonate (Preparation 202) in MeCN. |
| 133 | 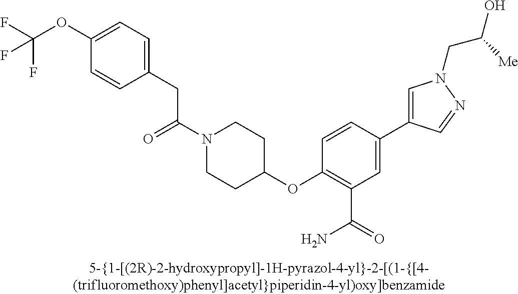 [SMILES: C[C@@H](O)Cn1cc(-c2ccc(OC3CCN(C(=O)Cc4ccc(OC(F)(F)F)cc4)CC3)c(C(N)=O)c2)cn1] | MS m/z 547 [M + H]⁺ ¹H NMR (400 MHz, DMSO-d₆): δ ppm 0.99 (d, 3H), 1.48 (m, 2H), 1.90 (m, 2H), 3.34-3.45 (m, 2H), 3.75-3.85 (m, 4H), 4.00 (m, 3H), 4.76 (m, 1H), 4.92 (m, 1H), 7.18 (dd, 1H), 7.29-7.37 (m, 4H), 7.53-7.62 (m, 3H), 7.80 (br s, 1H), 7.87 (m, 1H), 8.07 (br s, 1H). | (2S)-2- methyloxirane in DMF and purification using preparative TLC eluting with 5% MeOH in EtOAc |
| 134 | 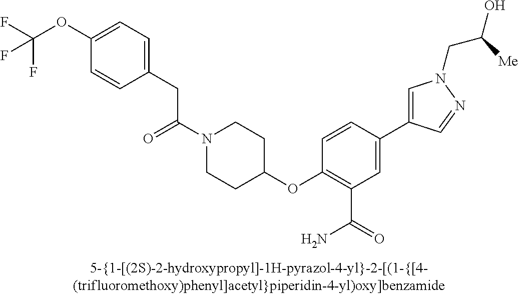 [SMILES: C[C@H](O)Cn1cc(-c2ccc(OC3CCN(C(=O)Cc4ccc(OC(F)(F)F)cc4)CC3)c(C(N)=O)c2)cn1] | MS m/z 547 [M + H]⁺ ¹H NMR (400 MHz, DMSO-d₆): δ ppm 1.05 (d, 3H), 1.66 (m, 2H), 1.90 (m, 2H), 3.32-3.45 (m, 2H), 3.75-3.85 (m, 4H), 4.00 (m, 3H), 4.76 (m, 1H), 4.93 (m, 1H), 7.19 (dd, 1H), 7.29-7.37 (m, 4H), 7.54-7.62 (m, 3H), 7.81 (br s, 1H), 7.87 (m, 1H), 8.08 (br s, 1H). | (2R)-2- methyloxirane in DMF and purification using preparative TLC eluting with 5% MeOH in EtOAc. |
| 135 | 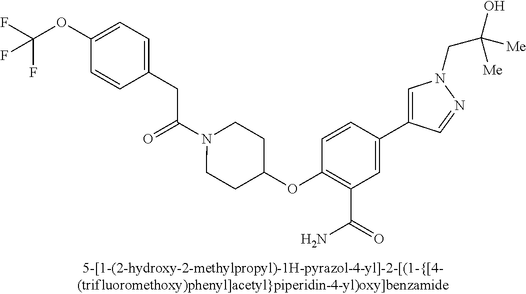 [SMILES: CC(C)(O)Cn1cc(-c2ccc(OC3CCN(C(=O)Cc4ccc(OC(F)(F)F)cc4)CC3)c(C(N)=O)c2)cn1] | MS m/z 561 [M + H]⁺ ¹H NMR (400 MHz, DMSO-d₆): δ ppm 1.08 (s, 6H), 1.65 (m, 2H), 1.89 (m, 2H), 3.31-3.50 (m, 2H), 3.70-3.85 (m, 4H), 4.01 (s, 2H), 4.71 (s, 1H), 4.77 (m, 1H), 7.20 (d, 1H), 7.30-7.38 (m, 4H), 7.54- 7.63 (m, 3H), 7.80 (br s, 1H), 7.87 (m, 1H), 8.04 (br s, 1H). | 2,2- dimethyloxirane in DMF and purification using preparative TLC eluting with 5% MeOH in EtOAc. |

### EXAMPLE 136

### 5-(1H-1,2,3-triazol-4-yl)-2-((1-(2-(4-(trifluoromethoxy)phenyl)acetyl)piperidin-4-yl)oxy)benzamide

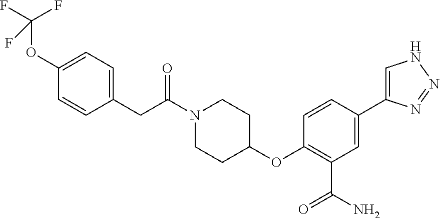 [SMILES: NC(=O)c1cc(-c2c[nH]nn2)ccc1OC1CCN(C(=O)Cc2ccc(OC(F)(F)F)cc2)CC1]

To a solution of (4-(3-carbamoyl-4-((1-(2-(4-(trifluoromethoxy)phenyl)acetyl)piperidin-4-yl)oxy)phenyl)-1H-1,2,3-triazol-1-yl)methyl pivalate (Preparation 20, 400 mg, 0.66 mmol) in MeOH (8 mL) was added 1N NaOH (1.6 mL) and the reaction was stirred at room temperature for 30 minutes. The reaction was concentrated in vacuo and partitioned between EtOAc and water. The organic layer was collected, washed with brine, dried over sodium sulphate and concentrated in vacuo. The residue was purified using silica gel column chromatography eluting with 0.5% MeOH in DCM) to afford the title compound.

¹H NMR (400 MHz, DMSO-d₆): δ ppm 1.61-1.69 (m, 2H), 1.88-1.96 (m, 2H), 3.34-3.47 (m, 2H), 3.75-3.82 (m, 4H), 4.79-4.85 (m, 1H), 7.28-7.37 (m, 5H), 7.55 (br s, 1H), 7.59 (br s, 1H), 7.90 (d, 1H), 8.19 (s, 1H), 8.29 (br s, 1H), 15.11 (br s, 1H).

MS m/z 490 [M+H]⁺

### EXAMPLE 137

### 5-(1-(2-hydroxyethyl)-1H-1,2,3-triazol-4-yl)-2-((1-(2-(4-(trifluoromethoxy)phenyl)acetyl)piperidin-4-yl)oxy)benzamide

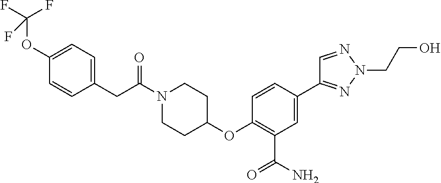 [SMILES: NC(=O)c1cc(-c2cnn(CCO)n2)ccc1OC1CCN(C(=O)Cc2ccc(OC(F)(F)F)cc2)CC1]

To a solution of 5-(1H-1,2,3-triazol-4-yl)-2-((1-(2-(4-(trifluoromethoxy)phenyl)acetyl)piperidin-4-yl)oxy)benzamide (Example 136, 200 mg, 0.41 mmol) in MeCN (10 mL) was added 2-(2-bromo-ethoxy)-tetrahydropyran (0.12 mL, 1.02 mmol) and potassium carbonate (170 mg, 1.23 mmol) and the reaction was heated to 110° C. for 16 hours. The reaction was cooled, diluted with EtOAc, washed with water, brine, dried over sodium sulphate and concentrated in vacuo. The residue was dissolved in 4M HCl in dioxane (10 mL) and stirred at room temperature for 3 hours. The reaction was concentrated in vacuo, basified with saturated aqueous sodium carbonate solution and extracted with EtOAc. The organic layer was collected, washed with brine, dried over sodium sulphate and concentrated in vacuo. The residue was purified using silica gel column chromatography eluting with 0-5% MeOH in DCM to afford the title compound.

¹H NMR (400 MHz, DMSO-d₆): δ ppm 1.62-1.71 (m, 2H), 1.92-1.98 (m, 2H), 3.31-3.48 (m, 2H), 3.72-3.84 (m, 4H), 3.90 (q, 2H), 4.46 (t, 2H), 4.81-4.85 (m, 1H), 4.93 (t, 1H), 7.28-7.37 (m, 5H), 7.55 (br s, 1H), 7.61 (br s, 1H), 7.86 (dd, 1H), 8.18-8.19 (m, 2H).

MS m/z 534 [M+H]⁺

### EXAMPLE 138

### 5-[1-(2-aminoethyl)-1H-pyrazol-4-yl]-2-[(1-{[4-(trifluoromethoxy)phenyl]acetyl}piperidin-4-yl)oxy]benzamide

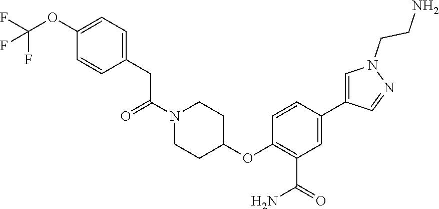 [SMILES: NCCn1cc(-c2ccc(OC3CCN(C(=O)Cc4ccc(OC(F)(F)F)cc4)CC3)c(C(N)=O)c2)cn1]

To a degassed solution of 5-{1-[2-(dibenzylamino)ethyl]-1H-pyrazol-4-yl}-2-[(1-{[4-(trifluoromethoxy)phenyl]acetyl}piperidin-4-yl)oxy]benzamide (Preparation 10, 200 mg, 0.281 mmol) in EtOAc (6 mL) was added 10% Pd/C (20 mg) and the reaction was hydrogenated under an atmosphere of hydrogen for 16 hours. The reaction was filtered through celite, concentrated in vacuo and dissolved in MeOH (6 mL). 10% Pd/C (20 mg) was added and the reaction again hydrogenated as before for 18 hours. The reaction was filtered through celite, concentrated in vacuo and purified using silica gel column chromatography eluting with 6-7% MeOH in DCM to afford the title compound (90 mg, 60%).

¹H NMR (400 MHz, DMSO-d₆): δ ppm 1.62 (m, 2H), 1.90 (m, 2H), 2.95 (t, 2H), 3.30-3.50 (m, 2H), 3.70-3.85 (m, 4H), 4.07 (t, 2H), 4.76 (m, 1H), 7.19 (d, 1H), 7.21-7.36 (m, 4H), 7.53 (m, 2H), 7.62 (m, 1H), 7.81 (s, 1H), 7.88 (br s, 1H), 8.12 (s, 1H).

MS m/z 532 [M+H]⁺

### EXAMPLE 139

### 5-(2-methyl-1H-imidazol-4-yl)-2-[(1-{[4-(trifluoromethoxy)phenyl]acetyl}piperidin-4-yl)oxy]pyridine-3-carboxamide

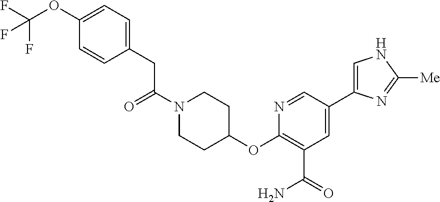 [SMILES: Cc1nc(-c2cnc(OC3CCN(C(=O)Cc4ccc(OC(F)(F)F)cc4)CC3)c(C(N)=O)c2)c[nH]1]

To a solution of 5-(2-methyl-1-{[2-(trimethylsilyl)ethoxy]methyl}-1H-imidazol-4-yl)-2-[(1-{[4-(trifluoromethoxy)phenyl]acetyl}piperidin-4-yl)oxy]pyridine-3-carboxamide (Preparation 9, 200 mg, 0.316 mmol) in DCM (0.3 mL) was added TFA (0.25 mL) and the reaction was stirred at room temperature for 4 hours. The reaction was concentrated in vacuo and partitioned between saturated aqueous sodium hydrogen carbonate solution and 10% MeOH in DCM. The organic layer was collected, dried over sodium sulphate and concentrated in vacuo. The residue was purified using preparative TLC eluting with 5% MeOH in DCM to afford the title compound (80 mg, 50%).

¹H NMR (400 MHz, DMSO-d₆): δ ppm 1.70 (m, 2H), 1.95 (m, 2H), 2.32 (s, 3H), 3.35-3.48 (m, 2H), 3.75 (m, 4H), 5.38-5.41 (m, 1H), 7.28-7.37 (m, 4H), 7.51 (d, 1H), 7.55 (s, 1H), 8.44 (d, 1H), 8.59 (d, 1H), 11.86 (s, 1H).

MS m/z 504 [M+H]⁺

### EXAMPLE 140

### 4-(2-carbamoyl-4-(1-methyl-1H-pyrazol-4-yl)phenoxy)-N-(2-fluoro-4-(trifluoromethoxy)phenyl)piperidine-1-carboxamide

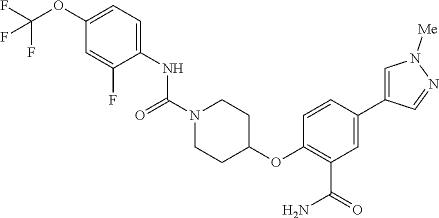 [SMILES: Cn1cc(-c2ccc(OC3CCN(C(=O)Nc4ccc(OC(F)(F)F)cc4F)CC3)c(C(N)=O)c2)cn1]

To a solution of 5-(1-methyl-1H-pyrazol-4-yl)-2-(piperidin-4-yloxy)benzamide (Preparation 69, 100 mg, 0.33 mmol) and 2-fluoro-4-trifluoromethoxyaniline (65 mg, 0.33 mmol) in THF (10 mL) was added DIPEA (0.29 mL, 1.66 mmol) followed by triphosgene (49 mg, 0.17 mmol) at 0° C. and the reaction was stirred warming slowly to room temperature for 18 hours. The reaction was concentrated in vacuo and purified using preparative HPLC to afford the title compound (57 mg, 33%).

¹H NMR (400 MHz, DMSO-d₆): δ ppm 1.72 (m, 2H), 1.98 (m, 2H), 3.38 (m, 2H), 3.73 (m, 2H), 3.85 (s, 3H), 4.78 (m, 1H), 7.17 (m, 1H), 7.24 (d, 1H), 7.39 (dd, 1H), 7.51-7.62 (m, 4H), 7.79 (br s, 1H), 7.88 (m, 1H), 8.10 (br s, 1H), 8.49 (br s, 1H).

MS m/z 522 [M+H]⁺

### EXAMPLE 141

### 2-{[(3R,4S)-3-fluoro-1-{[4-(trifluoromethoxy)phenyl]acetyl}piperidin-4-yl]oxy}-5-(1H-imidazol-4-yl)pyridine-3-carboxamide

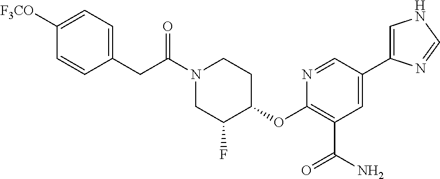 [SMILES: NC(=O)c1cc(-c2c[nH]cn2)cnc1O[C@H]1CCN(C(=O)Cc2ccc(OC(F)(F)F)cc2)C[C@H]1F]

To a solution of 2-{[(3R,4S)-3-fluoro-1-{[4-(trifluoromethoxy)phenyl]acetyl}piperidin-4-yl]oxy}-5-(1-{[2-(trimethylsilyl)ethoxy]methyl}-1H-imidazol-4-yl)pyridine-3-carboxamide (Preparation 217, 400 mg, 0.63 mmol) in dioxane (3 mL) was added 6N aqueous HCl (5 mL) followed by 4N HCl in dioxane (5 mL) and the reaction was stirred at room temperature for 16 hours. The reaction was basified with saturated aqueous NaHCO₃ solution and extracted into EtOAc. The organic layer was collected, washed with brine, dried over sodium sulphate and concentrated in vacuo. The residue was purified using silica gel column chromatography eluting with 5% MeOH in DCM. The residue was diluted with MeOH (2 mL) and ethylenediamine (0.035 mL) was added. The reaction was stirred at room temperature for 40 hours before concentrating in vacuo. The residue was diluted with EtOAc (30 mL), washed with water (2×10 mL), brine (10 mL), dried over sodium sulphate and concentrated in vacuo. The residue was purified using silica gel column chromatography eluting with 5% MeOH in DCM to afford the title compound (65 mg, 21%).

NMR exhibits rotameric behaviour: ¹H NMR (400 MHz, DMSO-d₆): δ ppm 1.77-1.91 (m, 1H), 2.03 (m, 1H), 2.95 (m, 0.5H), 3.16-3.25 (m, 0.5H), 3.58-3.73 (m, 0.5H), 3.84 (m, 2H), 4.04 (m, 0.5H), 4.34 (m, 1H), 4.60 (m, 1H), 5.03-5.18 (m, 1H), 5.49-5.60 (m, 1H), 7.31 (m, 4H), 7.53 (br s, 1H), 7.72 (m, 2H), 7.82 (br s, 1H), 8.58 (s, 1H), 8.69 (s, 1H), 12.26 (br s, 1H).

MS m/z 508 [M+H]⁺

### EXAMPLE 142

### Preparation 1

### Methyl(4-{3-carbamoyl-4-[(1-{[4-(trifluoromethoxy)phenyl]acetyl}piperidin-4-yl)oxy]phenyl}-1H-imidazol-1-yl)acetate

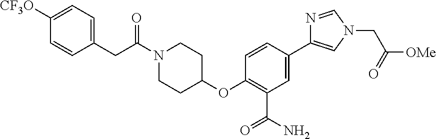

A mixture of methyl (4-iodo-1H-imidazol-1-yl)acetatemethyl (Preparation 197, 207 mg, 0.778 mmol), 5-(4,4,5,5-tetramethyl-1,3,2-dioxaborolan-2-yl)-2-[(1-{[4-(trifluoromethoxy)phenyl]acetyl}piperidin-4-yl)oxy]benzamide (Preparation 14, 254 mg, 0.46 mmol), tetrakis(triphenylphosphine)palladium(0) (63.1 mg, 0.054 mmol), dichlorobis(p-dimethylamino phenylditbutylphosphine)palladium(II) (15.8 mg, 0.022 mmol) and cesium fluoride (182 mg, 1.20 mmol) in methanol (4 mL) was heated to 120° C. for 10 hours under microwave irradiation. The reaction was cooled, concentrated in vacuo, and the residue purified using silica gel column chromatography eluting with 0 to 5% methanol in dichloromethane to afford the title compound as a colourless residue (62 mg, 24%).

¹H NMR (400 MHz, CDCl₃):δ ppm 1.76-1.86 (m, 1H), 1.87-1.95 (m, 1H), 2.03-2.11 (m, 1H), 3.34-3.41 (m, 1H), 3.46-3.54 (m, 1H), 3.67-3.73 (m, 4H), 3.79 (s, 3H), 3.95-4.01 (m, 1H), 4.67-4.72 (m, 3H), 5.72 (br.s, 1H), 6.99 (d, 1H), 7.17 (d, 2H), 7.26-7.29 (m, 3H), 7.54-7.59 (m, 2H), 8.03 (dd, 1H), 8.39 (d, 1H).

MS m/z 561 [M+H]⁺

Examples 143-150 were prepared and purified according to the method described for Library Protocol 2 using 5-(4,4,5,5-tetramethyl-1,3,2-dioxaborolan-2-yl)-2-[(1-{[4-(trifluoromethoxy)phenyl]acetyl}piperidin-4-yl)oxy]benzamide (Preparation 14) and compounds of formula (IV).

| Example | Structure | Name | MS Data and (IV) |
|---|---|---|---|
| 143 | 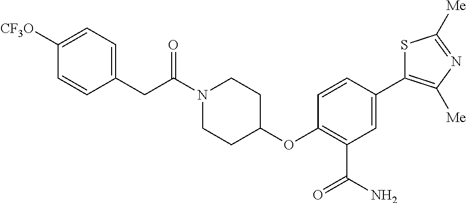 | 5-(2,4-dimethyl-1,3-thiazol- 5-yl)-2-[(1-{[4- (trifluoromethoxy)phenyl] acetyl}piperidin-4- yl)oxy]benzamide | MS m/z 534 [M + H]⁺ Rt = 1.71 minutes. Using 5-bromo-2,4- dimethyl-thiazole. |
| 144 | 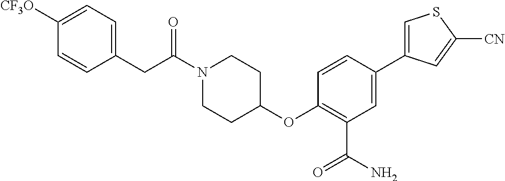 | 5-(5-cyanothiophen-3-yl)-2- [(1-{[4-(trifluoromethoxy) phenyl]acetyl}piperidin-4- yl)oxy]benzamide | MS m/z 530 [M + H]⁺ Rt = 1.71 minutes. Using 3-bromo-5- cyano-thiophene. |
| 145 | 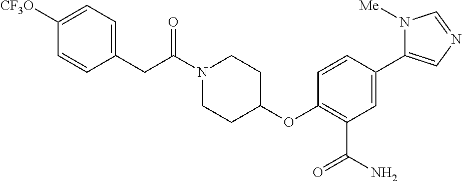 | 5-(1-methyl-1H-imidazol-5- yl)-2-[(1-{[4- (trifluoromethoxy)phenyl] acetyl}piperidin-4- yl)oxy]benzamide | MS m/z 503 [M + H]⁺ Rt = 1.40 minutes. Using 5-bromo-1- methyl-1H- imidazole. |
| 146 | 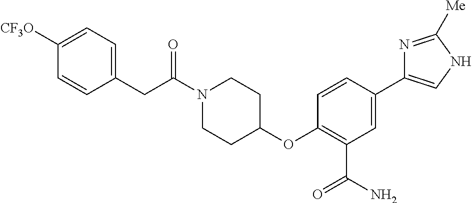 | 5-(2-methyl-1H-imidazol-4- yl)-2-[(1-{[4- (trifluoromethoxy)phenyl] acetyl}piperidin-4- yl)oxy]benzamide | MS m/z 503 [M + H]⁺ Rt = 1.40 minutes. Using 4-bromo-2- methyl-1H- imidazole. |
| 147 | 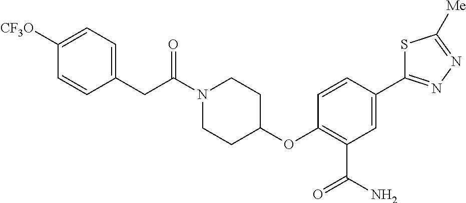 | 5-(5-methyl-1,3,4-thiadiazol- 2-yl)-2-[(1-{[4- (trifluoromethoxy)phenyl] acetyl}piperidin-4- yl)oxy]benzamide | MS m/z 521 [M + H]⁺ Rt = 1.62 minutes. Using 2-bromo-5- methyl- [1,3,4]thiadiazole. |
| 148 | 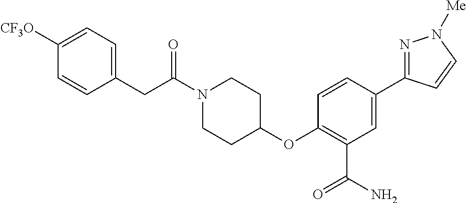 | 5-(1-methyl-1H-pyrazol-3- yl)-2-[(1-{[4- (trifluoromethoxy)phenyl] acetyl}piperidin-4- yl)oxy]benzamide | MS m/z 503 [M + H]⁺ Rt = 1.71 minutes. Using 3-iodo-1- methyl-1H- pyrazole. |
| 149 | 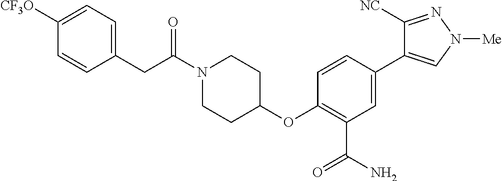 | 5-(3-cyano-1-methyl-1H- pyrazol-4-yl)-2-[(1-{[4- (trifluoromethoxy)phenyl] acetyl}piperidin-4- yl)oxy]benzamide | MS m/z 528 [M + H]⁺ Rt = 1.65 minutes. Using 3-cyano-4- iodo-1-methyl-1H- pyrazole. |
| 150 | 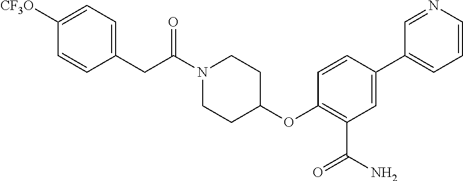 | 5-(pyridin-3-yl)-2-[(1-{[4- (trifluoromethoxy)phenyl] acetyl}piperidin-4- yl)oxy]benzamide | MS m/z 500 [M + H]⁺ Rt = 1.52 minutes. Using 3- iodopyridine. |

Examples 151-152 were prepared and purified according to the method described for Library Protocol 1 using 5-(4,4,5,5-tetramethyl-1,3,2-dioxaborolan-2-yl)-2-[(1-[4-(trifluoromethoxy)benzyl]piperidin-4-yl)oxy]pyridine-3-carboxamide (Preparation 13) and compounds of formula (IV). The following LCMS conditions were employed for QC:

LCMS QC:

A: 0.05% formic acid in water; B: MeCN

Column: RESTEK C18 30×2.1 mm×3 um

Gradient: From 98% [A] and 2% [B] to 90% [A] and 10% [B] in 1.0 min, further to 98% [B] in 2.0 min and finally back to initial condition in 3.00 min, 1.5 mL/min flow rate

| Exam- | | | |
|---|---|---|---|
| ple | Structure | Name | MS Data and (IV) |
| 151 | 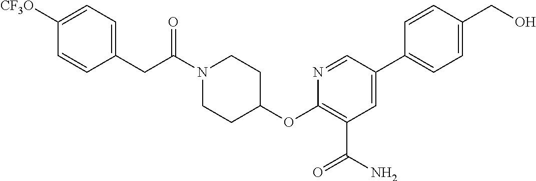 | 5-[4-(hydroxymethyl) phenyl]-2-[(1-{[4- (trifluoromethoxy)phenyl] acetyl}piperidin-4- yl)oxy]pyridine-3- carboxamide | MS m/z 530 [M + H]⁺ Rt = 1.64 minutes. Using 4- iodophenylmethanol. PM: Method B. |
| 152 | 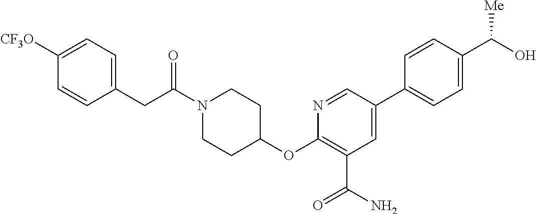 | 5-{4-[(1S)-1- hydroxyethyl]phenyl}-2- [(1-{[4-(trifluoromethoxy) phenyl]acetyl}piperidin-4- yl)oxy]pyridine-3- carboxamide | MS m/z 544 [M + H]⁺ Rt = 1.67 minutes. Using 4-bromo-(1S)- 1-hydroxyethylphenyl PM: Method A. |

### EXAMPLE 153

### 2-({1-[(4-ethylphenyl)acetyl]piperidin-4-yl}oxy)-5-(1-methyl-1H-pyrazol-4-yl)benzamide

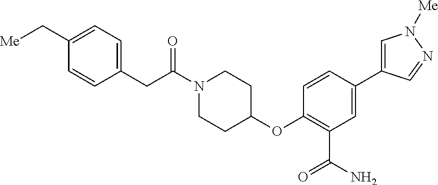 [SMILES: CCc1ccc(CC(=O)N2CCC(Oc3ccc(-c4cnn(C)c4)cc3C(N)=O)CC2)cc1]

The title compound was prepared and purified according to the method described for Library Protocol 4 using 5-(1-methyl-1H-pyrazol-4-yl)-2-(piperidin-4-yloxy)benzamide hydrochloride (Preparation 71) and 4-ethylphenylacetic acid.

LCMS Rt=1.64 minutes MS m/z 447 [M+H]⁺

### EXAMPLE 154

### 2-{[(3S,4R)-3-fluoro-1-{[4-(trifluoromethoxy)phenyl]acetyl}piperidin-4-yl]oxy}-5-(2-methyl-2H-1,2,3-triazol-4-yl)pyridine-3-carboxamide

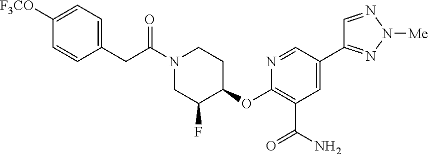 [SMILES: Cn1ncc(-c2cnc(O[C@@H]3CCN(C(=O)Cc4ccc(OC(F)(F)F)cc4)C[C@@H]3F)c(C(N)=O)c2)n1]

To a stirred suspension of 2-(((3S,4R)-3-fluoropiperidin-4-yl)oxy)-5-(2-methyl-2H-1,2,3-triazol-4-yl)nicotinamide hydrochloride (Preparation 226, 110 mg, 0.344 mmol) in DCM (5 mL) was added TEA (0.239 mL, 1.719 mmol) at 0° C. 4-trifluoromethoxyphenylacetic acid (75 mg, 0.344 mmol) followed by EDCI (98 mg, 0.516 mmol) and HOBt (69 mg, 0.516 mmol) were added and the reaction mixture was stirred at room temperature for 16 hours. The reaction was diluted with EtOAc, washed with water, brine, dried over sodium sulfate and concentrated in vacuo. The residue was purified using silica gel column chromatography eluting with 0-3% MeOH in DCM to afford the title compound as a white solid (25 mg, 14%).

¹H NMR (400 MHz, DMSO-d₆): δ ppm 1.82-2.02 (m, 2H), 2.93-3.40 (m, 2H), 3.50-4.00 (m, 2H), 4.21 (s, 3H), 4.20-4.35 (m, 1H), 4.40-4.59 (m, 1H), 5.00-5.30 (m, 1H), 5.53-5.58 (m, 1H), 7.31-7.36 (m, 4H), 7.36 (br s, 1H), 7.90 (br s, 1H), 8.32 (s, 1H), 8.60 (s, 1H), 8.75 (s, 1H).

MS m/z 523 [M+H]⁺

### EXAMPLE 155

### 2-[((3S,4R)-3-fluoro-1-{[4-(trifluoromethoxy)phenyl]acetyl}piperidin-4-yl)oxy]-5-(1-methyl-1H-1,2,3-triazol-4-yl)pyridine-3-carboxamide

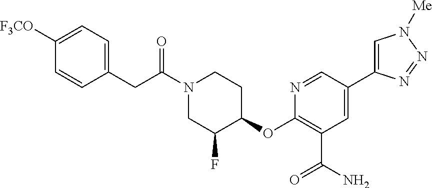 [SMILES: Cn1cc(-c2cnc(O[C@@H]3CCN(C(=O)Cc4ccc(OC(F)(F)F)cc4)C[C@@H]3F)c(C(N)=O)c2)nn1]

The title compound was prepared according to the method described for Example 154 using 2-(((3S,4R)-3-fluoropiperidin-4-yl)oxy)-5-(1-methyl-1H-1,2,3-triazol-4-yl)nicotinamide hydrochloride (Preparation 227). The residue was purified using preparative TLC eluting with 3% MeOH in DCM (40 mg, 27%).

¹H NMR (400 MHz, DMSO-d₆): δ ppm 1.88-2.66 (m, 2H), 2.90-3.39 (m, 1H), 3.55-3.90 (m, 2H), 4.00-4.07 (m, 4H), 4.20-4.30 (m, 1H), 4.50-4.70 (m, 1H), 5.00-5.19 (m, 1H), 5.52-5.59 (m, 1H), 7.29-7.37 (m, 4H), 7.54 (br s, 1H), 7.88 (br s, 1H), 8.02 (s, 1H), 8.74 (s, 1H).

MS m/z 523 [M+H]⁺

### EXAMPLE 156

### 3-{[(3R,4S)-3-fluoro-1-{[4-(trifluoromethoxy)phenyl]acetyl}piperidin-4-yl]oxy}-6-(1-methyl-1H-pyrazol-4-yl)pyridazine-4-carboxamide

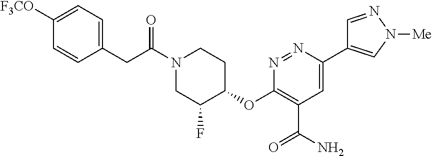 [SMILES: Cn1cc(-c2cc(C(N)=O)c(O[C@H]3CCN(C(=O)Cc4ccc(OC(F)(F)F)cc4)C[C@H]3F)nn2)cn1]

To a stirred solution of 6-chloro-3-(((3R,4S)-3-fluoro-1-(2-(4-(trifluoromethoxy)phenyl)acetyl)piperidin-4-yl)oxy)pyridazine-4-carboxamide (Preparation 220, 150 mg, 0.315 mmol) and 1-methyl-4(4,4,5,5-tetramethyl-1,3,2-dioxaborolan-2-yl)-1H-pyrazole (131 mg, 0.63 mmol) in dioxane:water (5:1; 5 mL) at ambient temperature was added sodium carbonate (83 mg, 0.788 mmol). The reaction was degassed with argon for 15 minutes then treated with Pd(PPh₃)₄ (18 mg, 0.016 mmol). The resulting solution was heated at 100° C. for 16 hours. The reaction was cooled to room temperature and filtered. The filtrate was diluted with water and extracted with EtOAc. The combined organic layers were washed with water, brine, dried over sodium sulfate and concentrated in vacuo. The residue was purified using silica gel column chromatography eluting with 0-3% MeOH in DCM to afford the title compound as a white solid (60 mg, 36%).

¹H NMR (400 MHz, DMSO-d₆): δ ppm 1.80-2.00 (m, 1H), 2.02-2.15 (m, 1H), 2.95-3.40 (m, 2H), 3.60-3.99 (m, 5H), 4.00-4.05 (m, 0.5H), 4.28-4.32 (m, 1H), 4.50-4.55 (m, 0.5H), 5.10-5.20 (m, 1H), 5.60-5.75 (m, 1H), 7.30-7.37 (m, 4H), 7.77 (br s, 1H), 8.01 (br s, 1H), 8.08-8.09 (m, 2H), 8.41 (s, 1H).

MS m/z 523 [M+H]⁺

### EXAMPLE 157

### 3-{[(3S,4R)-3-fluoro-1-{[4-(trifluoromethoxy)phenyl]acetyl}piperidin-4-yl]oxy}-6-(1-methyl-1H-pyrazol-4-yl)pyridazine-4-carboxamide

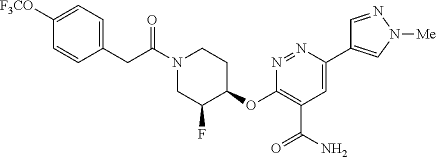 [SMILES: Cn1cc(-c2cc(C(N)=O)c(O[C@@H]3CCN(C(=O)Cc4ccc(OC(F)(F)F)cc4)C[C@@H]3F)nn2)cn1]

The title compound was prepared according to the method described for Example 156 using 6-chloro-3-(((3S,4R)-3-fluoro-1-(2-(4-(trifluoromethoxy)phenyl)acetyl)piperidin-4-yl)oxy)pyridazine-4-carboxamide (Preparation 219) to afford 98 mg, 60% as a white solid.

¹H NMR (400 MHz, DMSO-d₆): δ ppm 1.80-2.00 (m, 1H), 2.02-2.15 (m, 1H), 2.95-3.40 (m, 2H), 3.60-3.99 (m, 5H), 4.00-4.05 (m, 0.5H), 4.28-4.32 (m, 1H), 4.50-4.55 (m, 0.5H), 5.10-5.20 (m, 1H), 5.60-5.75 (m, 1H), 7.30-7.37 (m, 4H), 7.77 (br s, 1H), 8.01 (br s, 1H), 8.08-8.09 (m, 2H), 8.41 (s, 1H).

MS m/z 523 [M+H]⁺

### EXAMPLE 158

### 5-(1-methyl-1H-imidazol-4-yl)-2-{[(1S,5S,8S)-3-{[4-(trifluoromethoxy)phenyl]acetyl}-6-oxa-3-azabicyclo[3.2.1]oct-8-yl]oxy}benzamide

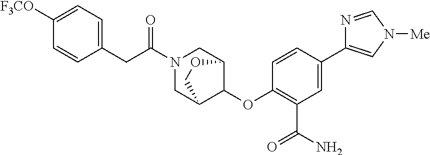 [SMILES: Cn1cnc(-c2ccc(OC3[C@@H]4CO[C@H]3CN(C(=O)Cc3ccc(OC(F)(F)F)cc3)C4)c(C(N)=O)c2)c1]

The title compound was prepared according to Method 5 (Example 5) using 5-(4,4,5,5-tetramethyl-1,3,2-dioxaborolan-2-yl)-2-(((1S,5S)-3-(2-(4-(trifluoromethoxy)phenyl)acetyl)-6-oxa-3-azabicyclo[3.2.1]octan-8-yl)oxy)benzamide (Preparation 221) and 4-iodo-1-methyl-1H-imidazole. The residue was purified using preparative TLC eluting with 10% MeOH in DCM.

¹H NMR (400 MHz, DMSO-d₆): δ ppm 2.60-2.70 (m, 1H), 2.75-2.80 (m, 1H), 3.00-3.20 (m, 1H), 3.40-3.55 (m, 1H), 3.60-4.10 (m, 8H), 4.34-4.35 (m, 1H), 4.90-5.05 (m, 1H), 7.27-7.35 (m, 5H), 7.48-8.10 (m, 6H).

MS m/z 531 [M+H]⁺

### EXAMPLE 159

### 5-(1-methyl-1H-imidazol-4-yl)-2-[(2-{[4-(trifluoromethoxy)phenyl]acetyl}-2-azabicyclo[4.1.0]hept-5-yl)oxy]benzamide

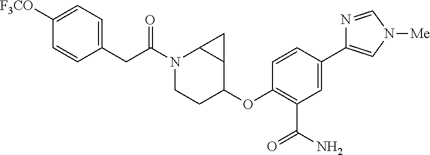 [SMILES: Cn1cnc(-c2ccc(OC3CCN(C(=O)Cc4ccc(OC(F)(F)F)cc4)C4CC34)c(C(N)=O)c2)c1]

The title compound was prepared according to Method 5 (Example 5) using 5-(4,4,5,5-tetramethyl-1,3,2-dioxaborolan-2-yl)-2-((2-(2-(4-(trifluoromethoxy)phenyl)acetyl)-2-azabicyclo[4.1.0]heptan-5-yl)oxy)benzamide (Preparation 222) and 4-iodo-1-methyl-1H-imidazole. The residue was purified using preparative TLC eluting with 5% MeOH in DCM. ¹H NMR (400 MHz, DMSO-d₆): δ ppm 0.85-0.89 (m, 1H), 1.02-1.41 (m, 3H), 1.80-1.84 (m, 1H), 2.09 (m, 1H), 2.59-2.66 (m, 1H), 3.83-3.87 (m, 3H), 3.94-3.98 (m, 2H), 4.13-4.16 (m, 1H), 5.21-5.25 (m, 1H), 7.28-7.33 (m, 3H), 7.37 (t, 2H), 7.53 (br s, 1H), 7.55 (s, 1H), 7.59 (br s, 1H), 7.60 (s, 1H), 7.80 (dd, 1H), 8.22 (d, 1H).

MS m/z 514 [M+H]⁺

### EXAMPLE 160

### 2-{[(3S,4R)-3-fluoro-1-{[4-(trifluoromethoxy)phenyl]acetyl}piperidin-4-yl]oxy}-5-[4-(hydroxymethyl)-1H-pyrazol-1-yl]pyridine-3-carboxamide

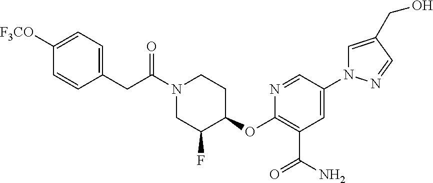 [SMILES: NC(=O)c1cc(-n2cc(CO)cn2)cnc1O[C@@H]1CCN(C(=O)Cc2ccc(OC(F)(F)F)cc2)C[C@@H]1F]

Ethyl 1-(5-carbamoyl-6-(((3S,4R)-3-fluoro-1-(2-(4-(trifluoromethoxy)phenyl)acetyl)piperidin-4-yl)oxy)pyridin-3-yl)-1H-pyrazole-4-carboxylate (Example 161, 40 mg, 0.069 mmol) was taken in a mixture of THF (4 mL) and water (1 mL) and to this was added LiOH.H₂O (8 mg, 0.207 mmol) at room temperature. The resultant mixture was stirred for 16 hours at ambient temperature. The reaction was concentrated in vacuo and the residue was diluted with water (5 mL). The pH was adjusted to 5 by the addition of 2N HCl and extracted with 20% IPA in DCM (thrice). The organic layer was dried over sodium sulfate and concentrated in vacuo. The residue (20 mg, 0.036 mmol) was taken in THF (3 mL) and cooled to 0° C. TEA (0.01 mL, 0.073 mmol) and isobutyl-chloroformate (0.007 mL, 0.055 mmol) were added at 0° C. The reaction was allowed to stir for 2 hours at room temperature. The reaction was filtered through celite and washed with THF (2 mL). To the filtrate was added sodium borohydride (2.7 mg, 0.073 mmol) dissolved in water (3 mL). The reaction was stirred for 1 hour. The reaction was diluted with ethyl acetate and washed with water followed by brine, dried over sodium sulphate and concentrated in vacuo. The residue was purified using preparative TLC eluting with 3% methanol in DCM to afford the title compound (5 mg, 26%).

HPLC (Zorbax SB C18; 50×4.6 mm, 1.8 micron; Mobile phase A: 0.05% TFA in water; mobile phase B: MeCN. 12 minute run). Rt=4.98 minutes MS m/z 538 [M+H]⁺

### EXAMPLE 161

### Ethyl 1-(5-carbamoyl-6-(((3S,4R)-3-fluoro-1-(2-(4-(trifluoromethoxy)phenyl)acetyl)piperidin-4-yl)oxy)pyridin-3-yl)-1H-pyrazole-4-carboxylate

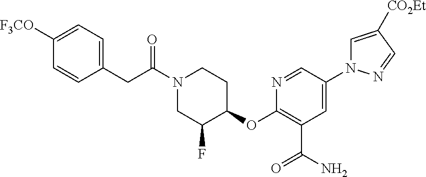 [SMILES: CCOC(=O)c1cnn(-c2cnc(O[C@@H]3CCN(C(=O)Cc4ccc(OC(F)(F)F)cc4)C[C@@H]3F)c(C(N)=O)c2)c1]

The title compound was prepared according to Method 11 (Example 12) using ethyl 1-(5-carbamoyl-6-(((3S,4R)-3-fluoropiperidin-4-yl)oxy)pyridin-3-yl)-1H-pyrazole-4-carboxylate hydrochloride (Preparation 230).

MS m/z 580 [M+H]⁺

### EXAMPLE 162

### 2-(((3S,4R)-3-fluoro-1-(2-(4-(trifluoromethoxy)phenyl)acetyl)piperidin-4-yl)oxy)-5-(4-methyl-1H-imidazol-1-yl)nicotinamide

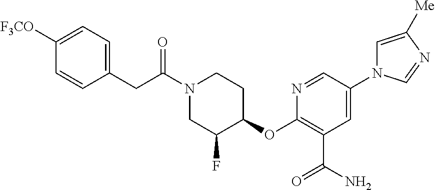 [SMILES: Cc1cn(-c2cnc(O[C@@H]3CCN(C(=O)Cc4ccc(OC(F)(F)F)cc4)C[C@@H]3F)c(C(N)=O)c2)cn1]

The title compound was prepared according to Method 11 (Example 12) using 2-(((3S,4R)-3-fluoropiperidin-4-yl)oxy)-5-(4-methyl-1H-imidazol-1-yl)nicotinamide (Preparation 231).

HPLC (Zorbax SB C18; 50×4.6 mm, 1.8 micron; Mobile phase A: 0.05% TFA in water; mobile phase B: MeCN. 8 minute run). Rt=3.81 minutes MS m/z 522 [M+H]⁺

### EXAMPLE 163

### 2-{[(3S,4S)-1-{[5-(cyclopropyloxy)pyridin-2-yl]acetyl}-3-fluoropiperidin-4-yl]oxy}-5-[1-(2-hydroxy-2-methylpropyl)-1H-pyrazol-4-yl]pyridine-3-carboxamide

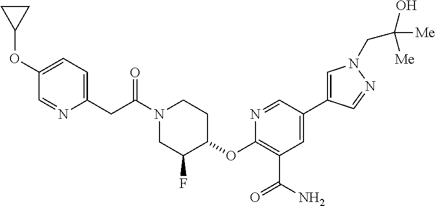 [SMILES: CC(C)(O)Cn1cc(-c2cnc(O[C@H]3CCN(C(=O)Cc4ccc(OC5CC5)cn4)C[C@@H]3F)c(C(N)=O)c2)cn1]

The title compound was prepared according to the methods described for the whole synthesis of Example 33 starting with (3S,4S)-4-[(5-bromo-3-carbamoylpyridin-2-yl)oxy]-3-fluoropiperidine-1-carboxylate (Preparation 133).

HPLC (Zorbax SB C18; 50×4.6 mm, 1.8 micron; Mobile phase A: 0.05% TFA in water; mobile phase B: MeCN. 8 minute run). Rt=3.35 minutes MS m/z 553 [M+H]⁺

### EXAMPLE 164

### 2-{[(3R,4R)-3-fluoro-1-{[4-(trifluoromethoxy)phenyl]acetyl}piperidin-4-yl]oxy}-5-(1-methyl-1H-imidazol-4-yl)pyridine-3-carboxamide

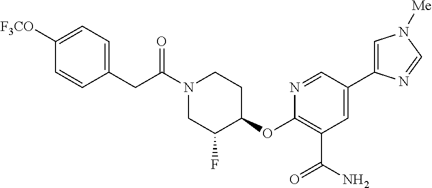 [SMILES: Cn1cnc(-c2cnc(O[C@@H]3CCN(C(=O)Cc4ccc(OC(F)(F)F)cc4)C[C@H]3F)c(C(N)=O)c2)c1]

The title compound was prepared according to the methods described for the whole synthesis of Example 2 starting with tert-butyl (3R,4R)-3-fluoro-4-hydroxypiperidine-1-carboxylate (WO 2013/011402 A1).

HPLC (Zorbax SB C18; 50×4.6 mm, 1.8 micron; Mobile phase A: 0.05% TFA in water; mobile phase B: MeCN. 8 minute run). Rt=3.45 minutes MS m/z 522 [M+H]⁺

### EXAMPLE 165

### 5-{[(3S,4S)-1-{[4-(cyclopropyloxy)phenyl]acetyl}-3-fluoropiperidin-4-yl]oxy}-2-{1-[(methylsulfonyl)methyl]-1H-pyrazol-4-yl}pyridine-4-carboxamide

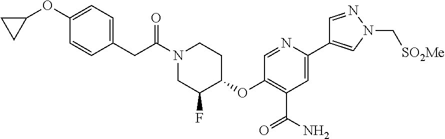 [SMILES: CS(=O)(=O)Cn1cc(-c2cc(C(N)=O)c(O[C@H]3CCN(C(=O)Cc4ccc(OC5CC5)cc4)C[C@@H]3F)cn2)cn1]

The title compound was prepared according to the methods described for the whole synthesis of Example 35 starting with tert-butyl (3S,4S)-4-[(6-bromo-4-carbamoylpyridin-3-yl)oxy]-3-fluoropiperidine-1-carboxylate (Preparation 124).

HPLC (Gemini NX-C18; 50×4.6 mm, 3 micron; Mobile phase A: 0.05% formic acid in water; mobile phase B: MeCN. 10 minute run). Rt=5.03 minutes MS m/z 572 [M+H]⁺

### EXAMPLE 166

### 5-{[(3S,4S)-3-fluoro-1-{[4-(trifluoromethoxy)phenyl]acetyl}piperidin-4-yl]oxy}-2-{1-[(methylsulfonyl)methyl]-1H-pyrazol-4-yl}pyridine-4-carboxamide

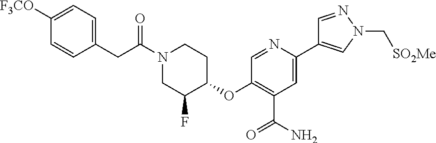 [SMILES: CS(=O)(=O)Cn1cc(-c2cc(C(N)=O)c(O[C@H]3CCN(C(=O)Cc4ccc(OC(F)(F)F)cc4)C[C@@H]3F)cn2)cn1]

The title compound was prepared according to the methods described for the whole synthesis of Example 36 starting with tert-butyl (3S,4S)-4-[(6-bromo-4-carbamoylpyridin-3-yl)oxy]-3-fluoropiperidine-1-carboxylate (Preparation 124).

HPLC (Gemini NX-C18; 50×4.6 mm, 3 micron; Mobile phase A: 0.05% formic acid in water; mobile phase B: MeCN. 10 minute run). Rt=5.42 minutes MS m/z 600 [M+H]⁺

### EXAMPLE 167

### 2-{[(3S,4S)-1-{[5-(cyclopropyloxy)pyridin-2-yl]acetyl}-3-fluoropiperidin-4-yl]oxy}-5-(3,5-dimethyl-1H-pyrazol-4-yl)pyridine-3-carboxamide

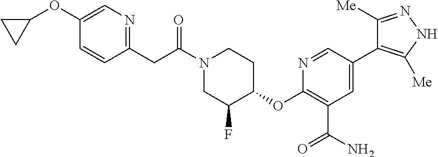 [SMILES: Cc1n[nH]c(C)c1-c1cnc(O[C@H]2CCN(C(=O)Cc3ccc(OC4CC4)cn3)C[C@@H]2F)c(C(N)=O)c1]

The title compound was prepared according to Method 4 (Example 4) using 5-bromo-2-{[(3S,4S)-1-{[5-(cyclopropyloxy)pyridin-2-yl]acetyl}-3-fluoropiperidin-4-yl]oxy}pyridine-3-carboxamide (Preparation 23) and 3,5-dimethylpyrazole-4-boronic acid pinacol ester.

HPLC (Inertsil ODS-3; 50×4.6 mm, 3 micron; Mobile phase A: 50:50 MeCN:MeOH; mobile phase B: 10 mM ammonium acetate in water; 15 minute run). Rt=6.31 minutes MS m/z 507 [M+H]⁺

### EXAMPLE 168

### 2-[(1-{[5-(1,1-difluoroethoxy)pyridin-2-yl]acetyl}piperidin-4-yl)oxy]-5-(1-methyl-1H-imidazol-4-yl)benzamide

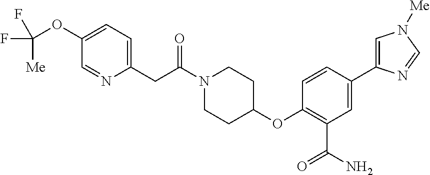 [SMILES: Cn1cnc(-c2ccc(OC3CCN(C(=O)Cc4ccc(OC(C)(F)F)cn4)CC3)c(C(N)=O)c2)c1]

The title compound may be prepared according to Methods 3, 7 or 12 using 5-(1-methyl-1H-imidazole-4-yl)-2-(piperidin-4-yloxy)benzamide (Preparation 45) and 2-(5-(1,1-difluoroethoxy)pyridin-2-yl)acetic acid (Preparation 252).

HPLC (Gemini NX-C18; 50×4.6 mm, 3 micron; Mobile phase A: 0.05% formic acid in water; mobile phase B: MeCN. 8 minute run). Rt=3.14 minutes MS m/z 500 [M+H]⁺

### EXAMPLE 169

### 5-[(1-{[3-fluoro-4-(trifluoromethoxy)phenyl]acetyl}piperidin-4-yl)oxy]-2-(1H-pyrazol-4-yl)pyridine-4-carboxamide

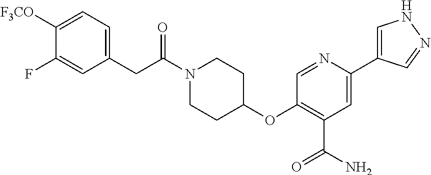 [SMILES: NC(=O)c1cc(-c2cn[nH]c2)ncc1OC1CCN(C(=O)Cc2ccc(OC(F)(F)F)c(F)c2)CC1]

The title compound was prepared according to Preparations 43, 28 and Method 4 (Example 4) using tert-butyl 4-((6-bromo-4-carbamoylpyridin-3-yl)oxy)piperidine-1-carboxylate (Preparation 130), 3-fluoro-4-(trifluoromethoxy) benzoic acid and 4-(4,4,5,5-tetramethyl-1,3,2-dioxaborolan-2-yl)-1H-pyrazole.

HPLC (Gemini NX-C18; 50×4.6 mm, 3 micron; Mobile phase A: 0.05% formic acid in water; mobile phase B: MeCN. 10 minute run). Rt=5.17 minutes MS m/z 508 [M+H]⁺

### EXAMPLE 170

### 4-methyl-5-(1-methyl-1H-imidazol-4-yl)-2-[(1-{[4-(trifluoromethoxy)phenyl]acetyl}piperidin-4-yl)oxy]benzamide

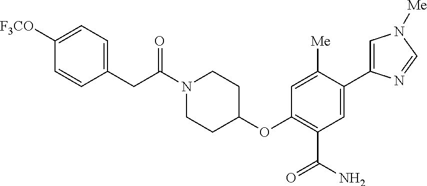 [SMILES: Cc1cc(OC2CCN(C(=O)Cc3ccc(OC(F)(F)F)cc3)CC2)c(C(N)=O)cc1-c1cn(C)cn1]

The title compound was prepared according to Preparations 120, 43, 28, 12 and Example 9 using 5-bromo-2-hydroxy-4-methylbenzamide (U.S. Pat. No. 3,958,002), 4-iodo-1-methyl-1H-imidazole and 4-trifluoromethoxyphenylacetic acid.

HPLC (Gemini NX-C18; 50×4.6 mm, 3 micron; Mobile phase A: 0.05% formic acid in water; mobile phase B: MeCN. 10 minute run). Rt=3.46 minutes MS m/z 517 [M+H]⁺

### EXAMPLES 171 AND 172

### 2-{[(2R,4R,5S)-2,5-dimethyl-1-{[4-(trifluoromethoxy)phenyl]acetyl}piperidin-4-yl]oxy}-5-(1-methyl-1H-imidazol-4-yl)benzamide and 2-{[(2R,4S,5S)-2,5-dimethyl-1-{[4-(trifluoromethoxy)phenyl]acetyl}piperidin-4-yl]oxy}-5-(1-methyl-1H-imidazol-4-yl)benzamide

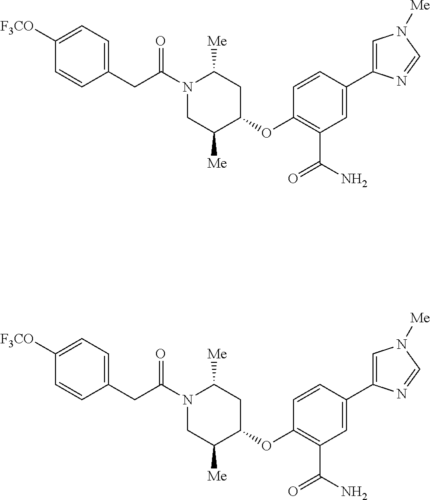 [SMILES: C[C@@H]1C[C@H](Oc2ccc(-c3cn(C)cn3)cc2C(N)=O)[C@@H](C)CN1C(=O)Cc1ccc(OC(F)(F)F)cc1 ; C[C@@H]1C[C@H](Oc2ccc(-c3cn(C)cn3)cc2C(N)=O)[C@@H](C)CN1C(=O)Cc1ccc(OC(F)(F)F)cc1]

The title compounds were prepared according to the method described for Preparations 157, 136, 43 and Method 11 using 2-hydroxy-5-(1-methyl-1H-imidazol-4-yl)benzamide (Preparation 160) and tert-butyl (2R,4S,5S)-4-hydroxy-2,5-dimethylpiperidine-1-carboxylate, tert-butyl (2R,4R,5S)-4-hydroxy-2,5-dimethylpiperidine-1-carboxylate (Preparation 242) and 4-trifluoromethoxyphenylacetic acid. The diastereomers were separated using preparative HPLC (no data).

First Eluting Isomer: Example 171

¹H NMR (400 MHz, DMSO-d₆): δ ppm 0.91 (d, 3H), 1.17 (d, 3H), 1.74-1.77 (m, 1H), 1.95-1.98 (m, 1H), 2.12-2.14 (m, 1H), 2.95-3.05 (m, 1H), 3.35-3.38 (m, 1H), 3.66 (s, 3H), 3.69-3.73 (m, 2H), 3.81 (m, 1H), 4.47 (br s, 1H), 7.06 (d, 1H), 7.30 (d, 2H), 7.36 (d, 2H), 7.53 (br s, 3H), 7.59 (s, 1H), 7.73 (dd, 1H), 7.95 (d, 1H).

MS m/z 530 [M+H]⁺

Second Eluting Isomer: Example 172

¹H NMR (400 MHz, DMSO-d₆): δ ppm 1.22-1.24 (m, 3H), 1.32-1.35 (m, 3H), 1.89-1.93 (m, 1H), 2.15-2.19 (m, 1H), 2.37-2.41 (m, 1H), 2.89-3.03 (m, 1H), 3.36-3.44 (m, 1H), 3.66 (s, 3H), 3.83-3.88 (m, 1H), 4.19-4.29 (m, 2H), 5.03-5.06 (m, 1H), 7.18 (d, 1H), 7.27-7.30 (m, 2H), 7.36 (d, 2H), 7.46-7.58 (m, 3H), 7.60 (s, 1H), 7.77 (dd, 1H), 8.13 (d, 1H).

MS m/z 530 [M+H]⁺

### EXAMPLE 173

### 2-[(1-{[5-(cyclopropyloxy)pyridin-2-yl]acetyl}piperidin-4-yl)oxy]-5-(1-methyl-1H-pyrazol-4-yl)benzamide

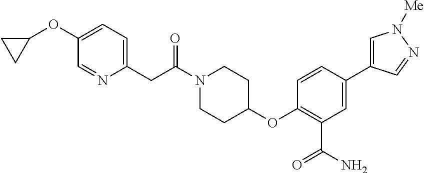 [SMILES: Cn1cc(-c2ccc(OC3CCN(C(=O)Cc4ccc(OC5CC5)cn4)CC3)c(C(N)=O)c2)cn1]

The title compound was prepared according to Method 11 (Example 12) using [5-(cyclopropyloxy)pyridine-2-yl]acetate (Preparation 211) and 5-(1-methyl-1H-pyrazol-4-yl)-2-(piperidin-4-yloxy)benzamide (Preparation 69).

Preparative HPLC: Xterra RP C18 250×4.6 mm, 5 micron; mobile phase A: 0.05% formic acid in acetonitrile, mobile phase B: water; run time=75 minutes, 0.8 mL/min, From 10% A to 95% A at 50 minutes then return to 10% A at 75 minutes.

Rt=31.54 minutes MS m/z 476 [M+H]⁺

### EXAMPLE 174

### 5-[1-(2-hydroxyethyl)-1H-1,2,3-triazol-4-yl]-2-[(1-{[4-(trifluoromethoxy)phenyl]acetyl}piperidin-4-yl)oxy]benzamide

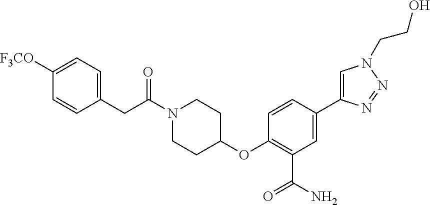 [SMILES: NC(=O)c1cc(-c2cn(CCO)nn2)ccc1OC1CCN(C(=O)Cc2ccc(OC(F)(F)F)cc2)CC1]

The title compound was prepared according to the method described for Example 137 and isolated as the title isomer.

HPLC (Gemini NX-C18; 50×4.6 mm, 3 micron; Mobile phase A: 0.05% formic acid in water; mobile phase B: MeCN. 10 minute run). Rt=5.85 minutes MS m/z 534 [M+H]⁺

### EXAMPLE 175

### 2-[1-(2-hydroxyethyl)-1H-pyrazol-4-yl]-5-[(1-{[4-(trifluoromethoxy)phenyl]acetyl}piperidin-4-yl)oxy]pyridine-4-carboxamide

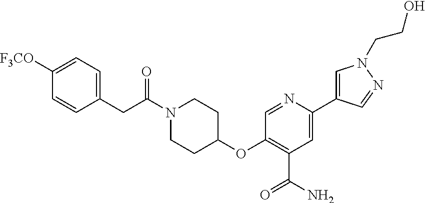 [SMILES: NC(=O)c1cc(-c2cnn(CCO)c2)ncc1OC1CCN(C(=O)Cc2ccc(OC(F)(F)F)cc2)CC1]

The title compound was prepared according to the method described for Preparation 43, 137, Method 5 (Example 5) and Example 137 using tert-butyl 4 ((6-bromo-4-carbamoylpyridin-3-yl)oxy)piperidine-1-carboxylate (Preparation 130), 4-trifluoromethoxyphenylacetic acid and 4-(4,4,5,5-tetramethyl-1,3,2-dioxaborolan-2-yl)-1H-pyrazole.

HPLC (Gemini NX-C18; 50×4.6 mm, 3 micron; Mobile phase A: 0.05% formic acid in water; mobile phase B: MeCN. 8 minute run). Rt=2.741 minutes MS m/z 534 [M+H]⁺

### EXAMPLE 176

### 2-[(1-{[4-(cyclopropyloxy)phenyl]acetyl}piperidin-4-yl)oxy]-5-[1-(2-hydroxyethyl)-1H-pyrazol-4-yl]pyridine-3-carboxamide

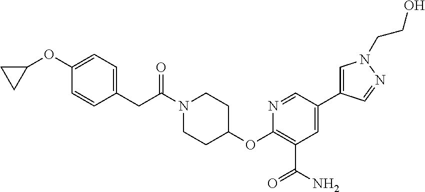 [SMILES: NC(=O)c1cc(-c2cnn(CCO)c2)cnc1OC1CCN(C(=O)Cc2ccc(OC3CC3)cc2)CC1]

The title compound was prepared according to Preparation 137, Method 5 (Example 5) and Example 137 using 5-bromo-2-(piperidin-4-yloxy)pyridine-3-carboxamide (Preparation 109), (4-cyclopropoxyphenyl)acetic acid (Preparation 173) and and 4-(4,4,5,5-tetramethyl-1,3,2-dioxaborolan-2-yl)-1H-pyrazole.

HPLC (Gemini NX-C18; 50×4.6 mm, 3 micron; Mobile phase A: 0.05% formic acid in water; mobile phase B: MeCN. 8 minute run). Rt=2.741 minutes MS m/z 534 [M+H]⁺

### EXAMPLE 177

### 5-(1H-pyrazol-3-yl)-2-[(1-{[4-(trifluoromethoxy)phenyl]acetyl}piperidin-4-yl)oxy]benzamide

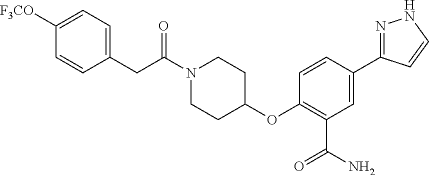 [SMILES: NC(=O)c1cc(-c2cc[nH]n2)ccc1OC1CCN(C(=O)Cc2ccc(OC(F)(F)F)cc2)CC1]

The title compound was prepared according to Method 10 (Example 11) using 5-bromo-2-[(1-{[4-(trifluoromethoxy)phenyl]acetyl}piperidin-4-yl)oxy]benzamide (Preparation 39) and 1H-pyrazol-3-yl boronic acid.

¹H NMR (400 MHz, DMSO-d₆): δ ppm 1.63-1.67 (m, 2H), 1.90-1.94 (m, 2H), 3.37-3.46 (m, 2H), 3.77-3.83 (m, 4H), 4.80 (m, 1H), 6.64 (s, 1H), 7.25 (d, 1H), 7.29 (d, 2H), 7.35 (d, 2H), 7.59 (s, 1H), 7.76 (s, 1H), 7.84 (d, 1H), 8.16 (s, 1H).

MS m/z 489 [M+H]⁺

### EXAMPLE 178

### 2-[(1-{[4-(cyclopropyloxy)phenyl]acetyl}piperidin-4-yl)oxy]-5-(1-methyl-1H-pyrazol-4-yl)benzamide

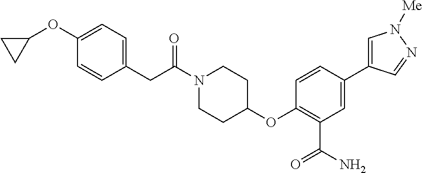 [SMILES: Cn1cc(-c2ccc(OC3CCN(C(=O)Cc4ccc(OC5CC5)cc4)CC3)c(C(N)=O)c2)cn1]

The title compound was prepared according to Method 11 (Example 12) using 5-(1-methyl-1H-pyrazol-4-yl)-2-(piperidin-4-yloxy)benzamide (Preparation 69) and (4-cyclopropoxyphenyl)acetate (Preparation 173).

HPLC (Zorbax SB C18; 50×4.6 mm, 1.8 micron; Mobile phase A: 0.05% TFA in water; mobile phase B: MeCN. 8 minute run). Rt=3.718 minutes MS m/z 475 [M+H]⁺

### EXAMPLE 179

### 5-(4-methyl-1H-imidazol-5-yl)-2-[(1-{[4-(trifluoromethoxy)phenyl]acetyl}piperidin-4-yl)oxy]pyridine-3-carboxamide

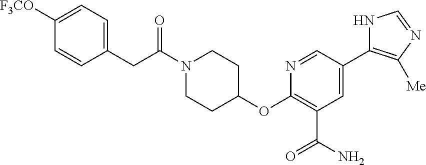 [SMILES: Cc1nc[nH]c1-c1cnc(OC2CCN(C(=O)Cc3ccc(OC(F)(F)F)cc3)CC2)c(C(N)=O)c1]

The title compound was prepared according to Method 2 (Example 2) using 5-(4,4,5,5-tetramethyl-1,3,2-dioxaborolan-2-yl)-2-({1-[4-(trifluoromethoxy)benzyl]piperidin-4-yl}oxy)pyridine-3-carboxamide (Preparation 13) and 5-bromo-4-methyl-1H-imidazole.

HPLC (Zorbax SB C18; 50×4.6 mm, 1.8 micron; Mobile phase A: 0.05% TFA in water; mobile phase B: MeCN. 8 minute run). Rt=4.569 minutes MS m/z 504 [M+H]⁺

### EXAMPLE 180

### 5-(1,3-thiazol-4-yl)-2-[(1-{[4trifluoromethoxy)phenyl]acetyl}piperidin-4-yl)oxy]pyridine-3-carboxamide

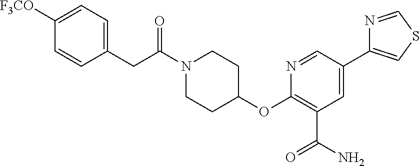 [SMILES: NC(=O)c1cc(-c2cscn2)cnc1OC1CCN(C(=O)Cc2ccc(OC(F)(F)F)cc2)CC1]

The title compound was prepared according to Method 2 (Example 2) using 5-(4,4,5,5-tetramethyl-1,3,2-dioxaborolan-2-yl)-2-({1-[4-(trifluoromethoxy)benzyl]piperidin-4-yl}oxy)pyridine-3-carboxamide (Preparation 13) and 4-bromo-1,3-thiazole.

HPLC (Gemini NX-C18; 50×4.6 mm, 3 micron; Mobile phase A: 0.05% formic acid in water; mobile phase B: MeCN. 12 minute run). Rt=5.536 minutes MS m/z 507 [M+H]⁺

### EXAMPLE 181

### 4-[2-carbamoyl-4-(1-methyl-1H-pyrazol-4-yl)phenoxy]-N-[4-(cyclopropyloxy)phenyl]piperidine-1-carboxamide

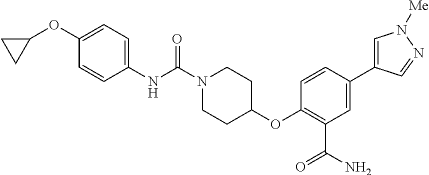 [SMILES: Cn1cc(-c2ccc(OC3CCN(C(=O)Nc4ccc(OC5CC5)cc4)CC3)c(C(N)=O)c2)cn1]

The title compound was prepared according to the method described for Example 140 using 4-cyclopropyloxyaniline.

HPLC (Zorbax SB C18; 50×4.6 mm, 1.8 micron; Mobile phase A: 0.05% TFA in water; mobile phase B: MeCN. 8 minute run). Rt=3.63 minutes MS m/z 476 [M+H]⁺

### EXAMPLE 182

### 5-(1-methyl-1H-pyrazol-4-yl)-2-[(1-{[4-(trifluoromethoxy)phenyl]acetyl}piperidin-4-yl)oxy]pyridine-3-carboxamide

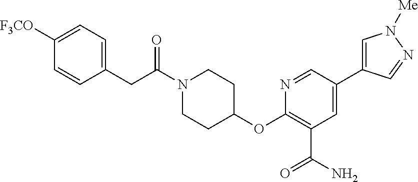 [SMILES: Cn1cc(-c2cnc(OC3CCN(C(=O)Cc4ccc(OC(F)(F)F)cc4)CC3)c(C(N)=O)c2)cn1]

The title compound was prepared according to the methods described for Preparations 79, 43 and Method 11 (Example 12) using tert-butyl 4-[(5-bromo-3-carbamoylpyridin-2-yl)oxy]piperidine-1-carboxylate (Preparation 149), 1-methyl-4-(4,4,5,5-tetramethyl-1,3,2-dioxaborolan-2-yl)-1H-pyrazole and 4-trifluoromethoxyphenylacetic acid.

HPLC (Zorbax SB C18; 50×4.6 mm, 1.8 micron; Mobile phase A: 0.05% TFA in water; mobile phase B: MeCN. 12 minute run). Rt=5.165 minutes MS m/z 504 [M+H]⁺

### EXAMPLE 183

### 2-{[(3S,4R)-3-fluoro-1-{[4-(trifluoromethoxy)phenyl]acetyl}piperidin-4-yl]oxy}-5-[2-(trifluoromethyl)-1H-imidazol-4-yl]pyridine-3-carboxamide

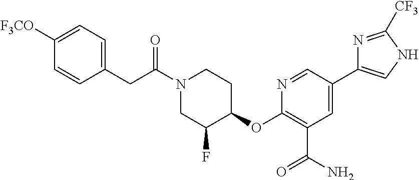 [SMILES: NC(=O)c1cc(-c2c[nH]c(C(F)(F)F)n2)cnc1O[C@@H]1CCN(C(=O)Cc2ccc(OC(F)(F)F)cc2)C[C@@H]1F]

The title compound was prepared according to the methods described for Preparations 138, Method 9 (Example 10), Preparation 76 and Method 10 (Example 11) using tert-butyl (3S,4R)-4-{[3-carbamoyl-5-(4,4,5,5-tetramethyl-1,3,2-dioxaborolan-2-yl)pyridin-2-yl]oxy}-3-fluoropiperidine-1-carboxylate (Preparation 99), 4-trifluoromethoxyphenylacetic acid and 1-benzyl-4-bromo-2-methyl-1H-imidazole.

HPLC (Xbridge C18; 150×4.6 mm, 5 micron; 12.5 minute run; Gradient: 5-100% MeCN in water modified with 0.1% TFA over 10 minuts then back to 5% at 12.5 minutes. Rt=8.084 minutes MS m/z 576 [M+H]⁺

### Preparation 2

### 2-{[(3R,4S)-3-fluoro-1-{[4-(trifluoromethoxy)phenyl]acetyl}piperidin-4-yl]oxy}-5-(2-methyl-1-{[2-(trimethylsilyl)ethoxy]methyl}-1H-imidazol-5-yl)pyridine-3-carboxamide and 2-{[(3R,4S)-3-fluoro-1-{[4-(trifluoromethoxy)phenyl]acetyl}piperidin-4-yl]oxy}-5-(2-methyl-1-{[2-(trimethylsilyl)ethoxy]methyl}-1H-imidazol-4-yl)pyridine-3-carboxamide

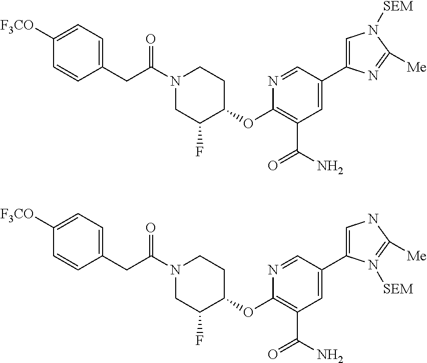

To a solution of 5-bromo-2-{[(3R,4S)-3-fluoro-1-{[4-(trifluoromethoxy)phenyl]acetyl}piperidin-4-yl]oxy}pyridine-3-carboxamide (Preparation 26, 540 mg, 0.95 mmol) and mixture of regioisomers 4-bromo-2-methyl-1-((2-(trimethylsilyl)ethoxy)methyl)-1H-imidazole and 5-bromo-2-methyl-1-((2-(trimethylsilyl)ethoxy)methyl)-1H-imidazole (Preparation 176, 553 mg, 1.91 mmol) in dioxane (8 mL) was added a solution of sodium carbonate (773 mg, 2.38 mmol) in water (2 mL). The reaction was degassed with argon for 15 minutes followed by the addition of tris(dibenzylideneacetone)dipalladium (0) (43 mg, 0.048 mmol) and tri-tertbutylphosphine tetrafluoroborate salt (56 mg, 0.19 mmol). The reaction was heated to 100° C. for 16 hours before cooling and diluting with EtOAc. The organic solution was washed with water, brine, dried over sodium sulphate and concentrated in vacuo. The residue was purified using silica gel column chromatography eluting with 4-5% MeOH in DCM to afford the title compounds as a mixture of regioisomers (320 mg, 52%).

MS m/z 652 [M+H]⁺

The following Preparations were prepared according to Methods 2, 5, 7 or 10 using the appropriate starting materials and Purification Method (PM) below if different from the methods described. Sodium, cesium or potassium carbonate may be used as base.

Purification Method A: Preparative TLC eluting with 5% MeOH in DCM.

Purification Method B: Silica gel column chromatography eluting with between 3-6% MeOH in DCM.

| Prepar- | | | Starting materials & |
|---|---|---|---|
| ation | Structure and Name | Data | PM |
| 3 | 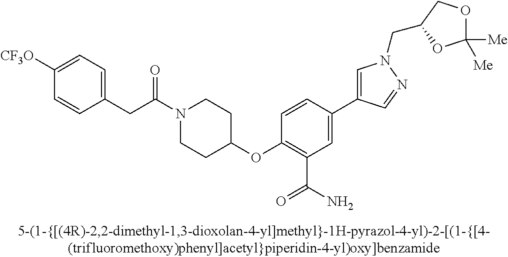 | MS m/z 603 [M + H]⁺ ¹H NMR (400 MHz, DMSO-d₆): δ ppm 1.25 (d, 6H), 1.64 (m, 2H), 1.90 (m, 2H), 3.40-3.45 (m, 1H), 3.75-3.79 (m, 5H), 4.00-4.04 (m, 1H), 4.21-4.23 (m, 2H), 4.40-4.43 (m, 1H), 4.77 (m, 1H), 7.19-7.20 (d, 1H), 7.28-7.30 (d, 2H), 7.34-7.36 (d, 2H), 7.54 (br s, 1H), 7.57 (br s, 1H), 7.60-7.62 (dd, 1H), 7.84 (s, 1H), 7.87 (d, 1H), 8.13 (s, 1H). | 1-{[(4R)-2,2-dimethyl- 1,3-dioxolan-4- yl]methyl}-4-(4,4,5,5- tetramethyl-1,3,2- dioxaborolan-2-yl)-1H- pyrazole (Preparation 190) and 5-bromo-2- [(1-{[4- (trifluoromethoxy) phenyl]acetyl} piperidin- 4-yl)oxy]benzamide (Preparation 16) and PM A. |
| 4 | 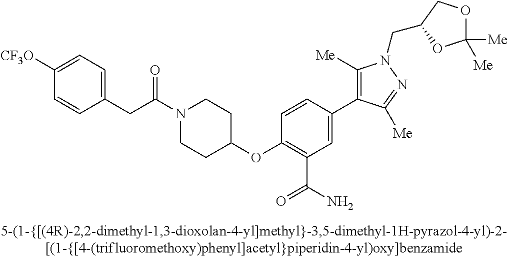 | ¹H NMR (400 MHz, DMSO-d₆): δ ppm 1.21- 1.28 (s, 6H), 1.66 (m, 2H), 1.93 (m, 2H), 2.10 (s, 3H), 2.22 (s, 3H), 3.31-3.46 (m, 2H), 3.79- 3.83 (m, 6H), 4.03-4.05 (m, 1H), 4.11-4.12 (m, 2H), 4.36-4.40(m, 1H), 4.78 (m, 1H), 7.23-7.25 (m, 1H), 7.28-7.31 (m, 3H), 7.35-7.37 (m, 2H), 7.54-7.57 (m, 3H). | 5-(4,4,5,5-tetramethyl- 1,3,2-dioxaborolan-2- yl)-2-[(1-{[4- (trifluoromethoxy) phenyl]acetyl} piperidin- 4-yl)oxy]benzamide (Preparation 14) and 4-bromo-1-{[(4R)-2,2- dimethyl-1,3-dioxolan- 4-yl]methyl}-3,5- dimethyl-1H-pyrazole (Preparation 148). |
| 5 | 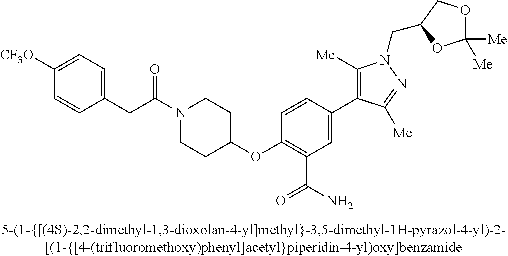 | ¹H NMR (400 MHz, DMSO-d₆): δ ppm 1.23- 1.25 (s, 6H), 1.66 (m, 2H), 1.93 (m, 2H), 2.10 (s, 3H), 2.32 (s, 3H), 3.31-3.46 (m, 2H), 3.79- 3.83 (m, 6H), 4.03-4.05 (m, 1H), 4.11-4.12 (m, 2H), 4.36-4.40(m, 1H), 4.78 (m, 1H), 7.23-7.25 (m, 1H), 7.28-7.31 (m, 3H), 7.35-7.37 (m, 2H), 7.54-7.57 (m, 3H). | 5-(4,4,5,5-tetramethyl- 1,3,2-dioxaborolan-2- yl)-2-[(1-{[4- (trifluoromethoxy) phenyl]acetyl} piperidin- 4-yl)oxy]benzamide (Preparation 14) and 4-bromo-1-{[(4S)-2,2- dimethyl-1,3-dioxolan- 4-yl]methyl}-3,5- dimethyl-1H-pyrazole (Preparation 192). |
| 6 | 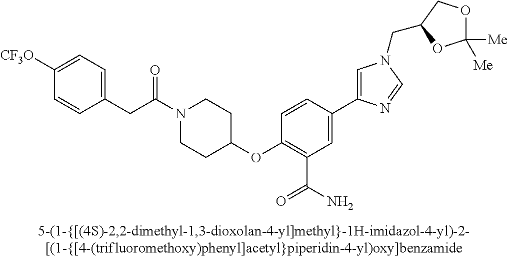 | MS m/z 603 [M + H]⁺ | 5-(4,4,5,5-tetramethyl- 1,3,2-dioxaborolan-2- yl)-2-[(1-{[4- (trifluoromethoxy) phenyl]acetyl} piperidin- 4-yl)oxy]benzamide (Preparation 14) and 4-bromo-1-{[(4S)-2,2- dimethyl-1,3-dioxolan- 4-yl]methyl}-1H- imidazole (Preparation 193). |
| 7 | 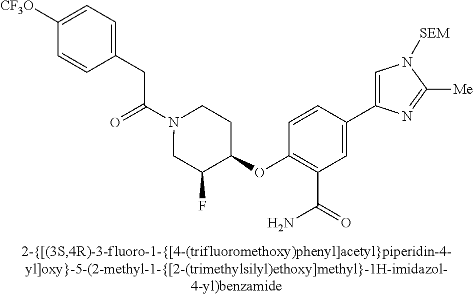 | MS m/z 651 [M + H]⁺ | 2-{[(3S,4R)-3- fluoropiperidin-4- yl]oxy}-5-(2-methyl-1- {[2-(trimethylsilyl) ethoxy]methyl}-1H- imidazol-4-yl) benzamide hydrochloride (Preparation 53) and 4-bromo-2-methyl-1- ((2-(trimethylsilyl) ethoxy)methyl)-1H- imidazole (Preparation 176). |
| 8 | 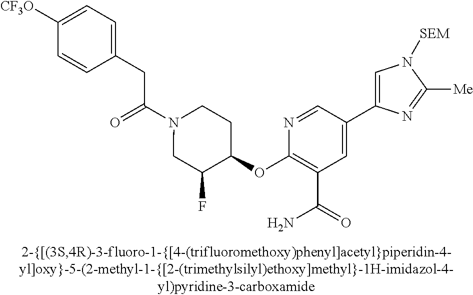 | MS m/z 652 [M + H]⁺ | 2-{[(3S,4R)-3- fluoropiperidin-4- yl]oxy}-5-(2-methyl-1- {[2-(trimethylsilyl) ethoxy]methyl}-1H- imidazol-4-yl)pyridine- 3-carboxamide hydrochloride (Preparation 52) and 4-bromo-2-methyl-1- ((2-(trimethylsilyl) ethoxy)methyl)-1H- imidazole (Preparation 176). |
| 9 | 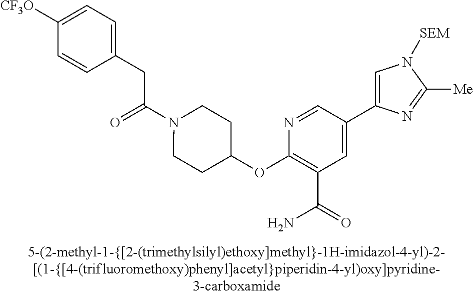 | MS m/z 634 [M + H]⁺ ¹H NMR (400 MHz, DMSO-d₆): δ ppm 0.00 (s, 9H), 0.84 (t, 2H), 1.71 (m, 2H), 1.98 (m, 2H), 2.37 (s, 3H), 3.32- 3.54 (m, 5H), 3.80-3.90 (m, 4H), 5.28 (s, 2H), 5.40 (m, 1H), 5.75 (m, 1H), 7.29-7.37 (m, 4H), 7.56 (m, 1H), 7.73 (m, 2H), 8.45 (s, 1H), 8.60 (s, 1H). | 4-bromo-2-methyl-1- {[2-(trimethylsilyl) ethoxy]methyl}-1H- imidazole (Preparation 176) and 5-(4,4,5,5- tetramethyl-1,3,2- dioxaborolan- 2-yl)-2-({1-[4- (trifluoromethoxy) benzyl]piperidin-4- yl}oxy)pyridine-3- carboxamide (Preparation 13). |
| 10 | 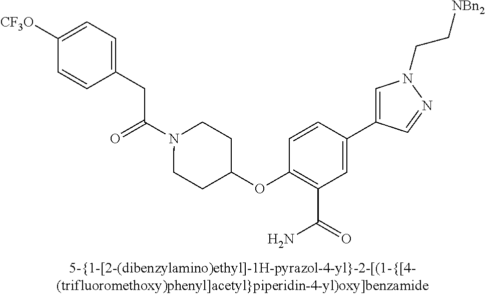 | MS m/z 712 [M + H]⁺ ¹H NMR (400 MHz, DMSO-d₆): δ ppm 1.66 (m, 2H), 1.92 (m, 2H), 2.79-2.82 (t, 2H), 3.35- 3.46 (m, 2H), 3.58 (s, 4H), 3.79 (br m, 4H), 4.23-4.26 (t, 2H), 4.78 (m, 1H), 7.18-7.37 (m, 15H), 7.55-7.61 (m, 3H), 7.78 (s, 1H), 7.90 (br s, 1H), 8.07 (br s, 1H). | 5-bromo-2-[(1-{[4- (trifluoromethoxy) phenyl]acetyl} piperidin- 4-yl)oxy]benzamide (Preparation 16) and N,N-dibenzyl-2-[4- (4,4,5,5-tetramethyl- 1,3,2-dioxaborolan-2- yl)-1H-pyrazol-1- yl]ethanamine (Preparation 201) and PM B. |

### Preparation 11

### 5-(1-{[(4S)-2,2-dimethyl-1,3-dioxolan-4-yl]methyl}-1H-pyrazol-4-yl)-2-[(1-{[4-(trifluoromethoxy)phenyl]acetyl}piperidin-4-yl)oxy]benzamide

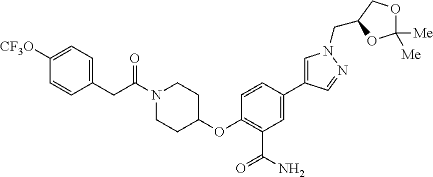

The title compound was prepared according to the method described for Example 131 using 5-(1H-pyrazol-4-yl)-2-[(1-{[4-(trifluoromethoxy)phenyl]acetyl}piperidin-4-yl)oxy]benzamide (Example 11) and [(4R)-2,2-dimethyl-1,3-dioxolan-4-yl]methyl methanesulfonate (Preparation 195) and purified using silica gel column chromatography eluting with between 3-6% MeOH in DCM.

¹H NMR (400 MHz, DMSO-d₆): δ ppm 1.25-1.30 (m, 6H), 1.64 (m, 2H), 1.90 (m, 2H), 3.40-3.45 (m, 2H), 3.75-3.79 (m, 5H), 4.00-4.04 (m, 1H), 4.21-4.23 (m, 2H), 4.40-4.43 (m, 1H), 4.77 (m, 1H), 7.19-7.20 (d, 1H), 7.28-7.30 (d, 2H), 7.34-7.36 (d, 2H), 7.54 (br s, 1H), 7.57 (br s, 1H), 7.60-7.62 (dd, 1H), 7.84 (s, 1H), 7.87 (d, 1H), 8.13 (s, 1H).

MS m/z 603 [M+H]⁺

### Preparation 12

### 2-{[(3S,4S)-3-fluoro-1-{[4-(trifluoromethoxy)phenyl]acetyl}piperidin-4-yl]oxy}-5-(4,4,5,5-tetramethyl-1,3,2-dioxaborolan-2-yl)pyridine-3-carboxamide

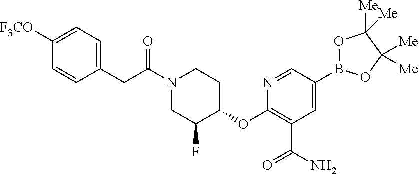 [SMILES: CC1(C)OB(c2cnc(O[C@H]3CCN(C(=O)Cc4ccc(OC(F)(F)F)cc4)C[C@@H]3F)c(C(N)=O)c2)OC1(C)C]

A suspension of 5-bromo-2-{[(3S,4S)-3-fluoro-1-{[4-(trifluoromethoxy)phenyl]acetyl}piperidin-4-yl]oxy}pyridine-3-carboxamide (Preparation 22, 600 mg, 1.15 mmol), bispinacolatodiboron (439 mg, 1.73 mmol) and potassium acetate (339 mg, 3.46 mmol) in dioxane (25 mL) was degassed with argon for 20 minutes. 1,1-bis(diphenylphosphino)ferrocene palladium (II) dichloride (47 mg, 0.06 mmol) was added and the reaction was heated at 100° C. for 16 hours. The reaction was cooled and filtered through celite, washing through with EtOAc. The filtrate was concentrated in vacuo and the residue triturated with heptanes to afford the title compound that was taken on directly to the next step.

The following Preparations were prepared according to the method described by Preparation 12 using the appropriate aryl halide and if required, purified using one of the purification methods described below:

Purification Method A: Silica gel column chromatography eluting with 0-5% MeOH in DCM and taken on directly to the next reaction.

| Prepar- | | | |
|---|---|---|---|
| ation | Structure | Name | Starting material/Data |
| 13 | 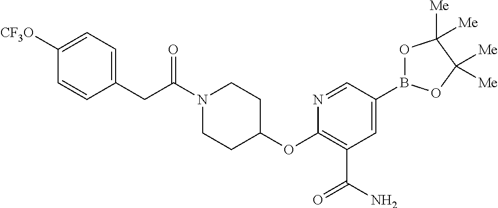 | 5-(4,4,5,5-tetramethyl- 1,3,2-dioxaborolan-2- yl)-2-({1-[4- (trifluoromethoxy) benzyl]piperidin-4- yl}oxy)pyridine-3- carboxamide | 5-bromo-2-({1-[4- (trifluoromethoxy) benzyl]piperidin-4- yl}oxy)pyridine-3- carboxamide (Preparation 37). Purification method A. |
| 14 | 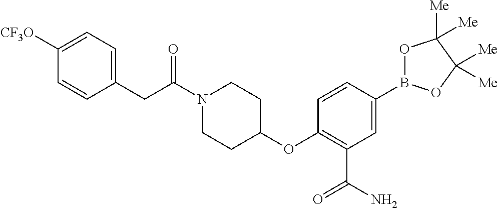 | 5-(4,4,5,5-tetramethyl- 1,3,2-dioxaborolan-2- yl)-2-[(1-{[4- (trifluoromethoxy) phenyl]acetyl} piperidin-4- yl)oxy]benzamide | 5-bromo-2-[(1-{[4- (trifluoromethoxy) phenyl]acetyl} piperidin-4- yl)oxy]benzamide (Preparation 16). Purification method A. |
| 15 | 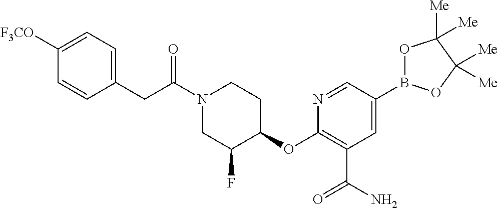 [SMILES: CC1(C)OB(c2cnc(O[C@@H]3CCN(C(=O)Cc4ccc(OC(F)(F)F)cc4)C[C@@H]3F)c(C(N)=O)c2)OC1(C)C] | 2-{[(3S,4R)-3-fluoro- 1-{[4- (trifluoromethoxy) phenyl]acetyl} piperidin-4- yl]oxy}-5-(4,4,5,5- tetramethyl-1,3,2- dioxaborolan-2- yl)pyridine-3- carboxamide | 5-bromo-2-{[(3S,4R)- 3-fluoro-1-{[4- (trifluoromethoxy) phenyl]acetyl} piperidin-4- yl]oxy}pyridine-3- carboxamide (Preparation 27). |
| 16 | 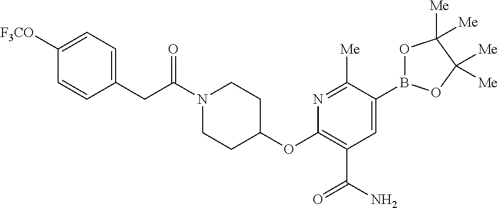 [SMILES: Cc1nc(OC2CCN(C(=O)Cc3ccc(OC(F)(F)F)cc3)CC2)c(C(N)=O)cc1B1OC(C)(C)C(C)(C)O1] | 2-{[(3R,4S)-3-fluoro- 1-{[4- (trifluoromethoxy) phenyl]acetyl} piperidin-4- yl]oxy}-5-(4,4,5,5- tetramethyl-1,3,2- dioxaborolan-2- yl)pyridine-3- carboxamide | MS m/z 564 [M + H]⁺ 5-bromo-6-methyl-2- [(1-{[4- (trifluoromethoxy) phenyl]acetyl} piperidin-4- yl)oxy]pyridine-3- carboxamide (Preparation 35). |
| 17 | 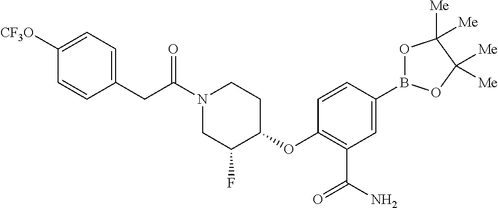 | 2-{[(3R,4S)-3-fluoro- 1-{[4- (trifluoromethoxy) phenyl]acetyl} piperidin-4- yl]oxy}-5-(4,4,5,5- tetramethyl-1,3,2- dioxaborolan-2- yl)benzamide | MS m/z 567 [M + H]⁺ 5-bromo-2-{[(3R,4S)- 3-fluoro-1-{[4- (trifluoromethoxy) phenyl]acetyl} piperidin-4- yl]oxy}benzamide (Preparation 36). |
| 18 | 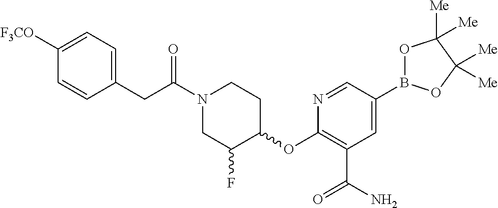 [SMILES: CC1(C)OB(c2cnc(OC3CCN(C(=O)Cc4ccc(OC(F)(F)F)cc4)CC3F)c(C(N)=O)c2)OC1(C)C] | Cis-racemic 2-((3- fluoro-1-(2-(4- (trifluoromethoxy) phenyl)acetyl) piperidin-4- yl)oxy)-5-(4,4,5,5- tetramethyl-1,3,2- dioxaborolan-2- yl)nicotinamide | MS m/z 568 [M + H]⁺ cis-racemic-5-bromo- 2-((3-fluoro-1-(2-(4- (trifluoromethoxy) phenyl)acetyl) piperidin-4- yl)oxy)nicotinamide (Preparation 29). |
| 19 | 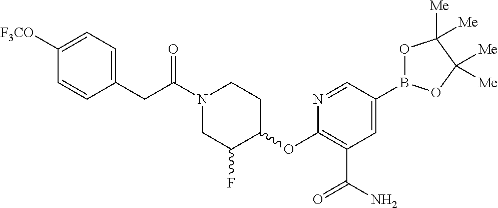 [SMILES: CC1(C)OB(c2cnc(OC3CCN(C(=O)Cc4ccc(OC(F)(F)F)cc4)CC3F)c(C(N)=O)c2)OC1(C)C] | Trans-racemic 2-((3- fluoro-1-(2-(4- (trifluoromethoxy) phenyl)acetyl) piperidin-4- yl)oxy)-5-(4,4,5,5- tetramethyl-1,3,2- dioxaborolan-2- yl)nicotinamide | MS m/z 568 [M + H]⁺ trans-racemic-5- bromo-2-((3-fluoro-1- (2-(4- (trifluoromethoxy) phenyl)acetyl) piperidin-4- yl)oxy)nicotinamide (Preparation 30). |

### Preparation 20

### (4-(3-carbamoyl-4-((1-(2-(4-(trifluoromethoxy)phenyl)acetyl)piperidin-4-yl)oxy)phenyl)-1H-1,2,3-triazol-1-yl)methyl pivalate

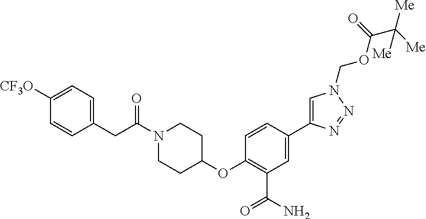

To a solution of 2-((1-(2-(4-(trifluoromethoxy)phenyl)acetyl)piperidin-4-yl)oxy)-5-((trimethylsilyl)ethynyl)benzamide (Preparation 21, 400 mg, 0.89 mmol) and azidomethylpivalate (Preparation 215, 142 mg, 0.89 mmol) in tBuOH/water (6 mL/6 mL) was added sodium ascorbate (106 mg, 0.54 mmol) and CuSO₄.5H₂O (11 mg, 0.04 mmol) and the reaction was stirred at room temperature for 16 hours. The reaction was diluted with EtOAc, washed with water, brine, dried over sodium sulphate and concentrated in vacuo. The residue was used directly in the next reaction as the title compound.

MS m/z 604 [M+H]⁺

### Preparation 21

### 2-((1-(2-(4-(trifluoromethoxy)phenyl)acetyl)piperidin-4-yl)oxy)-5-((trimethylsilyl)ethynyl)benzamide

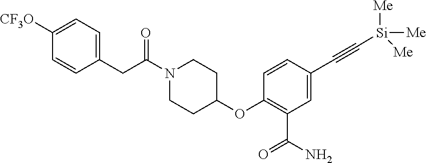

A solution of 5-bromo-2-[(1-{[4-(trifluoromethoxy)phenyl]acetyl}piperidin-4-yl)oxy]benzamide (Preparation 16, 500 mg, 0.99 mmol) and trimethyl-tributyl-stannylethynylsilane (1.85 g, 4.79 mmol) in toluene (10 mL) was purged with argon and treated with PdCl₂(PPh₃)₂ (35 mg, 0.05 mmol). The reaction was heated to 130° C. for 45 minutes before cooling, diluting with EtOAc, washing with water, brine, drying over sodium sulphate and concentrating in vacuo. The residue was used directly in the next reaction as the title compound.

MS m/z 519 [M+H]⁺

### Preparation 22

### 5-bromo-2-{[(3S,4S)-3-fluoro-1-{[4-(trifluoromethoxy)phenyl]acetyl}piperidin-4-yl]oxy}pyridine-3-carboxamide

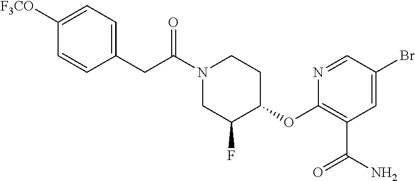 [SMILES: NC(=O)c1cc(Br)cnc1O[C@H]1CCN(C(=O)Cc2ccc(OC(F)(F)F)cc2)C[C@@H]1F]

To a suspension of 5-bromo-2-{[(3S,4S)-3-fluoropiperidin-4-yl]oxy}pyridine-3-carboxamide hydrochloride (Preparation 104, 730 mg, 1.87 mmol) in DCM (15 mL) was added DIPEA (1.67 mL, 9.34 mmol) at 0° C. followed by 4-trifluoromethoxyphenylacetic acid (410 mg, 1.87 mmol), EDCI (534 mg, 2.80 mmol) and HOBt (334 mg, 2.80 mmol). The reaction was stirred at room temperature for 16 hours before being diluted with EtOAc, washed with water, brine, dried over sodium sulphate and concentrated in vacuo. The residue was purified using silica gel column chromatography eluting with 3% MeOH in DCM to afford the title compound (600 mg, 62%).

¹H NMR (400 MHz, DMSO-d₆): δ ppm 1.72 (m, 1H), 2.06 (m, 1H), 3.39-3.82 (m, 6H), 4.80-4.98 (m, 1H), 5.44 (m, 1H), 7.28-7.40 (m, 4H), 7.64 (br s, 1H), 7.83 (m, 1H), 8.24 (br s, 1H), 8.45 (m, 1H).

MS m/z 520 [M⁷⁹Br+H]⁺

The following Preparations were prepared according to the method described by Preparation 22 using the appropriate amine and carboxylic acid as described below:

| Prepar- | | | |
|---|---|---|---|
| ation | Structure/Name | Data | Starting materials |
| 23 | 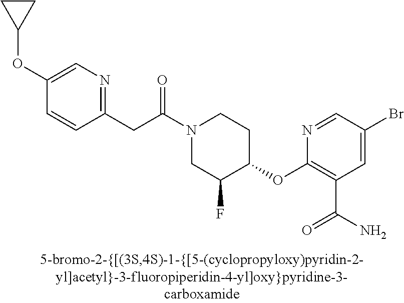 [SMILES: NC(=O)c1cc(Br)cnc1O[C@H]1CCN(C(=O)Cc2ccc(OC3CC3)cn2)C[C@@H]1F] | MS m/z 495 [M⁸¹Br + H]⁺ ¹H NMR (400 MHz, DMSO-d₆): δ ppm 0.69 (m, 2H), 0.80 (m, 2H), 1.98 (m, 1H), 2.07 (m, 1H), 3.53-4.09 (m, 7H), 4.78-4.90 (m, 1H), 5.44 (m, 1H), 7.25 (d, 1H), 7.46 (dd, 1H), 7.65 (br s, 1H), 7.84 (br s, 1H), 8.23 (m, 2H), 8.43 (d, 1H). | 5-bromo-2-{[(3S,4S)- 3-fluoropiperidin-4- yl]oxy}pyridine-3- carboxamide hydrochloride (Preparation 104) and [5-(cyclopropyloxy) pyridin-2-yl]acetic acid (Preparation 211). |
| 24 | 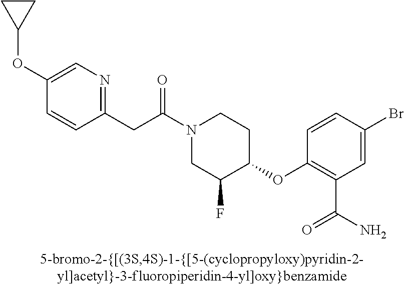 [SMILES: NC(=O)c1cc(Br)ccc1O[C@H]1CCN(C(=O)Cc2ccc(OC3CC3)cn2)C[C@@H]1F] | MS m/z 492 [M⁷⁹Br + H]⁺ | 5-bromo-2-{[(3S,4S)- 3-fluoropiperidin-4- yl]oxy}benzamide hydrochloride (Preparation 112) and [5-(cyclopropyloxy) pyridin-2-yl]acetic acid (Preparation 211). |
| 25 | 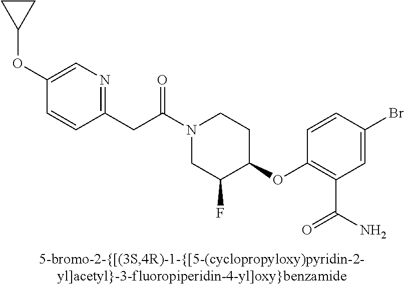 [SMILES: NC(=O)c1cc(Br)ccc1O[C@@H]1CCN(C(=O)Cc2ccc(OC3CC3)cn2)C[C@@H]1F] | MS m/z 492 [M⁷⁹Br + H]⁺ | 5-bromo-2-{[(3S,4R)- 3-fluoropiperidin-4- yl]oxy}benzamide hydrochloride (Preparation 107) and [5-(cyclopropyloxy) pyridin-2-yl]acetic acid (Preparation 211). |
| 26 | 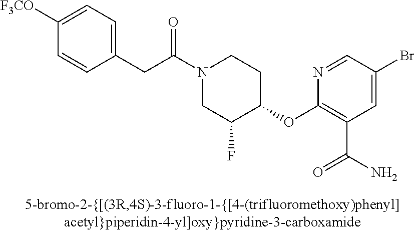 [SMILES: NC(=O)c1cc(Br)cnc1O[C@H]1CCN(C(=O)Cc2ccc(OC(F)(F)F)cc2)C[C@H]1F] | ¹H NMR (400 MHz, DMSO-d₆): δ ppm 1.85 (m, 1H), 1.97 (m, 1H), 2.95 (m, 1H), 3.56-3.97 (m, 3H), 4.29 (m, 1H), 4.55 (m, 1H), 5.00-5.15 (m, 1H), 5.49 (m, 1H), 7.30-7.39 (m, 4H), 7.52 (br s, 1H), 7.92 (br s, 1H), 8.27 (m, 1H), 8.43 (m, 1H). | 5-bromo-2-{[(3R,4S)- 3-fluoropiperidin-4- yl]oxy}pyridine-3- carboxamide hydrochloride (Preparation 106) with triethylamine. |
| 27 | 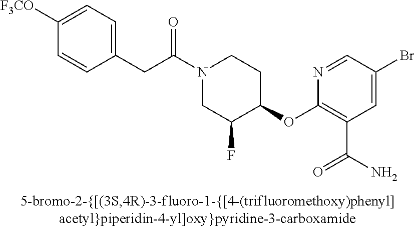 [SMILES: NC(=O)c1cc(Br)cnc1O[C@@H]1CCN(C(=O)Cc2ccc(OC(F)(F)F)cc2)C[C@@H]1F] | ¹H NMR (400 MHz, DMSO-d₆): δ ppm 1.78- 1.85 (m, 1H), 1.97-2.01 (m, 1H), 2.94-3.32 (m, 1H), 3.56-3.72 (m, 1H), 3.86 (s, 2H), 3.97-4.55 (m, 2H), 5.00-5.14 (m, 1H), 5.42-5.48 (m, 1H), 7.28-7.35 (m, 4H), 7.52 (br s, 1H), 7.92 (br s, 1H), 8.27 (t, 1H), 8.43 (t, 1H). | 5-bromo-2-{[(3S,4R)- 3-fluoropiperidin-4- yl]oxy}pyridine-3- carboxamide hydrochloride (Preparation 105) with triethylamine. The residue was purified using silica gel column chromatography eluting with 0-30% EtOAc in heptanes. |

### Preparation 28

### 5-bromo-2-{[(3S,4R)-3-fluoro-1-{[4-(trifluoromethoxy)phenyl]acetyl}piperidin-4-yl]oxy}benzamide

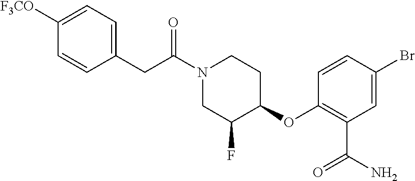 [SMILES: NC(=O)c1cc(Br)ccc1O[C@@H]1CCN(C(=O)Cc2ccc(OC(F)(F)F)cc2)C[C@@H]1F]

To a stirred suspension of 5-bromo-2-{[(3S,4R)-3-fluoropiperidin-4-yl]oxy}benzamide hydrochloride salt (Preparation 107, 800 mg, 2.26 mmol) in DCM (10 mL) at 0° C. was added triethylamine (1.59 mL, 11.33 mmol) followed by 4-trifluoromethoxyphenylacetic acid (498 mg, 2.26 mmol), EDCI (649 mg, 3.39 mmol) and HOBt (458 mg, 3.39 mmol). The reaction was stirred at room temperature for 16 hours. The reaction was diluted with EtOAc, washed with water, brine, dried over sodium sulphate and concentrated in vacuo. The residue was purified using silica gel column chromatography eluting with 2-3% MeOH in DCM to afford the title compound as a white solid (800 mg, 68%).

¹H NMR (400 MHz, DMSO-d₆): δ ppm 1.73-1.78 (m, 1H), 1.94-2.01 (m, 1H), 2.88-3.31 (m, 2H), 3.44-3.98 (m, 3H), 4.27-4.57 (m, 1H), 4.89-5.10 (m, 2H), 7.29 (m, 5H), 7.53 (br s, 1H), 7.63 (d, 1H), 7.76 (br s, 1H), 7.90 (s, 1H).

MS m/z 519 [M⁷⁹Br+H]⁺

The following Preparations were prepared according to the methods described by Preparation 28 using the appropriate piperidine and carboxylic acid as described below:

| Prepar- | | |
|---|---|---|
| ation | Name/Structure | Data and starting materials |
| 29 | 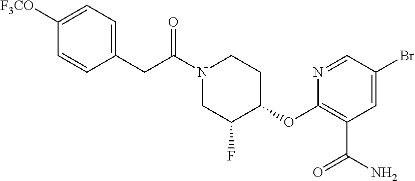 [SMILES: NC(=O)c1cc(Br)cnc1O[C@H]1CCN(C(=O)Cc2ccc(OC(F)(F)F)cc2)C[C@H]1F] 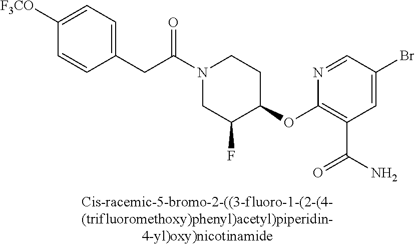 [SMILES: NC(=O)c1cc(Br)cnc1O[C@@H]1CCN(C(=O)Cc2ccc(OC(F)(F)F)cc2)C[C@@H]1F] | ¹H NMR (400 MHz, DMSO-d₆): δ ppm 1.82 (m, 1H), 1.98 (m, 1H), 2.94-3.25 (m, 1H), 3.56-4.01 (m, 4H), 4.29-4.55 (m, 1H), 5.00 (m, 0.5H), 5.12 (m, 0.5H), 5.42-5.48 (m, 1H), 7.28-7.36 (m, 4H), 7.53 (br s, 1H), 7.92 (br s, 1H), 8.27 (m, 1H), 8.44 (m, 1H). cis-racemic-5-bromo-2-{[3- fluoropiperidin-4-yl]oxy}pyridine- 3-carboxamide hydrochloride (Preparation 115) and 4- trifluoromethoxyphenylacetic acid. |
| 30 | 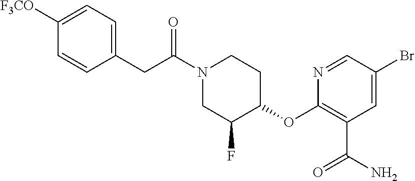 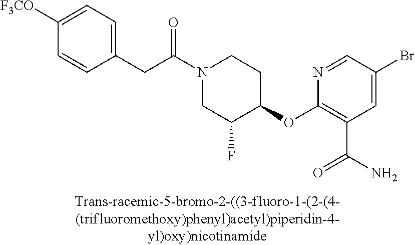 [SMILES: NC(=O)c1cc(Br)cnc1O[C@@H]1CCN(C(=O)Cc2ccc(OC(F)(F)F)cc2)C[C@H]1F] | ¹H NMR (400 MHz, DMSO-d₆): δ ppm 1.70-2.11 (m, 2H), 2.94 (m, 1H), 3.53-4.01 (m, 5H), 5.00 (m, 0.5H), 5.14 (m, 0.5H), 5.40-5.55 (m, 1H), 7.30-7.35 (m, 4H), 7.52 (br s, 1H), 7.92 (br s, 1H), 8.27 (d, 1H), 8.44 (d, 1H). trans-racemic-5-bromo-2-{[3- fluoropiperidin-4-yl]oxy}pyridine- 3-carboxamide hydrochloride (Preparation 116) and 4- trifluoromethoxyphenylacetic acid. |
| 31 | 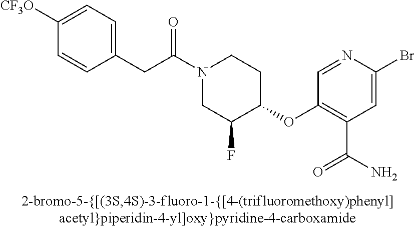 | MS m/z 520 [M⁷⁹Br + H]⁺ 2-bromo-5-{[(3S,4S)-3- fluoropiperidin-4-yl]oxy}pyridine- 4-carboxamide hydrochloride (Preparation 114) and 4- trifluoromethoxyphenylacetic acid. |
| 32 | 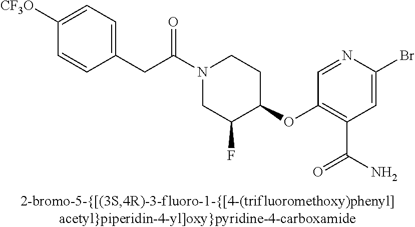 | MS m/z 520 [M⁷⁹Br + H]⁺ 2-bromo-5-{[(3S,4R)-3- fluoropiperidin-4-yl]oxy}pyridine- 4-carboxamide hydrochloride (Preparation 113) and 4- trifluoromethoxyphenylacetic acid. |
| 33 | 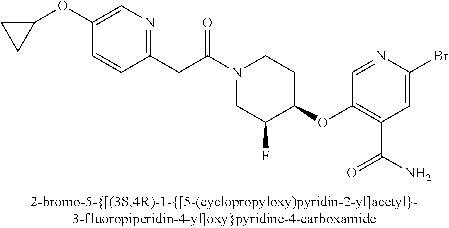 [SMILES: NC(=O)c1cc(Br)ncc1O[C@@H]1CCN(C(=O)Cc2ccc(OC3CC3)cn2)C[C@@H]1F] | Taken on directly to the next step. 2-bromo-5-{[(3S,4R)-3- fluoropiperidin-4-yl]oxy}pyridine- 4-carboxamide hydrochloride (Preparation 113) and [5- (cyclopropyloxy)pyridin-2-yl] acetic acid (Preparation 211). |
| 34 | 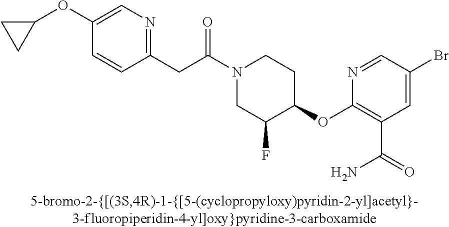 [SMILES: NC(=O)c1cc(Br)cnc1O[C@@H]1CCN(C(=O)Cc2ccc(OC3CC3)cn2)C[C@@H]1F] | MS m/z 493 [M⁷⁹Br + H]⁺ 5-bromo-2-{[(3S,4R)-3- fluoropiperidin-4-yl]oxy}pyridine- 3-carboxamide hydrochloride (Preparation 105) and [5- (cyclopropyloxy)pyridin-2-yl] acetic acid (Preparation 211). |
| 35 | 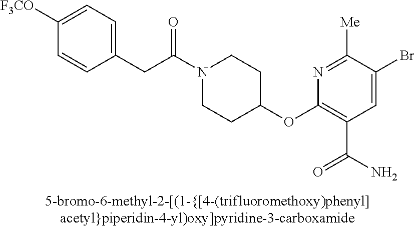 [SMILES: Cc1nc(OC2CCN(C(=O)Cc3ccc(OC(F)(F)F)cc3)CC2)c(C(N)=O)cc1Br] | MS m/z 518 [M⁸¹Br + H]⁺ ¹H NMR (400 MHz, DMSO-d₆): δ ppm 1.71 (m, 2H), 1.98 (m, 2H), 2.51 (s, 3H), 3.31-3.50 (m, 2H), 3.73-3.83 (m, 4H), 5.36 (m, 1H), 7.28-7.36 (m, 4H), 7.48 (br s, 1H), 7.77 (br s, 1H), 8.19 (s, 1H). 5-bromo-6-methyl-2-[(1-{[4- (trifluoromethoxy)phenyl]acetyl} piperidin-4-yl)oxy]pyridine-3- carboxamide hydrochloride (Preparation 111) with DIPEA as base and and 4- trifluoromethoxyphenylacetic acid. |
| 36 | 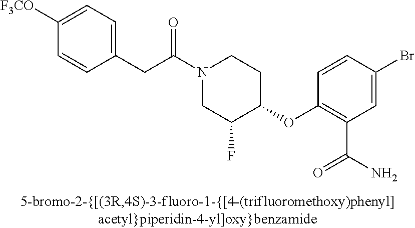 [SMILES: NC(=O)c1cc(Br)ccc1O[C@H]1CCN(C(=O)Cc2ccc(OC(F)(F)F)cc2)C[C@H]1F] | MS m/z 519 [M⁷⁹Br + H]⁺ ¹H NMR (400 MHz, DMSO-d₆): δ ppm 1.68-1.78 (m, 1H), 1.90-1.98 (m, 1H), 2.91 (m, 0.5H), 3.07-3.32 (m, 1H), 3.68 (m, 1H), 3.83 (m, 1.5H), 3.98 (m, 0.5H), 4.30 (m, 1H), 4.55 (m, 0.5H), 4.89-5.10 (m, 2H), 7.28-7.35 (m, 5H), 7.53 (br s, 1H), 7.64-7.66 (m, 1H), 7.76 (br s, 1H), 7.90 (m, 1H). 5-bromo-2-{[(3R,4S)-3- fluoropiperidin-4-yl]oxy}benzamide hydrochloride (Preparation 108) and 4-trifluoromethoxyphenylacetic |
| | | acid. |

### Preparation 37

### 5-bromo-2-({1-[4-(trifluoromethoxy)benzyl]piperidin-4-yl}oxy)pyridine-3-carboxamide

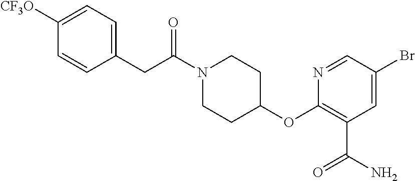

To a solution of 5-bromo-2-(piperidin-4-yloxy)pyridine-3-carboxamide hydrochloride (Preparation 109, 6.40 g, 21.13 mmol) and 4-(trifluoromethoxy)phenylacetic acid (4.70 g, 21.13 mmol) in DMF (40 mL), was added DIPEA (18.6 mL, 106.6 mL) followed by HATU (12.16 g, 31.19 mmol) and the reaction was stirred at room temperature for 16 hours. The reaction was diluted with EtOAc, washed with saturated aqueous NaHCO₃ solution, water, brine, dried over sodium sulphate and concentrated in vacuo. The residue was purified using silica gel column chromatography eluting with 3% MeOH in DCM to afford the title compound (6.90 g, 64%).

¹H NMR (400 MHz, DMSO-d₆): δ ppm 1.70 (m, 2H), 1.90 (m, 2H), 3.30-3.40 (m, 2H), 3.79 (m, 4H), 5.30 (m, 1H), 7.20 (d, 2H), 7.30 (d, 2H), 7.58 (br s, 1H), 7.80 (br s, 1H), 8.20 (d, 1H), 8.40 (d, 1H).

MS m/z 504 [M⁸¹Br+H]⁺

### Preparation 38

### 5-bromo-2-{[1-({4-[(trifluoromethyl)sulfanyl]phenyl}acetyl)piperidin-4-yl]oxy}pyridine-3-carboxamide

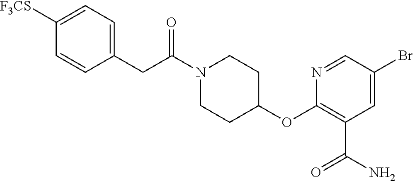

The title compound was prepared according to the method described for Preparation 37 using 4-(trifluoromethylthio)phenyl acetic acid.

¹H NMR (400 MHz, DMSO-d₆): δ ppm 1.70 (m, 2H), 1.90 (m, 2H), 3.30-3.79 (m, 4H), 3.83 (s, 2H), 5.30 (m, 1H), 7.40 (d, 2H), 7.57 (s, 1H), 7.60 (m, 2H), 7.79 (s, 1H), 8.20 (d, 1H), 8.40 (d, 1H).

### Preparation 39

### 5-bromo-2-[(1-{[4-(trifluoromethoxy)phenyl]acetyl}piperidin-4-yl)oxy]benzamide

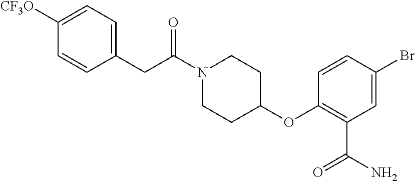

The title compound was prepared according to the method described for Preparation 23 using 5-bromo-2-(piperidin-4-yloxy)benzamide (Preparation 110).

¹H NMR (400 MHz, DMSO-d₆): δ ppm 1.64 (m, 2H), 1.88 (m, 2H), 3.31-3.44 (m, 2H), 3.78 (m, 4H), 4.77 (m, 1H), 7.19 (d, 1H), 7.28-7.36 (m, 4H), 7.53 (br s, 1H), 7.57-7.60 (m, 1H), 7.64 (br s, 1H), 7.77 (m, 1H).

### Preparation 40

### 5-bromo-2-((1-(2-(4-cyclopropoxyphenyl)acetyl)piperidin-4-yl)oxy)benzamide

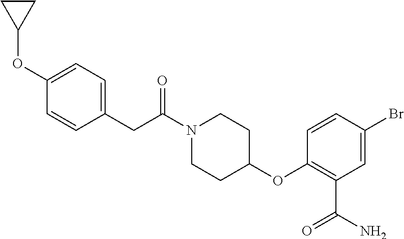 [SMILES: NC(=O)c1cc(Br)ccc1OC1CCN(C(=O)Cc2ccc(OC3CC3)cc2)CC1]

The title compound was prepared according to the method described for Preparation 37 using 4-cyclopropyloxyphenyl acetic acid. Taken on directly to the next step.

### Preparation 41

### 2-bromo-5-{[(3R,4S)-3-fluoro-1-{[4-(trifluoromethoxy)phenyl]acetyl}piperidin-4-yl]oxy}pyridine-4-carboxamide

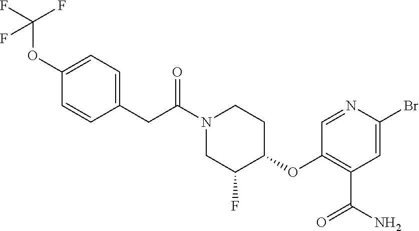 [SMILES: NC(=O)c1cc(Br)ncc1O[C@H]1CCN(C(=O)Cc2ccc(OC(F)(F)F)cc2)C[C@H]1F]

The title compound was prepared according to the methods described for Preparations 32, 113 and 125 using tert-butyl (3S,4R)-3-fluoro-4-hydroxypiperidine-1-carboxylate.

MS m/z 520 [M⁷⁹Br+H]⁺

### Preparation 42

### 2-{[(3R,4S)-3-fluoropiperidin-4-yl]oxy}-5-(1-methyl-1H-imidazol-4-yl)pyridine-3-carboxamide dihydrochloride salt

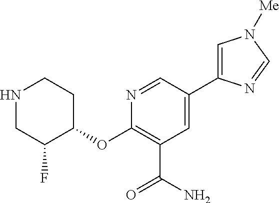 [SMILES: Cn1cnc(-c2cnc(O[C@H]3CCNC[C@H]3F)c(C(N)=O)c2)c1]

A solution of tert-butyl (3R,4S)-4-[(5-bromo-3-carbamoylpyridin-2-yl)oxy]-3-fluoropiperidine-1-carboxylate (Preparation 119, 12.7 g, 30.4 mmol) bispinacolatodiboron (11.6 g, 45.5 mmol) and potassium acetate (5.96 g, 60.7 mmol) in dioxane (300 mL) was degassed with nitrogen for 30 minutes. 1,1-bis(diphenylphosphino)ferrocene palladium (II) dichloride (496 mg, 0.61 mmol) was added and the reaction heated to 100° C. for 3 hours. The reaction was cooled and further degassed with nitrogen. 2M aqueous cesium carbonate solution (120 mL) was added followed by 4-iodo-1-methyl-1H-imidazole (6.52 g, 31.4 mmol), and the reaction heated to 100° C. for 18 hours. The reaction was cooled and quenched with water (100 mL) and extracted into EtOAc (2×400 mL). The combined extracts were washed with brine (300 mL), dried over sodium sulphate and concentrated in vacuo. The residue was dissolved in EtOAc (100 mL) and treated with 4N HCl in dioxane (15 mL) to afford a white precipitate that was filtered and washed with cold MeCN (50 ml). The solid was dissolved in methanol (300 mL) and stirred at room temperature for 2 hours. The reaction was concentrated in vacuo and the residue triturated with IPA (200 mL) and toluene (100 mL) to afford the title compound (8.33 g, 81%).

¹H NMR (400 MHz, DMSO-d₆): δ ppm 2.27-2.51 (m, 2H), 3.35 (dt, 1H), 3.51-3.64 (m, 2H), 3.81 (m, 1H), 4.01 (s, 3H), 5.39 (d, 1H), 5.75 (m, 1H), 8.03 (d, 1H), 8.60 (s, 1H), 8.66 (d, 1H), 8.70 (d, 1H), 9.05 (s, 1H).

MS m/z 320 [M+H]⁺

### Preparation 43

### 2-{[(3S,4R)-3-fluoropiperidin-4-yl]oxy}-5-(1-methyl-1H-imidazol-4-yl)benzamide dihydrochloride salt

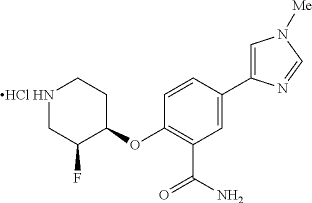

A solution of tert-butyl (3S,4R)-4-[2-carbamoyl-4-(1-methyl-1H-imidazol-4-yl)phenoxy]-3-fluoropiperidine-1-carboxylate (Preparation 96, 8.51 g, 20.40 mmol) in HCl (4M in dioxane, 20 mL) and MeOH (100 mL) was stirred at room temperature for 16 hours. The reaction was concentrated in vacuo to afford the title compound.

¹H NMR (400 MHz, DMSO-d₆): δ ppm 2.06 (m, 1H), 2.16 (m, 1H), 3.09 (m, 1H), 3.21-3.42 (m, 2H), 3.59 (m, 1H), 3.87 (s, 3H), 5.10 (m, 1H), 5.20-5.35 (m, 1H), 7.45 (m, 1H), 7.54 (br s, 1H), 7.78 (br s, 1H), 8.02 (m, 1H), 8.17 (m, 2H), 9.00 (br s, 1H), 9.18 (s, 1H), 10.00 (br s, 1H).

MS m/z 319 [M+H]⁺

The following Preparations were prepared according to the methods described by Preparation 43 using the appropriate boc protected piperidine as described in the table below. All products were isolated as their hydrochloride salts.

| Prepar- | | |
|---|---|---|
| ation | Name/Structure | Data and starting materials |
| 44 | 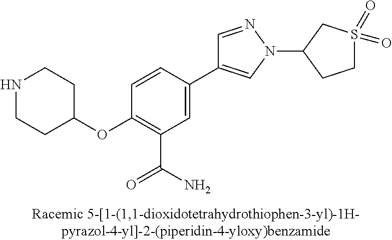 [SMILES: NC(=O)c1cc(-c2cnn(C3CCS(=O)(=O)C3)c2)ccc1OC1CCNCC1] | Used directly in the next reaction. Tert-butyl 4-{2-carbamoyl-4-[1-(1,1- dioxidotetrahydrothiophen-3-yl)-1H- pyrazol-4-yl]phenoxy}piperidine-1- carboxylate (Preparation 137). |
| 45 | 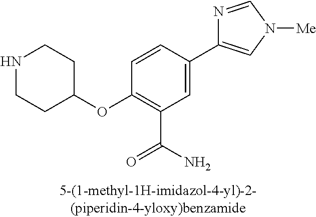 [SMILES: Cn1cnc(-c2ccc(OC3CCNCC3)c(C(N)=O)c2)c1] | ¹H NMR (400 MHz, DMSO-d₆): δ ppm 1.94-1.97 (m, 2H), 2.12-2.14 (m, 2H), 3.10 (br s, 2H), 3.21 (m, 2H), 3.87 (s, 3H), 4.88 (br s, 1H), 7.37 (m, 1H), 7.60 (br s, 1H), 7.69 (br s, 1H), 7.91-7.94 (m, 1H), 8.06 (d, 1H), 9.10 (br s, 1H), 9.17 (s, 1H). Tert-butyl 4-[2-carbamoyl-4-(1- methyl-1H-imidazol-4- yl)phenoxy]piperidine-1- carboxylate (Preparation 138). |
| 46 | 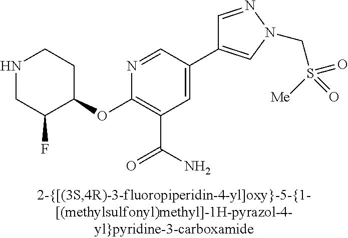 [SMILES: CS(=O)(=O)Cn1cc(-c2cnc(O[C@@H]3CCNC[C@@H]3F)c(C(N)=O)c2)cn1] | ¹H NMR (400 MHz, DMSO-d₆): δ ppm 2.04-2.10 (m, 1H), 2.12-2.18 (m, 1H), 3.06 (s, 3H), 3.16-3.25 (m, 1H), 3.30-3.38 (m, 1H), 3.48-3.68 (m, 2H), 5.25-5.37 (d, 1H), 5.42- 5.51 (m, 1H), 5.76 (s, 2H), 7.57 (br s, 1H), 7.88 (br s, 1H), 8.20 (s, 1H), 8.36 (d, 1H), 8.42 (s, 1H), 8.58 (d, 1H), 8.80 (br s, 1H), 9.41 (br s, 1H). MS m/z 398 [M + H]⁺ Tert-butyl (3S,4R)-4-[(3-carbamoyl- 5-{1-[(methylsulfonyl)methyl]-1H- pyrazol-4-yl}pyridin-2-yl)oxy]-3- fluoropiperidine-1-carboxylate |
| | | (Preparation 80). |
| 47 | 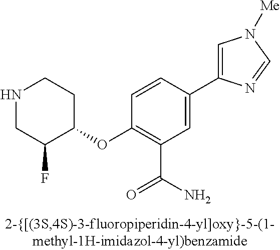 [SMILES: Cn1cnc(-c2ccc(O[C@H]3CCNC[C@@H]3F)c(C(N)=O)c2)c1] | MS m/z 319 [M + H]⁺ The title compound was triturated with ether to afford a white solid. Tert-butyl (3S,4S)-4-[2-carbamoyl- 4-(1-methyl-1H-imidazol-4- yl)phenoxy]-3-fluoropiperidine-1- carboxylate (Preparation 92). |
| 48 | 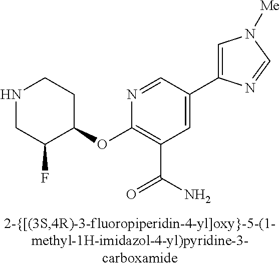 [SMILES: Cn1cnc(-c2cnc(O[C@@H]3CCNC[C@@H]3F)c(C(N)=O)c2)c1] | ¹H NMR (400 MHz, DMSO-d₆): δ ppm 2.09-2.12 (m, 1H), 2.20-2.25 (m, 1H), 3.11-3.20 (m, 1H), 3.30- 3.35 (m, 1H), 3.40-3.50 (m, 2H), 3.87 (s, 3H), 5.31 (d, 1H), 5.54- 5.64 (m, 1H), 7.58 (s, 1H), 7.97 (s, 1H), 8.22 (s, 1H), 8.57 (d, 1H), 8.80 (d, 1H), 8.90 (br s, 1H), 9.06 (br s, 1H), 9.46 (br s, 1H). Tert-butyl (3S,4R)-4-{[3-carbamoyl- 5-(1-methyl-1H-imidazol-4- yl)pyridin-2-yl]oxy}-3- fluoropiperidine-1-carboxylate (Preparation 78). |
| 49 | 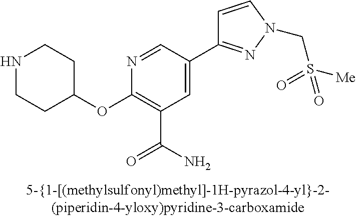 [SMILES: CS(=O)(=O)Cn1ccc(-c2cnc(OC3CCNCC3)c(C(N)=O)c2)n1] | MS m/z 380 [M + H]⁺ ¹H NMR (400 MHz, DMSO-d₆): δ ppm 1.99 (m, 2H), 2.15 (m, 2H), 3.05 (s, 3H), 3.15 (m, 2H), 3.22 (m, 2H), 5.38 (m, 1H), 5.75 (s, 2H), 7.63 (br s, 1H), 7.75 (br s, 1H), 8.17 (s, 1H), 8.26 (d, 1H), 8.38 (s, 1H), 8.56 (d, 1H). tert-butyl 4-[(3-carbamoyl-5-{1- [(methylsulfonyl)methyl]-1H- pyrazol-4-yl}pyridin-2- yl)oxy]piperidine-1-carboxylate (Preparation 87). |
| 50 | 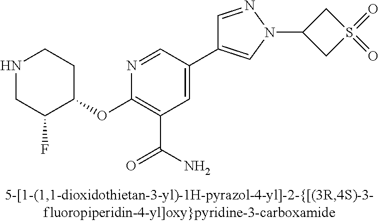 [SMILES: NC(=O)c1cc(-c2cnn(C3CS(=O)(=O)C3)c2)cnc1O[C@H]1CCNC[C@H]1F] | MS m/z 410 [M + H]⁺ tert-butyl (3R,4S)-4-({3-carbamoyl- 5-[1-(1,1-dioxidothietan-3-yl)-1H- pyrazol-4-yl]pyridin-2-yl}oxy)-3- fluoropiperidine-1-carboxylate (Preparation 79). |
| 51 | 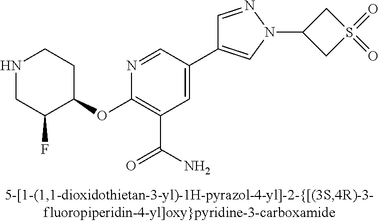 [SMILES: NC(=O)c1cc(-c2cnn(C3CS(=O)(=O)C3)c2)cnc1O[C@@H]1CCNC[C@@H]1F] | MS m/z 410 [M + H]⁺ tert-butyl (3S,4R)-4-({3-carbamoyl- 5-[1-(1,1-dioxidothietan-3-yl)-1H- pyrazol-4-yl]pyridin-2-yl}oxy)-3- fluoropiperidine-1-carboxylate (Preparation 86). |
| 52 | 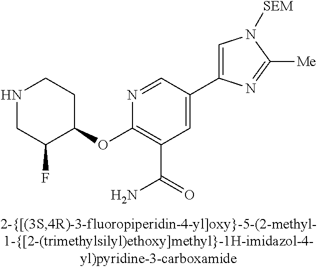 [SMILES: Cc1nc(-c2cnc(O[C@@H]3CCNC[C@@H]3F)c(C(N)=O)c2)cn1COCC[Si](C)(C)C] | MS m/z 450 [M + H]⁺ tert-butyl (3S,4R)-4-{[3-carbamoyl- 5-(2-methyl-1-{[2- (trimethylsilyl)ethoxy]methyl}-1H- imidazol-4-yl)pyridin-2-yl]oxy}-3- fluoropiperidine-1-carboxylate (Preparation 84). |
| 53 | 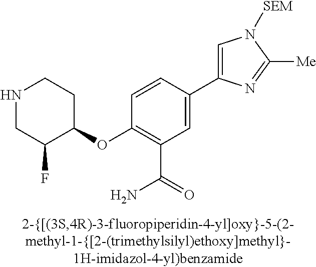 [SMILES: Cc1nc(-c2ccc(O[C@@H]3CCNC[C@@H]3F)c(C(N)=O)c2)cn1COCC[Si](C)(C)C] | MS m/z 449 [M + H]⁺ tert-butyl (3S,4R)-4-[2-carbamoyl- 4-(2-methyl-1-{[2- (trimethylsilyl)ethoxy]methyl}-1H- imidazol-4-yl)phenoxy]-3- fluoropiperidine-1-carboxylate (Preparation 85). |
| 54 | 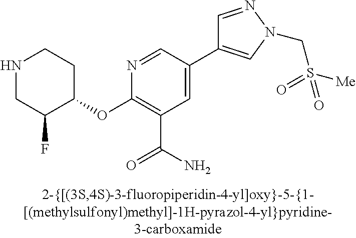 [SMILES: CS(=O)(=O)Cn1cc(-c2cnc(O[C@H]3CCNC[C@@H]3F)c(C(N)=O)c2)cn1] | MS m/z 398 [M + H]⁺ tert-butyl (3S,4S)-4-[(3-carbamoyl- 5-{1-[(methylsulfonyl)methyl]-1H- pyrazol-4-yl}pyridin-2-yl)oxy]-3- fluoropiperidine-1-carboxylate (Preparation 88). |
| 55 | 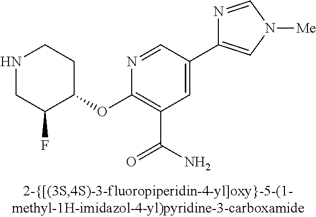 [SMILES: Cn1cnc(-c2cnc(O[C@H]3CCNC[C@@H]3F)c(C(N)=O)c2)c1] | MS m/z 320 [M + H]⁺ tert-butyl (3S,4S)-4-{[3-carbamoyl- 5-(1-methyl-1H-imidazol-4- yl)pyridin-2-yl]oxy}-3- fluoropiperidine-1-carboxylate (Preparation 89). |
| 56 | 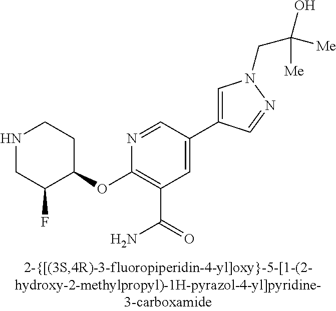 [SMILES: CC(C)(O)Cn1cc(-c2cnc(O[C@@H]3CCNC[C@@H]3F)c(C(N)=O)c2)cn1] | MS m/z 378 [M + H]⁺ tert-butyl (3S,4R)-4-({3-carbamoyl- 5-[1-(2-hydroxy-2-methylpropyl)- 1H-pyrazol-4-yl]pyridin-2-yl}oxy)- 3-fluoropiperidine-1-carboxylate (Preparation 82). |
| 57 | 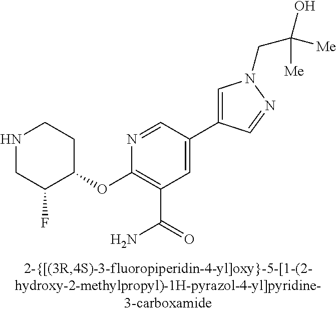 [SMILES: CC(C)(O)Cn1cc(-c2cnc(O[C@H]3CCNC[C@H]3F)c(C(N)=O)c2)cn1] | MS m/z 378 [M + H]⁺ tert-butyl (3R,4S)-4-({3-carbamoyl- 5-[1-(2-hydroxy-2-methylpropyl)- 1H-pyrazol-4-yl]pyridin-2-yl}oxy)- 3-fluoropiperidine-1-carboxylate (Preparation 83). |
| 58 | 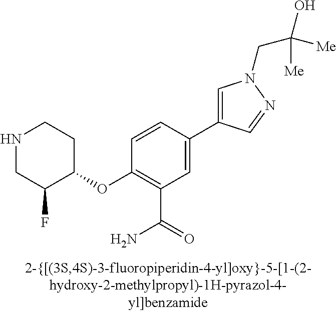 [SMILES: CC(C)(O)Cn1cc(-c2ccc(O[C@H]3CCNC[C@@H]3F)c(C(N)=O)c2)cn1] | MS m/z 377 [M + H]⁺ tert-butyl (3S,4S)-4-{2-carbamoyl- 4-[1-(2-hydroxy-2-methylpropyl)- 1H-pyrazol-4-yl]phenoxy}-3- fluoropiperidine-1-carboxylate (Preparation 98). |
| 59 | 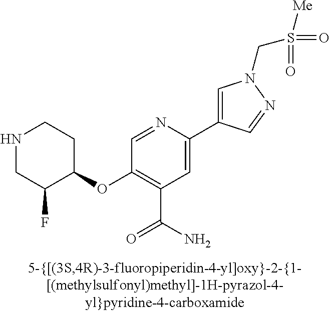 [SMILES: CS(=O)(=O)Cn1cc(-c2cc(C(N)=O)c(O[C@@H]3CCNC[C@@H]3F)cn2)cn1] | MS m/z 398 [M + H]⁺ tert-butyl (3S,4R)-4-[(4-carbamoyl- 6-{1-[(methylsulfonyl)methyl]-1H- pyrazol-4-yl}pyridin-3-yl)oxy]-3- fluoropiperidine-1-carboxylate (Preparation 91). |
| 60 | 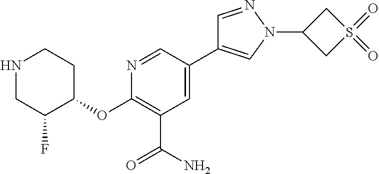 [SMILES: NC(=O)c1cc(-c2cnn(C3CS(=O)(=O)C3)c2)cnc1O[C@H]1CCNC[C@H]1F] 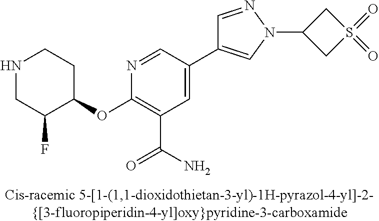 [SMILES: NC(=O)c1cc(-c2cnn(C3CS(=O)(=O)C3)c2)cnc1O[C@@H]1CCNC[C@@H]1F] | MS m/z 410 [M + H]⁺ Cis-racemic tert-butyl-4-({3- carbamoyl-5-[1-(1,1-dioxidothietan- 3-yl)-1H-pyrazol-4-yl]pyridin-2- yl}oxy)-3-fluoropiperidine-1- carboxylate (Preparation 81). |
| 61 | 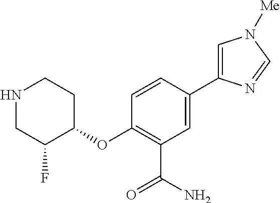 [SMILES: Cn1cnc(-c2ccc(O[C@H]3CCNC[C@H]3F)c(C(N)=O)c2)c1] 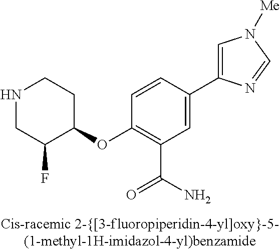 [SMILES: Cn1cnc(-c2ccc(O[C@@H]3CCNC[C@@H]3F)c(C(N)=O)c2)c1] | MS m/z 319 [M + H]⁺ Cis-racemic tert-butyl 4-(2- carbamoyl-4-(1-methyl-1H- imidazol-4-yl)phenoxy)-3- fluoropiperidine-1-carboxylate (Preparation 139). |
| 62 | 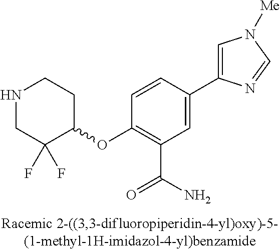 [SMILES: Cn1cnc(-c2ccc(OC3CCNCC3(F)F)c(C(N)=O)c2)c1] | MS m/z 337 [M + H]⁺ Racemic tert-butyl 4-(2-carbamoyl- 4-(1-methyl-1H-imidazol-4- yl)phenoxy)-3,3-difluoropiperidine- 1-carboxylate (Preparation 90). |
| 63 | 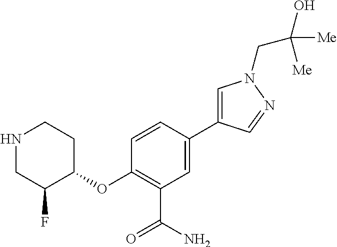 [SMILES: CC(C)(O)Cn1cc(-c2ccc(O[C@H]3CCNC[C@@H]3F)c(C(N)=O)c2)cn1] 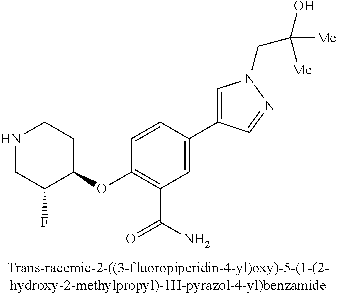 [SMILES: CC(C)(O)Cn1cc(-c2ccc(O[C@@H]3CCNC[C@H]3F)c(C(N)=O)c2)cn1] | MS m/z 377 [M + H]⁺ Trans-racemic-tert-butyl 4-(2- carbamoyl-4-(1-(2-hydroxy-2- methylpropyl)-1H-pyrazol-4- yl)phenoxy)-3-fluoropiperidine-1- carboxylate (Preparation 94). |
| 64 | 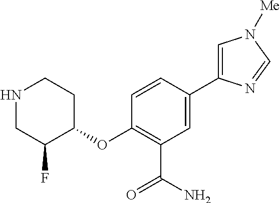 [SMILES: Cn1cnc(-c2ccc(O[C@H]3CCNC[C@@H]3F)c(C(N)=O)c2)c1] 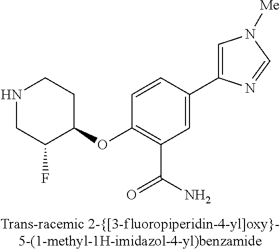 [SMILES: Cn1cnc(-c2ccc(O[C@@H]3CCNC[C@H]3F)c(C(N)=O)c2)c1] | MS m/z 319 [M + H]⁺ Trans-racemic-tert-butyl-4-[2- carbamoyl-4-(1-methyl-1H- imidazol-4-yl)phenoxy]-3- fluoropiperidine-1-carboxylate (Preparation 93). |
| 65 | 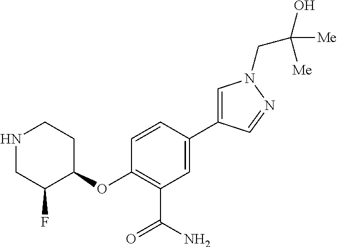 [SMILES: CC(C)(O)Cn1cc(-c2ccc(O[C@@H]3CCNC[C@@H]3F)c(C(N)=O)c2)cn1] 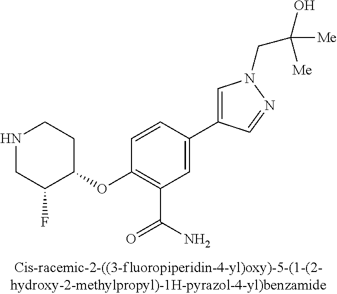 [SMILES: CC(C)(O)Cn1cc(-c2ccc(O[C@H]3CCNC[C@H]3F)c(C(N)=O)c2)cn1] | MS m/z 377 [M + H]⁺ Cis-racemic-tert-butyl 4-(2- carbamoyl-4-(1-(2-hydroxy-2- methylpropyl)-1H-pyrazol-4- yl)phenoxy)-3-fluoropiperidine-1- carboxylate (Preparation 95). |
| 66 | 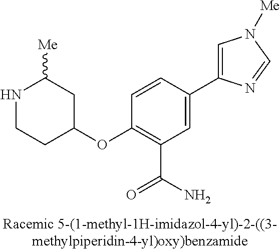 [SMILES: CC1CC(Oc2ccc(-c3cn(C)cn3)cc2C(N)=O)CCN1] | MS m/z 315 [M + H]⁺ Racemic tert-butyl 4-(2-carbamoyl- 4-(1-methyl-1H-imidazol-4- yl)phenoxy)-2-methylpiperidine-1- carboxylate (Preparation 142). |
| 67 | 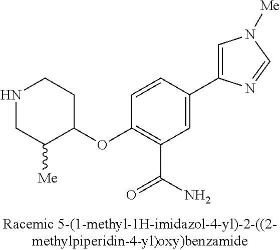 [SMILES: CC1CNCCC1Oc1ccc(-c2cn(C)cn2)cc1C(N)=O] | MS m/z 315 [M + H]⁺ Racemic tert-butyl 4-(2-carbamoyl- 4-(1-methyl-1H-imidazol-4- yl)phenoxy)-3-methylpiperidine-1- carboxylate (Preparation 141). |
| 68 | 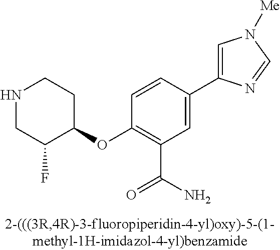 [SMILES: Cn1cnc(-c2ccc(O[C@@H]3CCNC[C@H]3F)c(C(N)=O)c2)c1] | MS m/z 319 [M + H]⁺ (3R,4R)-tert-butyl 4-(2-carbamoyl- 4-(1-methyl-1H-imidazol-4- yl)phenoxy)-3-fluoropiperidine-1- carboxylate (Preparation 143). |
| 69 | 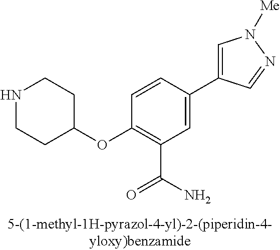 [SMILES: Cn1cc(-c2ccc(OC3CCNCC3)c(C(N)=O)c2)cn1] | MS m/z 301 [M + H]⁺ tert-butyl 4-(2-carbamoyl-4-(1- methyl-1H-pyrazol-4- yl)phenoxy)piperidine-1- carboxylate (Preparation 144). |
| 70 | 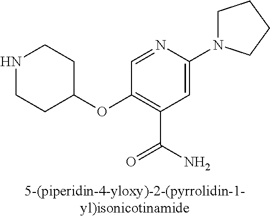 [SMILES: NC(=O)c1cc(N2CCCC2)ncc1OC1CCNCC1] | MS m/z 291 [M + H]⁺ tert-butyl 4-((4-carbamoyl-6- (pyrrolidin-1-yl)pyridin-3- yl)oxy)piperidine-1-carboxylate (Preparation 77). |
| 71 | 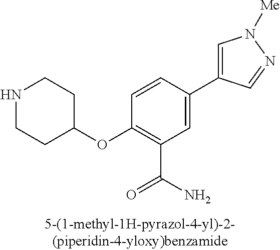 [SMILES: Cn1cc(-c2ccc(OC3CCNCC3)c(C(N)=O)c2)cn1] | MS m/z 301 [M + H]⁺ Tert-butyl 4-[2-carbamoyl-4-(1- methyl-1H-pyrazol-4- yl)phenoxy]piperidine-1- carboxylate (Preparation 136). |
| 72 | 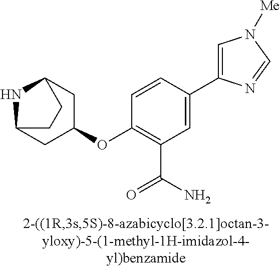 [SMILES: Cn1cnc(-c2ccc(O[C@@H]3C[C@H]4CC[C@@H](C3)N4)c(C(N)=O)c2)c1] | MS m/z 327 [M + H]⁺ (1R,3s,5S)-tert-butyl 3-(2- carbamoyl-4-(1-methyl-1H- imidazol-4-yl)phenoxy)-8- azabicyclo[3.2.1]octane-8- carboxylate (Preparation 140). |

### Preparation 73

### 2-((1R,5S,8r)-3-azabicyclo[3.2.1]octan-8-yloxy)-5-(1-methyl-1H-imidazol-4-yl)benzamide

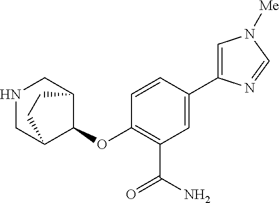 [SMILES: Cn1cnc(-c2ccc(O[C@H]3[C@@H]4CC[C@H]3CNC4)c(C(N)=O)c2)c1]

To a solution of 2-(((1R,5S,8r)-3-benzyl-3-azabicyclo[3.2.1]octan-8-yl)oxy)-5-(1-methyl-1H-imidazol-4-yl)benzamide (Preparation 145, 170 mg, 0.32 mmol) in acetic acid (10 mL) was added palladium on charcoal (20 wt %, 100 mg) and the reaction was hydrogenated under a balloon of hydrogen at room temperature for 16 hours. The reaction was filtered through celite, washing through with MeOH. The filtrate was concentrated in vacuo and partitioned between saturated aqueous sodium bicarbonate solution and 20% IPA in DCM. The organic layer was collected, dried over sodium sulphate and concentrated in vacuo. The residue was washed with ether to afford the title compound (100 mg, 94%).

¹H NMR (400 MHz, DMSO-d₆): δ ppm 1.70-1.87 (m, 4H), 2.19 (m, 2H), 2.38 (m, 2H), 3.02 (m, 2H), 3.66 (s, 3H), 4.68 (m, 1H), 7.15 (m, 1H), 7.49-7.77 (m, 5H), 8.14 (m, 1H).

MS m/z 327 [M+H]⁺

The following Preparations were prepared according to the methods described by Preparation 73 using the appropriate benzyl protected piperidine as described below:

| Preparation | Name/Structure | Data and starting materials |
|---|---|---|
| 74 | 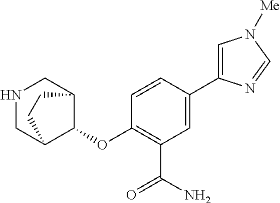 [SMILES: Cn1cnc(-c2ccc(O[C@@H]3[C@@H]4CC[C@H]3CNC4)c(C(N)=O)c2)c1] | ¹H NMR (400 MHz, DMSO-d₆): δ ppm 1.56-1.68 (m, 4H), 2.25-2.72 (m, 4H), 3.66 (s, 3H), 4.21 (m, 1H), 4.49 (m, 1H), 4.62 (m, 1H), 7.22 (m, 1H), 7.49-7.63 (m, 3H), 7.76 (m, 1H), 8.19 (m, 1H). 2-(((1R,5S,8s)-3-benzyl-3- azabicyclo[3.2.1]octan-8-yl)oxy)-5- (1-methyl-1H-imidazol-4- yl)benzamide (Preparation 146). |
| | 2-((1R,5S,8s)-3- | |
| | azabicyclo[3.2.1]octan-8-yloxy)-5-(1- | |
| | methyl-1H-imidazol-4-yl)benzamide | |
| 75 | 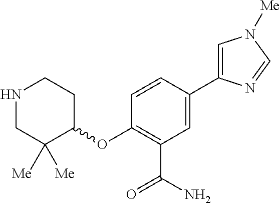 [SMILES: Cn1cnc(-c2ccc(OC3CCNCC3(C)C)c(C(N)=O)c2)c1] | MS m/z 329 [M + H]⁺ Racemic 2-((1-benzyl-3,3- dimethylpiperidin-4-yl)oxy)-5-(1- methyl-1H-imidazol-4-yl)benzamide (Preparation 147). |
| | Racemic 2-((3,3-dimethylpiperidin-4 | |
| | yl)oxy)-5-(1-methyl-1H-imidazol-4- | |
| | yl)benzamide | |

### Preparation 76

### 5-[1-(1,1-dioxidothietan-3-yl)-1H-pyrazol-4-yl]-2-(piperidin-4-yloxy)benzamide trifluoroacetate

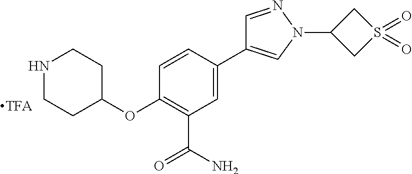 [SMILES: NC(=O)c1cc(-c2cnn(C3CS(=O)(=O)C3)c2)ccc1OC1CCNCC1]

Tert-butyl 4-(2-carbamoyl-4-(1-(1,1-dioxidothietan-3-yl)-1H-pyrazol-4-yl)phenoxy)piperidine-1-carboxylate (Preparation 148, 72 mg, 0.15 mmol) was dissolved in TFA (1.5 mL) and the reaction stirred at room temperature for 1 hour. The reaction was concentrated in vacuo azeotroping with MeOH to afford the title compound as the trifluoroacetate salt (83 mg, quant.). ¹H NMR (400 MHz, DMSO-d₆): δ ppm 1.88-2.00 (m, 2H), 2.04-2.16 (m, 2H), 3.06-3.16 (m, 2H), 3.22-3.34 (m, 2H), 4.68 (dd, 2H), 4.76-4.86 (m, 3H), 5.30-5.38 (m, 1H), 7.20 (d, 1H), 7.54 (br s, 1H), 7.62 (d, 1H), 7.80 (s, 1H), 8.02 (s, 1H), 8.38 (s, 1H) and 8.48 (br s, 2H).

MS m/z 391 [M+H]⁺

### Preparation 77

### tert-butyl 4-((4-carbamoyl-6-(pyrrolidin-1-yl)pyridin-3-yl)oxy)piperidine-1-carboxylate

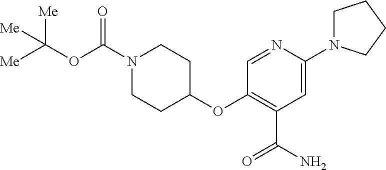 [SMILES: CC(C)(C)OC(=O)N1CCC(Oc2cnc(N3CCCC3)cc2C(N)=O)CC1]

Pyrrolidine (1.5 mL) and tert-butyl 4-((6-bromo-4-carbamoylpyridin-3-yl)oxy)piperidine-1-carboxylate (Preparation 130) were combined in a sealed tube and heated to 100° C. for 6 hours. The reaction was cooled, diluted with EtOAc, washed with water, brine, dried over sodium sulphate and concentrated in vacuo. The residue was purified using silica gel column chromatography eluting with 2% MeOH in DCM to afford the title compound.

¹H NMR (400 MHz, DMSO-d₆): δ ppm 1.39 (s, 9H), 1.51-1.56 (m, 2H), 1.83 (m, 2H), 1.92 (m, 4H), 3.11 (m, 2H), 3.31 (m, 4H), 3.62 (m, 2H), 4.34 (m, 1H), 6.57 (s, 1H), 7.67 (m, 2H), 8.01 (s, 1H).

### Preparation 78

### tert-butyl(3S,4R)-4-{[3-carbamoyl-5-(1-methyl-1H-imidazol-4-yl)pyridin-2-yl]oxy}-3-fluoropiperidine-1-carboxylate

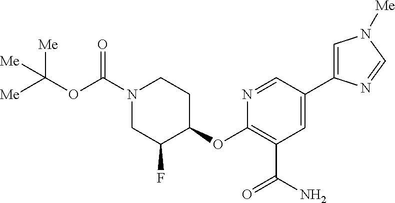 [SMILES: Cn1cnc(-c2cnc(O[C@@H]3CCN(C(=O)OC(C)(C)C)C[C@@H]3F)c(C(N)=O)c2)c1]

To a solution of tert-butyl (3S,4R)-4-{[3-carbamoyl-5-(4,4,5,5-tetramethyl-1,3,2-dioxaborolan-2-yl)pyridin-2-yl]oxy}-3-fluoropiperidine-1-carboxylate (Preparation 99, 900 mg, 1.94 mmol) and 4-iodo-1-methyl-1H-imidazole (403 mg, 1.94 mmol) in DMF (15 mL) was added a solution of potassium carbonate (534 mg, 3.87 mmol) in water (2 mL) and the mixture degassed with argon for 15 minutes. 1,1-bis(diphenylphosphino)ferrocene palladium (II) dichloride (79 mg, 0.097 mmol) was added and the reaction heated to 100° C. for 16 hours. The reaction was cooled, diluted with EtOAc, washed with water, brine, dried over sodium sulphate and concentrated in vacuo. The residue was purified using silica gel column chromatography eluting with 3-5% MeOH in DCM to afford the title compound (400 mg, 49%).

¹H NMR (400 MHz, DMSO-d₆): δ ppm 1.41 (s, 9H), 1.81-1.86 (m, 1H), 1.96-1.99 (m, 1H), 2.95-3.30 (m, 2H), 3.68 (s, 3H), 3.98-4.03 (m, 1H), 4.10-4.21 (m, 1H), 5.00 (d, 1H), 5.42-5.50 (m, 1H), 7.53 (br s, 1H), 7.67 (s, 1H), 7.69 (s, 1H), 7.82 (br s, 1H), 8.54 (d, 1H), 8.63 (d, 1H).

MS m/z 420 [M+H]⁺

### Preparation 79

### tert-butyl(3R,4S)-4-({3-carbamoyl-5-[1-(1,1-dioxidothietan-3-yl)-1H-pyrazol-4-yl]pyridin-2-yl}oxy)-3-fluoropiperidine-1-carboxylate

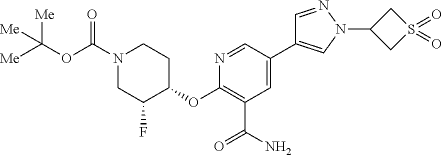 [SMILES: CC(C)(C)OC(=O)N1CC[C@H](Oc2ncc(-c3cnn(C4CS(=O)(=O)C4)c3)cc2C(N)=O)[C@H](F)C1]

To a solution of tert-butyl (3R,4S)-4-[(5-bromo-3-carbamoylpyridin-2-yl)oxy]-3-fluoropiperidine-1-carboxylate (Preparation 119, 300 mg, 0.717 mmol) and 1-(1,1-dioxidothietan-3-yl)-4-(4,4,5,5-tetramethyl-1,3,2-dioxaborolan-2-yl)-1H-pyrazole (Preparation 168, 278 mg, 0.932 mmol) in dioxane (12 mL) at ambient temperature was added Cs₂CO₃ (584 mg, 1.793 mmol) in water (3 mL). The reaction was degassed with argon for 15 minutes followed by the addition of Pd₂(dba)₃ (32 mg, 0.036 mmol) and t-Bu₃PHBF₄ (41 mg, 0.143 mmol). The resulting solution was heated at 110° C. for 16 hours. The mixture was diluted with EtOAc and washed with water, brine, dried over sodium sulphate and concentrated in vacuo. The residue was purified using silica gel column chromatography eluting with 5% MeOH in DCM to afford the title compound (310 mg, 85%). Taken on directly to the next step.

MS m/z 510 [M+H]⁺

### Preparation 80

### tert-butyl(3S,4R)-4-[(3-carbamoyl-5-{1-[(methylsulfonyl)methyl]-1H-pyrazol-4-yl}pyridin-2-yl)oxy]-3-fluoropiperidine-1-carboxylate

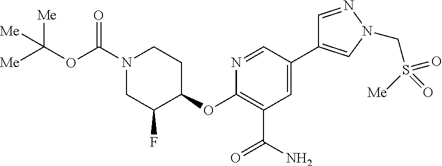 [SMILES: CC(C)(C)OC(=O)N1CC[C@@H](Oc2ncc(-c3cnn(CS(C)(=O)=O)c3)cc2C(N)=O)[C@@H](F)C1]

To a solution of tert-butyl (3S,4R)-4-{[3-carbamoyl-5-(4,4,5,5-tetramethyl-1,3,2-dioxaborolan-2-yl)pyridin-2-yl]oxy}-3-fluoropiperidine-1-carboxylate (Preparation 99, 1.50 g, 3.22 mmol) and 4-bromo-1-((methanesulfonyl)methyl)-1Hpyrazole (Preparation 170, 771 mg, 3.22 mmol) in DMF was added a solution of potassium carbonate (890 mg, 6.44 mmol) in water (2 mL). The mixture was degassed with argon for 15 minutes before the addition of 1,1-bis(diphenylphosphino)ferrocene palladium (II) dichloride (132 mg, 0.16 mmol) and heating to 100° C. for 16 hours. The reaction was cooled, diluted with EtOAc (100 mL), washed with water (2×30 mL), brine (20 mL), dried over sodium sulphate and concentrated in vacuo. The residue was purified using silica gel column chromatography eluting with 5% MeOH in DCM to afford the title compound (800 mg, 50%).

¹H NMR (400 MHz, DMSO-d₆): δ ppm 1.41 (s, 9H), 1.84-1.99 (m, 2H), 2.91-3.32 (m, 5H), 3.90-3.97 (m, 1H), 4.15-4.20 (m, 1H), 4.98-5.11 (d, 1H), 5.40-5.50 (m, 1H), 5.75 (s, 2H), 7.55 (br s, 1H), 7.86 (br s, 1H), 8.18 (s, 1H), 8.40 (s, 2H), 8.59 (d, 1H).

MS m/z 498 [M+H]⁺

The following Preparations were prepared according to Preparations 78, 79 or 80 using the appropriate boronic ester and halide and Purification Method (PM) below if different from the methods described. Sodium, cesium or potassium carbonate may be used as base.

| Preparation | Name/Structure | Data and starting materials |
|---|---|---|
| 81 | 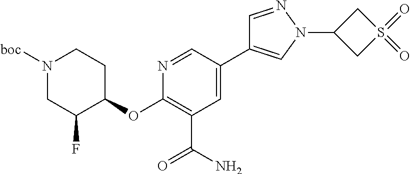 | MS m/z 510 [M + H]⁺ Cis-racemic tert-butyl-4-[(5-bromo- 3-carbamoylpyridin-2-yl)oxy]-3- fluoropiperidine-1-carboxylate (Preparation 121) and 1-(1,1- dioxidothietan-3-yl)-4-(4,4,5,5- tetramethyl-1,3,2-dioxaborolan-2- yl)-1H-pyrazole (Preparation 168). |
| | 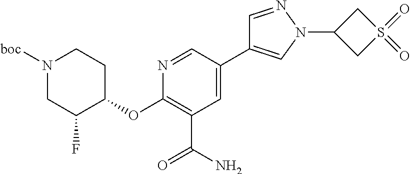 | |
| | Cis-racemic tert-butyl-4-({3- | |
| | carbamoyl-5-[1-(1,1-dioxidothietan- | |
| | 3-yl)-1H-pyrazol-4-yl]pyridin-2- | |
| | yl}oxy)-3-fluoropiperidine-1- | |
| | carboxylate | |
| 82 | 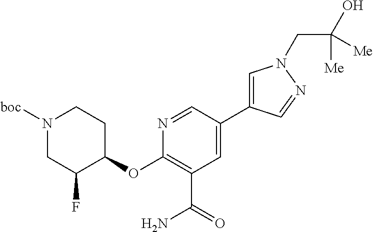 | MS m/z 478 [M + H]⁺ tert-butyl (3S,4R)-4-[(5-bromo-3- carbamoylpyridin-2-yl)oxy]-3- fluoropiperidine-1-carboxylate (Preparation 118) and 2-methyl-1- [3-(4,4,5,5-tetramethyl-1,3,2- dioxaborolan-2-yl)-1H-pyrazol-1- yl]propan-2-ol (Preparation 214). |
| | tert-butyl (3S,4R)-4-({3-carbamoyl-5- | |
| | [1-(2-hydroxy-2-methylpropyl)-1H- | |
| | pyrazol-4-yl]pyridin-2-yl}oxy)-3- | |
| | fluoropiperidine-1-carboxylate | |
| 83 | 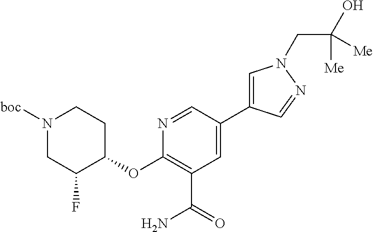 | MS m/z 478 [M + H]⁺ tert-butyl (3R,4S)-4-[(5-bromo-3- carbamoylpyridin-2-yl)oxy]-3- fluoropiperidine-1-carboxylate (Preparation 119) and 2-methyl-1- [3-(4,4,5,5-tetramethyl-1,3,2- dioxaborolan-2-yl)-1H-pyrazol-1- yl]propan-2-ol (Preparation 214). |
| | tert-butyl (3R,4S)-4-({3-carbamoyl-5- | |
| | [1-(2-hydroxy-2-methylpropyl)-1H- | |
| | pyrazol-4-yl]pyridin-2-yl}oxy)-3- | |
| | fluoropiperidine-1-carboxylate | |
| 84 | 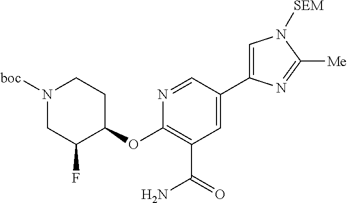 | MS m/z 550 [M + H]⁺ 4-Bromo-2-methyl-1-((2- (trimethylsilyl)ethoxy)methyl)-1H- imidazole (Preparation 176) and tert-butyl (3S,4R)-4-{[3-carbamoyl- 5-(4,4,5,5-tetramethyl-1,3,2- dioxaborolan-2-yl)pyridin-2-yl]oxy}- 3-fluoropiperidine-1-carboxylate (Preparation 99). |
| | tert-butyl (3S,4R)-4-{[3-carbamoyl-5- | |
| | (2-methyl-1-{[2- | |
| | (trimethylsilyl)ethoxy]methyl}-1H- | |
| | imidazol-4-yl)pyridin-2-yl]oxy}-3- | |
| | fluoropiperidine-1-carboxylate | |
| 85 | 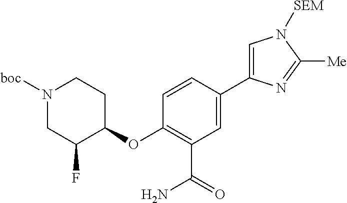 | MS m/z 549 [M + H]⁺ tert-butyl (3S,4R)-4-[2-carbamoyl- 4-(4,4,5,5-tetramethyl-1,3,2- dioxaborolan-2-yl)phenoxy]-3- fluoropiperidine-1-carboxylate (Preparation 101) and 4-Bromo-2- methyl-1-((2- (trimethylsilyl)ethoxy)methyl)-1H- imidazole (Preparation 176). |
| | tert-butyl (3S,4R)-4-[2-carbamoyl-4- | |
| | (2-methyl-1-{[2- | |
| | (trimethylsilyl)ethoxy]methyl}-1H- | |
| | imidazol-4-yl)phenoxy]-3- | |
| | fluoropiperidine-1-carboxylate | |
| 86 | 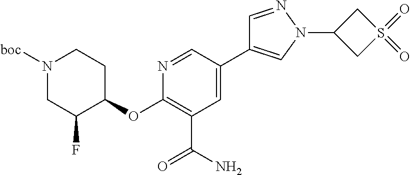 | Taken on directly to the next step, tert-butyl (3S,4R)-4-[(5-bromo-3- carbamoylpyridin-2-yl)oxy]-3- fluoropiperidine-1-carboxylate (Preparation 118) and 1-(1,1- dioxidothietan-3-yl)-4-(4,4,5,5- tetramethyl-1,3,2-dioxaborolan-2- yl)-1H-pyrazole (Preparation 168). |
| | tert-butyl (3S,4R)-4-({3-carbamoyl-5- | |
| | [1-(1,1-dioxidothietan-3-yl)-1H- | |
| | pyrazol-4-yl]pyridin-2-yl}oxy)-3- | |
| | fluoropiperidine-1-carboxylate | |
| 87 | 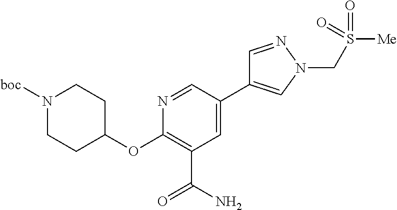 | MS m/z 480 [M + H]⁺ ¹H NMR (400 MHz, DMSO-d₆): δ ppm 1.41 (s, 9H), 1.67-1.73 (m, 2H), 1.96-1.98 (m, 2H), 3.05 (s, 3H), 3.24-3.25 (m, 2H), 3.63-3.67 (m, 1H), 5.36 (m, 1H), 5.75 (s, 2H), 7.59 (br s, 1H), 7.74 (br s, 1H), 8.16 (s, 1H), 8.32 (d, 1H), 8.36 (s, 1H), 8.57 (d, 1H). tert-butyl 4-{[3-carbamoyl-5- (4,4,5,5-tetramethyl-1,3,2- dioxaborolan-2-yl)pyridin-2- yl]oxy}piperidine-1-carboxylate |
| | tert-butyl 4-[(3-carbamoyl-5-{1- | (Preparation 100) and 4-bromo-1- |
| | [(methylsulfonyl)methyl]-1H-pyrazol- | ((methanesulfonyl)methyl)-1H- |
| | 4-yl}pyridin-2-yl)oxy]piperidine-1- | pyrazole (Preparation 170). |
| | carboxylate | |
| 88 | 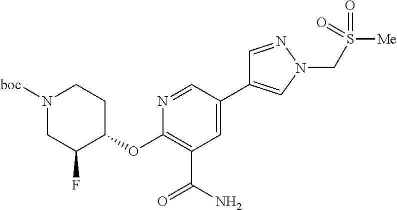 | MS m/z 498 [M + H]⁺ (3S,4S)-tert-butyl 4-((3-carbamoyl- 5-(4,4,5,5-tetramethyl-1,3,2- dioxaborolan-2-yl)pyridin-2-yl)oxy)- 3-fluoropiperidine-1-carboxylate (Preparation 102) and 4-Bromo-1- ((methanesulfonyl)methyl)-1H- pyrazole (Preparation 170). |
| | tert-butyl (3S,4S)-4-[(3-carbamoyl-5- | |
| | {1-[(methylsulfonyl)methyl]-1H- | |
| | pyrazol-4-yl}pyridin-2-yl)oxy]-3- | |
| | fluoropiperidine-1-carboxylate | |
| 89 | 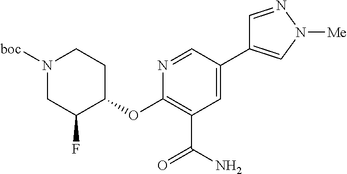 | MS m/z 420 [M + H]⁺ (3S,4S)-tert-butyl 4-((3-carbamoyl- 5-(4,4,5,5-tetramethyl-1,3,2- dioxaborolan-2-yl)pyridin-2-yl)oxy)- 3-fluoropiperidine-1-carboxylate (Preparation 102) and 4-iodo-1- methyl-1H-imidazole. |
| | tert-butyl (3S,4S)-4-{[3-carbamoyl-5- | |
| | (1-methyl-1H-imidazol-4-yl)pyridin-2- | |
| | yl]oxy}-3-fluoropiperidine-1- | |
| | carboxylate | |
| 90 | 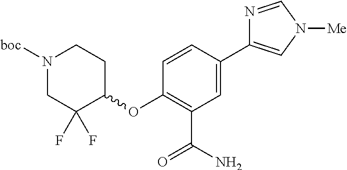 | MS m/z 437 [M + H]⁺ ¹H NMR (400 MHz, CDCl₃): δ ppm 1.45 (s, 9H), 2.00-2.10 (m, 2H), 3.35 (m, 1H), 3.58 (m, 1H), 3.75 (s, 3H), 3.80 (m, 1H), 4.05-4.15 (m, 2H), 4.70 (m, 1H), 5.75 (br s, 1H), 7.02 (m, 1H), 7.25 (m, 1H), 7.45 (m, 2H), 8.05 (m, 1H), 8.38 (s, 1H). Racemic tert-butyl 4-(2-carbamoyl- 4-(4,4,5,5-tetram ethyl-1,3,2- |
| | Racemic tert-butyl 4-(2-carbamoyl-4- | dioxaborolan-2-yl)phenoxy)-3,3- |
| | (1-methyl-1H-imidazol-4- | difluoropiperidine-1-carboxylate |
| | yl)phenoxy)-3,3-difluoropiperidine-1- | (Preparation 103) and 4-bromo-1- |
| | carboxylate | methyl-1H-imidazole. |

### Preparation 91

### tert-butyl(3S,4R)-4-[(4-carbamoyl-6-{1-[(methylsulfonyl)methyl]-1H-pyrazol-4-yl}pyridin-3-yl)oxy]-3-fluoropiperidine-1-carboxylate

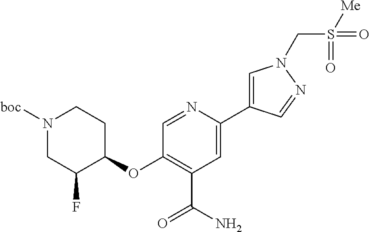

The title compound may be prepared according to the methods described for Preparations 99 and 80 using tert-butyl (3S,4R)-4-[(6-bromo-4-carbamoylpyridin-3-yl)oxy]-3-fluoropiperidine-1-carboxylate (Preparation 125) and 4-bromo-1-((methanesulfonyl)methyl)-1Hpyrazole (Preparation 170).

¹H NMR (400 MHz, DMSO-d₆): δ ppm 1.40 (s, 9H), 1.79 (m, 1H), 1.98 (m, 1H), 3.02 (m, 4H), 3.93-4.12 (m, 3H), 4.88-5.08 (m, 2H), 5.77 (m, 2H), 7.68 (br s, 1H), 7.88 (s, 2H), 8.20 (s, 1H), 8.41 (s, 1H), 8.59 (s, 1H).

MS m/z 498 [M+H]⁺

### Preparation 92

### tert-butyl(3S,4S)-4-[2-carbamoyl-4-(1-methyl-1H-imidazol-4-yl)phenoxy]-3-fluoropiperidine-1-carboxylate

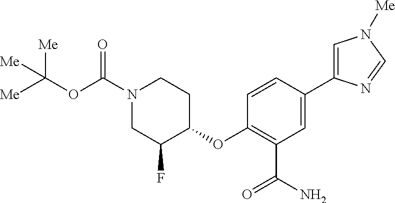 [SMILES: Cn1cnc(-c2ccc(O[C@H]3CCN(C(=O)OC(C)(C)C)C[C@@H]3F)c(C(N)=O)c2)c1]

To a solution of 2-hydroxy-5-(1-methyl-1H-imidazol-4-yl)benzamide (Preparation 160, 296 mg, 1.364 mmol) in DMF (5 mL) was added cesium carbonate (1.33 g, 4.09 mmol) followed by (3S,4R)-3-fluoro-4-methanesulfonyloxypiperidine-1-carboxylic acid tert-butyl ester (Preparation 157, 486 mg, 1.64 mmol). The reaction was heated to 110° C. for 16 hours in a sealed tube. The reaction was cooled and poured onto ice/water, extracting with EtOAc (2×30 mL). The combined organic layers were washed with water (2×15 mL), brine (15 mL), dried over sodium sulphate and concentrated in vacuo. The residue was purified using silica gel column chromatography eluting with 10% EtOAc in heptanes to afford the title compound with inversion of stereochemistry at the 4-position (280 mg, 49%).

MS m/z 419 [M+H]⁺

The following Preparations were prepared according to Preparation 92 using the appropriate phenol and mesylate as described below:

| Preparation | Name/Structure | Data and starting materials |
|---|---|---|
| 93 | 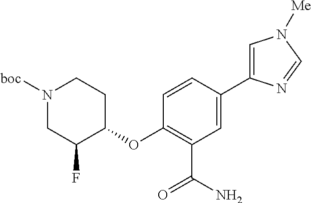 | MS m/z 419 [M + H]⁺ trans-racemic tert-butyl 3-fluoro-4- ((methylsulfonyl)oxy)piperidine-1- carboxylate (Preparation 178) and 2-hydroxy-5-(1-methyl-1H-imidazol- 4-yl)benzamide (Preparation 160). |
| | 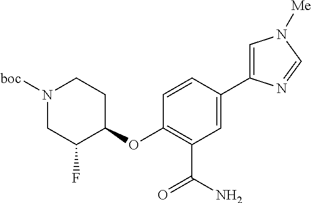 | |
| | Trans-racemic-tert-butyl-4-[2- | |
| | carbamoyl-4-(1-methyl-1H-imidazol- | |
| | 4-yl)phenoxy]-3-fluoropiperidine-1- | |
| | carboxylate | |
| 94 | 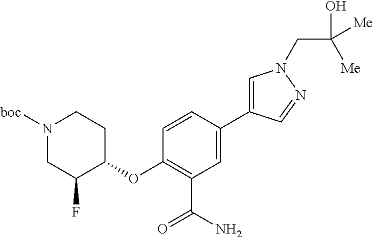 | MS m/z 477 [M + H]⁺ ¹H NMR (400 MHz, DMSO-d₆): δ ppm 1.07 (s, 6H), 1.41 (s, 9H), 1.71 (m, 1H), 1.98 (m, 1H), 3.51 (m, 1H), 3.83-4.01 (m, 4H), 4.69-4.84 (m, 4H), 7.25 (m, 1H), 7.54-7.63 (m, 3H), 7.82 (m, 2H), 8.05 (s, 1H). 2-hydroxy-5-(1-(2-hydroxy-2- methylpropyl)-1H-pyrazol-4- yl)benzamide (Preparation 166) and cis-racemic tert-butyl 3-fluoro- 4-((methylsulfonyl)oxy)piperidine-1- carboxylate (Preparation 178). |
| | 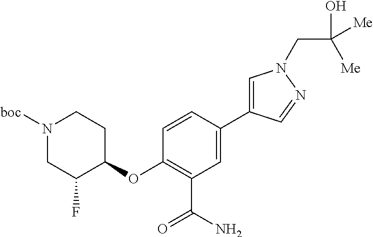 | |
| | T rans-racemic-tert-butyl 4-(2- | |
| | carbamoyl-4-(1-(2-hydroxy-2- | |
| | methylpropyl)-1H-pyrazol-4- | |
| | yl)phenoxy)-3-fluoropiperidine-1 | |
| | carboxylate | |
| 95 | 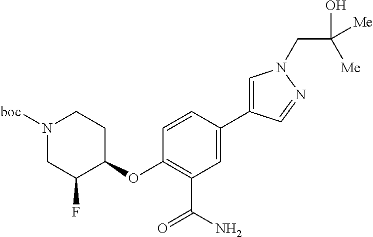 | MS m/z 477 [M + H]⁺ 2-hydroxy-5-(1-(2-hydroxy-2- methylpropyl)-1H-pyrazol-4- yl)benzamide (Preparation 166) and cis-racemic tert-butyl 3-fluoro- 4-((methylsulfonyl)oxy)piperidine-1- carboxylate (Preparation 180). |
| | 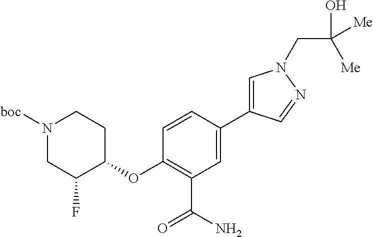 | |
| | Cis-racemic-tert-butyl 4-(2- | |
| | carbamoyl-4-(1-(2-hydroxy-2- | |
| | methylpropyl)-1H-pyrazol-4- | |
| | yl)phenoxy)-3-fluoropiperidine-1- | |
| | carboxylate | |

### Preparation 96

### (3S,4R)-4-[2-carbamoyl-4-(1-methyl-1H-imidazol-4-yl)phenoxy]-3-fluoropiperidine-1-carboxylate

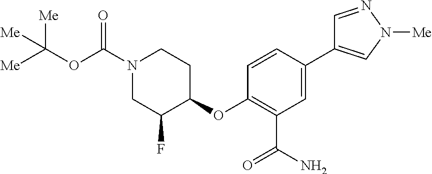 [SMILES: Cn1cc(-c2ccc(O[C@@H]3CCN(C(=O)OC(C)(C)C)C[C@@H]3F)c(C(N)=O)c2)cn1]

To a solution of tert-butyl (3S,4R)-4-[2-cyano-4-(1-methyl-1H-imidazol-4-yl)phenoxy]-3-fluoropiperidine-1-carboxylate (Preparation 97, 8.61 g, 21.5 mmol) and potassium carbonate (5.93 g, 43.0 mmol) in DMSO (80 mL) was added hydrogen peroxide (7.3 mL, 107.5 mmol) and the reaction was stirred at room temperature for 6 hours. Further hydrogen peroxide (29 mL, 430 mmol) and potassium carbonate (2.96 g, 21.5 mmol) were added and the reaction stirred for a further 48 hours. The reaction was diluted with water (500 mL) and extracted with EtOAc (3×400 mL). The organic extracts were combined, washed with brine (3×300 mL), dried over sodium sulphate and concentrated in vacuo to afford the title compound (8.51 g, 95%).

¹H NMR (400 MHz, DMSO-d₆): δ ppm 1.39 (s, 9H), 1.74 (m, 1H), 1.92 (m, 1H), 2.98 (br m, 1H), 3.17 (br m, 1H), 3.65 (s, 3H), 3.93 (m, 1H), 4.17 (m, 1H), 4.84 (m, 1H), 5.00 (d, 1H), 7.25 (d, 1H), 7.54-7.60 (m, 4H), 7.79 (m, 1H), 8.21 (s, 1H).

MS m/z 419 [M+H]⁺

### Preparation 97

### tert-butyl(3S,4R)-4-[2-cyano-4-(1-methyl-1H-imidazol-4-yl)phenoxy]-3-fluoropiperidine-1-carboxylate

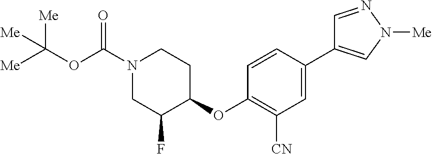 [SMILES: Cn1cc(-c2ccc(O[C@@H]3CCN(C(=O)OC(C)(C)C)C[C@@H]3F)c(C#N)c2)cn1]

A solution of tert-butyl (3S,4R)-4-[4-bromo-2-cyano-phenoxy]-3-fluoropiperidine-1-carboxylate (Preparation 120, 16.2 g, 40.6 mmol) bispinacolatodiboron (15.5 g, 60.9 mmol), potassium acetate (7.95 g, 81.2 mmol) in dioxane (350 mL) was degassed with nitrogen for 30 minutes before the addition of 1,1-bis(diphenylphosphino)ferrocene palladium (II) dichloride (665 mg, 0.810 mmol). The reaction was heated to 100° C. for 3 hours before cooling and further degassing with nitrogen. Potassium carbonate (2M aqueous solution, 150 mL) followed by 4-iodo-1-methyl-1H-imidazole (8.50 g, 40.9 mmol) was added and the reaction heated to 100° C. for 5 days. The reaction was cooled, diluted with water (150 mL) and extracted with EtOAc (2×300 mL). The organic extracts were combined and extracted into 1M H₂SO₄ (2×300 mL). The acidic extracts were basified to pH=7 using solid potassium carbonate and extracted into DCM (3×300 mL). The organic extracts were combined and concentrated in vacuo. The residue was purified using silica gel column chromatography eluting with 5% IPA in EtOAc to afford the title compound as an oil (8.61 g, 27%).

¹H NMR (400 MHz, DMSO-d₆): δ ppm 1.40 (s, 9H), 1.80-1.89 (m, 2H), 3.07 (m, 1H), 3.34 (m, 1H), 3.65 (s, 3H), 3.82 (m, 1H), 4.04 (m, 1H), 4.82-5.01 (m, 2H), 7.39 (d, 1H), 7.61-7.65 (m, 2H), 7.97-8.02 (m, 2H).

MS m/z 401 [M+H]⁺

### Preparation 98

### tert-butyl(3S,4S)-4-{2-carbamoyl-4-[1-(2-hydroxy-2-methylpropyl)-1H-pyrazol-4-yl]phenoxy}-3-fluoropiperidine-1-carboxylate

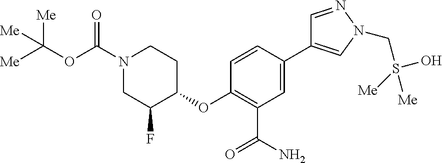 [SMILES: CC(C)(C)OC(=O)N1CC[C@H](Oc2ccc(-c3cnn(CS(C)(C)O)c3)cc2C(N)=O)[C@@H](F)C1]

The title compound was prepared according to the method described for Preparation 97 using tert-butyl (3S,4S)-4-(4-bromo-2-carbamoylphenoxy)-3-fluoropiperidine-1-carboxylate (Preparation 132) and 2-methyl-4-[3-(4,4,5,5-tetramethyl-1,3,2-dioxaborolan-2-yl)-1H-pyrazol-1-yl]propan-2-ol (Preparation 214).

¹H NMR (400 MHz, DMSO-d₆): δ ppm 1.08 (s, 6H), 1.41 (s, 9H), 1.71 (m, 1H), 2.08 (m, 1H), 3.51 (m, 3H), 3.82 (m, 1H), 4.01 (s, 2H), 4.72-4.84 (m, 3H), 7.26 (d, 1H), 7.54 (br s, 2H), 7.63 (m, 1H), 7.82 (m, 2H), 8.05 (s, 1H).

MS m/z 477 [M+H]⁺

### Preparation 99

### tert-butyl(3S,4R)-4-{[3-carbamoyl-5-(4,4,5,5-tetramethyl-1,3,2-dioxaborolan-2-yl)pyridin-2-yl]oxy}-3-fluoropiperidine-1-carboxylate

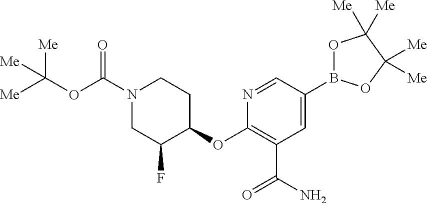 [SMILES: CC(C)(C)OC(=O)N1CC[C@@H](Oc2ncc(B3OC(C)(C)C(C)(C)O3)cc2C(N)=O)[C@@H](F)C1]

A suspension of tert-butyl (3S,4R)-4-[(5-bromo-3-carbamoylpyridin-2-yl)oxy]-3-fluoropiperidine-1-carboxylate (Preparation 118, 2.5 g, 5.98 mmol), bispinacolatodiboron (1.67 g, 6.57 mmol) and potassium acetate (1.76 g, 17.94 mmol) in dioxane (60 mL) was degassed with argon for 15 minutes. 1,1-bis(diphenylphosphino)ferrocene palladium (II) dichloride (244 mg, 0.299 mmol) was added and the reaction heated to 100° C. for 14 hours. The reaction was cooled, diluted with EtOAc and filtered through celite, washing through with EtOAc. The filtrate was concentrated in vacuo and triturated with heptanes to afford the title compound (2.40 g, 86%).

¹H NMR (400 MHz, DMSO-d₆): δ ppm 1.30 (s, 12H), 1.40 (s, 9H), 1.80-1.86 (m, 1H), 1.94-1.99 (m, 1H), 2.94-3.33 (m, 2H), 3.90-4.03 (m, 1H), 4.13-4.18 (m, 1H), 4.96-5.09 (d, 1H), 5.47-5.56 (dd, 1H), 7.47 (br s, 1H), 7.83 (br s, 1H), 8.44 (s, 1H), 8.46 (s, 1H).

The following Preparations were prepared according to Preparation 99 using the appropriate aryl halide as described below:

| Preparation | Name/Structure | Data and starting materials |
|---|---|---|
| 100 | 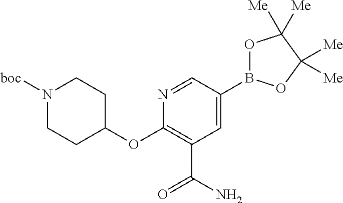 | ¹H NMR (400 MHz, DMSO-d₆): δ ppm 1.30-1.40 (m, 23H), 1.65-1.74 (m, 2H), 1.90-1.98 (m, 2H), 3.23- 3.32 (m, 2H), 3.62-3.65 (m, 2H), 5.42 (m, 1H), 7.51 (br s, 1H), 7.70 (br s, 1H), 8.36 (d, 1H), 8.45 (d, 1H). tert-butyl 4-[(5-bromo-3- carbamoylpyridin-2- yl)oxy]piperidine-1-carboxylate (Preparation 149). |
| | tert-butyl 4-{[3-carbamoyl-5-(4,4,5,5- | |
| | tetramethyl-1,3,2-dioxaborolan-2- | |
| | yl)pyridin-2-yl]oxy}piperidine-1- | |
| | carboxylate | |
| 101 | 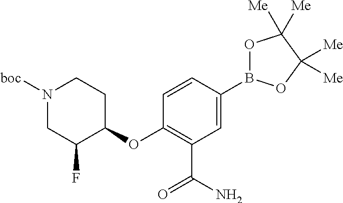 | ¹H NMR (400 MHz, DMSO-d₆): δ ppm 1.29 (s, 12H), 1.40 (s, 9H), 1.74 (m, 1H), 1.94 (m, 1H), 2.98 (m, 1H), 3.21 (m, 1H), 3.94 (m, 1H), 4.21 (m, 1H), 4.94-5.07 (m, 2H), 7.30 (m, 1H), 7.49 (br s, 1H), 7.55 (br s, 1H), 7.72 (m, 1H), 8.22 (s, 1H). tert-butyl (3S,4R)-4-[4-bromo-2- carbamoyl-phenoxy]-3- fluoropiperidine-1-carboxylate (Preparation 134). |
| | tert-butyl (3S,4R)-4-[2-carbamoyl-4- | |
| | (4,4,5,5-tetramethyl-1,3,2- | |
| | dioxaborolan-2-yl)phenoxy]-3- | |
| | fluoropiperidine-1-carboxylate | |
| 102 | 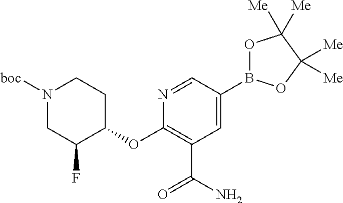 | ¹H NMR (400 MHz, DMSO-d₆): δ ppm 1.32 (s, 12H), 1.40 (s, 9H), 1.82 (m, 1H), 1.95 (m, 1H), 2.98 (m, 2H), 3.93 (m, 1H), 4.16 (m, 1H), 4.96-5.09 (m, 1H), 5.47-5.56 (m, 1H), 7.47 (br s, 1H), 7.83 (br s, 1H), 8.46 (m, 2H). tert-butyl (3S,4S)-4-[(5-bromo-3- carbamoylpyridin-2-yl)oxy]-3- fluoropiperidine-1-carboxylate (Preparation 133). |
| | (3S,4S)-tert-butyl 4-((3-carbamoyl-5- | |
| | (4,4,5,5-tetramethyl-1,3,2- | |
| | dioxaborolan-2-yl)pyridin-2-yl)oxy)-3- | |
| | fluoropiperidine-1-carboxylate | |
| 103 | 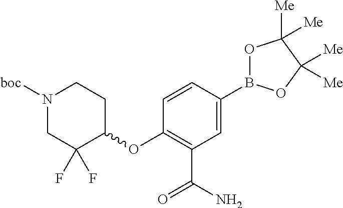 | Taken directly on to the next step. Racemic tert-butyl 4-(4-bromo-2- carbamoylphenoxy)-3,3- difluoropiperidine-1-carboxylate (Preparation 131). |
| | Racemic tert-butyl 4-(2-carbamoyl-4- | |
| | (4,4,5,5-tetramethyl-1,3,2- | |
| | dioxaborolan-2-yl)phenoxy)-3,3- | |
| | difluoropiperidine-1-carboxylate | |

The following Preparations were prepared according to the methods described by Preparation 43 using the appropriate boc protected piperidine and using one of the purification methods below if required. All compounds were isolated as their hydrochloride salts.

Purification Method A: Trituration with ether.

| Preparation | Name/Structure | Data | Starting materials |
|---|---|---|---|
| 104 | 5-bromo-2-{[(3S,4S)-3- | MS m/z 320 [M⁸¹Br + H]⁺ | tert-butyl (3S,4S)-4- |
| | fluoropiperidin-4- | | [(5-bromo-3- |
| | yl]oxy}pyridine-3- | | carbamoylpyridin-2- |
| | carboxamide | | yl)oxy]-3- |
| | 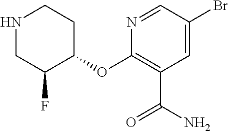 [SMILES: NC(=O)c1cc(Br)cnc1O[C@H]1CCNC[C@@H]1F] | | fluoropiperidine-1- carboxylate (Preparation 133). Purification method A. |
| 105 | 5-bromo-2-{[(3S,4R)-3- | ¹H NMR (400 MHz, | tert-butyl (3S,4R)-4- |
| | fluoropiperidin-4- | DMSO-d₆): δ ppm 2.05- | [(5-bromo-3- |
| | yl]oxy}pyridine-3- | 2.20 (m, 2H), 3.16-3.71 | carbamoylpyridin-2- |
| | carboxamide | (m, 4H), 5.28 (d, 1H), | yl)oxy]-3- |
| | 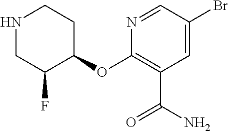 [SMILES: NC(=O)c1cc(Br)cnc1O[C@@H]1CCNC[C@@H]1F] | 5.43-5.52 (m, 1H), 7.56 (br s, 1H), 7.92 (br s, 1H), 8.25 (d, 1H), 8.42 (d, 1H), 8.78 (br s, 1H), 9.35 (br s, 1H). MS m/z 318 [M⁷⁹Br + H]⁺ | fluoropiperidine-1- carboxylate (Preparation 118). |
| 106 | 5-bromo-2-{[(3R,4S)-3- | ¹H NMR (400 MHz, | Tert-butyl (3R,4S)-4- |
| | fluoropiperidin-4- | DMSO-d₆): δ ppm 1.80 | [(5-bromo-3- |
| | yl]oxy}pyridine-3- | (m, 1H), 2.06-2.20 (m, | carbamoylpyridin-2- |
| | carboxamide | 1H), 3.17-3.61 (m, 5H), | yl)oxy]-3- |
| | 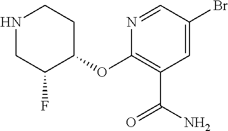 [SMILES: NC(=O)c1cc(Br)cnc1O[C@H]1CCNC[C@H]1F] | 5.22-5.34 (m, 1H), 5.44- 5.53 (m, 1H), 7.56 (br s, 1H), 7.92 (br s, 1H), 8.27 (s, 1H), 8.42 (s, 1H), 8.81 (br s, 1H), 9.38 (br s, 1H). MS m/z 318 [M⁷⁹Br + H]⁺ | fluoropiperidine-1- carboxylate (Preparation 119). |
| 107 | 5-bromo-2-{[(3S,4R)-3- | ¹H NMR (400 MHz, | tert-butyl (3S,4R)-4- |
| | fluoropiperidin-4- | DMSO-d₆): δ ppm 2.00- | [(4-bromo-2- |
| | yl]oxy}benzamide | 2.15 (m, 2H), 3.10 (m, | carbamoyl-phenoxy]- |
| | 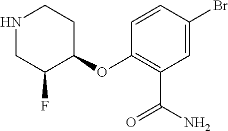 [SMILES: NC(=O)c1cc(Br)ccc1O[C@@H]1CCNC[C@@H]1F] | 1H), 3.25-3.37 (m, 3H), 4.65 (br s, 2H), 4.97-5.02 (m, 1H), 5.18-5.30 (m, 1H), 7.27 (d, 1H), 7.54 (br s, 1H), 7.66 (dd, 1H), 7.77 (br s, 1H), 7.84 (s, 1H). MS m/z 316 [M⁷⁹Br − H]⁻ | 3-fluoropiperidine-1- carboxylate (Preparation 134). Purification Method A. |

### Preparation 117

### tert-butyl 4-[(5-bromo-3-carbamoyl-6-methylpyridin-2-yl)oxy]piperidine-1-carboxylate

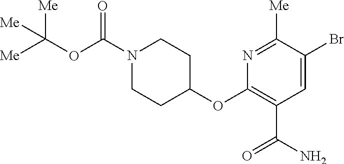 [SMILES: Cc1nc(OC2CCN(C(=O)OC(C)(C)C)CC2)c(C(N)=O)cc1Br]

To a solution of tert-butyl 4-[(5-bromo-3-cyano-6-methylpyridin-2-yl)oxy]piperidine-1-carboxylate (Preparation 123, 214 mg, 0.54 mmol), in t-butyl alcohol (5 mL) was added KOH powder (45 mg, 0.811 mmol) and the reaction was allowed to stir at 80° C. for 16 hours. The reaction mixture was cooled, diluted with ethyl acetate, washed water, brine, dried over sodium sulfate and concentrated in vacuo to afford the title compound as light yellow solid (210 mg, 93%).

¹H NMR (400 MHz, DMSO-d₆): δ ppm 1.40 (s, 9H), 1.67 (m, 2H), 1.93 (m, 2H), 3.24-3.31 (m, 2H), 3.62 (m, 2H), 5.34 (m, 1H), 7.49 (br s, 1H), 7.76 (br s, 1H), 8.20 (s, 1H).

### Preparation 118

### tert-butyl(3S,4R)-4-[(5-bromo-3-carbamoylpyridin-2-yl)oxy]-3-fluoropiperidine-1-carboxylate

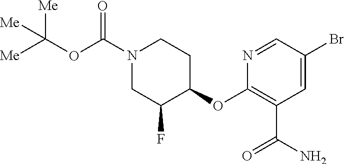 [SMILES: CC(C)(C)OC(=O)N1CC[C@@H](Oc2ncc(Br)cc2C(N)=O)[C@@H](F)C1]

To a solution of tert-butyl-(3S,4R)-3-fluoro-4-hydroxy-piperidine-1-carboxylate (WO 2013/011402 A1), 3.5 g, 15.98 mmol) in DMSO (20 mL) was added potassium tert-butoxide (2.68 g, 23.97 mmol) and the mixture was stirred at room temperature for 30 minutes. 5-bromo-2-chloropyridine-3-carboxamide (Preparation 177, 2.75 g, 15.98 mmol) was added and the reaction stirred at room temperature for 16 hours. The reaction was quenched by the addition of water (20 mL) and extracted into EtOAc (2×100 mL). The organic layers were combined, washed with water (2×50 mL), dried over sodium sulphate and concentrated in vacuo. The residue was purified using silica gel column chromatography eluting with 20% EtOAc in heptanes to afford the title compound (5.00 g, 75%).

¹H NMR (400 MHz, DMSO-d₆): δ ppm 1.40 (s, 9H), 1.80-1.86 (m, 1H), 1.92-1.97 (m, 1H), 2.93-3.00 (m, 1H), 3.07-3.12 (m, 1H), 3.90-4.18 (m, 2H), 4.95-5.08 (m, 1H), 5.35-5.43 (m, 1H), 7.52 (br s, 1H), 7.92 (br s, 1H), 8.27 (d, 1H), 8.43 (d, 1H).

### Preparation 119

### tert-butyl(3R,4S)-4-[(5-bromo-3-carbamoylpyridin-2-yl)oxy]-3-fluoropiperidine-1-carboxylate

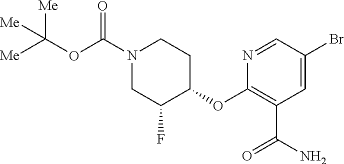 [SMILES: CC(C)(C)OC(=O)N1CC[C@H](Oc2ncc(Br)cc2C(N)=O)[C@H](F)C1]

A solution of 5-bromo-2-chloronicotinamide (Preparation 177, 11.7 g, 49.8 mmol), tert-butyl (3R,4S)-3-fluoro-4-hydroxypiperidine-1-carboxylate (WO 2013/011402 A1, 10.9 g, 49.8 mmol) and potassium tert-butoxide (6.72 g, 60 mmol) in DMSO (165 mL) was stirred at room temperature for 16 hours. The reaction was quenched by the addition of water (500 mL) and extracted into EtOAc (3×500 mL). The organic layers were combined, concentrated in vacuo and the residue triturated with diethylether to afford the title compound as a pale yellow powder (17.8 g, 85%).

¹H NMR (400 MHz, DMSO-d₆): δ ppm 1.39 (s, 9H), 1.82 (m, 1H), 1.94 (m, 1H), 3.01 (m, 1H), 3.27 (m, 1H), 3.93 (s, 1H), 4.14 (s, 1H), 4.98 (d, 1H), 5.38 (dddd, 1H), 7.51 (s, 1H), 7.90 (s, 1H), 8.26 (d, 1H), 8.41 (d, 1H).

MS m/z 418 [M⁷⁹Br+H]⁺

### Preparation 120

### tert-butyl(3S,4R)-4-[4-bromo-2-cyanophenoxy]-3-fluoropiperidine-1-carboxylate

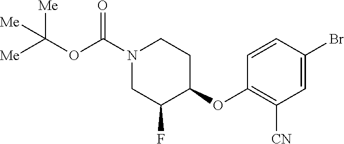 [SMILES: CC(C)(C)OC(=O)N1CC[C@@H](Oc2ccc(Br)cc2C#N)[C@@H](F)C1]

To a solution of tert-butyl-(3S,4R)-3-fluoro-4-hydroxypiperidine-1-carboxylate (WO 2013/011402 A1, 1.00 g, 4.56 mmol) in DMF (20 mL) was added cesium carbonate (4.47 g, 13.68 mmol) followed by 5-bromo-2-fluorobenzonitrile (912 mg, 4.56 mmol) and the reaction was heated to 110° C. for 16 hours in a sealed tube. The reaction was cooled, poured onto ice water and extracted into EtOAc twice. The combined organic layers were washed with water, brine, dried over sodium sulphate and concentrated in vacuo. The residue was purified using silica gel column chromatography eluting with 10% EtOAc in heptanes to afford the title compound (1.50 g, 82%).

¹H NMR (400 MHz, DMSO-d₆): δ ppm 1.40 (s, 9H), 1.86 (m, 2H), 3.00-3.20 (m, 1H), 3.20-3.40 (m, 1H), 3.75-3.85 (m, 1H), 4.00-4.07 (m, 1H), 4.87-4.99 (m, 2H), 7.40 (d, 1H), 7.85 (dd, 1H), 8.04 (s, 1H).

MS m/z 301 [M-Boc₈₁Br+H]⁺

The following Preparations were prepared according to Preparations 118, 119 or 120 using the appropriate aryl halide and hydroxypiperidine as described below:

| Preparation | Name/Structure | Data and starting materials |
|---|---|---|
| 121 | 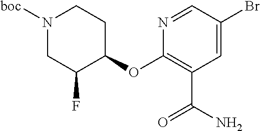 | ¹H NMR (400 MHz, DMSO-d₆): δ ppm 1.40 (s, 9H), 1.82-1.94 (m, 2H), 2.97-3.32 (m, 2H), 3.93-4.00 (m, 1H), 4.15-4.20 (m, 1H), 4.95 (m, 0.5H), 5.08 (m, 0.5H), 5.37 (m, 0.5H), 5.43 (m, 0.5H), 7.52 (br s, 1H), 7.92 (br s, 1H), 8.28 (d, 1H), 8.43 (d, 1H). |
| | 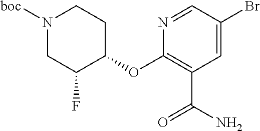 | 5-bromo-2-chloronicotinamide (Preparation 177) and cis-racemic tert-butyl-3-fluoro-4-hydroxy- piperidine-1-carboxylate. |
| | Cis-racemic tert-butyl-4-[(5-bromo-3- | |
| | carbamoylpyridin-2-yl)oxy]-3- | |
| | fluoropiperidine-1-carboxylate | |
| 122 | 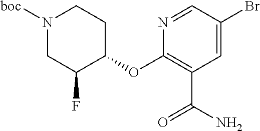 | ¹H NMR (400 MHz, DMSO-d₆): δ ppm 1.40 (s, 9H), 1.85 (m, 1H), 1.97 (m, 1H), 2.90-3.15 (m, 2H), 3.95 (m, 1H), 4.15 (m, 1H), 4.95 (m, 0.5H), 5.08 (m, 0.5H), 5.35- 5.44 (m, 1H), 7.52 (br s, 1H), 7.91 (br s, 1H), 8.28 (d, 1H), 8.43 (d, 1H). |
| | 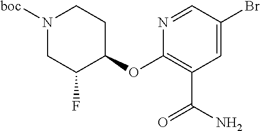 | 5-bromo-2-chloropyridine-3- carboxamide (Preparation 177) and trans-racemic tert-butyl-3- fluoro-4-hydroxy-piperidine-1- carboxylate. |
| | Trans-racemic tert-butyl-4-[(5- | |
| | bromo-3-carbamoylpyridin-2-yl)oxy]- | |
| | 3-fluoropiperidine-1-carboxylate | |
| 123 | 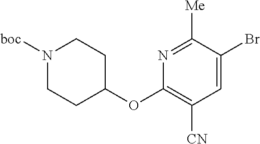 | ¹H NMR (400 MHz, DMSO-d₆): δ ppm 1.40 (s, 9H), 1.63 (m, 2H), 1.91 (m, 2H), 2.55 (s, 3H), 3.30 (m, 2H), 3.58 (m, 2H), 5.30 (m, 1H), 8.53 (s, 1H). 5-bromo-2-chloro-6-methylpyridine- 3-carbonitrile (Preparation 208) and tert-butyl-4-hydroxypiperidine- 1-carboxylate. |
| | tert-butyl 4-[(5-bromo-3-cyano-6- | |
| | methylpyridin-2-yl)oxy]piperidine-1- | |
| | carboxylate | |
| 124 | 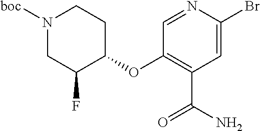 | ¹H NMR (400 MHz, DMSO-d₆): δ ppm 1.40 (s, 9H), 1.70 (m, 1H), 1.98 (m, 1H), 3.25-3.85 (m, 4H), 4.69-4.95 (m, 2H), 7.65 (s, 1H), 7.79 (br s, 1H), 7.86 (br s, 1H), 8.46 (s, 1H). 2-bromo-5-fluoropyridine-4- carboxamide and tert-butyl (3S,4S)-3-fluoro-4- |
| | tert-butyl (3S,4S)-4-[(6-bromo-4- | hydroxypiperidine-1-carboxylate. |
| | carbamoylpyridin-3-yl)oxy]-3- | The residue was purified using |
| | fluoropiperidine-1-carboxylate | silica gel column chromatography |
| | | eluting with 20% EtOAc in |
| | | hexanes. |
| 125 | 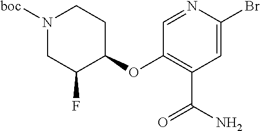 | ¹H NMR (400 MHz, DMSO-d₆): δ ppm 1.40 (s, 9H), 1.78 (m, 1H), 1.93 (m, 1H), 2.80-3.30 (m, 2H), 3.80-4.15 (m, 2H), 4.80 (m, 2H), 7.69 (br s, 1H), 7.71 (s, 1H), 7.94 (br s, 1H), 8.47 (s, 1H). 2-bromo-5-fluoropyridine-4- carboxamide and tert-butyl (3S,4R)-3-fluoro-4- |
| | tert-butyl (3S,4R)-4-[(6-bromo-4- | hydroxypiperidine-1-carboxylate. |
| | carbamoylpyridin-3-yl)oxy]-3- | The residue was purified using |
| | fluoropiperidine-1-carboxylate | silica gel column chromatography |
| | | eluting with 20% EtOAc in |
| | | hexanes. |
| 126 | 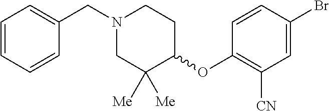 [SMILES: CC1(C)CN(Cc2ccccc2)CCC1Oc1ccc(Br)cc1C#N] | ¹H NMR (400 MHz, DMSO-d₆): δ ppm 0.93 (s, 3H), 1.08 (s, 3H), 1.64-1.73 (m, 1H), 1.89-1.97 (m, 2H), 2.25 (br s, 1H), 2.40 (d, 1H), 2.50 (br s, 1H), 3.42 (d, 1H), 3.50 (d, 1H), 4.21-4.24 (m, 1H), 7.22- 7.34 (m, 5H), 7.77 (dd, 1H), 7.98 |
| | Racemic 2-((1-benzyl-3,3- | (d, 1H). |
| | dimethylpiperidin-4-yl)oxy)-5- | Racemic 1-benzyl-3,3- |
| | bromobenzonitrile | dimethylpiperidin-4-ol |
| | | (WO2001009122) and 5-bromo-2- |
| | | fluorobenzonitrile. |
| 127 | 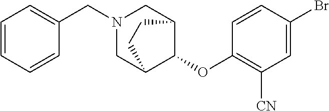 [SMILES: N#Cc1cc(Br)ccc1O[C@@H]1[C@@H]2CC[C@H]1CN(Cc1ccccc1)C2] | ¹H NMR (400 MHz, DMSO-d₆): δ ppm 1.69-1.76 (m, 2H), 2.23-2.33 (m, 4H), 2.67 (m, 2H), 3.50 (s, 2H), 4.61 (m, 1H), 7.23 (m, 1H), 7.29- 7.34 (m, 5H), 7.78 (m, 1H), 7.98 (m, 1H). (1R,5S,8s)-3-benzyl-3- |
| | 2-(((1R,5S,8s)-3-benzyl-3- | azabicyclo[3.2.1]octan-8-ol and 5- |
| | azabicyclo[3.2.1]octan-8-yl)oxy)-5- | bromo-2-fluorobenzonitrile. |
| | bromobenzonitrile | |
| 128 | 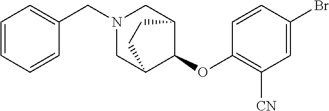 [SMILES: N#Cc1cc(Br)ccc1O[C@H]1[C@@H]2CC[C@H]1CN(Cc1ccccc1)C2] | ¹H NMR (400 MHz, DMSO-d₆): δ ppm 1.71-1.79 (m, 4H), 2.28 (m, 2H), 2.41 (m, 2H), 2.50 (m, 2H), 3.48 (s, 2H), 4.70 (m, 1H), 7.20- 7.23 (m, 1H), 7.30-7.35 (m, 5H), 7.78-7.81 (dd, 1H), 8.02 (d, 1H). (1R,5S,8r)-3-benzyl-3- |
| | 2-(((1R,5S,8r)-3-benzyl-3- | azabicyclo[3.2.1]octan-8-ol and 5- |
| | azabicyclo[3.2.1]octan-8-yl)oxy)-5- | bromo-2-fluorobenzonitrile. |
| | bromobenzonitrile | |
| 129 | 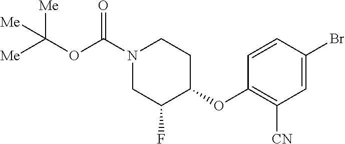 [SMILES: CC(C)(C)OC(=O)N1CC[C@H](Oc2ccc(Br)cc2C#N)[C@H](F)C1] | MS m/z 299 [M − Boc⁷⁹Br + H]⁺ ¹H NMR (400 MHz, DMSO-d₆): δ ppm 1.86 (m, 2H), 3.06-3.37 (m, 2H), 3.80 (m, 1H), 4.00 (m, 1H), 4.87-4.99 (m, 2H), 7.40 (m, 1H), 7.85 (m, 1H), 8.03 (s, 1H). tert-butyl-(3R,4S)-3-fluoro-4- hydroxypiperidine-1-carboxylate (WO 2013/011402 A1) and 5- |
| | tert-butyl (3R,4S)-4-[4-bromo-2- | bromo-2-fluorobenzonitrile. |
| | cyanophenoxy]-3-fluoropiperidine-1- | |
| | carboxylate | |
| 130 | 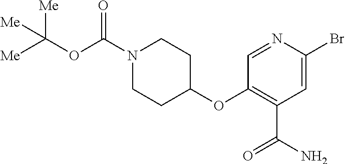 [SMILES: CC(C)(C)OC(=O)N1CCC(Oc2cnc(Br)cc2C(N)=O)CC1] | MS m/z 400 [M⁷⁹Br + H]⁺ ¹H NMR (400 MHz, DMSO-d₆): δ ppm 1.39-1.40 (s, 9H), 1.61-1.63 (m, 2H), 1.89 (m, 2H), 3.23 (m, 2H), 3.55-3.57 (m, 2H), 4.80 (m, 1H), 7.63 (s, 1H), 7.74 (s, 1H), 7.94 (S, 1H), 8.39 (S, 1H). 1-boc- methanesulfonyloxypiperidine and 2-bromo-5-fluoroisonicotinamide. |
| | tert-butyl 4-((6-bromo-4- | |
| | carbamoylpyridin-3-yl)oxy)piperidine- | |
| | 1-carboxylate | |
| 131 | 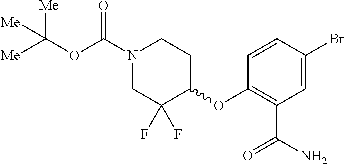 [SMILES: CC(C)(C)OC(=O)N1CCC(Oc2ccc(Br)cc2C(N)=O)C(F)(F)C1] | ¹H NMR (400 MHz, CDCl₃): δ ppm 1.50 (s, 9H), 2.02 (m, 1H), 2.15 (m, 1H), 3.32 (m, 1H), 3.55 (m, 1H), 3.80 (br m, 1H), 4.10 (br m, 1H), 4.60 (m, 1H), 5.80 (br s, 1H), 6.90 (m, 1H), 7.35 (br s, 1H), 7.55 (m, 1H), 8.28 (s, 1H). Racemic tert-butyl-3,3-difluoro-4- hydroxypiperidine-1-carboxylate and 5-bromo-2-fluorobenzamide. |
| | Racemic tert-butyl 4-(4-bromo-2- | |
| | carbamoylphenoxy)-3,3- | |
| | difluoropiperidine-1-carboxylate | |
| 132 | 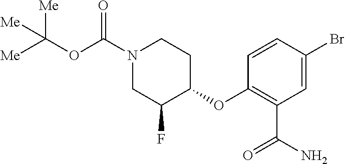 [SMILES: CC(C)(C)OC(=O)N1CC[C@H](Oc2ccc(Br)cc2C(N)=O)[C@@H](F)C1] | MS m/z 419 [M⁸¹Br + H]⁺ ¹H NMR (400 MHz, DMSO-d₆): δ ppm 1.40 (s, 9H), 1.67 (m, 1H), 1.98 (m, 1H), 3.23-3.60 (m, 3H), 3.83 (m, 1H), 4.71-4.84 (m, 2H), 7.24 (d, 1H), 7.57-7.63 (m, 3H), 7.75 (d, 1H). (3S,4R)-3-fluoro-4- methanesulfonyloxypiperidine-1- carboxylic acid tert butyl ester |
| | tert-butyl (3S,4S)-4-(4-bromo-2- | (Preparation 157) and 5-bromo-2- |
| | carbamoylphenoxy)-3- | hydroxybenzamide. |
| | fluoropiperidine-1-carboxylate | |

### Preparation 133

### (3S,4S)-4-[(5-bromo-3-carbamoylpyridin-2-yl)oxy]-3-fluoropiperidine-1-carboxylate

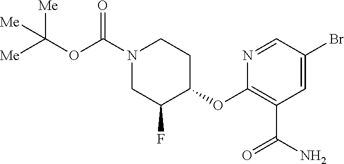 [SMILES: CC(C)(C)OC(=O)N1CC[C@H](Oc2ncc(Br)cc2C(N)=O)[C@@H](F)C1]

To a solution of tert-butyl (3S,4S)-3-fluoro-4-hydroxypiperidine-1-carboxylate (WO 2013/011402 A1, 605 mg, 2.76 mmol) in DMSO (4 mL) was added potassium tert-butoxide (464 mg, 4.14 mmol) and the mixture stirred at room temperature for 30 minutes. 5-bromo-2-chloronicotinamide (Preparation 177, 650 mg, 2.76 mmol) was added and the reaction stirred at room temperature for 16 hours. The reaction was quenched by the addition of water and extracted into EtOAc. The organic layer was collected, washed with water, dried over sodium sulphate and concentrated in vacuo. The residue was purified using silica gel column chromatography eluting with 30% EtOAc in heptanes to afford the title compound (800 mg, 69%).

¹H NMR (400 MHz, DMSO-d₆): δ ppm 1.41 (s, 9H), 1.74 (m, 1H), 2.06 (m, 1H), 3.28-3.83 (m, 4H), 4.80-4.93 (m, 1H), 5.41 (m, 1H), 7.64 (br s, 1H), 7.68-7.82 (m, 2H), 8.21 (m, 1H), 8.42 (s, 1H).

MS m/z 318 [M⁷⁹Br-Boc+H]⁺

### Preparation 134

### tert-butyl(3S,4R)-4-[4-bromo-2-carbamoylphenoxy]-3-fluoropiperidine-1-carboxylate

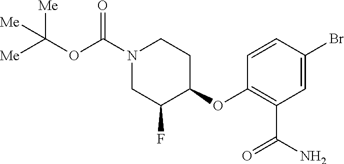 [SMILES: CC(C)(C)OC(=O)N1CC[C@@H](Oc2ccc(Br)cc2C(N)=O)[C@@H](F)C1]

To a solution of tert-butyl (3S,4R)-4-[4-bromo-2-cyanophenoxy]-3-fluoropiperidine-1-carboxylate (Preparation 120, 1.50 g, 3.76 mmol) in tBuOH (15 mL) was added powdered KOH (316 mg, 5.63 mmol) portionwise at 0° C. The reaction was warmed slowly to 80° C. and stirred at this temperature for 3 hours. The reaction was cooled, quenched by the addition of water and extracted into EtOAc twice. The combined organic layers were washed with brine, dried over sodium sulphate and concentrated in vacuo. The residue was purified using silica gel column chromatography eluting with 20% EtOAc in heptanes to afford the title compound as a white solid (1.40 g, 80%).

¹H NMR (400 MHz, DMSO-d₆): δ ppm 1.40 (s, 9H), 1.75 (m, 1H), 1.90 (m, 1H), 2.95 (m, 1H), 3.17 (m, 1H), 3.93 (m, 1H), 4.16 (m, 1H), 4.84-5.05 (m, 2H), 7.27 (d, 1H), 7.54 (br s, 1H), 7.65 (dd, 1H), 7.76 (br s, 1H), 7.90 (s, 1H).

MS m/z 319 [M-Boc⁸¹Br+H]⁺

### Preparation 135

### tert-butyl(3R,4S)-4-[4-bromo-2-carbamoylphenoxy]-3-fluoropiperidine-1-carboxylate

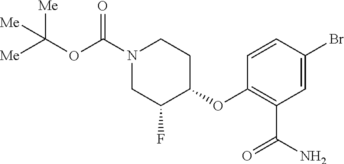 [SMILES: CC(C)(C)OC(=O)N1CC[C@H](Oc2ccc(Br)cc2C(N)=O)[C@H](F)C1]

The title compound was prepared according to the method described for Preparation 134 using tert-butyl (3R,4S)-4-[4-bromo-2-cyanophenoxy]-3-fluoropiperidine-1-carboxylate (Preparation 129).

¹H NMR (400 MHz, DMSO-d₆): δ ppm 1.40 (s, 9H), 1.76 (m, 1H), 1.92 (m, 1H), 2.80-3.32 (m, 2H), 3.93 (m, 1H), 4.16 (m, 1H), 4.84-5.05 (m, 2H), 7.30 (m, 1H), 7.54 (br s, 1H), 7.63-7.66 (m, 1H), 7.76 (br s, 1H), 7.90 (m, 1H).

MS m/z 419 [M⁸¹Br+H]⁺

### Preparation 136

### tert-butyl 4-[2-carbamoyl-4-(1-methyl-1H-pyrazol-4-yl)phenoxy]piperidine-1-carboxylate

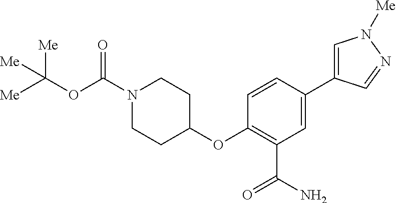 [SMILES: Cn1cc(-c2ccc(OC3CCN(C(=O)OC(C)(C)C)CC3)c(C(N)=O)c2)cn1]

A suspension of 1-boc-4-methanesulfonyloxy-piperidine (2.30 g, 8.29 mmol), 2-hydroxy-5-(1-methyl-1H-pyrazol-4-yl)benzamide (Preparation 163, 1.50 g, 6.90 mmol) and cesium carbonate (2.70 g, 8.29 mmol) in DMF (30 mL) was heated to 80° C. for 4 hours. The reaction was cooled, concentrated in vacuo and diluted with EtOAc. The organic solution was washed with water, brine, dried over sodium sulphate and concentrated in vacuo. The residue was purified using silica gel column chromatography eluting with 5% MeOH in DCM to afford the title compound.

¹H NMR (400 MHz, DMSO-d₆): δ ppm 1.40 (s, 9H), 1.60 (m, 2H), 1.90 (m, 2H), 3.20 (m, 2H), 3.60 (m, 2H), 3.80 (s, 3H), 4.70 (m, 1H), 7.20 (d, 1H), 7.50 (d, 2H), 7.58 (d, 1H), 7.70 (s, 1H), 7.80 (d, 1H), 8.00 (s, 1H).

MS m/z 401 [M+H]⁺

The following Preparations were prepared according to the methods described by Preparation 136 using the appropriate phenol from between 80-100° C. as required as described in the table below:

| | | | Data & Starting |
|---|---|---|---|
| Preparation | Structure | Name | material |
| 137 | 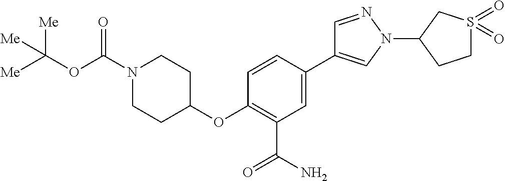 [SMILES: CC(C)(C)OC(=O)N1CCC(Oc2ccc(-c3cnn(C4CCS(=O)(=O)C4)c3)cc2C(N)=O)CC1] | Racemic tert-butyl 4- {2-carbamoyl-4-[1- (1,1- dioxidotetrahydro- thiophen-3-yl)- 1H-pyrazol-4- yl]phenoxy}piperidine- 1-carboxylate | MS m/z 505 [M + H]⁺ 5-[1-(1,1- dioxidotetrahydro- thiophen-3-yl)-1H- pyrazol-4-yl]-2- hydroxybenzamide (Preparation 165). |
| 138 | 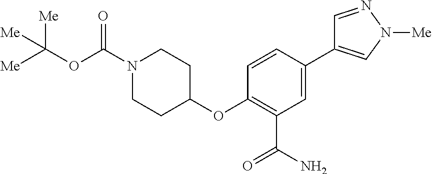 [SMILES: Cn1cc(-c2ccc(OC3CCN(C(=O)OC(C)(C)C)CC3)c(C(N)=O)c2)cn1] | tert-butyl 4-[2- carbamoyl-4-(1- methyl-1H-imidazol-4- yl)phenoxy]piperidine- 1-carboxylate | MS m/z 401 [M + H]⁺ 2-hydroxy-5-(1- methyl-1H-imidazol-4- yl)benzamide (Preparation 160). |
| 139 | 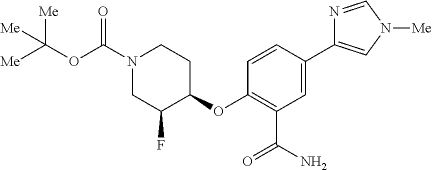 [SMILES: Cn1cnc(-c2ccc(O[C@@H]3CCN(C(=O)OC(C)(C)C)C[C@@H]3F)c(C(N)=O)c2)c1] | Cis-racemic tert-butyl 4-(2-carbamoyl-4-(1- methyl-1H-imidazol-4- yl)phenoxy)-3- fluoropiperidine-1- carboxylate | MS m/z 419 [M + H]⁺ Using 2-hydroxy-5-(1- methyl-1H-imidazol-4- yl)benzamide (Preparation 160) and cis-racemic tert- butyl 3-fluoro-4- ((methylsulfonyl)oxy) piperidine-1- carboxylate |
| | 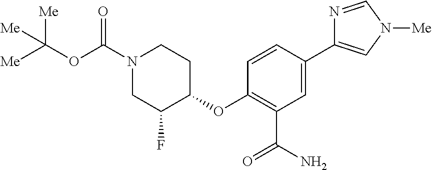 [SMILES: Cn1cnc(-c2ccc(O[C@H]3CCN(C(=O)OC(C)(C)C)C[C@H]3F)c(C(N)=O)c2)c1] | | (Preparation 178). |
| 140 | 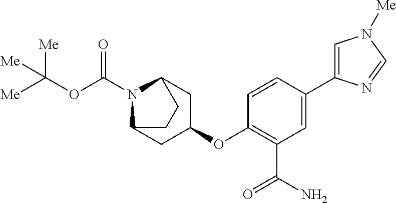 | (1R,3s,5S)-tert-butyl 3-(2-carbamoyl-4-(1- methyl-1H-imidazol-4- yl)phenoxy)-8- azabicyclo[3.2.1] octane-8-carboxylate | MS m/z 427 [M + H]⁺ Using (1R,3s,5S)-tert- butyl 3- ((methylsulfonyl)oxy)- 8- azabicyclo[3.2.1] octane-8-carboxylate (Preparation 179) and 2-hydroxy-5-(1- methyl-1H-imidazol-4- yl)benzamide (Preparation 160). |
| 141 | 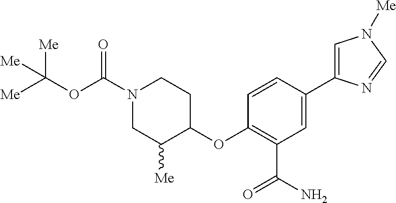 [SMILES: CC1CN(C(=O)OC(C)(C)C)CCC1Oc1ccc(-c2cn(C)cn2)cc1C(N)=O] | Racemic tert-butyl 4- (2-carbamoyl-4-(1- methyl-1H-imidazol-4- yl)phenoxy)-3- methylpiperidine-1- carboxylate | MS m/z 415 [M + H]⁺ Using 2-hydroxy-5-(1- methyl-1H-imidazol-4- yl)benzamide (Preparation 160) and racemic tert-butyl- 3-methyl- 4((methylsulfonyl)oxy)- piperidine-1- carboxylate (WO2009013211). |
| 142 | 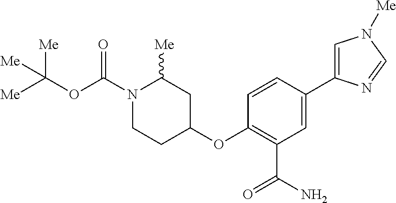 [SMILES: CC1CC(Oc2ccc(-c3cn(C)cn3)cc2C(N)=O)CCN1C(=O)OC(C)(C)C] | Racemic tert-butyl 4- (2-carbamoyl-4-(1- methyl-1H-imidazol-4- yl)phenoxy)-2- methylpiperidine-1- carboxylate | MS m/z 415 [M + H]⁺ Using 2-hydroxy-5-(1- methyl-1H-imidazol-4- yl)benzamide (Preparation 160) and racemic tert-butyl- 2-methyl- 4((methylsulfonyl)oxy)- piperidine-1- carboxylate (PCT Intl. Appl. 2010009183). |
| 143 | 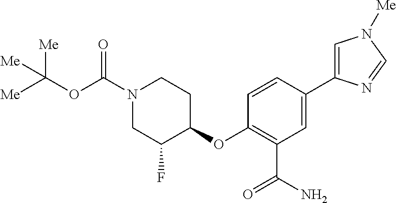 [SMILES: Cn1cnc(-c2ccc(O[C@@H]3CCN(C(=O)OC(C)(C)C)C[C@H]3F)c(C(N)=O)c2)c1] | (3R,4R)-tert-butyl 4- (2-carbamoyl-4-(1- methyl-1H-imidazol-4- yl)phenoxy)-3- fluoropiperidine-1- carboxylate | MS m/z 419 [M + H]⁺ Using (3R,4S)-3- fluoro-4- methanesulfonyloxy- piperidine-1-carboxylic acid tert butyl ester (Preparation 158) and 2-hydroxy-5-(1- methyl-1H-imidazol-4- yl)benzamide (Preparation 160) |
| 144 | 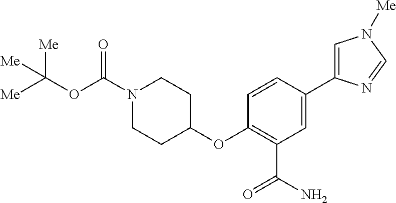 [SMILES: Cn1cnc(-c2ccc(OC3CCN(C(=O)OC(C)(C)C)CC3)c(C(N)=O)c2)c1] | tert-butyl 4-(2- carbamoyl-4-(1- methyl-1H-pyrazol-4- yl)phenoxy)piperidine- 1-carboxylate | MS m/z 401 [M + H]⁺ 2-hydroxy-5-(1- methyl-1H-pyrazol-4- yl)benzamide (Preparation 163) and 1-Boc-4- ((methanesulfonyl) oxy)piperidine. |

### Preparation 145

### 2-(((1R,5S,8r)-3-benzyl-3-azabicyclo[3.2.1]octan-8-yl)oxy)-5-(1-methyl-1H-imidazol-4-yl)benzamide

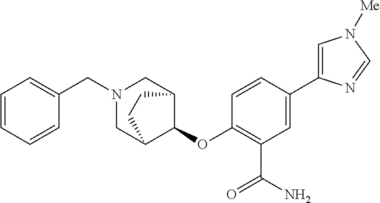 [SMILES: Cn1cnc(-c2ccc(O[C@H]3[C@@H]4CC[C@H]3CN(Cc3ccccc3)C4)c(C(N)=O)c2)c1]

To a solution of 2-(((1R,5S,8r)-3-benzyl-3-azabicyclo[3.2.1]octan-8-yl)oxy)-5-(4,4,5,5-tetramethyl-1,3,2-dioxaborolan-2-yl)benzamide (Preparation 151, 300 mg, 0.65 mmol) and 4-bromo-1-methyl-1H-imidazole (0.16 mL, 0.97 mmol) in dioxane (8 mL) was added a solution of sodium carbonate (172 mg, 1.62 mmol) in water (2 mL) and the mixture was degassed with argon for 15 minutes. Pd₂(dba)₃ (29 mg, 0.032 mmol) followed by tBu₃HPBF₄ (37 mg, 0.13 mmol) were added and the reaction was heated at 100° C. in a sealed tube for 16 hours. The reaction was cooled, washed with water, brine, dried over sodium sulphate and concentrated in vacuo. The residue was purified using silica gel column chromatography eluting with 6% MeOH in DCM to afford the title compound (170 mg, 62%).

MS m/z 417 [M+H]⁺

### Preparation 146

### 2-(((1R,5S,8s)-3-benzyl-3-azabicyclo[3.2.1]octan-8-yl)oxy)-5-(1-methyl-1H-imidazol-4-yl)benzamide

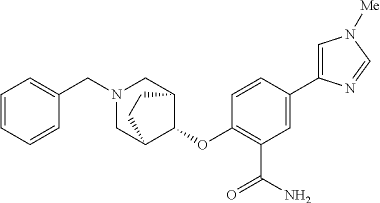 [SMILES: Cn1cnc(-c2ccc(O[C@@H]3[C@@H]4CC[C@H]3CN(Cc3ccccc3)C4)c(C(N)=O)c2)c1]

The title compound was prepared according to the method described for Preparation 145 using 2-(((1R,5S,8s)-3-benzyl-3-azabicyclo[3.2.1]octan-8-yl)oxy)-5-(4,4,5,5-tetramethyl-1,3,2-dioxaborolan-2-yl)benzamide (Preparation 152) and 4-bromo-1-methyl-1H-imidazole.

MS m/z 417 [M+H]⁺

### Preparation 147

### Racemic 2-((1-benzyl-3,3-dimethylpiperidin-4-yl)oxy)-5-(1-methyl-1H-imidazol-4-yl)benzamide

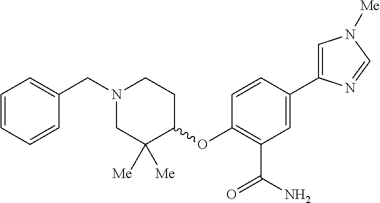 [SMILES: Cn1cnc(-c2ccc(OC3CCN(Cc4ccccc4)CC3(C)C)c(C(N)=O)c2)c1]

The title compound was prepared according to the method described for Preparation 145 using racemic 2-((1-benzyl-3,3-dimethylpiperidin-4-yl)oxy)-5-(4,4,5,5-tetramethyl-1,3,2-dioxaborolan-2-yl)benzamide (Preparation 153) and 4-bromo-1-methyl-1H-imidazole.

¹H NMR (400 MHz, DMSO-d₆): δ ppm 0.98 (s, 3H), 1.08 (s, 3H), 1.7 (m, 1H), 1.90 (m, 1H), 2.00 (m, 1H), 2.25 (m, 1H), 2.30-2.40 (m, 1H), 2.65 (br s, 1H), 3.46 (q, 2H), 3.66 (s, 3H), 4.25 (m, 1H), 7.18 (d, 1H), 7.25 (m, 1H), 7.31 (m, 4H), 7.50 (br s, 1H), 7.52 (s, 1H), 7.59 (s, 1H), 7.65 (br s, 1H), 7.74 (dd, 1H), 8.12 (d, 1H).

### Preparation 148

### tert-butyl 4-{2-carbamoyl-4-[1-(1,1-dioxidothietan-3-yl)-1H-pyrazol-4-yl]phenoxy}piperidine-1-carboxylate

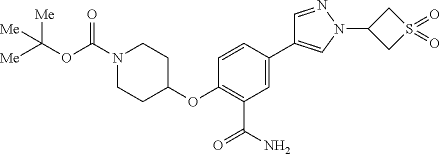 [SMILES: CC(C)(C)OC(=O)N1CCC(Oc2ccc(-c3cnn(C4CS(=O)(=O)C4)c3)cc2C(N)=O)CC1]

To a stirred solution of 1-boc-4-(methylsulfonyl)oxypiperidine (218 mg, 0.78 mmol) and 5-(1-(1,1-dioxidothietan-3-yl)-1H-pyrazol-4-yl)-2-hydroxybenzamide (Preparation 164, mg, 0.39 mmol) in DMF (2 mL) was added cesium carbonate (254 mg, 0.78 mmol) and the reaction heated at 70° C. for 30 hours. The reaction mixture was cooled to room temperature and EtOAc (30 mL) and water (20 mL) were added. The two phases were separated and the aqueous phase extracted with EtOAc (20 mL). The combined organic extracts were washed with brine (15 mL), dried (MgSO₄) and the solvent removed under reduced pressure. The crude product was triturated with EtOAc (10 mL) and filtered, washing with EtOAc to afford the title compound (78 mg, 41%) as a pale tan solid.

¹H NMR (400 MHz, CD₃OD): δ ppm 1.46 (s, 9H), 1.72-1.86 (m, 2H), 2.02-2.10 (m, 2H), 3.32-3.38 (m, 2H), 3.74-3.82 (m, 2H), 4.72 (d, 4H), 4.76-4.84 (m, 1H), 5.36 (m, 1H), 7.22 (d, 1H), 7.68 (d, 1H), 7.94 (s, 1H), 8.08 (s, 1H), 8.16 (s, 1H).

MS m/z 491 [M+H]⁺

### Preparation 149

### tert-butyl 4-[(5-bromo-3-carbamoylpyridin-2-yl)oxy]piperidine-1-carboxylate

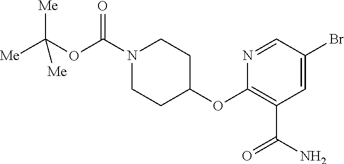 [SMILES: CC(C)(C)OC(=O)N1CCC(Oc2ncc(Br)cc2C(N)=O)CC1]

The title compound was prepared according to the method described for Preparation 133 using 5-bromo-2-chloronictinamide (Preparation 177) and tert-butyl 4-hydroxypiperidine-1-carboxylate. The residue was purified using silica gel column chromatography eluting with 0-2% MeOH in DCM.

¹H NMR (400 MHz, DMSO-d₆): δ ppm 1.40 (m, 9H), 1.68 (m, 2H), 1.93 (m, 2H), 3.17 (m, 2H), 3.65 (m, 2H), 5.28 (t, 1H), 7.58 (br s, 1H), 7.81 (br s, 1H), 8.20 (d, 1H), 8.40 (d, 1H).

### Preparation 150

### tert-butyl 4-(4-bromo-2-carbamoylphenoxy)piperidine-1-carboxylate

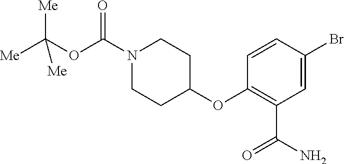 [SMILES: CC(C)(C)OC(=O)N1CCC(Oc2ccc(Br)cc2C(N)=O)CC1]

The title compound was prepared according to the method described for Preparation 92 using 5-bromo-2-hydroxybenzamide and 1-boc-4-methanesulfonyloxypiperidine (Preparation 159).

¹H NMR (400 MHz, DMSO-d₆): δ ppm 1.40 (s, 9H), 1.62 (m, 2H), 1.89 (m, 2H), 3.21 (m, 2H), 3.62 (m, 2H), 4.71 (m, 1H), 7.21 (d, 1H), 7.54-7.65 (m, 3H), 7.78 (d, 1H).

MS m/z 399 [M⁷⁹Br+H]⁺

### Preparation 151

### 2-(((1R,5S,8r)-3-benzyl-3-azabicyclo[3.2.1]octan-8-yl)oxy)-5-(4,4,5,5-tetramethyl-1,3,2-dioxaborolan-2-yl)benzamide

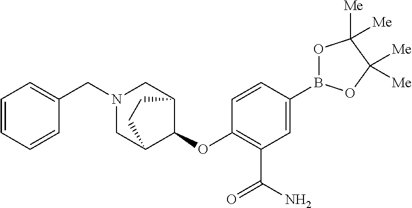 [SMILES: CC1(C)OB(c2ccc(O[C@H]3[C@@H]4CC[C@H]3CN(Cc3ccccc3)C4)c(C(N)=O)c2)OC1(C)C]

The title compound was prepared according to the method described for Preparation 99 using 2-(((1R,5S,8r)-3-benzyl-3-azabicyclo[3.2.1]octan-8-yl)oxy)-5-bromobenzamide (Preparation 154).

MS m/z 463 [M+H]⁺

### Preparation 152

### 2-(((1R,5S,8s)-3-benzyl-3-azabicyclo[3.2.1]octan-8-yl)oxy)-5-(4,4,5,5-tetramethyl-1,3,2-dioxaborolan-2-yl)benzamide

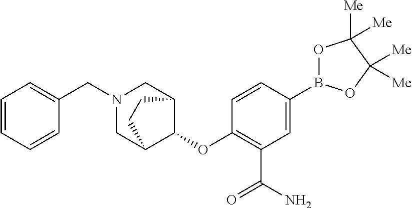 [SMILES: CC1(C)OB(c2ccc(O[C@@H]3[C@@H]4CC[C@H]3CN(Cc3ccccc3)C4)c(C(N)=O)c2)OC1(C)C]

The title compound was prepared according to the method described for Preparation 99 using 2-(((1R,5S,8s)-3-benzyl-3-azabicyclo[3.2.1]octan-8-yl)oxy)-5-bromobenzamide (Preparation 155).

MS m/z 463 [M+H]⁺

### Preparation 153

### Racemic 2-((1-benzyl-3,3-dimethylpiperidin-4-yl)oxy)-5-(4,4,5,5-tetramethyl-1,3,2-dioxaborolan-2-yl)benzamide

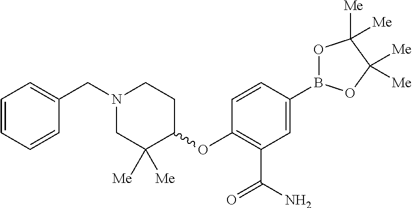 [SMILES: CC1(C)CN(Cc2ccccc2)CCC1Oc1ccc(B2OC(C)(C)C(C)(C)O2)cc1C(N)=O]

The title compound was prepared according to the method described for Preparation 99 using racemic 2-((1-benzyl-3,3-dimethylpiperidin-4-yl)oxy)-5-bromobenzamide (Preparation 156).

MS m/z 465 [M+H]⁺

### Preparation 154

### 2-(((1R,5S,8r)-3-benzyl-3-azabicyclo[3.2.1]octan-8-yl)oxy)-5-bromobenzamide

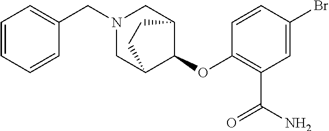 [SMILES: NC(=O)c1cc(Br)ccc1O[C@H]1[C@@H]2CC[C@H]1CN(Cc1ccccc1)C2]

To a solution of 2-(((1R,5S,8r)-3-benzyl-3-azabicyclo[3.2.1]octan-8-yl)oxy)-5-bromobenzonitrile (Preparation 128, 150 mg, 0.70 mmol) in tert-butyl alcohol (3 mL) was added powdered potassium hydroxide (32 mg, 1.04 mmol) and the reaction was heated to 80° C. for 16 hours. The reaction was cooled, diluted with EtOAc, washed with water, brine, dried over sodium sulphate and concentrated in vacuo The residue was purified using silica gel column chromatography eluting with 25-35% EtOAc in heptanes to afford the title compound (90 mg, 57%).

¹H NMR (400 MHz, DMSO-d₆): δ ppm 1.71-1.77 (m, 4H), 2.30 (m, 2H), 2.41 (m, 2H), 2.49 (m, 2H), 3.47 (s, 2H), 4.62 (m, 1H), 7.22 (m, 2H), 7.30 (m, 4H), 7.55-7.58 (m, 2H), 7.79 (m, 2H).

### Preparation 155

### 2-(((1R,5S,8s)-3-benzyl-3-azabicyclo[3.2.1]octan-8-yl)oxy)-5-bromobenzamide

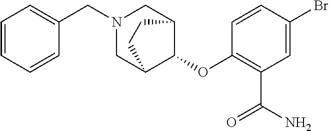 [SMILES: NC(=O)c1cc(Br)ccc1O[C@@H]1[C@@H]2CC[C@H]1CN(Cc1ccccc1)C2]

The title compound was prepared according to the method described for Preparation 154 using 2-(((1R,5S,8s)-3-benzyl-3-azabicyclo[3.2.1 ]octan-8-yl)oxy)-5-bromobenzonitrile (Preparation 127).

¹H NMR (400 MHz, DMSO-d₆): δ ppm 1.70 (m, 4H), 2.24-2.27 (m, 2H), 2.34 (m, 2H), 2.64-2.68 (m, 2H), 3.50 (s, 2H), 4.56 (m, 1H), 7.20-7.25 (m, 2H), 7.29-7.34 (m, 4H), 7.44 (br s, 1H), 7.57-7.60 (dd, 1H), 7.69 (br s, 1H), 7.83 (d, 1H).

### Preparation 156

### Racemic 2-((1-benzyl-3,3-dimethylpiperidin-4-yl)oxy)-5-bromobenzamide

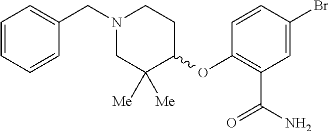 [SMILES: CC1(C)CN(Cc2ccccc2)CCC1Oc1ccc(Br)cc1C(N)=O]

The title compound was prepared according to the method described for Preparation 154 using racemic 2-((1-benzyl-3,3-dimethylpiperidin-4-yl)oxy)-5-bromobenzonitrile (Preparation 126).

¹H NMR (400 MHz, DMSO-d₆): δ ppm 0.95 (s, 3H), 1.05 (s, 3H), 1.68-1.72 (m, 1H), 1.86-1.89 (m, 1H), 1.98 (d, 1H), 2.22 (m, 1H), 2.35 (d, 1H), 2.66 (m, 1H), 3.45 (q, 2H), 4.22 (m, 1H), 7.19 (d, 1H), 7.22-7.25 (m, 1H), 7.31-7.34 (m, 4H), 7.49 (br s, 1H), 7.56 (dd, 1H), 7.76 (br s, 1H), 7.77 (d, 1H).

### Preparation 157

### (3S,4R)-3-fluoro-4-methanesulfonyloxypiperidine-1-carboxylic acid tert butyl ester

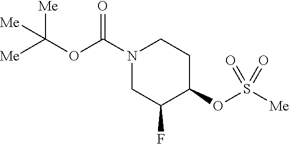 [SMILES: CC(C)(C)OC(=O)N1CC[C@@H](OS(C)(=O)=O)[C@@H](F)C1]

To a solution of tert-butyl-(3S,4R)-3-fluoro-4-hydroxypiperidine-1-carboxylate (WO 2013/011402 A1, 300 mg, 1.37 mmol) in DCM (10 mL) was added triethylamine (0.286 mL, 2.06 mmol) followed by methanesulfonyl chloride (0.128 mL, 1.64 mmol) at −30° C. The reaction was stirred at this temperature for 3 hours before diluting with DCM (30 mL). The solution was washed with saturated aqueous NaHCO₃ solution (10 mL), water (10 mL), brine (10 mL), dried over sodium sulphate and concentrated in vacuo to afford the title compound that was used directly in the next step (400 mg, 98%).

### Preparation 158

### (3R,4S)-3-fluoro-4-methanesulfonyloxypiperidine-1-carboxylic acid tert butyl ester

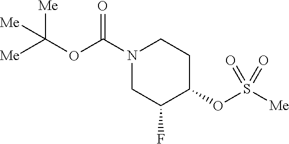 [SMILES: CC(C)(C)OC(=O)N1CC[C@H](OS(C)(=O)=O)[C@H](F)C1]

The title compound was prepared according to the method described for Preparation 157 using tert-butyl-(3R,4S)-3-fluoro-4-hydroxypiperidine-1-carboxylate (WO 2013/011402 A1). Taken on directly to the next step.

### Preparation 159

### 4-methanesulfonyloxypiperidine-1-carboxylic acid tert butyl ester

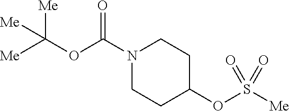 [SMILES: CC(C)(C)OC(=O)N1CCC(OS(C)(=O)=O)CC1]

The title compound was prepared according to the method described for Preparation 157 using tert-butyl-4-hydroxypiperidine-1-carboxylate. Used directly in the next reaction.

¹H NMR (400 MHz, DMSO-d₆): δ ppm 1.40 (s, 9H), 1.56-1.64 (m, 2H), 1.88-1.93 (m, 2H), 3.15-3.19 (m, 5H), 3.57-3.62 (m, 2H), 4.80-4.84 (m, 1H).

### Preparation 160

### 2-hydroxy-5-(1-methyl-1H-imidazol-4-yl)benzamide

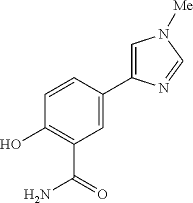 [SMILES: Cn1cnc(-c2ccc(O)c(C(N)=O)c2)c1]

A solution of 2-benzyloxy-5-(1-methyl-1H-imidazol-4-yl)benzamide (Preparation 161, 1 g, 3.26 mmol) in MeOH (20 mL) was degassed with argon for 10 minutes followed by the addition of palladium hydroxide on carbon (250 mg). The reaction was stirred under a balloon of hydrogen for 3 hours at room temperature. The reaction was filtered through celite, washed through with methanol (100 mL) and concentrated in vacuo to afford the title compound as a brown solid (560 mg, 79%).

¹H NMR (400 MHz, DMSO-d₆): δ ppm 3.64 (s, 3H), 6.86 (d, 1H), 7.44 (s, 1H), 7.60 (s, 1H), 7.77 (d, 1H), 7.86 (br s, 1H), 8.19 (s, 1H), 8.45 (br s, 1H), 12.88 (s, 1H).

MS m/z 218 [M+H]⁺

### Preparation 161

### 2-Benzyloxy-5-(1-methyl-1H-imidazol-4-yl)-benzamide

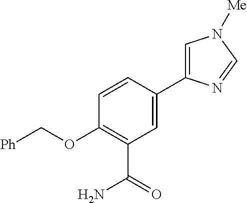 [SMILES: Cn1cnc(-c2ccc(OCc3ccccc3)c(C(N)=O)c2)c1]

To a solution of 2-benzyloxy-5-(4,4,5,5-tetramethyl-[1,3,2]dioxaborolan-2-yl)benzamide (Preparation 162, 5 g, 14.16 mmol) and 4-iodo-1-methyl-1H-imidazole (2.94 g, 14.16 mmol) in DMF (60 mL) was added a solution of potassium carbonate (4.89 g, 35.41 mmol) in water (10 mL). The solution was degassed with argon for 15 minutes followed by the addition of 1,1-bis(diphenylphosphino)ferrocene palladium (II) dichloride (578 mg, 0.71 mmol). The reaction was heated to 110° C. for 16 hours before cooling and diluting with EtOAc (300 mL). The organic solution was washed with water (2×100 mL), brine (75 mL), dried over sodium sulphate and concentrated in vacuo. The residue was purified using silica gel column chromatography eluting with 0-4% MeOH in DCM to afford the title compound (1 g, 23%).

¹H NMR (400 MHz, DMSO-d₆): δ ppm 3.66 (s, 3H), 5.25 (s, 2H), 7.20 (d, 1H), 7.34 (t, 1H), 7.41 (t, 2H), 7.50-7.54 (m, 4H), 7.59-7.61 (m, 2H), 7.77 (dd, 1H), 8.15 (d, 1H).

MS m/z 308 [M+H]⁺

### Preparation 162

### 2-benzyloxy-5-(4,4,5,5-tetramethyl-[1,3,2]dioxaborolan-2-yl)benzamide

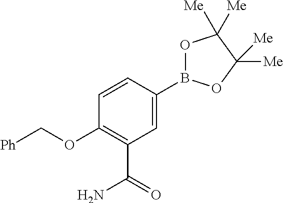 [SMILES: CC1(C)OB(c2ccc(OCc3ccccc3)c(C(N)=O)c2)OC1(C)C]

A suspension of 2-benzyloxy-5-bromobenzamide (Preparation 172, 5 g, 16.34 mmol), bispinacolatodiboron (6.22 g, 24.51 mmol) and potassium acetate (4.81 g, 49.02 mmol) in dioxane (30 mL) was degassed with argon for 20 minutes followed by the addition of 1,1-bis(diphenylphosphino)ferrocene palladium (II) dichloride (667 mg, 0.82 mmol). The reaction was heated to 110° C. for 14 hours before cooling to room temperature and filtering through celite. The filter cake was washed with further EtOAc and the combined filtrate concentrated in vacuo. The crude residue was purified using silica gel column chromatography eluting with 0-20% EtOAc in hexane to afford the title compound (5 g, 87%).

¹H NMR (400 MHz, DMSO-d₆): δ ppm 1.28 (s, 12H), 5.29 (s, 2H), 7.21 (d, 1H), 7.36 (t, 1H), 7.40 (t, 2H), 7.49 (d, 2H), 7.54 (br s, 1H), 7.58 (br s, 1H), 7.71 (dd, 1H), 8.12 (d, 1H).

MS m/z 354 [M+H]⁺

### Preparation 163

### 2-hydroxy-5-(1-methyl-1H-pyrazol-4-yl)benzamide

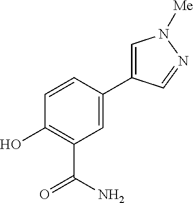 [SMILES: Cn1cc(-c2ccc(O)c(C(N)=O)c2)cn1]

A solution of 5-bromo-2-hydroxybenzamide (50 g, 231 mmol), 1-methyl-4-(4,4,5,5-tetramethyl-1,3,2-dioxaborolan-2-yl)-1H-pyrazole (57.78 g, 277 mmol) and sodium carbonate (78.59 g, 740 mmol) in dioxane/water (400 mL/250 mL) was degassed with nitrogen. 1,1-bis(diphenylphosphino)ferrocene palladium (II) dichloride (14.19 g, 17.3 mmol) was added and the reaction heated to 100° C. for 26 hours. The reaction was cooled, concentrated in vacuo and purified using silica gel column chromatography eluting with 10% MeOH in DCM to afford the title compound (40 g, 67%).

¹H NMR (400 MHz, DMSO-d₆): δ ppm 3.80 (s, 3H), 6.80 (d, 1H), 7.50 (m, 1H), 7.80 (s, 1H), 7.90 (m, 1H), 8.00 (s, 1H), 8.00 (d, 2H), 8.40 (s, 1H).

MS m/z 216 [M−H]⁻

### Preparation 164

### 5-[1(1,1-dioxidothietan-3-yl)-1H-pyrazol-4-yl]-2-hydroxybenzamide

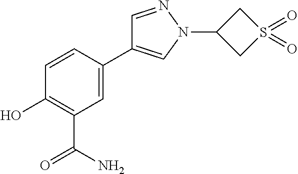 [SMILES: NC(=O)c1cc(-c2cnn(C3CS(=O)(=O)C3)c2)ccc1O]

5-Bromo-2-hydroxybenzamide (290 mg, 1.34 mmol), 1-(1,1-dioxidothietan-3-yl)-4-(4,4,5,5-tetramethyl-1,3,2-dioxaborolan-2-yl)-1H-pyrazole (Preparation 168, 400 mg, 1.34 mmol) and sodium carbonate (426 mg, 4.02 mmol) in a solvent mixture of dioxane (15 mL) and water (3 mL) was degassed. Tetrakis(triphenylphosphine)palladium(0) (77.4 mg, 0.07 mmol) was added and the reaction further degassed before heating to reflux for 3 hours. The mixture was cooled, filtered through celite washing through with ethyl acetate. The filtrate was concentrated in vacuo and the residue triturated with dichloromethane to afford the title compound as a cream solid (240 mg, 58%).

¹H NMR (400 MHz, DMSO-d₆): δ ppm 4.64-4.69 (m, 2H), 4.77-4.83 (m, 2H), 5.34-5.39 (m, 1H), 6.99 (d, 1H), 7.61-7.64 (m, 1H), 7.96 (br s, 1H), 7.99 (s, 1H), 8.09 (m, 1H), 8.25 (s, 1H), 8.44 (br s, 1H), 12.92 (s, 1H).

### Preparation 165

### Racemic 5-[1-(1,1-dioxidotetrahydrothiophen-3-yl)-1H-pyrazol-4-yl]-2-hydroxybenzamide

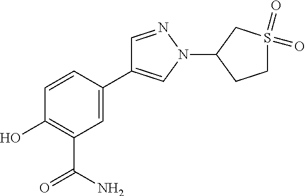 [SMILES: NC(=O)c1cc(-c2cnn(C3CCS(=O)(=O)C3)c2)ccc1O]

A solution of 1-(1,1-dioxidotetrahydrothiophen-3-yl)-4-(4,4,5,5-tetramethyl-1,3,2-dioxaborolan-2-yl)-1H-pyrazole (Preparation 185, 400 mg, 1.282 mmol) and 5-bromo-2-hydroxybenzamide (276 mg, 1.282 mmol) in dioxane (8 mL) was degassed before the addition of sodium carbonate (339 mg, 3.205 mmol) in water (2 mL). The solution was further degassed with argon followed by the addition of tris(dibenzylideneacetone)dipalladium (0) (58 mg, 0.064 mmol) and tri-tertbutylphosphine tetrafluoroborate salt (30 mg, 0.10 mmol). The reaction was heated to 100° C. for 16 hours, before cooling and diluting with EtOAc. The solution was washed with water, brine, dried over sodium sulphate and concentrated in vacuo. The residue was purified using silica gel column chromatography eluting with 4-5% MeOH in DCM to afford the title compound as an off white solid (270 mg, 66%).

MS m/z 320 [M−H]⁻

### Preparation 166

### 2-hydroxy-5-(1-(2-hydroxy-2-methylpropyl)-1H-pyrazol-4-yl)benzamide

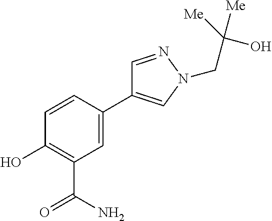 [SMILES: CC(C)(O)Cn1cc(-c2ccc(O)c(C(N)=O)c2)cn1]

The title compound was prepared according to the method described for Preparation 165 using 5-bromo-2-hydroxybenzamide and 2-methyl-1-(4-(4,4,5,5-tetramethyl-1,3,2-dioxaborolan-2-yl)-1H-pyrazol-1-yl)propan-2-ol (Preparation 216).

MS m/z 274 [M−H]⁻

### Preparation 167

### 2-hydroxy-5-{1-[(3-hydroxyoxetan-3-yl)methyl]-1H-pyrazol-4-yl}benzamide

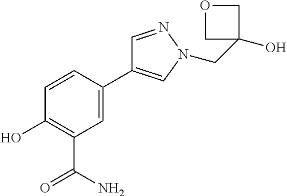 [SMILES: NC(=O)c1cc(-c2cnn(CC3(O)COC3)c2)ccc1O]

The title compound was prepared according to the method described for Preparation 163 using 2-hydroxy-5-(4,4,5,5-tetramethyl-1,3,2-dioxaborolan-2-yl)benzamide (Preparation 183), 3-[(4-bromo-1H-pyrazol-1-yl)methyl]oxetan-3-ol (Preparation 184) and potassium carbonate as base.

¹H NMR (400 MHz, CD₃OD): δ ppm 4.23 (br s, 4H), 4.54 (br s, 2H), 6.87 (d, 1H), 7.47 (dd, 1H), 7.72 (s, 1H), 7.79 (s, 1H), 7.86 (d, 1H).

MS m/z 290 [M+H]⁺

### Preparation 168

### 1-(1,1-dioxidothietan-3-yl)-4-(4,4,5,5-tetramethyl-1,3,2-dioxaborolan-2-yl)-1H-pyrazole

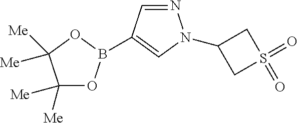 [SMILES: CC1(C)OB(c2cnn(C3CS(=O)(=O)C3)c2)OC1(C)C]

To a solution of 4-(4,4,5,5-tetramethyl-1,3,2-dioxaborolan-2-yl)-1H-pyrazole (1.26 g, 6.49 mmol) in acetonitrile (40 mL) was added 3-chlorothietane 1,1-dioxide (1.19 g, 8.44 mmol) and cesium carbonate (7.40 g, 22.71 mmol) and the reaction was heated to reflux for 18 hours. The reaction was cooled, concentrated in vacuo and diluted with EtOAc (100 mL). The solution was washed with water (100 mL), the organic layer collected, dried over MgSO₄ and concentrated in vacuo. The residue was triturated with heptanes to afford the title compound as a brown solid (900 mg, 46%).

¹H NMR (400 MHz, CDCl₃): δ ppm 1.32 (s, 12H), 4.63-4.57 (m, 2H), 4.78-4.73 (m, 2H), 5.22-5.14 (m, 1H), 7.85 (s, 1H), 7.87 (s, 1H).

MS m/z 299 [M+H]⁺

### Preparation 169

### [4-(trifluoromethoxy)phenyl]acetyl chloride

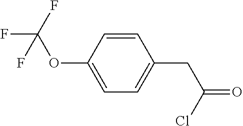 [SMILES: O=C(Cl)Cc1ccc(OC(F)(F)F)cc1]

A solution of 2-(4-trifluoromethoxy)phenyl)acetic acid (3.50 g, 15.9 mmol) and oxalyl chloride (2.22 g, 17.5 mmol) in DCM (63.6 mL) was stirred at room temperature. Catalytic DMF (116 mg, 1.59 mmol) was added and the reaction stirred at room temperature for 1 hour to afford the title compound as a 0.25M solution in DCM that was used directly in the next reaction.

### Preparation 170

### 4-Bromo-1-((methanesulfonyl)methyl)-1H-pyrazole

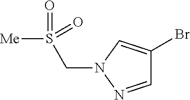 [SMILES: CS(=O)(=O)Cn1cc(Br)cn1]

To a solution of 4-bromo-1-((methylthio)methyl)-1H-pyrazole (Preparation 171, 7.0 g, 33.80 mmol) in MeOH (196 mL) and water (49 mL) was added oxone (62 g, 101.41 mmol) portionwise. The reaction was stirred at room temperature for 16 hours before quenching with water (30 mL) and extracting into EtOAc (3×200 mL). The organic layers were combined, dried over sodium sulphate and concentrated in vacuo to afford the title compound as a white solid (7 g, 87%).

¹H NMR (400 MHz, DMSO-d₆): δ ppm 3.01 (s, 3H), 5.73 (s, 2H), 7.77 (s, 1H), 8.09 (s, 1H).

MS m/z 239 [M⁷⁹Br+H]⁺

### Preparation 171

### 4-Bromo-1-((methylthio)methyl)-1H-pyrazole

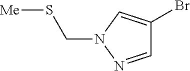

To a solution of 4-bromo-1H-pyrazole) 5 g, 34.02 mmol) in MeCN (50 mL) was added potassium carbonate (9.4 g, 68.04 mmol) followed by chloromethylsulfanylmethane (3.94 g, 40.82 mmol). The reaction was heated to 100° C. for 16 hours. The reaction was quenched by the addition of water (10 mL) and extracted into EtOAc (2×100 mL). The organic layers were combined, dried over sodium sulphate and concentrated in vacuo to afford the title compound as a yellow oil (7.2 g, 99%).

¹H NMR (400 MHz, DMSO-d₆): δ ppm 2.08 (s, 3H), 5.24 (s, 2H), 7.59 (s, 1H), 8.06 (s, 1H).

### Preparation 172

### 2-Benzyloxy-5-bromo-benzamide

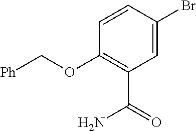 [SMILES: NC(=O)c1cc(Br)ccc1OCc1ccccc1]

To a solution of 5-bromo-2-hydroxybenzamide (8 g, 37.03 mmol) in acetone (100 mL) was added potassium carbonate (10.2 g, 74.07 mmol) followed by benzyl bromide (4.08 mL, 40.74 mmol) and the reaction was heated to 60° C. for 3 hours. The reaction was cooled and concentrated in vacuo. The residue was diluted with EtOAc (200 mL), washed with water (50 mL), brine (30 mL), dried over sodium sulphate and concentrated in vacuo to afford the title compound (11.5 g, 97%).

¹H NMR (400 MHz, DMSO-d₆): δ ppm 5.25 (s, 2H), 7.18 (d, 1H), 7.34 (t, 1H), 7.40 (t, 2H), 7.48 (d, 2H), 7.60 (dd, 1H), 7.66 (br s, 2H), 7.81 (d, 1H).

MS m/z 306 [M+⁷⁹Br+H]⁺

### Preparation 173

### (4-Cyclopropoxyphenyl)acetic acid

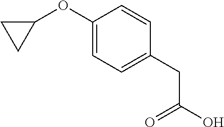 [SMILES: O=C(O)Cc1ccc(OC2CC2)cc1]

To a solution of methyl 2-(4-cyclopropoxyphenyl)acetate (Preparation 174, 1.78 g, 8.5 mmol) in THF/water (3:1, 20 mL) was added lithium hydroxide (1.08 g, 25.92 mmol) and the reaction was stirred at room temperature for 3 hours. The reaction was acidified with 1N HCl and extracted into EtOAc. The organic layer was collected, dried over sodium sulphate and concentrated in vacuo to afford the title compound (1.60 g, 93%).

¹H NMR (400 MHz, DMSO-d₆): δ ppm 0.61-0.64 (m, 2H), 0.73-0.78 (m, 2H), 3.48 (s, 2H), 3.77-3.81 (m, 1H), 6.97 (d, 2H), 7.16 (d, 2H), 12.22 (br s, 1H).

### Preparation 174

### Methyl 2-(4-cyclopropoxyphenyl)acetate

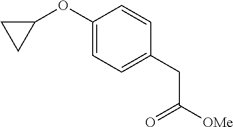 [SMILES: COC(=O)Cc1ccc(OC2CC2)cc1]

To a stirred solution of methyl-1-2-(4-(vinyloxy)phenyl)acetate (Preparation 175, 2 g, 10.4 mmol) in DCE (20 mL) was added chloroiodomethane (1.89 mL, 26 mmol). The reaction was cooled to 0° C. and diethylzinc (1M solution in hexane, 23 mL, 23 mmol) was added. The reaction was stirred at room temperature for 16 hours before quenching with an aqueous solution of ammonia and extracting into EtOAc. The organic layer was collected, washed with water, brine, dried over sodium sulphate and concentrated in vacuo. The residue was purified using silica gel column chromatography eluting with 3-5% EtOAc in hexane to afford the title compound as an oil (1.78 g, 83%).

¹H NMR (400 MHz, DMSO-d₆): δ ppm 0.74-0.76 (m, 4H), 3.56 (s, 2H), 3.67 (s, 3H), 3.68-3.72 (m, 1H), 6.99 (d, 2H), 7.18 (d, 2H).

MS m/z 207 [M+H]⁺

### Preparation 175

### Methyl-1-2-(4-(vinyloxy)phenyl)acetate

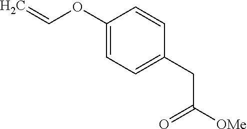

To a stirred solution of methyl-2-(4-hydroxyphenyl)acetate (2 g, 12 mmol) in MeCN (20 mL) was added copper acetate (2.62 g, 14.4 mmol) and the mixture was degassed with oxygen for 15 minutes. Tetravinyl tin (2.64 mL, 14.4 mmol) was added and the reaction stirred at room temperature for 16 hours. The reaction was quenched by the addition of aqueous ammonia solution, filtered through celite and washed through with EtOAc. The combined filtrate was washed with aqueous sodium hydroxide solution, water, brine, dried over sodium sulphate and concentrated in vacuo to afford the title compound as an oil (2 g, 87%).

¹H NMR (400 MHz, DMSO-d₆): δ ppm 3.58 (s, 2H), 3.68 (s, 3H), 4.42 (d, 1H), 4.75 (d, 1H), 6.61 (dd, 1H), 6.95 (d, 2H), 7.22 (d, 2H).

MS m/z 193 [M+H]⁺

### Preparation 176

### 4-Bromo-2-methyl-1-((2-(trimethylsilyl)ethoxy)methyl)-1H-imidazole and 5-Bromo-2-methyl-1-((2-(trimethylsilyl)ethoxy)methyl)-1H-imidazole

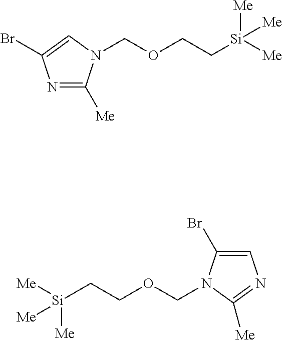 [SMILES: Cc1nc(Br)cn1COCC[Si](C)(C)C ; Cc1ncc(Br)n1COCC[Si](C)(C)C]

To a stirred solution of 4-bromo-2-methyl-1H-imidazole (2 g, 12.42 mmol) in THF (40 mL) was added sodium hydride (60% dispersion in mineral oil, 596 mg, 14.91 mmol) portionwise at 0° C. The reaction was stirred at room temperature for 15 minutes followed by the addition of silylethoxy-methyl-chloride (2.42 mL, 13.66 mmol) dropwise, and then stirred for a further 3 hours. The reaction was quenched by the addition of crushed ice and extracted into EtOAc. The organic layer was collected, washed with water (30 mL), brine (30 mL), dried over sodium sulphate and concentrated in vacuo. The residue was purified using silica gel column chromatography eluting with 1% MeOH in DCM to afford the title compounds as a mixture of regioisomers (3.5 g, 96%).

¹H NMR (400 MHz, DMSO-d₆): δ ppm −0.04 (s, 9H), 0.81-0.86 (m, 2H), 2.29 and 2.36 (2×s, 3H), 3.45-3.53 (m, 2H), 5.22 and 5.26 (2×s, 2H), 6.86 and 7.31 (s, 1H).

4-Bromo-2-methyl-1-((2-(trimethylsilyl)ethoxy)methyl)-1H-imidazole may be isolated as the first eluting major isomer by purification using silica gel column chromatography eluting with 50% EtOAc in hexanes.

¹H NMR (400 MHz, DMSO-d₆): δ ppm −0.02 (s, 9H), 0.84 (t, 3H), 2.29 (s, 3H), 3.49 (t, 2H), 5.22 (s, 2H), 7.31 (s, 1H).

### Preparation 177

### 5-Bromo-2-chloronicotinamide

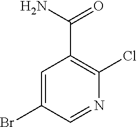 [SMILES: NC(=O)c1cc(Br)cnc1Cl]

To a suspension of 5-bromo-2-chloronicotinic acid (20 g, 84.6 mmol) and oxalyl chloride (12.9 g, 101.5 mmol) in DCM (500 mL) was added DMF (618 mg, 8.46 mmol) at room temperature. The reaction effervesced and was stirred for 3 hours before concentrating to half volume in vacuo. The resulting solution was added carefully to a solution of 7N methanolic ammonia (50 mL) in DCM (200 mL) at −10° C., and the reaction was stirred for 18 hours at room temperature. The reaction was concentrated in vacuo, diluted with EtOAc (1 L), washed with water (500 mL), dried over sodium sulphate and concentrated in vacuo. The residue was triturated with cold DCM (200 mL) to afford the title compound as a colourless solid (16.3 g, 82%).

¹H NMR (400 MHz, DMSO-d₆): δ ppm 7.81 (s, 1H), 8.08 (s, 1H), 8.21 (d, 1H), 8.63 (d, 1H).

### Preparation 178

### Cis-racemic tert-butyl 3-fluoro-4-((methylsulfonyl)oxy)piperidine-1-carboxylate

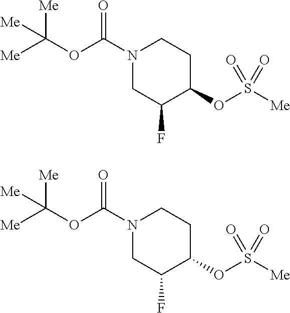 [SMILES: CC(C)(C)OC(=O)N1CC[C@@H](OS(C)(=O)=O)[C@@H](F)C1 ; CC(C)(C)OC(=O)N1CC[C@H](OS(C)(=O)=O)[C@H](F)C1]

To a solution of cis-racemic tert-butyl 3-fluoro-4-hydroxypiperidine-1-carboxylate (500 mg, 2.28 mmol) in DCM (10 mL) was added methanesulfonyl chloride (0.212 mL, 2.74 mmol) followed by triethylamine (0.48 mL, 3.42 mmol) and the reaction was stirred at room temperature for 3 hours. The reaction was diluted with water and extracted into DCM. The organic layer was collected, washed with saturated aqueous NaHCO₃ solution, brine, dried over sodium sulphate and concentrated in vacuo to afford the title compound that was used directly in the next reaction.

### Preparation 179

### (1R,3s,5S)-tert-butyl 3-((methylsulfonyl)oxy)-8-azabicyclo[3.2.1]octane-8-carboxylate

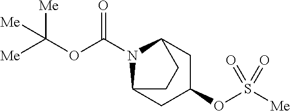 [SMILES: CC(C)(C)OC(=O)N1[C@@H]2CC[C@H]1C[C@H](OS(C)(=O)=O)C2]

The title compound was prepared according to the method described for Preparation 178 using (1R,3s,5S)-tert-butyl-3-hydroxy-8-azabicyclo[3.2.1]octane-8-carboxylate. Taken on directly to the next step.

### Preparation 180

### Trans-racemic tert-butyl 3-fluoro-4-((methylsulfonyl)oxy)piperidine-1-carboxylate

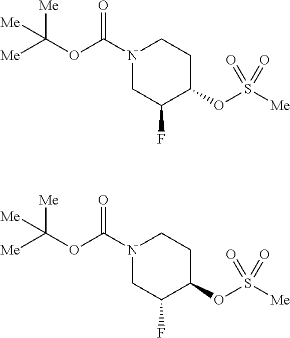 [SMILES: CC(C)(C)OC(=O)N1CC[C@H](OS(C)(=O)=O)[C@@H](F)C1 ; CC(C)(C)OC(=O)N1CC[C@@H](OS(C)(=O)=O)[C@H](F)C1]

The title compound was prepared according to the method described for Preparation 178 using trans-racemic tert-butyl 3-fluoro-4-hydroxypiperidine-1-carboxylate. Taken on directly to the next step.

### Preparation 181

### 1-{[4-(trifluoromethoxy)phenyl]acetyl}piperidin-4-yl methanesulfonate

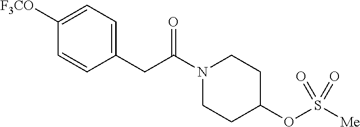 [SMILES: CS(=O)(=O)OC1CCN(C(=O)Cc2ccc(OC(F)(F)F)cc2)CC1]

The title compound was prepared according to the method described for Preparation 178 using 1-(4-hydroxypiperidin-1-yl)-2-(4-(trifluoromethoxy)phenyl)ethanone (Preparation 182). Taken on directly to the next step.

### Preparation 182

### 1-(4-hydroxypiperidin-1-yl)-2-(4-(trifluoromethoxy)phenyl)ethanone

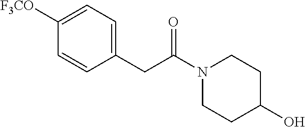 [SMILES: O=C(Cc1ccc(OC(F)(F)F)cc1)N1CCC(O)CC1]

To a solution of 4-hydroxypiperidine (14.4 g, 143 mmol) in DCM (200 mL) at 0° C. was added triethylamine (20 mL, 143 mmol) followed by 2-(4-(trifluoromethoxy)phenyl)acetyl chloride (17.0 g, 71.3 mmol) drop-wise keeping the temperature below 10° C. Once the addition was complete the cold bath was removed and the reaction stirred for 3 hours. The reaction was quenched with water (200 mL). The organic layer was separated, washed with 2M HCl (200 mL), saturated NaHCO₃ (200 mL) and brine (200 mL), dried over Na₂SO₄ and concentrated in vacuo. The residue was purified by silica gel column chromatography eluting with on silica 10% MeOH in DCM to afford the title compound (7.35 g, 34%).

¹H NMR (400 MHz, DMSO-d₆): δ ppm 1.29-1.41 (m, 1H), 1.42-1.54 (m, 1H), 1.67-1.70 (m, 1H), 1.79-1.90 (m, 1H), 3.15-3.31 (m, 2H), 3.64-3.76 (m, 3H), 3.85-3.94 (m, 1H), 2.02-4.10 (m, 1H), 7.16 (d, 2H), 7.26 (d, 2H).

### Preparation 183

### 2-hydroxy-5-(4,4,5,5-tetramethyl-1,3,2-dioxaborolan-2-yl)benzamide

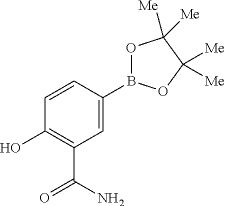 [SMILES: CC1(C)OB(c2ccc(O)c(C(N)=O)c2)OC1(C)C]

The title compound was prepared according to the method described for Preparation 12 using 5-bromo-2-hydroxybenzamide.

¹H NMR (400 MHz, CD₃OD): δ ppm 1.34 (s, 12H), 6.89 (d, 1H), 7.72-7.79 (m, 1H), 8.18 (d, 1H).

### Preparation 184

### 3-[(4-bromo-1H-pyrazol-1-yl)methyl]oxetan-3-ol

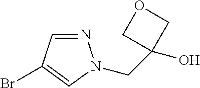 [SMILES: OC1(Cn2cc(Br)cn2)COC1]

To a suspension of sodium hydride (400 mg, 60% dispersion in oil, 10 mmol) in dry dimethyl sulfoxide (7 ml) was added trimethylsulfoxonium iodide (2.2 g, 10 mmol). The mixture was stirred at room temperature for 1 hour, then cooled to 0° C. and oxetane-3-one (720 mg, 10 mmol) was added. The reaction was stirred at 0° C. for 1 hour, then 4-bromopyrazole (1.47 g, 10 mmol) was added and the mixture allowed to warm to room temperature over 3 hours. The reaction was poured into ethyl acetate (30 mL) and water (30 mL), the organic layer was washed with water (10 mL), brine (10 mL) dried over sodium sulphate and concentrated in vacuo. The residue was purified using silica gel column chromatography eluting with 5% methanol in dichloromethane followed a second chromatography eluting with 50% ethyl acetate in heptane to afford the title compound as a clear colourless oil. (480 mg, 21%).

¹H NMR (400 MHz, CDCl₃): δ ppm 4.40 (d, 2H), 4.50 (s, 2H), 4.65 (d, 2H), 7.50, (s, 1H), 7.55 (s, 1H).

MS m/z 233 [M⁷⁹Br+H]⁺

### Preparation 185

### Racemic 1-(1,1-dioxidotetrahydrothiophen-3-yl)-4-(4,4,5,5-tetramethyl-1,3,2-dioxaborolan-2-yl)-1H-pyrazole

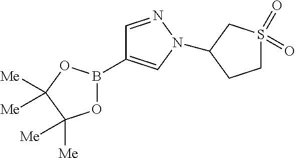 [SMILES: CC1(C)OB(c2cnn(C3CCS(=O)(=O)C3)c2)OC1(C)C]

The title compound was prepared according to the method described for Preparation 169 using 3-bromotetrahydrothiophene1,1-dioxide.

¹H NMR (400 MHz, DMSO-d₆): δ ppm 1.22 (s, 12H), 2.49-2.66 (m, 2H), 3.21-3.25 (m, 1H), 3.37-3.48 (m, 2H), 3.79 (m, 1H), 5.25 (m, 1H), 7.67 (s, 1H), 8.08 (s, 1H).

MS m/z 313 [M+H]⁺

### Preparation 186

### 3-(5-bromo-1H-imidazol-2-yl)oxetan-3-ol

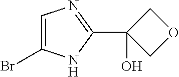 [SMILES: OC1(c2ncc(Br)[nH]2)COC1]

Acrylic acid (25 mL, 364 mmol) was added to a suspension of 3-{5-bromo-1-[(4-methoxyphenyl)(diphenyl)methyl]-1H-imidazol-2-yl}oxetan-3-ol (Preparation 187, 11.8 g, 24.01 mmol) in dichloromethane (100 mL) and the reaction was stirred at room temperature for 18 hours. The resulting white solid was filtered and dried to afford the title compound (3.78 g, 72%).

¹H NMR (400 MHz, DMSO-d₆): δ ppm 4.59-4.84 (m, 4H), 6.69 (s, 1H), 7.17 (s, 1H), 12.34 (s, 1H).

MS m/z 219 [M⁷⁹Br+H]⁺

### Preparation 187

### 3-{5-bromo-1-[(4-methoxyphenyl)(diphenyl)methyl]-1H-imidazol-2-yl}oxetan-3-ol

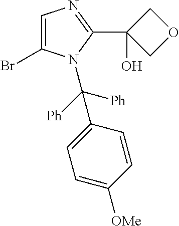 [SMILES: COc1ccc(C(c2ccccc2)(c2ccccc2)n2c(Br)cnc2C2(O)COC2)cc1]

To a solution of 5-bromo-1-[(4-methoxyphenyl)(diphenyl)methyl]-1H-imidazole (Preparation 188, 24 g, 57.23 mmol) in THF (300 mL) was added a 2.5M solution of n-butyl lithium in THF (25.2 mL, 62.96 mmol) at 0° C. and the reaction was stirred at this temperature for 30 minutes. A solution of 3-oxetanone (3.7 mL, 62.96 mmol) in THF (10 mL) was added and the reaction was stirred for a further 30 minutes at 0° C. followed by room temperature for 1.5 hours. The reaction mixture was quenched with saturated NH₄Cl (200 mL), diluted with water (500 mL) and extracted into ethyl acetate (2×500 mL). The combined organic layers were dried over MgSO₄ and concentrated in vacuo. The residue was purified using silica gel column chromatography eluting with 50% ethyl acetate in heptanes to afford the title compound as cream solid (11.84 g, 42%).

¹H NMR (400 MHz, CDCl₃): δ ppm 3.65 (d, 2H), 3.81 (s, 3H), 4.81 (d, 2H), 6.77 (s, 1H), 6.84-6.87 (m, 2H), 6.99-7.02 (m, 2H), 7.10-7.14 (m, 4H), 7.34-7.37 (m, 6H).

### Preparation 188

### 5-bromo-1-[(4-methoxyphenyl)(diphenyl)methyl]-1H-imidazole

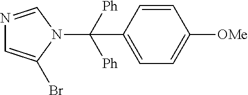 [SMILES: COc1ccc(C(c2ccccc2)(c2ccccc2)n2cncc2Br)cc1]

(Chloro(4-methoxyphenyl)methylene)dibenzene (24 g, 78 mmol) was added portion-wise to a solution of 4-bromo-1H-imidazole (10 g, 68 mmol) in DMF (100 mL) and the reaction was stirred at room temperature for 1 hour. The reaction mixture was diluted with water (3×400 mL), extracted with dichloromethane (400 mL), dried over MgSO₄ and concentrated in vacuo. The residue was triturated with mixture of TBDME and heptanes (1:4) to afford the title compound as off white solid (24.04 g, 84%).

¹H NMR (400 MHz, DMSO-d₆): δ ppm 3.75 (s, 3H), 6.94-7.00(m, 5H), 7.06-7.08 (m, 4H), 7.35-7.42 (m, 7H).

### Preparation 189

### 4-bromo-1-(oxetan-3-yl)-1H-pyrazole

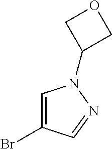

To a suspension of 4-bromopyrazole (3.35 g, 22.8 mmol) and cesium carbonate (8.17 g, 25.2 mmol) in DMF (40 mL) was added a solution of oxetan-3-yl trifluoromethanesulfonate (4.7 g, 22.8 mmol) in DMF (10 mL) at 10° C. The reaction was warmed to room temperature for 48 hours. The reaction was partitioned between ethyl acetate (100 mL) and water (100 mL). The aqueous layer was extracted with ethyl acetate (75 mL), the organic layers combined, washed with brine (2×75 mL), water (2×70 mL), dried over sodium sulfate and concentrated in vacuo. The residue was suspended in TBME:heptane (1:1, 30 mL) and cooled to 5° C. for 1.5 hours. The resulting solid was filtered, washed with cold heptane (5 mL) and dried to afford the title compound (0.85 g, 19%).

¹H NMR (400 MHz, CDCl₃): δ ppm 4.92-5.10 (m, 4H), 5.37-5.48 (m, 1H), 7.56 (s, 1H) and 7.62 (s, 1H).

MS m/z 203 [M⁷⁹Br+H]⁺

### Preparation 190

### 1-{[(4R)-2,2-dimethyl-1,3-dioxolan-4-yl]methyl}-4-(4,4,5,5-tetramethyl-1,3,2-dioxaborolan-2-yl)-1H-pyrazole

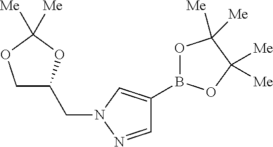 [SMILES: CC1(C)OC[C@@H](Cn2cc(B3OC(C)(C)C(C)(C)O3)cn2)O1]

The title compound was prepared according to the method described for Preparation 169 using [(4S)-2,2-dimethyl-1,3-dioxolan-4-yl]methyl methanesulfonate (Preparation 194).

MS m/z 309 [M+H]⁺

### Preparation 191

### 4-bromo-1-{[(4R)-2,2-dimethyl-1,3-dioxolan-4-yl]methyl}-3,5-dimethyl-1H-pyrazole

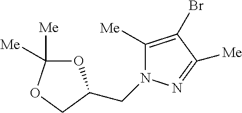 [SMILES: Cc1nn(C[C@@H]2COC(C)(C)O2)c(C)c1Br]

The title compound was prepared according to the method described for Preparation 169 using [(4S)-2,2-dimethyl-1,3-dioxolan-4-yl]methyl methanesulfonate (Preparation 194) and 4-4-bromo-3,5-dimethyl-1H-pyrazole in DMF at 100° C.

¹H NMR (400 MHz, DMSO-d₆): δ ppm 1.23 (s, 3H), 1.26 (s, 3H), 2.06 (s, 3H), 2.22 (s, 3H), 3.73-3.76 (m, 1H), 4.00-4.04 (m, 1H), 4.06-4.16 (m, 2H), 4.23-4.34 (m, 1H).

### Preparation MGX2 192

### 4-bromo-1-{[(4S)-2,2-dimethyl-1,3-dioxolan-4-yl]methyl}-3,5-dimethyl-1H-pyrazole

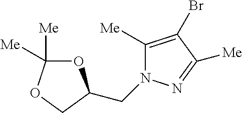 [SMILES: Cc1nn(C[C@H]2COC(C)(C)O2)c(C)c1Br]

The title compound was prepared according to the method described for Preparation 169 using [(4R)-2,2-dimethyl-1,3-dioxolan-4-yl]methyl methanesulfonate (Preparation 195) and 4-bromo-3,5-dimethyl-1H-pyrazole in DMF at 100° C.

¹H NMR (400 MHz, DMSO-d₆): δ ppm 1.23 (s, 3H), 1.26 (s, 3H), 2.06 (s, 3H), 2.22 (s, 3H), 3.73-3.76 (m, 1H), 4.00-4.04 (m, 1H), 4.06-4.16 (m, 2H), 4.23-4.34 (m, 1H).

### Preparation 193

### 4-bromo-1-{[(4S)-2,2-dimethyl-1,3-dioxolan-4-yl]methyl}-1H-imidazole

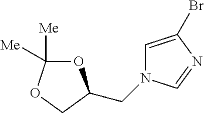 [SMILES: CC1(C)OC[C@H](Cn2cnc(Br)c2)O1]

The title compound was prepared according to the method described for Preparation 169 using [(4R)-2,2-dimethyl-1,3-dioxolan-4-yl]methyl methanesulfonate (Preparation 195) and 4-bromoimidazole.

¹H NMR (400 MHz, DMSO-d₆): δ ppm 1.24 (s, 3H), 1.30 (s, 3H), 3.62-3.66 (m, 1H), 3.98-4.05 (m, 1H), 4.11-4.16 (m, 1H), 4.29 (s, 1H), 4.31-4.35 (m, 1H), 7.30 (s, 1H), 7.61 (s, 1H).

### Preparation 194

### [(4S)-2,2-dimethyl-1,3-dioxolan-4-yl]methyl methanesulfonate

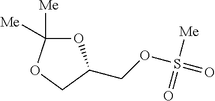 [SMILES: CC1(C)OC[C@@H](COS(C)(=O)=O)O1]

To a stirred solution of (R)-2,2-dimethyl-1,3-dioxolane-4-methanol (500 mg, 3.79 mmol) and triethylamine (0.8 mL, 5.68 mmol) in anhydrous DCM (10 mL) was added methanesulfonyl chloride (0.5 mL, 4.54 mmol) drop-wise at 0° C. under nitrogen. The reaction was warmed to room temperature and stirred for 3 hours before diluting with DCM (10 mL). The solution was washed with saturated NaHCO₃ solution (2×10 mL), water (10 mL), brine (10 mL), dried over sodium sulfate and concentrated in vacuo to afford the title compound as light yellow oil (750 mg, 94%).

¹H NMR (400 MHz, DMSO-d₆): δ ppm 1.28 (s, 3H), 1.35 (s, 3H), 3.19 (s, 3H), 3.68-3.72 (m, 1H), 4.01-4.05 (m, 1H), 4.12-4.16 (m, 1H), 4.23-4.27 (m, 1H), 4.31-4.33 (m, 1H).

### Preparation 195

### [(4R)-2,2-dimethyl-1,3-dioxolan-4-yl]methyl methanesulfonate

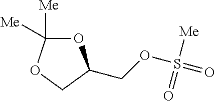 [SMILES: CC1(C)OC[C@H](COS(C)(=O)=O)O1]

The title compound was prepared according to the method described for Preparation 194 using (S)-2,2-dimethyl-1,3-dioxolane-4-methanol.

¹H NMR (400 MHz, DMSO-d₆): δ ppm 1.28 (s, 3H), 1.35 (s, 3H), 3.19 (s, 3H), 3.68-3.72 (m, 1H), 4.01-4.05 (m, 1H), 4.12-4.16 (m, 1H), 4.23-4.27 (m, 1H), 4.31-4.33 (m, 1H).

### Preparation 196

### 5-bromo-1-{[2-(trimethylsilyl)ethoxy]methyl}-1H-imidazole and 4-bromo-1-{[2-(trimethylsilyl)ethoxy]methyl}-1H-imidazole

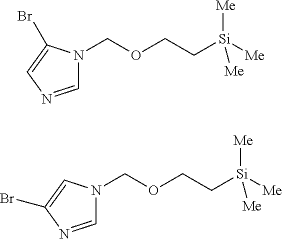 [SMILES: C[Si](C)(C)CCOCn1cncc1Br ; C[Si](C)(C)CCOCn1cnc(Br)c1]

The title compounds were prepared as a mixture of isomers according to the method described for Preparation 177 using 5-bromoimidazole.

¹H NMR (400 MHz, DMSO-d₆): δ ppm (mixture of regioisomers) −0.03 (s, 9H), 0.83 (t, 2H), 3.45-3.51 (m, 2H), 5.29 (s, 2H), 7.02 (s, 0.45H), 7.43 (s, 0.55H), 7.79 (s, 0.55H), 7.98 (s, 0.45H).

### Preparation 197

### Methyl(4-iodo-1H-imidazol-1-yl)acetate

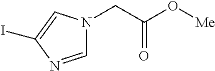 [SMILES: COC(=O)Cn1cnc(I)c1]

Concentrated sulphuric acid (25.7 mL) was added to a solution of (4-iodo-1H-imidazol-1-yl)acetic acid (Preparation 198, 3.38 g, 13.4 mmol) in methanol (220 mL). The reaction was heated to 100° C. under nitrogen for 2 hours. The reaction was cooled to room temperature and concentrated to ˜50 mL and diluted with water (100 mL). The resulting solution was neutralised by the careful addition of sodium bicarbonate until pH=6. The solution was extracted with ethyl acetate (2×100 mL) and dichloromethane (100 mL). The organic extract was dried over magnesium sulfate, filtered and concentrated in vacuo. The residue was purified using reverse phase column chromatography eluting with 20-40% acetonitrile in water with 0.1% formic acid to afford the title compound as a yellow oil (701 mg, 20%).

¹H NMR (400 MHz, CDCl₃):δ ppm 3.78 (s, 3H), 4.68 (s, 2H), 7.03 (s, 1H), 7.46 (s, 1H).

### Preparation 198

### (4-iodo-1H-imidazol-1-yl)acetic acid

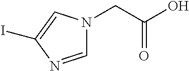

A mixture of tert-butyl (4,5-diiodo-1H-imidazol-1-yl)acetate (Preparation 199, 6.31 g, 14.5 mmol) and sodium sulphite (36.8 g, 292 mmol) in water (40 mL) and methanol (40 mL) was heated to 100° C. for 4 days under nitrogen. The reaction was cooled to room temperature and concentrated in vacuo. The resulting solid was slurried in isopropyl alcohol (600 mL) at room temperature for 16 hours. The suspension was filtered and the organic filtrate concentrated in vacuo to afford the title compound that was used directly in the next reaction.

### Preparation 199

### tert-butyl(4,5-diiodo-1H-imidazol-1-yl)acetate

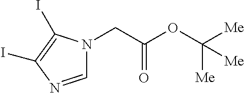 [SMILES: CC(C)(C)OC(=O)Cn1cnc(I)c1I]

To a mixture of 4,5-diiodo-1H-imidazole (5.01 g, 15.7 mmol) in acetonitrile (100 mL) was added potassium carbonate (4.34 g, 31.4 mmol) followed by tert-butyl bromoacetate (2.52 mmol, 17.2 mmol). The reaction was stirred for 2 hours at room temperature under nitrogen. The reaction was filtered and the organic filtrate was concentrated in vacuo. The residue was diluted with water (100 mL) and extracted with ethyl acetate (3×50 mL). The combined organic extract was washed with water (50 mL) and brine (50 mL), dried over magnesium sulfate, filtered and concentrated in vacuo to afford oil which solidified upon standing. The solid was then stirred with heptane (30 mL) for 24 hours before filtering and drying to afford filtered off and air dried to afford the title compound as a colourless solid (6.31 g, 93%).

¹H NMR (400 MHz, CDCl₃): δ ppm 1.46 (s, 9H), 4.60 (s, 2H), 7.62 (s, 1H).

### Preparation 200

### [5-(trifluoromethoxy)pyridin-2-yl]acetic acid

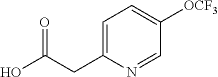 [SMILES: O=C(O)Cc1ccc(OC(F)(F)F)cn1]

A solution of tert-butyl cyano[5-(trifluoromethoxy)pyridin-2-yl]acetate (Preparation 201, 16 mg, 0.053 mmol) in acetic acid (0.2 mL) and concentrated aqueous HCl (0.3 mL) was heated at 100° C. for 1 hour. The reaction was cooled and concentrated in vacuo, azeotroping with toluene and DCM to afford the title compound (12 mg, 89%).

MS m/z 222 [M+H]⁺

### Preparation 201

### tert-butyl cyano[5-(trifluoromethoxy)pyridin-2-yl]acetate

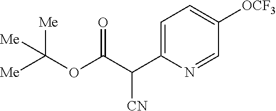 [SMILES: CC(C)(C)OC(=O)C(C#N)c1ccc(OC(F)(F)F)cn1]

To a solution of 2-chloro-5-(trifluoromethoxy)pyridine (94 mg, 0.44 mmol) in dioxane (1 mL) was added tris(dibenzylideneacetone)dipalladium (0) (8.20 mg, 0.009 mmol). 2,8,9-tris(2-methylpropyl)-2,5,8,9-tetraaza-1-phosphabicyclo[3.3.3]undecane (12 mg, 13 uL, 0.036 mmol) followed by tert-butylcyanoacetate (70 uL, 0.488 mmol) were added and the reaction degassed with nitrogen before heating to 90° C. for 18 hours. The reaction was cooled, concentrated in vacuo and diluted with DCM. The solution was washed with water, dried over sodium sulphate and concentrated in vacuo. The residue was purified using silica gel column chromatography eluting with 0-40% EtOAc in heptanes to afford the title compound (32 mg, 24%).

MS m/z 301 [M−H]⁻

### Preparation 202

### (2S)-1-{[tert-butyl(diphenyl)silyl]oxy}propan-2-yl methanesulfonate

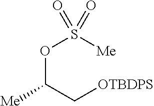

To a solution of (2S)-1-{[tert-butyl(diphenyl)silyl]oxy}propan-2-ol (280 mg, 0.891 mmol) in DCM (5 mL) at 0° C. was added triethylamine (186 uL, 1.336 mmol) followed by mesyl chloride (83 uL, 1.07 mmol) and the reaction was stirred at room temperature for 2 hours. The reaction was diluted with DCM, washed with water, brine, dried over sodium sulphate and concentrated in vacuo. The residue was used directly in the next step as the title compound.

¹H NMR (400 MHz, DMSO-d₆): δ ppm 1.01 (s, 9H), 1.33 (d, 3H), 3.12 (s, 3H), 3.71 (m, 2H), 4.82 (m, 1H), 7.38-7.50 (m, 6H), 7.63-7.69 (m, 4H).

### Preparation 203

### (2R)-1-{[tert-butyl(diphenyl)silyl]oxy}propan-2-yl methanesulfonate

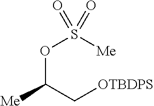

The title compound was prepared according to the method described for Preparation 202 using of (2R)-1-{[tert-butyl(diphenyl)silyl]oxy}propan-2-ol.

¹H NMR (400 MHz, DMSO-d₆): δ ppm 1.01 (s, 9H), 1.33 (d, 3H), 3.12 (s, 3H), 3.71 (m, 2H), 4.82 (m, 1H), 7.38-7.50 (m, 6H), 7.63-7.69 (m, 4H).

### Preparation 204

### N,N-dibenzyl-2-[4-(4,4,5,5-tetramethyl-1,3,2-dioxaborolan-2-yl)-1H-pyrazol-1-yl]ethanamine

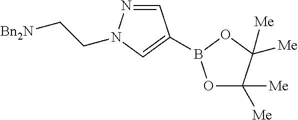

To a mixture of 2-(dibenzylamino)ethyl methanesulfonate (Preparation 205, 592 mg, 1.86 mmol) and 4-(4,4,5,5-tetramethyl-1,3,2-dioxaborolan-2-yl)-1H-pyrazole (300 mg, 1.54 mmol) in DMF (6 mL) was added cesium carbonate (755 mg, 2.31 mmol) and the reaction was heated to 100° C. for 12 hours. The reaction was cooled, diluted with EtOAc, washed with water, brine, dried over sodium sulphate and concentrated in vacuo. The residue was used directly in the next step.

### Preparation 205

### 2-(dibenzylamino)ethyl methanesulfonate

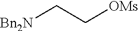

The title compound was prepared according to the method described for Preparation 202 using 2-(dibenzylamino)ethanol.

¹H NMR (400 MHz, DMSO-d₆): δ ppm 2.36 (s, 3H), 3.30 (m, 2H), 3.96 (m, 2H), 4.38-4.42 (m, 4H), 7.47 (m, 6H), 7.59 (m, 4H).

### Preparation 206

### 2-(4-bromo-1H-pyrazol-1-yl)-2-methylpropan-1-ol

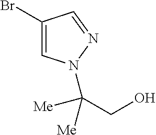 [SMILES: CC(C)(CO)n1cc(Br)cn1]

To a solution of methyl 2-(4-bromo-1H-pyrazol-1-yl)-2-methylpropanoate (Preparation 207, 650 mg, 2.63 mmol) in EtOH (13 mL) was added sodium borohydride (299 mg, 7.89 mmol) and the reaction was stirred at room temperature for 2 hours before concentrating in vacuo. The residue was dissolved in EtOAc, washed with water, brine, dried over sodium sulphate and concentrated in vacuo to afford the title compound as a colourless solid (500 mg, 93%).

¹H NMR (400 MHz, DMSO-d₆): δ ppm 1.43 (s, 6H), 3.58 (d, 2H), 4.96 (t, 1H), 7.51 (s, 1H), 7.96 (s, 1H).

### Preparation 207

### Methyl 2-(4-bromo-1H-pyrazol-1-yl)-2-methylpropanoate

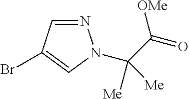 [SMILES: COC(=O)C(C)(C)n1cc(Br)cn1]

To a solution of 4-bromopyrazole (500 mg, 3.42 mmol) in DMF (10 mL) was added methyl 2-bromo-2-methylpropanoate (930 mg, 5.14 mmol) and cesium carbonate (3.34 g, 10.28 mmol). The reaction was heated to 80° C. for 18 hours before cooling and diluting with EtOAc. The solution was washed with water, brine, dried over sodium sulphate and concentrated in vacuo. The residue was purified using silica gel column chromatography eluting with 10% EtOAc in hexane to afford the title compound as a light yellow foam (650 mg, 77%).

¹H NMR (400 MHz, DMSO-d₆): δ ppm 1.74 (s, 6H), 3.61 (s, 3H), 7.59 (s, 1H), 8.19 (s, 1H).

### Preparation 208

### 5-bromo-2-chloro-6-methylpyridine-3-carbonitrile

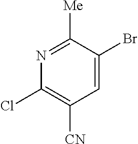 [SMILES: Cc1nc(Cl)c(C#N)cc1Br]

Phosphorous oxychloride (10 mL) was added dropwise to 5-bromo-2-hydroxy-6-methylpyridine-3-carboxamide (Preparation 209, 1.2 g, 5.194 mmol) and the resultant reaction was stirred at 80° C. for 16 hours. The reaction was cooled, concentrated in vacuo and basified with saturated aqueous sodium bicarbonate solution with cooling. The solution was extracted with EtOAc twice, the organic layers were collected, combined, washed with water, brine, dried over sodium sulphate and concentrated in vacuo to afford the title compound as a brown solid (900 mg, 75%).

¹H NMR (400 MHz, DMSO-d₆): δ ppm 2.63 (s, 3H), 8.79 (s, 1H).

### Preparation 209

### 5-Bromo-2-hydroxy-6-methylpyridine-3-carboxamide

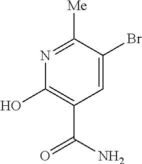 [SMILES: Cc1nc(O)c(C(N)=O)cc1Br]

To a solution of ethyl 5-bromo-2-hydroxy-6-methylpyridine-3-carboxylate (Preparation 210, 1.1 g, 4.231 mmol) in MeOH (7 mL) was added methanolic ammonia (15 mL) dropwise with cooling. The reaction was stirred at room temperature for 16 hours. The reaction was concentrated in vacuo and the residue washed with hexane to afford the title compound as a light brown solid (900 mg, 92%).

¹H NMR (400 MHz, DMSO-d₆): δ ppm 2.36 (s, 3H), 7.64 (br s, 1H), 8.25 (s, 1H), 8.89 (br s, 1H).

### Preparation 210

### Ethyl 5-bromo-2-hydroxy-6-methylpyridine-3-carboxylate

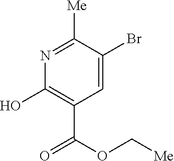 [SMILES: CCOC(=O)c1cc(Br)c(C)nc1O]

To a stirred solution of ethyl 2-hydroxy-6-methylpyridine-3-carboxylate (Preparation 211, 1.08 g, 5.967 mmol) in DMF (10 mL) was added NBS (1.16 g, 6.564 mmol) portionwise and the reaction was allowed to stir at room temperature for 1 hour. The reaction mixture was diluted with ethyl acetate, washed with water, brine, dried over sodium sulfate and concentrated in vacuo to afford the title compound as a white solid (1.2 g, 77%).

¹H NMR (400 MHz, DMSO-d₆): δ ppm 1.25 (t, 3H), 2.33 (s, 3H), 4.18 (q, 2H), 8.05 (s, 1H), 12.50 (br s, 1H).

### Preparation 211

### ethyl 2-hydroxy-6-methylpyridine-3-carboxylate

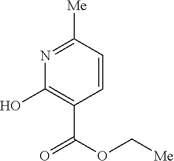 [SMILES: CCOC(=O)c1ccc(C)nc1O]

To a solution of 2-hydroxy-6-methylpyridine-3-carboxylic acid (2.0 g, 13.06 mmol) in ethanol (20 mL), was added HCl in dioxane (10 mL) dropwise and the reaction was refluxed for 16 hours. The reaction was concentrated in vacuo and basified with saturated aqueous sodium bicarbonate solution. The mixture was extracted with ethyl acetate (twice), the organic layers were combined, washed with water, brine, dried over sodium sulfate and concentrated in vacuo to afford the title compound as a white solid (650 mg, 27%).

¹H NMR (400 MHz, DMSO-d₆): δ ppm 1.24 (t, 3H), 2.22 (s, 3H), 4.16 (q, 2H), 6.08 (d, 1H), 7.97 (d, 1H).

### Preparation 211

### [5-(cyclopropyloxy)pyridin-2-yl]acetic acid

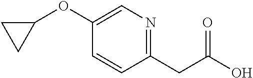 [SMILES: O=C(O)Cc1ccc(OC2CC2)cn1]

To a solution of ethyl [5-(cyclopropyloxy)pyridin-2-yl]acetate (Preparation 213, 1.16 g, 5.249 mmol) in MeOH:THF (1:1, 17 mL) was added water (17 mL) and LiOH.H₂O (726 mg, 17.321 mmol) at room temperature. The reaction was stirred at room temperature for 1 hour. The reaction was concentrated in vacuo, diluted with water and extracted with DCM (twice). The aqueous layer was then acidified to pH 4-5 with 1N HCl solution and extracted with 10% IPA/DCM (twice). The combined organic layers were washed with brine, dried over sodium sulfate and concentrated in vacuo to afford the title compound (700 mg, 69%).

¹H NMR (400 MHz, DMSO-d₆): δ ppm 0.69 (m, 2H), 0.82 (m, 2H), 3.66 (s, 2H), 3.94 (m, 1H), 7.29 (d, 1H), 4.46 (dd, 1H), 8.25 (d, 1H).

MS m/z 194 [M+H]⁺

### Preparation 213

### Ethyl [5-(cyclopropyloxy)pyridin-2-yl]acetate

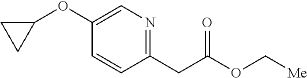 [SMILES: CCOC(=O)Cc1ccc(OC2CC2)cn1]

n-BuLi (1.5M, 15 mL) was added drop wise to a solution of diisopropylamine (3.32 mL, 23.80 mmol) in THF (40 mL) at 0° C. The solution was stirred for 10 minutes and transferred drop-wise via cannula to a solution of 5-(cyclopropyloxy)-2-methylpyridine (PCT Intl. Appl. 2010125811, 960 mg, 6.435 mmol) and diethyl carbonate (4.1 mL, 32.174 mmol) at −78° C. The orange solution was stirred at −78° C. for 2.5 hours. The mixture was quenched with saturated aqueous NH₄Cl solution at −78° C. and the mixture was extracted with EtOAc (3×150 mL). The combined organic layers were washed with brine, dried over sodium sulfate and concentrated in vacuo. The residue was purified by silica gel column chromatography eluting with 10-15% Ethyl acetate in hexane to afford the title compound (1.16 g, 81%).

¹H NMR (400 MHz, DMSO-d₆): δ ppm 0.67 (m, 2H), 0.80 (m, 2H), 1.17 (t, 3H), 3.75 (s, 2H), 3.94 (m, 1H), 4.10 (q, 2H), 7.28-7.30 (d, 1H), 7.45-7.48 (dd, 1H), 8.26 (d, 1H).

MS m/z 222 [M+H]⁺

### Preparation 214

### 2-methyl-1-[3-(4,4,5,5-tetramethyl-1,3,2-dioxaborolan-2-yl)-1H-pyrazol-1-yl]propan-2-ol

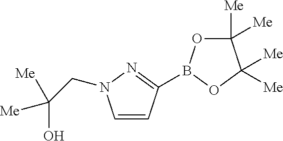 [SMILES: CC(C)(O)Cn1ccc(B2OC(C)(C)C(C)(C)O2)n1]

To a solution of 4-(4,4,5,5-tetramethyl-1,3,2-dioxaborolan-2-yl)-1H-pyrazole (2.0 g, 10.308 mmol) in acetonitrile (40 mL) were added Cs₂CO₃ (6.7 g, 20.615 mmol) and isobutylene oxide (3.71 g, 51.538 mmol) at room temperature. The reaction was heated in a sealed tube at 80° C. for 16 hours. The reaction was poured onto ice water and extracted with ethyl acetate (twice). The combined ethyl acetate layers were washed with water, brine, dried over sodium sulfate and concentrated in vacuo to afford the title compound (1.2 g, 43%).

¹H NMR (400 MHz, DMSO-d₆): δ ppm 1.04 (s, 6H), 1.25 (s, 12H), 4.03 (s, 2H), 4.65 (s, 1H), 7.55 (d, 1H), 7.83 (d, 1H).

### Preparation 215

### Azidomethylpivalate

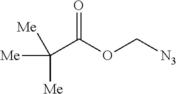 [SMILES: CC(C)(C)C(=O)OCN=[N+]=[N-]]

To a solution of chloromethylpivalate (1.46 mL, 10.13 mmol) in water (2 mL) was added sodium azide (1 g, 15.31 mmol) and the reaction was stirred at 90° C. for 16 hours. The reaction was cooled, diluted with DCM, washed with water, brine, dried over sodium sulphate and concentrated in vacuo to afford the title compound that was used directly in the next reaction (760 mg, 47%).

¹H NMR (400 MHz, CDCl₃): δ ppm 1.22 (s, 9H), 5.12 (s, 2H).

### Preparation 216

### 2-methyl-1-(4-(4,4,5,5-tetramethyl-1,3,2-dioxaborolan-2-yl)-1H-pyrazol-1-yl)propan-2-ol

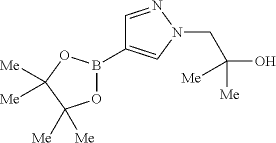 [SMILES: CC(C)(O)Cn1cc(B2OC(C)(C)C(C)(C)O2)cn1]

A suspension of 4-(4,4,5,5-tetramethyl-1,3,2-dioxaborolan-2-yl)-1H-pyrazole (2 g, 10.31 mmol), 2,2-dimethyloxirane (3.70 g, 51 mmol) and cesium carbonate (6.70 g, 20.6 mmol) in MeCN (40 mL) was heated to 60° C. for 16 hours. The reaction was cooled and concentrated in vacuo. The residue was dissolved in EtOAc, washed with brine, dried over sodium sulphate and concentrated in vacuo to afford the title compound that was used directly in the next reaction.

### Preparation 217

### 2-{[(3R,4S)-3-fluoro-1-{[4-(trifluoromethoxy)phenyl]acetyl}piperidin-4-yl]oxy}-5-(1-{[2-(trimethylsilyl)ethoxy]methyl}-1H-imidazol-4-yl)pyridine-3-carboxamide

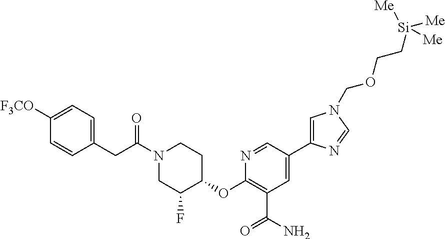 [SMILES: C[Si](C)(C)CCOCn1cnc(-c2cnc(O[C@H]3CCN(C(=O)Cc4ccc(OC(F)(F)F)cc4)C[C@H]3F)c(C(N)=O)c2)c1]

The title compound was prepared as described in the method for Preparation 2 using 2-{[(3R,4S)-3-fluoro-1-{[4-(trifluoromethoxy)phenyl]acetyl}piperidin-4-yl]oxy}-5-(4,4,5,5-tetramethyl-1,3,2-dioxaborolan-2-yl)pyridine-3-carboxamide (Preparation 218) and 4-bromo-1-(2-trimethylsilanyl-ethoxymethyl)-1H-imidazole (WO2007/072080). Taken on directly to the next step.

MS m/z 638 [M+H]⁺

### Preparation 218

### 2-{[(3R,4S)-3-fluoro-1-{[4-(trifluoromethoxy)phenyl]acetyl}piperidin-4-yl]oxy}-5-(4,4,5,5-tetramethyl-1,3,2-dioxaborolan-2-yl)pyridine-3-carboxamide

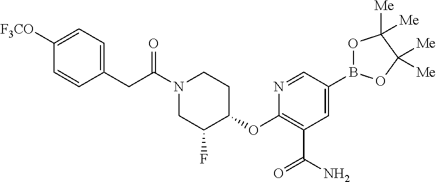 [SMILES: CC1(C)OB(c2cnc(O[C@H]3CCN(C(=O)Cc4ccc(OC(F)(F)F)cc4)C[C@H]3F)c(C(N)=O)c2)OC1(C)C]

The title compound was prepared as described in the method for Preparation 12 using 5-bromo-2-{[(3R,4S)-3-fluoro-1-{[4-(trifluoromethoxy)phenyl]acetyl}piperidin-4-yl]oxy}pyridine-3-carboxamide (Preparation 26).

MS m/z 568 [M+H]⁺

### Preparation 219

### 6-chloro-3-(((3S,4R)-3-fluoro-1-(2-(4-(trifluoromethoxy)phenyl)acetyl)piperidin-4-yl)oxy)pyridazine-4-carboxamide

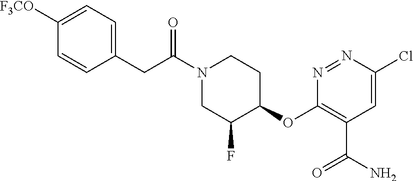 [SMILES: NC(=O)c1cc(Cl)nnc1O[C@@H]1CCN(C(=O)Cc2ccc(OC(F)(F)F)cc2)C[C@@H]1F]

The title compound was prepared according to the methods described for Preparations 725B, 725A and 725 using tert-butyl (3S,4R)-3-fluoro-4-hydroxypiperidine-1-carboxylate (WO 2013/011402 A1).

MS m/z 477 [M+H]⁺

### Preparation 220

### 6-chloro-3-(((3R,4S)-3-fluoro-1-(2-(4-(trifluoromethoxy)phenyl)acetyl)piperidin-4-yl)oxy)pyridazine-4-carboxamide

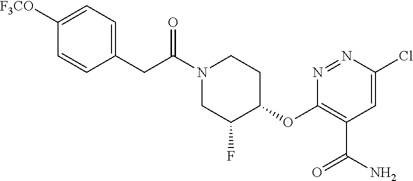 [SMILES: NC(=O)c1cc(Cl)nnc1O[C@H]1CCN(C(=O)Cc2ccc(OC(F)(F)F)cc2)C[C@H]1F]

The title compound was prepared according to the method described for Preparation 28 using 6-chloro-3-(((3R,4S)-3-fluoropiperidin-4-yl)oxy)pyridazine-4-carboxamide hydrochloride (Preparation 228) and 4-trifluoromethoxyphenylacetic acid.

MS m/z 477 [M+H]⁺

### Preparation 221

### 5-(4,4,5,5-tetramethyl-1,3,2-dioxaborolan-2-yl)-2-(((1S,5S)-3-(2-(4-(trifluoromethoxy)phenyl)acetyl)-6-oxa-3-azabicyclo[3.2.1]octan-8-yl)oxy)benzamide

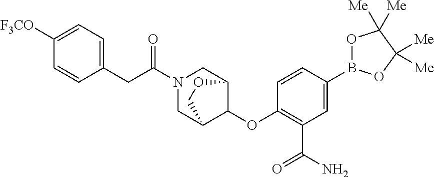 [SMILES: CC1(C)OB(c2ccc(OC3[C@@H]4CO[C@H]3CN(C(=O)Cc3ccc(OC(F)(F)F)cc3)C4)c(C(N)=O)c2)OC1(C)C]

The title compound was prepared according to the method described for Preparation 12 using 5-bromo-2-(((1S, 5S)-3-(2-(4-(trifluoromethoxy)phenyl)acetyl)-6-oxa-3-azabicyclo[3.2.1 ]octan-8-yl)oxy)benzamide (Preparation 223) and was taken on directly to the next step.

### Preparation 222

### 5-(4,4,5,5-tetramethyl-1,3,2-dioxaborolan-2-yl)-2-((2-(2-(4-(trifluoromethoxy)phenyl)acetyl)-2-azabicyclo[4.1.0]heptan-5-yl)oxy)benzamide

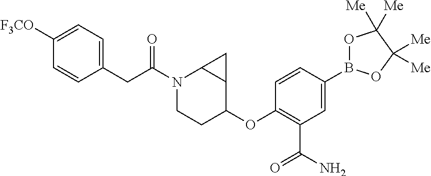 [SMILES: CC1(C)OB(c2ccc(OC3CCN(C(=O)Cc4ccc(OC(F)(F)F)cc4)C4CC34)c(C(N)=O)c2)OC1(C)C]

The title compound was prepared according to the method described for Preparation 12 using 5-bromo-2-((2-(2-(4-(trifluoromethoxy)phenyl)acetyl)-2-azabicyclo[4.1.0]heptan-5-yl)oxy)benzamide (Preparation 224). Taken on directly to the next step.

### Preparation 223

### 5-bromo-2-(((1S,5S)-3-(2-(4-(trifluoromethoxy)phenyl)acetyl)-6-oxa-3-azabicyclo[3.2.1]octan-8-yl)oxy)benzamide

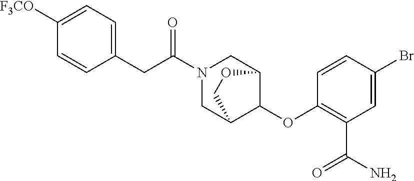 [SMILES: NC(=O)c1cc(Br)ccc1OC1[C@@H]2CO[C@H]1CN(C(=O)Cc1ccc(OC(F)(F)F)cc1)C2]

The title compound was prepared according to the method described for Preparation 28 using 2-(((1S,5S)-6-oxa-3-azabicyclo[3.2.1]octan-8-yl)oxy)-5-bromobenzamide hydrochloride (Preparation 229) and 4-trifluoromethoxyphenylacetic acid.

¹H NMR (400 MHz, DMSO-d₆): δ ppm 2.75-2.80 (m, 1H), 2.95-3.15 (m, 1H), 3.30-3.50 (m, 1H), 3.60-4.10 (m, 6H), 4.30-4.35 (m, 1H), 4.95-5.05 (m, 1H), 7.27-7.34 (m, 5H), 7.57-7.64 (m, 4H).

MS m/z 529 [M⁷⁹Br+H]⁺

### Preparation 224

### 5-bromo-2-((2-(2-(4-(trifluoromethoxy)phenyl)acetyl)-2-azabicyclo[4.1.0]heptan-5-yl)oxy)benzamide

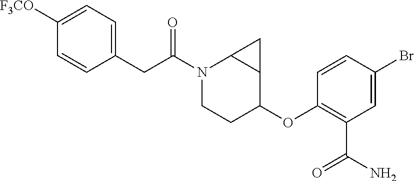 [SMILES: NC(=O)c1cc(Br)ccc1OC1CCN(C(=O)Cc2ccc(OC(F)(F)F)cc2)C2CC12]

The title compound was prepared according to the method described for Preparation 28 using 2-((2-azabicyclo[4.1.0]heptan-5-yl)oxy)-5-bromobenzamide hydrobromide (Preparation 225).

¹H NMR (400 MHz, DMSO-d₆): δ ppm 0.88 (m, 1H), 1.00-1.05 (m, 1H), 1.38-1.41 (m, 1H), 1.78-1.81 (m, 1H), 2.07-2.10 (m, 1H), 2.57-2.66 (m, 1H), 3.21-3.25 (m, 1H), 3.82-3.85 (m, 1H), 3.93-3.97 (m, 1H), 4.11-4.14 (m, 1H), 5.22-5.27 (m, 1H), 7.29 (d, 2H), 7.32-7.38 (m, 3H), 7.56 (br s, 1H), 7.60 (dd, 1H), 7.68 (br s, 1H), 7.89 (d, 1H).

### Preparation 225

### 2-((2-azabicyclo[4.1.0]heptan-5-yl)oxy)-5-bromobenzamide hydrobromide

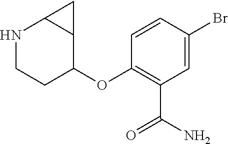 [SMILES: NC(=O)c1cc(Br)ccc1OC1CCNC2CC21]

A mixture of benzyl 5-(4-bromo-2-carbamoylphenoxy)-2-azabicyclo[4.1.0]heptane-2-carboxylate (Preparation 239, 250 mg, 0.56 mmol) and 30% HBr in AcOH (2.5 mL) was stirred at room temperature for 2 hours. The reaction mixture was evaporated in vacuo and azeotroped with toluene to afford the title compound as brown solid (200 mg, 91%).

¹H NMR (400 MHz, DMSO-d₆): δ ppm_0.84-0.90 (q, 1H), 1.10-1.14 (q, 1H), 1.65-1.70 (m, 1H), 1.72-1.76 (m, 1H), 1.95-1.98 (m, 1H), 2.95-2.99 (m, 3H), 5.18 (q, 1H), 7.28 (d, 1H), 7.57 (br s, 1H), 7.62 (dd, 1H), 7.66 (br s, 1H), 7.76 (d, 1H), 8.95 (br s, 1H), 9.13 (br s, 1H).

### Preparation 226

### 2-(((3S,4R)-3-fluoropiperidin-4-yl)oxy)-5-(2-methyl-2H-1,2,3-triazol-4-yl)nicotinamide hydrochloride

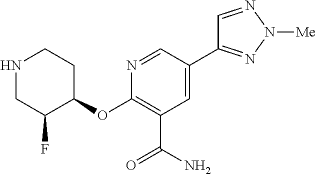 [SMILES: Cn1ncc(-c2cnc(O[C@@H]3CCNC[C@@H]3F)c(C(N)=O)c2)n1]

The title compound was prepared according to the method described for Preparation 43 using tert-butyl (3S,4R)-4-((3-carbamoyl-5-(2-methyl-2H-1,2,3-triazol-4-yl)pyridin-2-yl)oxy)-3-fluoropiperidine-1-carboxylate (Preparation 233).

MS m/z 321 [M+H]⁺

### Preparation 227

### 2-(((3S,4R)-3-fluoropiperidin-4-yl)oxy)-5-(1-methyl-1H-1,2,3-triazol-4-yl)nicotinamide hydrochloride

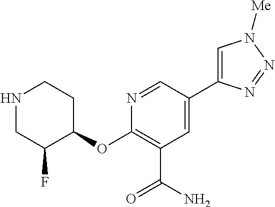 [SMILES: Cn1cc(-c2cnc(O[C@@H]3CCNC[C@@H]3F)c(C(N)=O)c2)nn1]

The title compound was prepared according to the method described for Preparation 43 using tert-butyl (3S,4R)-4-((3-carbamoyl-5-(1-methyl-1H-1,2,3-triazol-4-yl)pyridin-2-yl)oxy)-3-fluoropiperidine-1-carboxylate (Preparation 234).

MS m/z 321 [M+H]⁺

### Preparation 228

### 6-chloro-3-(((3R,4S)-3-fluoropiperidin-4-yl)oxy)pyridazine-4-carboxamide hydrochloride

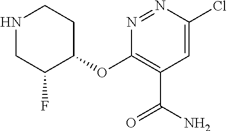 [SMILES: NC(=O)c1cc(Cl)nnc1O[C@H]1CCNC[C@H]1F]

The title compound was prepared according to the method described for Preparation 43 using tert-butyl (3R,4S)-4-((4-carbamoyl-6-chloropyridazin-3-yl)oxy)-3-fluoropiperidine-1-carboxylate (Preparation 232).

¹H NMR (400 MHz, DMSO-d₆): δ ppm 1.80-1.90 (m, 1H), 2.10-2.30 (m, 1H), 2.90-3.90 (m, 3H), 4.70-4.88 (m, 1H), 5.20-5.37 (m, 1H), 5.61-5.70 (m, 1H), 7.90 (br s, 1H), 7.99 (s, 1H), 8.07 (br s, 1H).

MS m/z 275 [M+H]⁺

### Preparation 229

### 2-(((1S,5S)-6-oxa-3-azabicyclo[3.2.1]octan-8-yl)oxy)-5-bromobenzamide hydrochloride

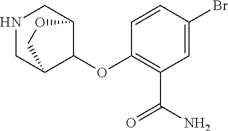 [SMILES: NC(=O)c1cc(Br)ccc1OC1[C@H]2CNC[C@@H]1OC2]

The title compound was prepared according to the method described for Preparation 43 using tert-butyl (1S,5S)-8-(4-bromo-2-carbamoylphenoxy)-6-oxa-3-azabicyclo[3.2.1]octane-3-carboxylate (Preparation 237).

MS m/z 329 [M⁸¹Br+H]⁺

### Preparation 230

### Ethyl 1-(5-carbamoyl-6-(((3S,4R)-3-fluoropiperidin-4-yl)oxy)pyridin-3-yl)-1H-pyrazole-4-carboxylate hydrochloride

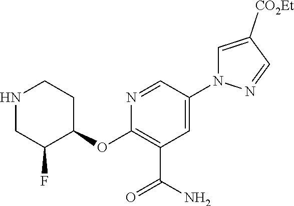 [SMILES: CCOC(=O)c1cnn(-c2cnc(O[C@@H]3CCNC[C@@H]3F)c(C(N)=O)c2)c1]

The title compound was prepared according to the method described for Preparation 43 using tert-butyl (3S,4R)-4-((3-carbamoyl-5-(4-(ethoxycarbonyl)-1H-pyrazol-1-yl)pyridin-2-yl)oxy)-3-fluoropiperidine-1-carboxylate (Preparation 235).

MS m/z 378 [M+H]⁺

### Preparation 231

### 2-(((3S,4R)-3-fluoropiperidin-4-yl)oxy)-5-(4-methyl-1H-imidazol-1-yl)nicotinamide

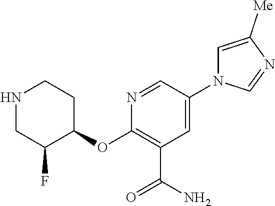 [SMILES: Cc1cn(-c2cnc(O[C@@H]3CCNC[C@@H]3F)c(C(N)=O)c2)cn1]

The title compound was prepared according to the method described for Preparation 43 using tert-butyl (3S,4R)-4-((3-carbamoyl-5-(4-methyl-1H-imidazol-1-yl) pyridin-2-yl)oxy)-3-fluoropiperidine-1-carboxylate (Preparation 236).

MS m/z 320 [M+H]⁺

### Preparation 232

### tert-butyl (3R,4S)-4-((4-carbamoyl-6-chloropyridazin-3-yl)oxy)-3-fluoropiperidine-1-carboxylate

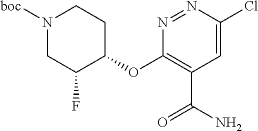

The title compound was prepared according to the method described for Preparation 118 using tert-butyl (3R,4S)-3-fluoro-4-hydroxypiperidine-1-carboxylate (WO 2013/011402 A1) and 3,6-dichloropyridazine-4-carboxamide in dioxane. The residue was purified using silica gel column chromatography eluting with 2-3% MeOH in DCM.

MS m/z 375 [M+H]⁺

### Preparation 233

### tert-butyl(3S,4R)-4-((3-carbamoyl-5-(2-methyl-2H-1,2,3-triazol-4-yl)pyridin-2-yl)oxy)-3-fluoropiperidine-1-carboxylate

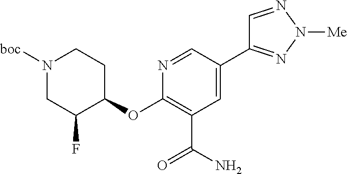

The title compound was prepared according to the method described for Preparation 136 using tert-butyl (3S,4S)-3-fluoro-4-(((trifluoromethyl)sulfonyl)oxy)piperidine-1-carboxylate (Preparation 241) and 2-hydroxy-5-(2-methyl-2H-1,2,3-triazol-4-yl)nicotinamide (Preparation 243).

MS m/z 421 [M+H]⁺

Preparation 234

tert-butyl(3S,4R)-4-((3-carbamoyl-5-(1-methyl-1H-1,2,3-triazol-4-yl)pyridin-2-yl)oxy)-3-fluoropiperidine-1-carboxylate

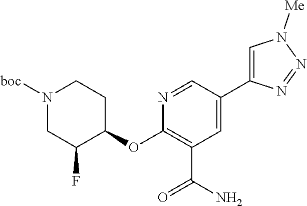

The title compound was prepared according to the method described for Preparation 136 using tert-butyl (3S,4S)-3-fluoro-4-(((trifluoromethyl)sulfonyl)oxy)piperidine-1-carboxylate (Preparation 241) and 2-hydroxy-5-(1-methyl-1H-1,2,3-triazol-4-yl)nicotinamide (Preparation 244).

MS m/z 421 [M+H]⁺

### Preparation 235

### tert-butyl(3S,4R)-4-((3-carbamoyl-5-(4-(ethoxycarbonyl)-1H-pyrazol-1-yl)pyridin-2-yl)oxy)-3-fluoropiperidine-1-carboxylate

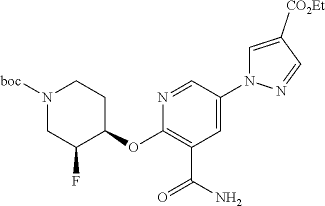

The title compound was prepared according to the method described for Preparation 136 using ethyl 1-(5-carbamoyl-6-hydroxypyridin-3-yl)-1H-pyrazole-4-carboxylate (Preparation 235 and Ethyl 1-(5-carbamoyl-6-hydroxypyridin-3-yl)-1H-pyrazole-4-carboxylate (Preparation 249). Taken on directly to the next step.

### Preparation 236

### tert-butyl(3S,4R)-4-((3-carbamoyl-5-(4-methyl-1H-imidazol-1-yl)pyridin-2-yl)oxy)-3-fluoropiperidine-1-carboxylate

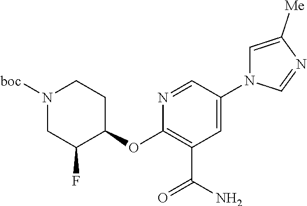

The title compound was prepared according to the method described for Preparation 136 using 2-hydroxy-5-(4-methyl-1H-imidazol-1-yl)nicotinamide and 2-hydroxy-5-(4-methyl-1H-imidazol-1-yl)nicotinamide (Preparation 250).

¹H NMR (400 MHz, DMSO-d₆): δ ppm 1.40 (s, 9H), 1.87-2.00 (m, 2H), 2.16 (s, 3H), 3.00-3.20 (m, 2H), 3.80-4.10 (m, 1H), 4.15-4.30 (m, 1H), 5.00-5.15 (m, 1H), 5.40-5.50 (m, 1H), 7.46 (s, 1H), 7.59 (br s, 1H), 7.94 (br s, 1H), 8.13 (s, 1H), 8.34 (s, 1H), 8.58 (s, 1H).

MS m/z 420 [M+H]⁺

### Preparation 237

### tert-butyl(1S,5S)-8-(4-bromo-2-carbamoylphenoxy)-6-oxa-3-azabicyclo[3.2.1]octane-3-carboxylate

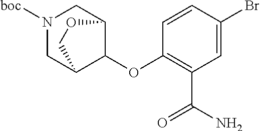

To a cooled solution of tert-butyl (1S,5S)-8-(4-bromo-2-cyanophenoxy)-6-oxa-3-azabicyclo[3.2.1]octane-3-carboxylate (Preparation 238, 420 mg, 1.026 mmol) in t-butyl alcohol (8 mL) was added KOH powder (86 mg, 1.539 mmol) portion-wise. The temperature was slowly warmed to 80° C. and stirred for 8 hours. The reaction was quenched with water and extracted into EtOAc twice. The combined organic layers were washed with brine, dried over sodium sulfate and concentrated in vacuo to afford the title compound as a white solid (330 mg, 75%).

¹H NMR (400 MHz, DMSO-d₆): δ ppm 1.40 (s, 9H), 2.73-2.80 (m, 1H), 3.02-3.31 (m, 2H), 3.61-4.28 (m, 5H), 4.85-4.90 (m, 1H), 7.29-7.31 (m, 1H), 7.56-7.64 (m, 4H).

### Preparation 238

### tert-butyl(1S,5S)-8-(4-bromo-2-cyanophenoxy)-6-oxa-3-azabicyclo[3.2.1]octane-3-carboxylate

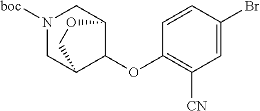

The title compound was prepared according to the method described for Preparation 120 using tert-butyl (1S,5S)-8-hydroxy-6-oxa-3-azabicyclo[3.2.1]octane-3-carboxylate (Tet. Lett (51) 22, (2010), 2998-3001). The residue was purified using silica gel column chromatography eluting with 30% EtOAc in hexanes.

¹H NMR (400 MHz, DMSO-d₆): δ ppm 1.40 (s, 9H), 2.73-2.98 (m, 1H), 3.00-3.30 (m, 2H), 3.65-4.03 (m, 4H), 4.27-4.32 (m, 1H), 5.06-5.07 (m, 1H), 7.48-7.51 (m, 1H), 7.84-7.87 (m, 1H), 8.06-8.07 (m, 1H).

MS m/z 409 [M+H]⁺

### Preparation 239

### Benzyl 5-(4-bromo-2-carbamoylphenoxy)-2-azabicyclo[4.1.0]heptane-2-carboxylate

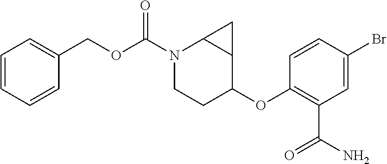 [SMILES: NC(=O)c1cc(Br)ccc1OC1CCN(C(=O)OCc2ccccc2)C2CC12]

To a stirred solution of benzyl 5-hydroxy-2-azabicyclo[4.1.0]heptane-2-carboxylate_(Preparation 240, 200 mg, 0.81 mmol) and 5-bromo-2-fluorobenzamide (176 mg, 0.81 mmol) in DMF (4 mL) was added Cs₂CO₃ (790 mg, 2.43 mmol) and the resulting reaction mixture was heated at 80° C. for 16 hours. The reaction was diluted with EtOAc (25 mL), washed with water (3×10 mL) and brine (10 mL), dried over sodium sulfate and evaporated in vacuo. The crude material was purified by silica gel column chromatography eluting with 50% EtOAc in Hexane to afford the title compound as light yellow gum (250 mg, 69%).

MS m/z 445 [M+2H]⁺

### Preparation 240

### Benzyl 5-hydroxy-2-azabicyclo[4.1.0]heptane-2-carboxylate

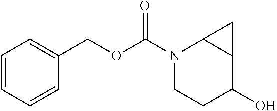 [SMILES: O=C(OCc1ccccc1)N1CCC(O)C2CC21]

To a solution of benzyl 4-hydroxy-3,4-dihydropyridine-1(2H)-carboxylate (J.O.C. 70, 19, (2005), 7715-7720, 2 g, 8.58 mmol) in anhydrous diethyl ether (30 mL) was added a solution of diethylzinc (1M in hexane, 20.5 mL, 20.5 mmol) followed by drop-wise addition of CH₂I₂ (9.18 g, 34.3 mmol). The mixture was stirred at room temperature for 4 hours. The reaction mixture was diluted with diethyl ether (50 mL) and 1N HCl (25 mL) was added drop-wise before stirring for 15 minutes. The layers were separated and the organic layer washed with saturated aqueous NaHCO₃ solution followed by water. The organic layer was dried over sodium sulfate and concentrated in vacuo. The residue was purified by silica gel column chromatography eluting with 80% EtOAc in petroleum ether to afford the title compound.

¹H NMR (300 MHz, DMSO-d₆): δ ppm 0.49-0.54 (m, 1H), 0.69-0.76 (m, 1H), 1.05-1.18 (m, 1H), 1.34-1.40 (m, 1H), 1.67-1.72 (m, 1H), 2.62-2.80 (m, 1H), 2.94-3.00 (m, 1H), 3.64-3.68 (m, 1H), 4.10 (br s, 1H), 4.43 (s, 1H), 5.10 (s, 2H), 7.29-7.38 (m, 5H).

MS m/z 248 [M+H]⁺

### Preparation 241

### tert-butyl (3S,4S)-3-fluoro-4-(((trifluoromethyl)sulfonyl)oxy)piperidine-1-carboxylate

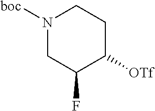

To a solution of tert-butyl-(3S,4S)-3-fluoro-4-hydroxypiperidine-1-carboxylate (320 mg, 1.461 mmol) in DCM (5 mL) was added pyridine (0.295 mL, 3.653 mmol) and the reaction mixture was cooled to −20°. Triflic anhydride (0.364 mL, 2.192 mmol) was added and the reaction mixture was stirred at −20° C. for 1 hour. The reaction was diluted with DCM, washed with saturated NaHCO₃ solution, water and brine, dried over Na₂SO₄ and concentrated in vacuo to afford the title compound (500 mg, 97%) as light brown solid. Taken on directly to the next step.

### Preparation 242

### tert-butyl(2R,4S,5S)-4-hydroxy-2,5-dimethylpiperidine-1-carboxylate and tert-butyl (2R,4R,5S)-4-hydroxy-2,5-dimethylpiperidine-1-carboxylate

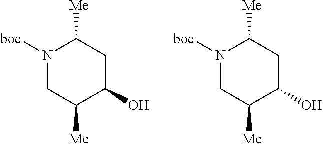

To a stirred solution of tert-butyl (2R,5S)-2,5-dimethyl-4-oxopiperidine-1-carboxylate (PCT 2004009550, 400 mg, 1.76 mmol) in MeOH (7.5 mL) at 0° C. was added NaBH₄ (133 mg, 3.52 mmol) and the reaction was stirred at room temperature for 30 minutes. The reaction mixture was quenched with ice water and extracted with DCM. The organic layer was dried over sodium sulfate and concentrated in vacuo to afford the title compound as a mixture of hydroxy-isomers as colorless gum (300 mg, 74%).

¹H NMR (300 MHz, DMSO-d₆): δ ppm 0.77 (d, 1.5H), 0.85 (d, 1.5H), 1.07 (d, 1.5H), 1.20 (d, 1.5H), 1.38 (s, 9H), 1.41-1.83 (m, 3H), 2.97 (dd, 0.5H), 3.27-3.38 (m, 1H), 3.34-3.44 (m, 0.5H), 3.66 (d, 0.5H), 3.84-3.88 (m, 0.5H), 4.00-4.05 (m, 0.5H), 4.30-4.33 (m, 0.5H), 4.56 (d, 0.5H), 4.70 (d, 0.5H).

### Preparation 243

### 2-hydroxy-5-(2-methyl-2H-1,2, 3-triazol-4-yl)nicotinamide

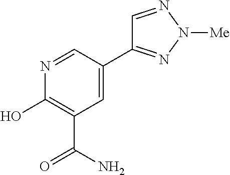 [SMILES: Cn1ncc(-c2cnc(O)c(C(N)=O)c2)n1]

A solution of methyl 2-(benzyloxy)-5-(2-methyl-2H-1,2,3-triazol-4-yl)nicotinate (Preparation 245, 250 mg, 0.772 mmol) in MeOH (10 mL) was degassed with argon for about 10 minutes followed by the addition of Pd/C (10 wt %, 250 mg) and 4M HCl in dioxane (1.5 mL). The reaction was stirred under a balloon of hydrogen for 2 hours at ambient temperature. The reaction mixture was filtered through celite and washed with MeOH. The filtrate was concentrated in vacuo and then dissolved in a smaller volume of MeOH (3 mL), Methanolic ammonia (6 mL) was added and the reaction was stirred in a sealed tube at room temperature for 16 hours. The reaction was evaporated to dryness and triturated with ether to afford the title compound as yellow solid (130 mg, 86%). Taken on directly to the next step.

### Preparation 244

### 2-hydroxy-5-(1-methyl-1H-1,2,3-triazol-4-yl)nicotinamide

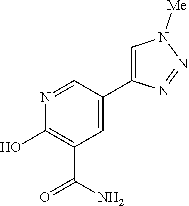 [SMILES: Cn1cc(-c2cnc(O)c(C(N)=O)c2)nn1]

The title compound was prepared as described for Preparation 243 using methyl 2-(benzyloxy)-5-(1-methyl-1H-1,2,3-triazol-4-yl)nicotinate (Preparation 245A). Taken on directly to the next step.

### Preparation 245 and 245A

### Methyl 2-(benzyloxy)-5-(2-methyl-2H-1,2,3-triazol-4-yl)nicotinate and Methyl 2-(benzyloxy)-5-(1-methyl-1H-1,2,3-triazol-4-yl)nicotinate

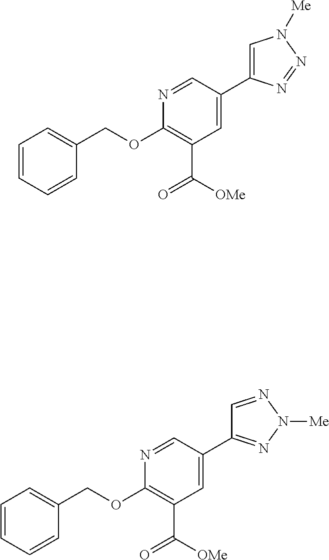 [SMILES: COC(=O)c1cc(-c2cn(C)nn2)cnc1OCc1ccccc1 ; COC(=O)c1cc(-c2cnn(C)n2)cnc1OCc1ccccc1]

To a stirred solution of methyl 2-(benzyloxy)-5-(2H-1,2,3-triazol-4-yl)nicotinate (Preparation 246, 600 mg, 1.93 mmol) in acetone (15 mL) was added potassium carbonate (1.06 mg, 7.72 mmol) and methyl iodide (0.119 mL, 1.93 mmol) and the reaction was heated at 40° C. for 16 hours. The reaction was diluted with ethyl acetate and washed with water and brine, dried over sodium sulphate and concentrated in vacuo. The two regioisomers were isolated by silica gel column chromatography eluting first with 40% EtOAc in hexanes to afford methyl 2-(benzyloxy)-5-(2-methyl-2H-1,2,3-triazol-4-yl)nicotinate (200 mg, 32%) followed by elution with 3% MeOH in DCM to afford methyl 2-(benzyloxy)-5-(1-methyl-1H-1,2,3-triazol-4-yl)nicotinate (170 mg, 27%).

### Methyl 2-(benzyloxy)-5-(2-methyl-2H-1,2,3-triazol-4-yl)nicotinate (Preparation 245)

¹H NMR (400 MHz, DMSO-d₆): δ ppm 3.77 (s, 3H), 4.16 (s, 3H), 5.22 (s, 2H), 7.29-7.36 (m, 5H), 8.12 (s, 1H), 8.44 (s, 1H), 8.74 (s, 1H). No NOe observed between the triazole-H and the N-Me.

### Methyl 2-(benzyloxy)-5-(1-methyl-1H-1,2,3-triazol-4-yl)nicotinate (Preparation 245A)

¹H NMR (400 MHz, DMSO-d₆): δ ppm 3.77 (s, 3H), 4.10 (s, 3H), 5.23 (s, 2H), 7.29-7.36 (m, 5H), 8.41 (s, 1H), 8.46 (s, 1H), 8.69 (s, 1H). NOe is observed between the triazole-H and the N-Me.

### Preparation 246

### Methyl 2-(benzyloxy)-5-(2H-1,2,3-triazol-4-yl)nicotinate

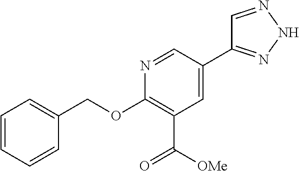 [SMILES: COC(=O)c1cc(-c2cn[nH]n2)cnc1OCc1ccccc1]

To a stirred solution of methyl 2-(benzyloxy)-5-ethynylnicotinate (Preparation 247, 3.9 g, 12.037 mmol) in t-BuOH-water (1:1, 30 mL) was added sodium azide (782 mg, 12.037 mmol), sodium ascorbate (238 mg, 1.204 mmol) and CuSO₄ (30 mg, 0.12 mmol). The reaction mixture was heated at 100° C. for 48 hours. Water was added to the reaction mixture and the solid was filtered off. The aqueous layer was extracted with ethyl acetate thrice. The organic layer was dried over sodium sulphate and combined with the solid and evaporated. The crude compound was purified by silica gel column chromatography eluting with 3% methanol in DCM to afford the title compound as brown solid (900 mg, 24%).

¹H NMR (400 MHz, DMSO-d₆): δ ppm 3.77 (s, 3H), 5.22 (s, 2H), 7.28-7.36 (m, 5H), 8.49 (s, 1H), 8.75 (s, 1H), 15.10 (br s, 1H).

MS m/z 311 [M+H]⁺

### Preparation 247

### Methyl 2-(benzyloxy)-5-ethynylnicotinate

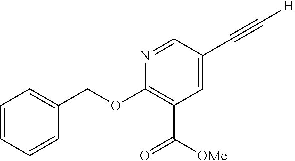 [SMILES: C#Cc1cnc(OCc2ccccc2)c(C(=O)OC)c1]

To a stirred solution of methyl 2-(benzyloxy)-5-bromonicotinate (Preparation 248, 3.0 g, 9.31 mmol) in acetonitrile (30 mL) was added DIPEA (16.1 mL, 93.12 mmol) and the reaction mixture was degassed for 15 minutes. CuI (353 mg, 1.86 mmol), PdCl₂(PPh₃)₂ (650 mg, 0.93 mmol) and TMS acetylene (9.01 mL, 65.187 mmol) were added and the reaction was heated at 80° C. in a sealed tube for 16 hours. The reaction was diluted with ethyl acetate and washed with water and brine, dried over sodium sulphate and concentrated in vacuo. The residue was purified by silica gel column chromatography eluting with 15% ethyl acetate in hexanes. The residue (5.0 g, 15.432 mmol) was stirred in THF (40 mL) and cooled to 0° C. TBAF (30 mL, 30.864 mmol) was added drop-wise at 0° C. and stirred for 30 minutes. The reaction was diluted with ethyl acetate and washed with water followed by brine solution, dried over sodium sulphate and concentrated in vacuo to afford the title compound as brown gum (3.90 g, 94% yield). Taken on directly to the next step.

### Preparation 248

### Methyl 2-(benzyloxy)-5-bromonicotinate

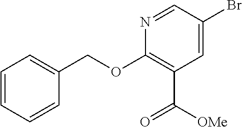 [SMILES: COC(=O)c1cc(Br)cnc1OCc1ccccc1]

To a solution of methyl-5-bromo-2-hydroxynicotinate (2.7 g, 11.63 mmol) in DMF (30 mL) was added silver carbonate (4.8 g, 17.45 mmol) and benzyl bromide (1.39 mL, 11.638 mmol). The reaction mixture was stirred at room temperature for 16 hours under darkness. The reaction was diluted with ethyl acetate and washed with water and brine, dried over sodium sulfate and concentrated in vacuo. The residue was purified by silica gel column chromatography eluting with 2% MeOH in DCM to afford the title compound as colorless liquid (2.3 g, 61%).

¹H NMR (400 MHz, DMSO-d₆): δ ppm 3.73 (s, 3H), 5.16 (s, 2H), 7.29-7.45 (m, 5H), 7.95 (s, 1H), 8.10 (s, 1H).

### Preparation 249

### Ethyl 1-(5-carbamoyl-6-hydroxypyridin-3-yl)-1H-pyrazole-4-carboxylate

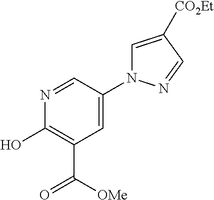 [SMILES: CCOC(=O)c1cnn(-c2cnc(O)c(C(=O)OC)c2)c1]

A stirred suspension of 5-bromo-2-hydroxynicotinamide (Preparation 251, 2 g, 9.21 mmol), ethyl 1H-pyrazole-4-carboxylate (1.94 g, 13.82 mmol) and K₂CO₃ (3.18 g, 23.04 mmol) in DMF (40 mL) was degassed with argon for 20 minutes. CuI (175 mg, 0.92 mmol) followed by [trans-N,N-dimethylcyclohexane-1,2-diamine] (262 mg, 1.84 mmol) was added and the reaction was further degassed for 5 minutes. The reaction was stirred at 110° C. for 32 hours in a sealed tube. The reaction was filtered through a sintered funnel and the filtrate was concentrated in vacuo. The residue was purified using silica gel column chromatography eluting with 10-15% MeOH in DCM/NH₃ to afford the title compound (500 mg, 20%).

¹H NMR (400 MHz, DMSO-d₆): δ ppm 1.29 (t, 3H), 4.24 (q, 2H), 7.72-7.75 (m, 1H), 7.99-8.00 (m, 1H), 8.27 (s, 1H), 8.96-9.03 (m, 1H), 12.80 (br s, 1H).

### Preparation 250

### 2-hydroxy-5-(4-methyl-1H-imidazol-1-yl)nicotinamide

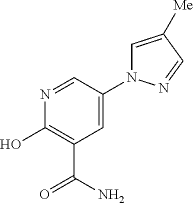 [SMILES: Cc1cnn(-c2cnc(O)c(C(N)=O)c2)c1]

The title compound was prepared according to the method described for Preparation 249 using 5-bromo-2-hydroxynicotinamide (Preparation 251) and 4-methyl-1H-imidazole.

### Preparation 251

### 5-bromo-2-hydroxynicotinamide

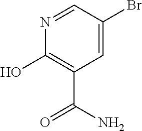 [SMILES: NC(=O)c1cc(Br)cnc1O]

A solution of methyl-5-bromo-2-hydroxynicotinate (8 g, 34.48 mmol) in MeOH/NH₃ (140 mL) was stirred for 16 hours at 60° C. in a sealed tube. The reaction mixture was allowed to cool and was concentrated under reduced pressure. The residue was azeotroped with toluene and triturated with n-pentane to afford the title compound (7.2 g, 96%) as an off-white solid.

¹H NMR (400 MHz, DMSO-d₆): δ ppm 7.12 (br s, 1H), 7.99 (s, 1H), 8.26 (s, 1H), 8.79 (br s, 1H), 12.37 (br s, 1H).

### Preparation 252

### 2-(5-(1,1-difluoroethoxy)pyridin-2-yl)acetic acid

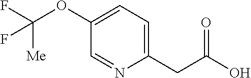 [SMILES: CC(F)(F)Oc1ccc(CC(=O)O)nc1]

At 0° C., nBuLi (2.1 M, 5.71 mL, 12 mmol) was added drop-wise to a solution of diisopropylamine (1.85 mL, 13.2 mmol) in THF (4.44 mL). The solution was stirred at 0° C. for 10 minutes to afford 12 mL of 1M LDA solution in THF which was used in the reaction.

To a solution of 5-(1,1-difluoroethoxy)-2-methylpyridine (Preparation 253, 300 mg, 1.73 mmol) and diethyl carbonate (1.1 mL, 8.66 mmol) in THF (10 mL) was added the 1M LDA solution (6.06 mL, 6.06 mmol) drop-wise at −78° C. The reaction was stirred at that temperature for 3 hours. The reaction was quenched with saturated aqueous NH₄Cl solution and extracted with EtOAc (2 times). The combined organic layers were washed with brine, dried over sodium sulfate and concentrated in vacuo. The residue was purified by silica gel column chromatography eluting with 0-15% ethyl acetate in hexanes.

The residue (570 mg, 2.32 mmol) was dissolved in MeOH:THF(6 mL, 1:1) and water (6 mL), and to this was added LiOH.H₂O (321 mg, 7.678 mmol) and the reaction mixture was stirred at room temperature for 1 hour. The reaction was concentrated in vacuo and the residue was diluted with water and washed with DCM (twice). This DCM layer was kept aside and the aqueous layer was acidified to pH=4-5 with 2N HCl solution and extracted with 10% IPA/DCM (twice). The combined organic layers were dried over sodium sulfate and concentrated in vacuo to afford the title compound as yellow solid (410 mg, 33% over 2 steps).

¹H NMR (400 MHz, DMSO-d₆): δ ppm 2.03 (t, 3H), 3.76 (s, 2H), 7.41 (d, 1H), 7.63 (d, 1H), 8.37 (s, 1H), 12.45 (br s, 1H).

MS m/z 218 [M+H]⁺

### Preparation 253

### 5-(1,1-difluoroethoxy)-2-methylpyridine

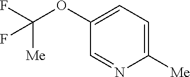 [SMILES: Cc1ccc(OC(C)(F)F)cn1]

To a solution of 6-methylpyridin-3-ol (1.0 g, 9.17 mmol) and KOH (1.54 g, 27 mmol) in acetonitrile (30 mL) and water (3 mL) was bubbled 2-bromo-1,1-difluoro-ethylene (gas) at 0° C. The reaction was stirred at 50° C. in a sealed tube for 16 hours. The reaction mixture was cooled to room temperature, diluted with water and extracted with ethyl acetate (twice). The combined organic layers were further washed with water, brine, dried over sodium sulphate and concentrated in vacuo. The residue was purified using silica gel column chromatography eluting with 10% ethyl acetate in hexanes. The residue (700 mg, 2.778 mmol) was heated to reflux in anhydrous toluene (1 mL) and AIBN (22 mg, 0.139 mmol) was added. A solution of tri-butyltinhydride (0.971 mL, 3.6 mmol) in toluene (1 mL) was added drop-wise and the resultant mixture was refluxed for 16 hours. The reaction was cooled to room temperature and quenched with 10% aqueous KF solution and stirred overnight. The solution was extracted with ethyl acetate twice and the layers were separated. The organic layer was washed with brine, dried over sodium sulfate and concentrated in vacuo. The residue was purified by silica gel column chromatography eluting with 0-5% ethyl acetate in hexanes to afford the title compound (325 mg, 23% over two steps).

¹H NMR (400 MHz, CDCl₃): δ ppm 1.89 (t, 3H), 2.53 (s, 3H), 7.10 (d, 1H), 7.39 (d, 1H), 8.34 (s, 1H).

### Biological Activity

Isolated TRK Enzyme assays use the HTRF KinEASE-TK kit (Cisbio Cat #62TK0PEJ) with recombinant His-tagged cytoplasmic domains of TRKA receptor sourced from Invitrogen (see table below). This activity-assay measures the phosphorylation of tyrosine residues within a substrate from the HTRF kit which has been validated by Cisbio for a variety of tyrosine kinases including the TRK receptors.

Assay Details:

| | Invitrogen | Amino | FAC | FAC | Assay Reaction |
|---|---|---|---|---|---|
| Target | Cat# | acids | enzyme | ATP | Time |
| TRKA | PV3144 | aa 441-796 | 4 nM | 40 uM | 35 min |
| | (NTRK1) | | | | |

0.5 mM stock solutions of test compounds are prepared and serially diluted in 100% DMSO. A standard curve using the compound of Example 135 disclosed in WO2005/116035 of 150 uM is also prepared on each test plate. High percentage effect (HPE) is defined by 150 uM (using the compound of Example 135 as disclosed in WO2005/116035) and 0% effect (ZPE) is defined by 100% DMSO. Greiner low volume black plates containing 0.2 ul of serially diluted compound, standard and HPE/ZPE are created using the Bravo nanolitre dispenser.

1× enzyme buffer is prepared from 5× Enzymatic Buffer from the Cisbio KinEASE TK kit using MilliQ water. The buffer is then supplemented with 10 mM MgCl and 2 mM DTT (both from Sigma). In the case of TRKB, the buffer is also supplemented with 125 nM Supplement Enzymatic Buffer (SEB) from the Cisbio kit.

2× FAC of enzyme and 2× FAC ATP diluted in 1× complete enzyme buffer is incubated at room temperature for 20 minutes to preactivate the enzyme. Following this preactivation step, 5 ul/well of enzyme+ATP mix is added using a Multidrop Micro to the assay plate, spotted with 0.2 ul 100% DMSO compound. This is left for 20 mins at room temperature before adding 5 ul of 2 uM TK-substrate-Biotin (from the Cisbio kit) diluted in 1× enzyme buffer (1 uM FAC) using the Multidrop Micro. The reaction is incubated at room temperature for the optimized assay reaction time (see table). The reaction is stopped by adding 10 ul/well HTRF Detection Buffer containing 0.25 uM Streptavidin-XL665 (0.125 uM FAC) and 1:200 TK Antibody-Cryptate using a Multidrop.

After the Detection Reagent addition, plates are covered and incubated at room temperature for 60 minutes. HTRF signal is read using an Envision reader, measured as a ratio of emissions at two different wavelengths, 620 nm and 665 nm. Any compound that inhibits the action of the TRK kinase will have a lower fluorescence ratio value 665/620 nM than compounds which do not inhibit the TRK kinase. Test compound data are expressed as percentage inhibition defined by HPE and ZPE values for each plate. Percentage inhibition in the presence of test compound is plotted against compound concentration on a log scale to determine an IC₅₀ from the resultant sigmoid curve.

Cell Based Assays were carried out using Cell lines from DiscoveRx utilising their PathHunter technology and reagents in an antagonist assay:

| Target | DiscoveRx cell line Cat# | Cognate Neurotrophin |
|---|---|---|
| TRKA | 93-0462C3 | NGF |
| TRKA co expressed | 93-0529C3 | NGF |
| with p75 | | |

The assays are based upon DiscoveRx's proprietary Enzyme Fragment Complementation (EFC) technology. In the case of the TRK cell lines, the enzyme acceptor (EA) protein is fused to a SH2 protein and the TRK receptor of interest has been tagged with a Prolink tag.

Upon neurotrophin binding, the TRKA receptor becomes phosphorylated, and the tagged SH2 protein binds. This results in functional complementation and restored β-Galactosidase activity which is can be measured using the luminescent Galacton Star substrate within the PathHunter reagent kits.

Generally, small molecule inhibitors bind to the kinase domain so are not competing with the neurotrophin (agonist) which binds to an extracellular site. This means that the IC₅₀ is a good measure of affinity and should be unaffected by concentration neurotrophin stimulant.

Cryopreserved PathHunter cells are used from either in-house produced batches or bulk batches bought directly from DiscoveRx. Cryopreserved cells are resuscitated, spun 1000 rpm for 4 min to remove freezing media, and resuspended in MEM+0.5% horse serum (both Invitrogen) to 5e⁵ cells/ml. The cells are then plated using a Multidrop into Greiner white tissue culture treated plates at 20 ul/well and incubated for 24 h at 37° C., 5% CO₂, high humidity. On the day of the assay, the cell plates are allowed to cool to room temperature for 30 min prior to the assay.

4 mM stock solutions of test compounds are prepared and serially diluted in 100% DMSO. A standard curve using the compound of Example 135, WO2005/116035 at a top concentration of 150 uM is also prepared on each test plate. High percentage effect (HPE) is defined by 150 uM of the compound of Example 135, WO2005/116035 and 0% effect (ZPE) is defined by 100% DMSO. Plates containing 1 ul of serially diluted compound, standard and HPE/ZPE are diluted 1/66 in assay buffer (PBS minus Ca²⁺, minus Mg²⁺ with 0.05% pluronic F127) using a Wellmate. Using a Platemate Plus, 5 ul of 1/66 diluted test compounds is then transferred to the cell plate and allowed to reach equilibrium by incubating for 30 min at room temperature before addition of agonist stimulus: 10 ul/well of 2 nM (0.571 nM FAC) of the cognate neurotrophin (Peprotech) diluted in agonist buffer (HBSS with 0.25% BSA). Final assay concentration of the test compounds is 8.66 μM, (the compound of Example 135, WO2005/116035 FAC is 0.325 uM). The plates are left at room temperature for a further 2 hours before addition of 10 ul of the DiscoveRx PathHunter detection reagent (made up by adding 1 part Galacton Star, 5 parts Emerald II and 19 parts Cell Assay Buffer as per the manufacturer's instructions).

After reagent addition, plates are covered and incubated at room temperature for 60 minutes. Luminescence signal is read using an Envision. Test compound data are expressed as percentage inhibition defined by HPE and ZPE values for each plate. Percentage inhibition in the presence of test compound is plotted against compound concentration on a log scale to determine an IC₅₀ from the resultant sigmoid curve.

Below are TrkA IC₅₀ data generated using the PV3144 TrkA enzyme assay. Where more than one reading was taken, the arithmetic mean is presented.

| Example | IC₅₀ (nM) |
|---|---|
| 1 | 0.4 |
| 2 | 1.7 |
| 3 | 1.3 |
| 4 | 1.2 |
| 5 | 0.9 |
| 6 | 1.5 |
| 7 | 1.4 |
| 8 | 1.5 |
| 9 | 1.5 |
| 10 | 9.9 |
| 11 | — |
| 12 | 55 |
| 13 | 2.2 |
| 14 | 2.4 |
| 15 | 6.6 |
| 16 | 10.5 |
| 17 | 10.4 |
| 18 | 8.9 |
| 19 | 6.1 |
| 20 | 5.7 |
| 21 | 11.0 |
| 22 | 1.2 |
| 23 | 1.4 |
| 24 | 2.9 |
| 25 | 3.7 |
| 26 | 8.9 |
| 27 | 2.1 |
| 28 | 2.3 |
| 29 | 4.3 |
| 30 | 4.8 |
| 31 | 9.0 |
| 32 | 7.2 |
| 33 | 4.9 |
| 34 | 7.7 |
| 35 | 7.7 |
| 36 | 3.4 |
| 37 | 7.4 |
| 38 | 2.0 |
| 39 | 7.0 |
| 40 | 1.0 |
| 41 | 2.7 |
| 42 | 3.1 |
| 43 | 6.9 |
| 44 | 3.8 |
| 45 | 1.8 |
| 46 | 2.2 |
| 47 | 5.2 |
| 48 | 3.6 |
| 49 | 3.7 |
| 50 | 3.6 |
| 51 | 6.1 |
| 52 | 4.5 |
| 53 | 1.2 |
| 54 | 42.4 |
| 55 | 1.8 |
| 56 | 5.1 |
| 57 | 2.6 |
| 58 | 5.5 |
| 59 | 10.0 |
| 60 | 10.0 |
| 62 | 4.0 |
| 63 | 6.7 |
| 64 | 2.8 |
| 65 | 3.5 |
| 66 | 2.8 |
| 67 | 2.2 |
| 68 | 7.3 |
| 69 | 6.1 |
| 70 | 1.6 |
| 71 | 1.3 |
| 72 | 1.3 |
| 73 | 0.9 |
| 74 | 0.9 |
| 75 | 0.9 |
| 76 | 1.2 |
| 77 | 1.6 |
| 78 | 2.6 |
| 79 | 6.9 |
| 80 | 9.9 |
| 81 | 2.5 |
| 82 | 4.2 |
| 83 | 1.4 |
| 84 | 4.4 |
| 85 | 1.7 |
| 86 | 10.5 |
| 87 | 6.8 |
| 88 | 1.5 |
| 89 | 5.4 |
| 90 | 4.2 |
| 91 | 1.4 |
| 92 | 2.2 |
| 93 | — |
| 94 | 8.8 |
| 95 | 5.9 |
| 96 | 5.0 |
| 97 | 7.4 |
| 98 | 10.5 |
| 99 | 7.7 |
| 100 | 8.5 |
| 101 | 9.4 |
| 102 | 10.1 |
| 103 | 2.7 |
| 104 | 4.0 |
| 105 | 5.0 |
| 106 | 5.3 |
| 107 | 9.8 |
| 108 | 2.7 |
| 109 | 7.1 |
| 110 | 110 |
| 111 | 4.5 |
| 112 | 6.4 |
| 113 | 6.0 |
| 114 | 285.7 |
| 115 | 4.4 |
| 116 | 8.1 |
| 117 | 10.8 |
| 118 | 1.1 |
| 119 | 8.0 |
| 120 | 1.3 |
| 121 | 1.5 |
| 122 | 4.4 |
| 123 | 3.8 |
| 124 | 7.4 |
| 125 | 6.6 |
| 126 | 2.9 |
| 127 | 5.5 |
| 128 | 6.1 |
| 129 | 6.9 |
| 130 | 5.5 |
| 131 | 4.4 |
| 132 | 5.1 |
| 133 | 2.6 |
| 134 | 2.5 |
| 135 | 3.9 |
| 136 | 5.3 |
| 137 | 5.4 |
| 138 | 7.0 |
| 139 | 10.7 |
| 140 | 10.3 |
| 141 | 1.4 |
| 143 | 16.5 |
| 144 | 16.7 |
| 145 | 17.1 |
| 146 | 11.6 |
| 147 | 12.6 |
| 148 | 12.6 |
| 149 | 13.7 |
| 150 | 11.0 |
| 151 | 12.0 |
| 152 | 12.7 |
| 153 | 19.3 |
| 154 | 2.93 |
| 155 | 2.45 |
| 156 | 4.16 |
| 157 | 7.24 |
| 158 | 18.4 |
| 159 | 17.8 |
| 160 | 8.46 |
| 161 | — |
| 162 | 12.3 |
| 163 | 16.6 |
| 164 | 12.3 |
| 165 | 15.9 |
| 166 | 12.8 |
| 167 | 19.4 |
| 168 | 17.3 |
| 169 | 11.8 |
| 170 | 17.6 |
| 171 | — |
| 172 | 16.3 |
| 173 | 11.4 |
| 174 | 19.1 |
| 175 | 17.1 |
| 176 | 12.2 |
| 177 | 12.3 |
| 178 | 8.57 |
| 179 | 13.2 |
| 180 | 8.26 |
| 181 | 19.3 |
| 182 | 15.1 |
| 183 | 7.12 |

All publications cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention.

Accordingly, the invention is limited only by the following claims.

1. A compound of Formula I: 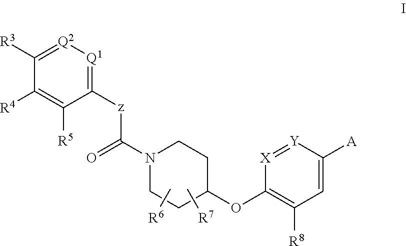
wherein
Q¹ is N or CR¹,
Q² is N or CR²,
R¹, R², R⁴ and R⁵ are each independently H, F, CN, OH, NH₂, C₁₋₃ alkyl optionally substituted by one or more F, or C₁₋₃ alkoxy optionally substituted by one or more F,
R³ is H, F, Cl, CN, C₁₋₄ alkyl optionally substituted by one or more F, C₁₋₄ alkoxy optionally substituted by one or more F, or C₃₋₇ cycloalkyloxy optionally substituted by one or more F, or C₁₋₄ alkylthio optionally substituted by one or more F,
with the proviso that at least 2 of R¹, R², R³, R⁴ and R⁵ are H,
R⁶ and R⁷ can be attached at any point on the piperidine ring and are independently H, F, CN, OH, NH₂, C₁₋₃ alkyl optionally substituted by one or more F, or C₁₋₃ alkoxy optionally substituted by one or more F,
or R⁶ and R⁷ can be taken together, with the atoms to which they are attached, to form a 3- to 7-membered cycloalkane ring or a 3- to 7-membered saturated heterocyclic ring (containing 1 ring hetero atom selected from O, S and N),
R⁸ is CONR¹⁰¹R¹⁰²,
X is CR¹⁰¹ or N,
Y is CR¹⁰² or N,
Z is CH₂, CH(CH₃), NH or O,
A is a phenyl or a 5- or 6-membered saturated or unsaturated heterocyclic ring containing 1, 2 or 3 hetero-atoms selected from S, N and O,
each of which is optionally fused to a further 5- or 6-membered saturated or unsaturated heterocyclic ring containing 1, 2 or 3 hetero-atoms selected from S, N and O,
and which phenyl or heterocyclic ring or fused ring system is optionally substituted by 1, 2 or 3 substituents independently selected from ═O, CN and C₀₋₆ alkyl optionally substituted by 1 or more F or by 1 or 2 substituents independently selected from OH, CO₂R⁹, NH₂, SO₂CH₃, C₁₋₄ alkoxy, CON(R¹⁰³)(R¹⁰⁴)and a group selected from 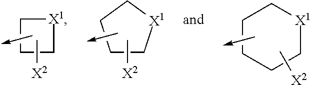
where X¹ is selected from NR¹⁰¹, O and SO₂,
X² is H, OH or F,
R⁹ is H or C₁₋₆ alkyl,
R¹⁰¹ and R¹⁰² are each independently selected from H and C₁₋₃ alkyl,
R¹⁰³ and R¹⁰⁴ are each independently selected from H, (C₁₋₆ alkyl optionally substituted by OH, C₁₋₆ alkoxy or by one or more F), and (C₃₋₇ cycloalkyl optionally substituted by OH, C₁₋₆ alkoxy or by one or more F);
or a pharmaceutically acceptable salt thereof.

2. A compound or salt according to claim 1 wherein X is CH or N.

3. A compound or salt according to claim 1 wherein Y is CH, N or C—CH₃.

4. A compound or salt according to claim 1 wherein Z is CH₂, CH(CH₃) or NH.

5. A compound or salt according to claim 1 wherein R⁸ is CONH₂.

6. A compound or salt according to claim 1 wherein R⁶ is H, F or CH₃.

7. A compound or salt according to claim 1 wherein Q¹ is CH or N.

8. A compound or salt according to claim 1 wherein Q² is CH or N.

9. A compound or salt according to claim 1 wherein R⁷ is F, H or CH₃.

10. A compound or salt according to claim 1 wherein R³ is OCF₃, CF₃, C(CH₃)₃, SCF₃, CH(CH₃)₂ or cyclopropyloxy.

11. A compound or salt according to claim 1 wherein A is an imidazolyl, pyrrolidinyl, thiazolyl, pyridyl, phenyl, or pyrazolyl group optionally substituted by 1 or 2 substituents independently selected from CO₂R⁹ and C₀₋₆ alkyl optionally substituted by 1 or 2 substituents independently selected from OH, NH₂, SO₂CH₃, C₁₋₄ alkoxy, CON(R¹⁰³)(R¹⁰⁴) and a group selected from

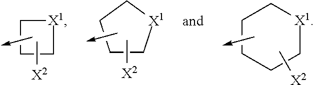

12. A compound or salt according to claim 11 where A is an imidazolyl, pyrrolidinyl, thiazolyl, pyridyl, phenyl, or pyrazolyl group optionally substituted by CH₃, CH₂SO₂CH₃ or by

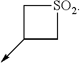

13. A compound according to claim 1 which has the formula IA 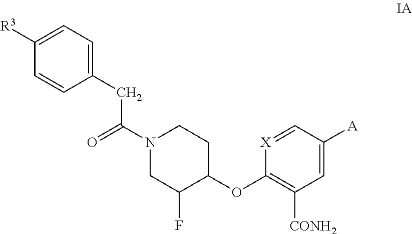
wherein
R³ is OCF₃ or cyclopropyloxy,
and X is CH or N;
or a pharmaceutically acceptable salt thereof.

14. A compound or salt according to claim 12 wherein
A is a C-linked imidazolyl or pyrazolyl group optionally substituted by CH₃, CH₂SO₂CH₃ or by

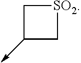

15. (canceled)

16. A compound according to claim 1, selected from the group consisting of:
5-[1-(1,1-dioxidothietan-3-yl)-1H-pyrazol-4-yl]-2-{[(3S,4R)-3-fluoro-1-{[4-(trifluoromethoxy)phenyl]acetyl}piperidin-4-yl]oxy}benzamide;
2-{[(3S,4S)-3-fluoro-1-{[4-(trifluoromethoxy)phenyl]acetyl}piperidin-4-yl]oxy}-5-(1-methyl-1H-imidazol-4-yl)pyridine-3-carboxamide;
2-{[(3R,4S)-3-fluoro-1-{[4-(trifluoromethoxy)phenyl]acetyl}piperidin-4-yl]oxy}-5-(1-methyl-1H-imidazol-4-yl)pyridine-3-carboxamide;
2-{[(3R,4S)-3-fluoro-1-{[4-(trifluoromethoxy)phenyl]acetyl}piperidin-4-yl]oxy}-5-(1-methyl-1H-pyrazol-4-yl)pyridine-3-carboxamide;
2-{[(3R,4S)-3-fluoro-1-{[4-(trifluoromethoxy)phenyl]acetyl}piperidin-4-yl]oxy}-5-(1H-pyrazol-4-yl)benzamide;
2-{[(3S,4R)-3-fluoro-1-(2-(4-(trifluoromethoxy)phenyl)acetyl)piperidin-4-yl]oxy}-5-(1-methyl-1H-imidazol-4-yl)benzamide;
2-{[(3S,4R)-1-{[4-(cyclopropyloxy)phenyl]acetyl}-3-fluoropiperidin-4-yl]oxy}-5-(1-methyl-1H-imidazol-4-yl)pyridine-3-carboxamide;
2-{[(3S,4S)-3-fluoro-1-{[4-(trifluoromethoxy)phenyl]acetyl}piperidin-4-yl]oxy}-5-(1-methyl-1H-imidazol-4-yl)benzamide;
2-{[(3S,4R)-3-fluoro-1-{[4-(trifluoromethoxy)phenyl]acetyl}piperidin-4-yl]oxy}-5-(1-methyl-1H-imidazol-4-yl)pyridine-3-carboxamide;
2-(pyrrolidin-1-yl)-5-((1-(2-(4-(trifluoromethoxy)phenyl)acetyl)piperidin-4-yl)oxy)isonicotinamide,
2-{[(3R,4S)-3-fluoro-1-{[4-(trifluoromethoxy)phenyl]acetyl}piperidin-4-yl]oxy}-5-(2-methyl-1H-imidazol-4-yl)pyridine-3-carboxamide;
or a pharmaceutically acceptable salt thereof of any of the above listed compounds.

17. A pharmaceutical composition comprising a compound of formula I or a pharmaceutically acceptable salt thereof, as defined in claim 1, and a pharmaceutically acceptable carrier.

18. (canceled)

19. (canceled)

20. (canceled)

21. (canceled)

22. (canceled)

23. A method of treatment of a mammal, to treat a disease for which a Trk receptor antagonist is indicated, comprising treating said mammal with an effective amount of a compound of formula I or a pharmaceutically acceptable salt thereof, as defined in claim 1.

24. A method of treatment of pain or cancer in a mammal, comprising treating said mammal with an effective amount of a compound of formula I or a pharmaceutically acceptable salt thereof, as defined in claim 1.

25. (canceled)
